SEQUENCE LISTING (164 sequences)

SEQ ID NO: 1 - DNA, 2688 bp, Artificial Sequence
  hVK3LP-[hDNase1 N74K-G105R-A114F]-(g4s)4-[SSShinge-P238S-P331S Fc]-NGS-[PON1 Q192K] DNA
  sig_peptide (1)..(60): human VK3 leader peptide
  misc_feature (61)..(840): human DNase 1 (N74K, G105R, A114F) without leader peptide
  misc_feature (280)..(283): human DNase 1 N74K substitution
  misc_feature (373)..(375): human DNase 1 G105R substitution
  misc_feature (399)..(401): human DNase 1 A114F substitution
  misc_feature (841)..(918): (gly4ser)4 linker peptide sequence with additional restriction site amino acids
  misc_feature (919)..(1614): human IgG1 variant Fc (SSS hinge, P238S, P331S)
  misc_feature (931)..(933): human IgG1 hinge C220S substitution
  misc_feature (949)..(951): human IgG1 hinge C226S substitution
  misc_feature (958)..(960): human IgG1 hinge C229S substitution
  misc_feature (985)..(987): human IgG1 lower hinge-CH2 P238S substitution
  misc_feature (1264)..(1266): human IgG1 Fc P331S substitution
  misc_feature (1615)..(1672): peptide linker containing N-linked glycosylation site
  misc_feature (1673)..(2688): human PON1 Q192K without uncleaved leader peptide
  misc_feature (2197)..(2199): PON1 Q192K substitution
atggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcaggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctc

SEQ ID NO: 2 - protein, 896 aa, Artificial Sequence
  hVK3LP-[hDNase1 N74K-G105R-A114F]-(g4s)4-[SSShinge-P238S-P331S Fc]-NGS-[PON1 Q192K]
  SIGNAL (1)..(20): human VK3 leader peptide
  MISC_FEATURE (21)..(280): human DNase 1 (N74K, G105R, A114F) without leader peptide
  MISC_FEATURE (94)..(94): human DNase 1 N74K substitution
  MISC_FEATURE (125)..(125): human DNase 1 G105R substitution
  MISC_FEATURE (134)..(134): human DNase 1 A114F substitution
  MISC_FEATURE (281)..(306): (gly4ser)4 linker peptide with restriction site amino acids
  MISC_FEATURE (307)..(538): human IgG1 variant Fc (SSS hinge, P238S, P331S)
  MISC_FEATURE (311)..(311): human IgG1 C220S substitution
  MISC_FEATURE (317)..(317): human IgG1 C226S substitution
  MISC_FEATURE (320)..(320): human IgG1 C229S substitution
  MISC_FEATURE (329)..(329): P238S lower hinge-CH2 mutation
  MISC_FEATURE (422)..(422): human IgG1 P331S substitution
  MISC_FEATURE (539)..(557): peptide linker containing N-linked glycosylation site
  MISC_FEATURE (558)..(896): human PON1 Q192K without uncleaved leader peptide
  MISC_FEATURE (733)..(733): PON1 Q192K substitution
METPAQLLFLLLLWLPDTTGLKIAAFNIQTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 3 - DNA, 1065 bp, Homo sapiens
atggcgaagctgattgcgctcaccctcttggggatgggactggcactcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttacaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctc

SEQ ID NO: 4 - protein, 355 aa, Homo sapiens [sequence omitted]

SEQ ID NO: 5 - DNA, 1065 bp, Artificial Sequence
  Human PON1 Q192K DNA
atggcgaagctgattgcgctcaccctcttggggatgggactggcactcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctc

SEQ ID NO: 6 - protein, 355 aa, Artificial Sequence
  Human PON1 Q192K
  SIGNAL (1)..(16): PON1 uncleaved leader sequence
  mat_peptide (1)..(355): PON1 mature polypeptide with uncleaved leader
  MISC_FEATURE (17)..(355): PON1 sequence without uncleaved signal peptide
  MISC_FEATURE (192)..(192): PON1 mutant Q192K
MAKLIALTLLGMGLALFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 7 - DNA, 1065 bp, Homo sapiens
atggcgaagctgattgcgctcaccctcttggggatgggactggcactcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaagatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctc

SEQ ID NO: 8 - protein, 355 aa, Homo sapiens [sequence omitted]

SEQ ID NO: 9 - DNA, 52 bp, Artificial Sequence
  (gly4ser)2 linker DNA
agatctctccggaggaggtggctcaggtggtggaggatctaccggtctcgag

SEQ ID NO: 10 - protein, 16 aa, Artificial Sequence
  (gly4ser)2 linker
DLSGGGGSGGGGSTGL

SEQ ID NO: 11 - DNA, 82 bp, Artificial Sequence
  (gly4ser)4 linker DNA
agatctctccggaggaggtggctcaggtggtggaggatctggaggaggtgggagtggtggaggtggttctaccggtctcgag

SEQ ID NO: 12 - protein, 26 aa, Artificial Sequence
  (gly4ser)4 linker
DLSGGGGSGGGGSGGGGSGGGGSTGL

SEQ ID NO: 13 - DNA, 112 bp, Artificial Sequence
  (gly4ser)6 linker DNA
agatctctccggaggaggtggctcaggtggtggaggatctggaggaggtgggagtggtggaggtggttctggaggaggtggtagtggaggtggaggttctaccggtctcgag

SEQ ID NO: 14 - protein, 36 aa, Artificial Sequence
  (gly4ser)6 linker
DLSGGGGSGGGGSGGGGSGGGGSGGGGSGGGGSTGL

SEQ ID NO: 15 - DNA, 846 bp, Homo sapiens
atgaggggcatgaagctgctgggggcgctgctggcactggcggccctactgcagggggccgtgtccctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaag

SEQ ID NO: 16 - protein, 282 aa, Homo sapiens [sequence omitted]

SEQ ID NO: 17 - DNA, 840 bp, Artificial Sequence
  hVK3LP-[hDNase1 N74K-G105R-A114F] DNA
atggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcaggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaa

SEQ ID NO: 18 - protein, 280 aa, Artificial Sequence
  hVK3LP-[hDNase1 N74K-G105R-A114F]
  SIGNAL (1)..(20): human VK3 leader peptide
  MISC_FEATURE (94)..(94): N74K substitution
  MISC_FEATURE (125)..(125): G105R substitution
  MISC_FEATURE (134)..(134): A114F substitution
METPAQLLFLLLLWLPDTTGLKIAAFNIQTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 19 - DNA, 840 bp, Artificial Sequence
  hVK3LP-[hDNase1 G105R-A114F] DNA
atggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcaggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaa

SEQ ID NO: 20 - protein, 280 aa, Artificial Sequence
  hVK3LP-[hDNase1 G105R-A114F]
  SIGNAL (1)..(20): human VK3 leader peptide
  MISC_FEATURE (125)..(125): G105R substitution
  MISC_FEATURE (134)..(134): A114F substitution
METPAQLLFLLLLWLPDTTGLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 21 - DNA, 468 bp, Homo sapiens
atggctctggagaagtctcttgtccggctccttctgcttgtcctgatactgctggtgctgggctgggtccagccttccctgggcaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacc

SEQ ID NO: 22 - protein, 156 aa, Homo sapiens
  SIGNAL (1)..(28): human RNase signal peptide
MALEKSLVRLLLLVLILLVLGWVQPSLGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 23 - DNA, 444 bp, Artificial Sequence
  hVK3LP-RNase1 DNA
atggaaaccccagcgcagcttctcttcctcctgctactctggctcccagataccaccggtaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacgactccaggtaccccaactgtgcataccggaccagcccgaaggagagacacatcattgtggcctgtgaagggagcccatatgtgccagtccactttgatgcttctgtggaggactctaca

SEQ ID NO: 24 - protein, 148 aa, Artificial Sequence
  hVK3LP-RNase1
  SIGNAL (1)..(20): human VK3 leader peptide
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 25 - DNA, 696 bp, Artificial Sequence
  Human IgG1 variant Fc DNA (SCC hinge, wild-type CH2 and CH3)
gagcccaaatcttctgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaa

SEQ ID NO: 26 - protein, 232 aa, Artificial Sequence
  Human IgG1 variant Fc (SCC hinge, wild-type CH2 and CH3)
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - DNA, 696 bp, Artificial Sequence
  Human IgG1 variant Fc DNA (SSS hinge, P238S, P331S)
gagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaa

SEQ ID NO: 28 - protein, 232 aa, Artificial Sequence
  Human IgG1 variant Fc (SSS hinge, P238S, P331S)
  MISC_FEATURE (5)..(5): IgG1 hinge C220S substitution
  MISC_FEATURE (11)..(11): IgG1 hinge C226S substitution
  MISC_FEATURE (14)..(14): IgG1 hinge C229S substitution
  MISC_FEATURE (23)..(23): IgG1 lower hinge-CH2 P238S substitution
  MISC_FEATURE (116)..(116): IgG1 Fc P331S substitution
EPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - DNA, 774 bp, Artificial Sequence
  (g4s)4-[SSShinge-P238S-P331S Fc] DNA
gatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaa

SEQ ID NO: 30 - protein, 258 aa, Artificial Sequence
  (g4s)4-[SSShinge-P238S-P331S Fc]
  MISC_FEATURE (1)..(26): (Gly4Ser)4 linker (including additional restriction site amino acids)
  MISC_FEATURE (27)..(258): human IgG1 hinge-CH2-CH3 with substituted amino acids
  MISC_FEATURE (31)..(31): human IgG1 hinge C220S substitution
  MISC_FEATURE (37)..(37): human IgG1 hinge C226S substitution
  MISC_FEATURE (40)..(40): human IgG1 hinge C229S substitution
  MISC_FEATURE (49)..(49): human IgG1 lower hinge-CH2 P238S substitution
  MISC_FEATURE (142)..(142): human IgG1 P331S substitution
DLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 462 bp, Homo sapiens
atggcgacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaa

SEQ ID NO: 32 - protein, 154 aa, Homo sapiens [sequence omitted]

SEQ ID NO: 33 - DNA, 1242 bp, Artificial Sequence
  hRNase1-(g4s)4-[SSShinge-P238S Fc] DNA
atggctctggagaagtctcttgtccggctccttctgcttgtcctgatactgctggtgctgggctgggtccagccttccctgggcaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacagatctctccggaggaggtggctcaggtggtggaggatctggaggaggtgggagtggtggaggtggttctaccggtctcgagcccaaatcttctgacaaaactcacacatctccaccgtccccagcacctgaactcctggggggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaa

SEQ ID NO: 34 - protein, 414 aa, Artificial Sequence
  hRNase1-(g4s)4-[SSShinge-P238S Fc]
  SIGNAL (1)..(28): human RNase signal sequence
  MISC_FEATURE (29)..(166): human RNase1 mature polypeptide
  MISC_FEATURE (167)..(182): human (gly4ser)4 linker peptide with additional restriction site amino acids
  MISC_FEATURE (183)..(414): human IgG1 variant Fc (SSS hinge, P238S)
  MISC_FEATURE (187)..(187): human IgG1 hinge C220S substitution
  MISC_FEATURE (193)..(193): human IgG1 hinge C226S substitution
  MISC_FEATURE (196)..(196): human IgG1 hinge C229S substitution
  MISC_FEATURE (205)..(205): human IgG1lower hinge-CH2 P238S substitution
MALEKSLVRLLLLVLILLVLGWVQPSLGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 1218 bp, Artificial Sequence
  hVK3LP-hRNase1-(g4s)4-[SSShinge-P238S Fc] DNA
atggaaaccccagcgcagcttctcttcctcctgctactctggctcccagataccaccggtaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacgactccaggtaccccaactgtgcataccggaccagcccgaaggagagacacatcattgtggcctgtgaagggagcccatatgtgccagtccactttgatgcttctgtggaggactctacagatctctccggaggaggtggctcaggtggtggaggatctggaggaggtgggagtggtggaggtggttctaccggtctcgagcccaaatcttctgacaaaactcacacatctccaccgtccccagcacctgaactcctggggggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaa

SEQ ID NO: 36 - protein, 406 aa, Artificial Sequence
  hVK3LP-hRNase1-(g4s)4-[SSShinge-P238S Fc]
  SIGNAL (1)..(20): human VK3 signal peptide
  MISC_FEATURE (21)..(158): human RNase 1 mature polypeptide
  MISC_FEATURE (159)..(174): (gly4ser)4 linker peptide with additional restriction site amino acids
  MISC_FEATURE (175)..(406): human IgG1 variant Fc (SSS hinge, P238S)
  MISC_FEATURE (179)..(179): human IgG1 hinge C220S substitution
  MISC_FEATURE (185)..(185): human IgG1 hinge C226S substitution
  MISC_FEATURE (188)..(188): human IgG1 hinge C229S substitution
  MISC_FEATURE (197)..(197): human IgG1 lower hinge-CH2 P238S substitution
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNDSRYPNCAYRTSPKERHIIVACEGSPYVPVHFDASVEDSTDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 2316 bp, Artificial Sequence
  hRNase1-(g4s)4-[SSSH-P238S Fc]-NGS-[PON1 Q192K) DNA
atggctctggagaagtctcttgtccggctccttctgcttgtcctgatactgctggtgctgggctgggtccagccttccctgggcaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctc

SEQ ID NO: 38 - protein, 772 aa, Artificial Sequence
  hRNase1-(g4s)4-[SSSH-P238S Fc]-NGS-[PON1 Q192K)
  SIGNAL (1)..(28): human RNase 1 signal peptide
  MISC_FEATURE (29)..(156): human RNase 1 mature polypeptide
  MISC_FEATURE (157)..(182): (gly4ser)4 linker peptide with additional restriction site amino acids
  MISC_FEATURE (183)..(414): human IgG1 variant Fc (SSS hinge, P238S)
  MISC_FEATURE (187)..(187): Human IgG1 hinge C220S substitution
  MISC_FEATURE (193)..(193): Human IgG1 hinge C226S substitution
  MISC_FEATURE (196)..(196): Human IgG1 hinge C229S substitution
  MISC_FEATURE (205)..(205): Human IgG1 lower hinge-CH2 P238S substitution
  MISC_FEATURE (415)..(433): peptide linker containing N-linked glyscosylation site
  MISC_FEATURE (434)..(772): human PON1 Q192K without uncleaved leader peptide
  MISC_FEATURE (609)..(609): PON1 Q192K amino acid substitution
MALEKSLVRLLLLVLILLVLGWVQPSLGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 39 - DNA, 2292 bp, Artificial Sequence
  hVK3LP-hRNase1-(g4s)4-[SSShinge-P238S Fc]-NGS-[PON1 Q192K] DNA
atggaaaccccagcgcagcttctcttcctcctgctactctggctcccagataccaccggtaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctc

SEQ ID NO: 40 - protein, 764 aa, Artificial Sequence
  hVK3LP-hRNase1-(g4s)4-[SSShinge-P238S Fc]-NGS-[PON1 Q192K]
  SIGNAL (1)..(20): human VK3 signal peptide
  MISC_FEATURE (21)..(148): human RNase1 mature polypeptide
  MISC_FEATURE (149)..(174): (gly4ser)4 linker peptide with addtional restriction site amino acids
  MISC_FEATURE (175)..(406): human IgG1 variant Fc (SSS hinge, P238S)
  MISC_FEATURE (179)..(179): human IgG1 hinge C220S amino acid substitution
  MISC_FEATURE (185)..(185): human IgG1 hinge C226S amino acid substitution
  MISC_FEATURE (188)..(188): human IgG1 hinge C229S amino acid substitution
  MISC_FEATURE (197)..(197): human IgG1 lower hinge-CH2 P238S amino acid substitution
  MISC_FEATURE (407)..(425): peptide linker containing N linked glyscosylation site
  MISC_FEATURE (426)..(764): human PON1 Q192K without uncleaved leader peptide
  MISC_FEATURE (601)..(601): PON1 Q192K amino acid substitution
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 41 - DNA, 1170 bp, Artificial Sequence
  hRNase1-[SSShinge-P238S Fc] DNA
atggctctggagaagtctcttgtccggctccttctgcttgtcctgatactgctggtgctgggctgggtccagccttccctgggcaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacagatctcgagcccaaatcttctgacaaaactcacacatctccaccgtccccagcacctgaactcctggggggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaa

SEQ ID NO: 42 - protein, 390 aa, Artificial Sequence
  hRNase1-[SSShinge-P238S Fc]
  SIGNAL (1)..(28): human RNase1 signal peptide
  MISC_FEATURE (29)..(156): human RNase1 mature polypeptide
  MISC_FEATURE (157)..(158): restriction site amino acids
  MISC_FEATURE (159)..(390): human IgG1 variant Fc (SSS hinge, P238S)
  MISC_FEATURE (163)..(163): human IgG1 hinge domain C220S substitution
  MISC_FEATURE (169)..(169): human IgG1 hinge domain C226S substitution
  MISC_FEATURE (172)..(172): human IgG1 hinge domain C229S substitution
  MISC_FEATURE (181)..(181): human IgG1 lower hinge-CH2 domain P238S substitution
MALEKSLVRLLLLVLILLVLGWVQPSLGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 1146 bp, Artificial Sequence
  hVK3LP-hRNase1-[SSShinge-P238S Fc] DNA
atggaaaccccagcgcagcttctcttcctcctgctactctggctcccagataccaccggtaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacgactccaggtaccccaactgtgcataccggaccagcccgaaggagagacacatcattgtggcctgtgaagggagcccatatgtgccagtccactttgatgcttctgtggaggactctacagatctcgagcccaaatcttctgacaaaactcacacatctccaccgtccccagcacctgaactcctggggggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaa

SEQ ID NO: 44 - protein, 382 aa, Artificial Sequence
  hVK3LP-hRNase1-[SSShinge-P238S Fc]
  SIGNAL (1)..(20): Human VK3 leader peptide
  MISC_FEATURE (21)..(148): human RNase 1 mature polytpeptide
  MISC_FEATURE (149)..(150): restriction site amino acids
  MISC_FEATURE (151)..(382): human IgG1 variant Fc (SSS hinge, P238S)
  MISC_FEATURE (155)..(155): human IgG1 hinge C220S amino acid substitution
  MISC_FEATURE (161)..(161): human IgG1 hinge C226S amino acid substitution
  MISC_FEATURE (164)..(164): human IgG1 hinge C229S amino acid substitution
  MISC_FEATURE (173)..(173): human IgG1 lower hinge-CH2 P238S amino acid substitution
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNDSRYPNCAYRTSPKERHIIVACEGSPYVPVHFDASVEDSTDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - DNA, 2244 bp, Artificial Sequence
  hRNase1-[SSShinge-P238S Fc]-NGS-[PON1 Q192K] DNA
atggctctggagaagtctcttgtccggctccttctgcttgtcctgatactgctggtgctgggctgggtccagccttccctgggcaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacagatctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctc

SEQ ID NO: 46 - protein, 748 aa, Artificial Sequence
  hRNase1-[SSShinge-P238S Fc]-NGS-[PON1 Q192K]
  SIGNAL (1)..(28): human RNase 1 signal peptide
  MISC_FEATURE (29)..(156): human RNase 1 mature polypeptide
  MISC_FEATURE (157)..(158): restriction site amino acids
  MISC_FEATURE (159)..(390): human IgG1 variant Fc (SSS hinge, P238S)
  MISC_FEATURE (163)..(163): human IgG1 hinge C220S amino acid substitution
  MISC_FEATURE (169)..(169): human IgG1 hinge C226S amino acid substitution
  MISC_FEATURE (172)..(172): human IgG1 hinge C229S amino acid substitution
  MISC_FEATURE (182)..(182): human IgG1lower hinge-CH2 P238S amino acid substitution
  MISC_FEATURE (391)..(409): peptide linker containing N-linked glycosylation site
  MISC_FEATURE (410)..(748): human PON1 Q192K without uncleaved leader peptide
  MISC_FEATURE (585)..(585): human PON1 Q192K amino acid substitution
MALEKSLVRLLLLVLILLVLGWVQPSLGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 47 - DNA, 2220 bp, Artificial Sequence
  hVK3LP-hRNase1-[SSShinge-P238S Fc]-NGS-[PON1 Q192K] DNA
atggaaaccccagcgcagcttctcttcctcctgctactctggctcccagataccaccggtaaggaatcccgggccaagaaattccagcggcagcatatggactcagacagttcccccagcagcagctccacctactgtaaccaaatgatgaggcgccggaatatgacacaggggcggtgcaaaccagtgaacacctttgtgcacgagcccctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaaggagagacacatcattgtggcctgtgaagggagcccatatgtgccagtccactttgatgcttctgtggaggactctacagatctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctc

SEQ ID NO: 48 - protein, 740 aa, Artificial Sequence
  hVK3LP-hRNase1-[SSShinge-P238S Fc]-NGS-[PON1 Q192K]
  SIGNAL (1)..(20): human VK3 leader peptide
  MISC_FEATURE (21)..(148): human RNase 1 mature polypeptide
  MISC_FEATURE (149)..(150): restriction site amino acids
  MISC_FEATURE (151)..(382): human IgG1 variant Fc (SSS hinge, P238S)
  MISC_FEATURE (155)..(155): human IgG1 hinge C220S amino acid substitution
  MISC_FEATURE (161)..(161): human IgG1 hinge C226S amino acid substitution
  MISC_FEATURE (164)..(164): human IgG1 hinge C229S amino acid substitution
  MISC_FEATURE (173)..(173): human IgG1 lower hinge-CH2 P238S amino acid substitution
  MISC_FEATURE (383)..(401): peptide linker containing N-linked glycosylation site
  MISC_FEATURE (402)..(740): human PON1 Q192K without uncleaved leader peptide
  MISC_FEATURE (577)..(577): human PON1 Q192K amino acid substitution
METPAQLLFLLLLWLPDTTGKESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDSTDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 49 - DNA, 2343 bp, Artificial Sequence
  hVK3LP-hSOD1-(g4s)2-[SSShinge-P238S-P331S Fc]-NGS-[PON1 Q192K] DNA
atggaaaccccagcgcagcttctcttcctcctgctactctggctcccagataccaccggtatggcggcaacgaaggccgtgtgcgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattgcatcattggccgcacactggtggtccatgaaaaagcagatgacttgggcaaaggtggaaatgaagaaagtacaaagacaggaaacgctggaagtcgtttggcttgtggtgtaattgggatcgcccaagatctctccggaggaggtggctcaggtggtggaggatctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctc

SEQ ID NO: 50 - protein, 781 aa, Artificial Sequence
  hVK3LP-hSOD1-(g4s)2-[SSShinge-P238S-P331S Fc]-NGS-[PON1 Q192K]
  SIGNAL (1)..(22): human VK3 leader peptide with 2 additional amino acids to drive correct cleavage
  MISC_FEATURE (23)..(175): human SOD 1 without N-terminal methionine
  MISC_FEATURE (176)..(191): (gly4ser)2 linker peptide with additional restriction site amino acids
  MISC_FEATURE (192)..(483): human IgG1 variant Fc (SSS hinge, P238S, P331S)
  MISC_FEATURE (196)..(196): human IgG1 hinge C220S amino acid substitution
  MISC_FEATURE (202)..(202): human IgG1 hinge C226S amino acid substitution
  MISC_FEATURE (205)..(205): human IgG1 hinge C229S amino acid substitution
  MISC_FEATURE (224)..(224): human IgG1 lower hinge-CH2 P238S amino acid substitution
  MISC_FEATURE (307)..(307): human IgG1 Fc P331S amino acid substitution
  MISC_FEATURE (424)..(442): peptide linker containing N-linked glycosylation site
  MISC_FEATURE (443)..(781): human PON1 Q192K without uncleaved leader peptide
  MISC_FEATURE (618)..(618): human PON1 Q192K amino acid substitution
METPAQLLFLLLLWLPDTTGMAATKAVCVLKGDGPVQGIINFEQKESNGPVKVWGSIKGLTEGLHGFHVHEFGDNTAGCTSAGPHFNPLSRKHGGPKDEERHVGDLGNVTADKDGVADVSIEDSVISLSGDHCIIGRTLVVHEKADDLGKGGNEESTKTGNAGSRLACGVIGIAQDLSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 51 - DNA, 2343 bp, Artificial Sequence
  hVK3LP-[hSOD1 C6A C111S]-(g4s)2-[SSShinge-P238S-P331S Fc]-NGS-[PON1 Q192K] DNA
atggaaaccccagcgcagcttctcttcctcctgctactctggctcccagataccaccggtatggcggcaacgaaggccgtggctgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattcaatcattggccgcacactggtggtccatgaaaaagcagatgacttgggcaaaggtggaaatgaagaaagtacaaagacaggaaacgctggaagtcgtttggcttgtggtgtaattgggatcgcccaagatctctccggaggaggtggctcaggtggtggaggatctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctc

SEQ ID NO: 52 - protein, 781 aa, Artificial Sequence
  hVK3LP-[hSOD1 C6A C111S]-(g4s)2-[SSShinge-P238S-P331S Fc]-NGS-[PON1 Q192K]
  SIGNAL (1)..(22): human VK3 leader peptide with 2 additional amino acids to drive correct cleavage
  MISC_FEATURE (23)..(175): human SOD1 C6A-C111S without N-terminal methionine
  MISC_FEATURE (28)..(28): human SOD 1 C6A amino acid substitution
  MISC_FEATURE (133)..(133): human SOD 1 C111S amino acid substitution
  MISC_FEATURE (176)..(191): (gly4ser)2 linker peptide with additional restriction site amino acids
  MISC_FEATURE (192)..(423): human IgG1 variant Fc (SSS hinge, P238S, P331S)
  MISC_FEATURE (196)..(196): human IgG1 hinge C220S amino acid substitution
  MISC_FEATURE (202)..(202): human IgG1 hinge C226S amino acid substitution
  MISC_FEATURE (205)..(205): human IgG1 hinge C229S amino acid substitution
  MISC_FEATURE (215)..(215): human IgG1 lower hinge-CH2 P238S amino acid substitution
  MISC_FEATURE (307)..(307): human IgG1 Fc P331S amino acid substitution
  MISC_FEATURE (424)..(442): linker peptide containing N-linked glycosylation site
  MISC_FEATURE (443)..(781): human PON1 Q192K without uncleaved leader peptide
  MISC_FEATURE (618)..(618): human PON1 Q192K amino acid substitution
METPAQLLFLLLLWLPDTTGMAATKAVAVLKGDGPVQGIINFEQKESNGPVKVWGSIKGLTEGLHGFHVHEFGDNTAGCTSAGPHFNPLSRKHGGPKDEERHVGDLGNVTADKDGVADVSIEDSVISLSGDHSIIGRTLVVHEKADDLGKGGNEESTKTGNAGSRLACGVIGIAQDLSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 53 - DNA, 2373 bp, Artificial Sequence
  hVK3LP-[hSOD1 C6A C111S]-(g4s)4-[SSShinge-P238S-P331S Fc]-NGS-[PON1 Q192K] DNA
atggaaaccccagcgcagcttctcttcctcctgctactctggctcccagataccaccggtatggcggcaacgaaggccgtggctgtgctgaagggcgacggcccagtgcagggcatcatcaatttcgagcagaaggaaagtaatggaccagtgaaggtgtggggaagcattaaaggactgactgaaggcctgcatggattccatgttcatgagtttggagataatacagcaggctgtaccagtgcaggtcctcactttaatcctctatccagaaaacacggtgggccaaaggatgaagagaggcatgttggagacttgggcaatgtgactgctgacaaagatggtgtggccgatgtgtctattgaagattctgtgatctcactctcaggagaccattcaatcattggccgcacactggtggtccatgaaaaagcagatgacttgggcaaaggtggaaatgaagaaagtacaaagacaggaaacgctggaagtcgtttggcttctggtgtaattgggatcgcccaagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctc

SEQ ID NO: 54 - protein, 791 aa, Artificial Sequence
  hVK3LP-[hSOD1 C6A C111S]-(g4s)4-[SSShinge-P238S-P331S Fc]-NGS-[PON1 Q192K]
  SIGNAL (1)..(22): human VK3 leader peptide with 2 additional amino acids to drive correct cleavage
  MISC_FEATURE (23)..(175): human SOD1 C6A-C111S without N-terminal methionine
  MISC_FEATURE (28)..(28): human SOD 1 C6A amino acid substitution
  MISC_FEATURE (133)..(133): human SOD 1 C111S amino acid substitution
  MISC_FEATURE (176)..(201): (gly4ser)4 linker peptide with additional restriction site amino acids
  MISC_FEATURE (202)..(433): human IgG1 variant Fc (SSS hinge, P238S, P331S)
  MISC_FEATURE (206)..(206): human IgG1 hinge C220S amino acid substitution
  MISC_FEATURE (212)..(212): human IgG1 hinge C226S amino acid substitution
  MISC_FEATURE (215)..(215): human IgG1 hinge C229S amino acid substitution
  MISC_FEATURE (225)..(225): human IgG1 lower hinge-CH2 P238S amino acid substitution
  MISC_FEATURE (317)..(317): human IgG1 Fc P331S amino acid substitution
  MISC_FEATURE (434)..(452): peptide linker containing N-linked glycosylation site
  MISC_FEATURE (453)..(791): human PON1 Q192K without uncleaved leader peptide
  MISC_FEATURE (628)..(628): human PON1 Q192K amino acid substitution
METPAQLLFLLLLWLPDTTGMAATKAVAVLKGDGPVQGIINFEQKESNGPVKVWGSIKGLTEGLHGFHVHEFGDNTAGCTSAGPHFNPLSRKHGGPKDEERHVGDLGNVTADKDGVADVSIEDSVISLSGDHSIIGRTLVVHEKADDLGKGGNEESTKTGNAGSRLASGVIGIAQDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 55 - DNA, 54 bp, Artificial Sequence
  Peptide linker containing N-linked glycosylation site (NGSlinker) DNA
gtcgacggagctagcagccccgtgaacgtgagcagccccagcgtgcaggatatc

SEQ ID NO: 56 - protein, 18 aa, Artificial Sequence
  Peptide linker containing N-linked glycosylation site (NGS linker)
VDGASSPVNVSSPSVQDI

SEQ ID NO: 57 - DNA, 72 bp, Homo sapiens
aagcttgccgccatggaaaccccagcgcagcttctcttcctcctgctactctggctcccagataccaccggt

SEQ ID NO: 58 - protein, 20 aa, Homo sapiens
METPAQLLFLLLLWLPDTTG

SEQ ID NO: 59 - DNA, 1614 bp, Artificial Sequence
  hVK3LP-[hDNase1 N74K-G105R-A114F]-(g4s)4-[SSShinge-P238S-P331S Fc] DNA
atggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcaggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaa

SEQ ID NO: 60 - protein, 538 aa, Artificial Sequence
  hVK3LP-[hDNase1 N74K-G105R-A114F]-(g4s)4-[SSShinge-P238S-P331S Fc]
METPAQLLFLLLLWLPDTTGLKIAAFNIQTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - DNA, 1644 bp, Artificial Sequence
  hVK3LP-[hDNase1 N74K-G105R-A114F]-(g4s)6-[SSShinge-P238S-P331S Fc] DNA
atggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcaggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctctccggaggaggtggctcaggtggtggaggatctggaggaggtgggagtggtggaggtggttctggaggaggtggtagtggaggtggaggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaa

SEQ ID NO: 62 - protein, 548 aa, Artificial Sequence
  hVK3LP-[hDNase1 N74K-G105R-A114F]-(g4s)6-[SSShinge-P238S-P331S Fc]
METPAQLLFLLLLWLPDTTGLKIAAFNIQTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKDLSGGGGSGGGGSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - DNA, 1137 bp, Artificial Sequence
  hVK3LP-[hCTLA-4 EC]-[SSShinge-P238S-P331S Fc] DNA
atggaaaccccagcgcagcttctcttcctcctgctactctggctcccggataccaccggtgcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattcagatctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaataa

SEQ ID NO: 64 - protein, 378 aa, Artificial Sequence
  hVK3LP-[hCTLA-4 EC]-[SSShinge-P238S-P331S Fc]
METPAQLLFLLLLWLPDTTGAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 2211 bp, Artificial Sequence
  hVK3LP-[hCTLA-4 EC]-[SSShinge-P238S-P331S Fc]-NGS-[PON1 Q192K] DNA
atggaaaccccagcgcagcttctcttcctcctgctactctggctcccggataccaccggtgcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattcagatctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaa

SEQ ID NO: 66 - protein, 736 aa, Artificial Sequence
  hVK3LP-[hCTLA-4 EC]-[SSShinge-P238S-P331S Fc]-NGS-[PON1 Q192K]
METPAQLLFLLLLWLPDTTGAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 67 - DNA, 835 bp, Homo sapiens
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtgag

SEQ ID NO: 68 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 69 - DNA, 1351 bp, Artificial Sequence
  [hCD40 EC]-(g4s)4-[SSShinge-P238S Fc] DNA
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaataataatctagaa

SEQ ID NO: 70 - protein, 446 aa, Artificial Sequence
  [hCD40 EC]-(g4s)4-[SSShinge-P238S Fc]
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - DNA, 1341 bp, Artificial Sequence
  hVK3LP-[hCD40 EC]-(g4s)4-[SSShinge-P238S Fc] DNA
atggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaataa

SEQ ID NO: 72 - protein, 446 aa, Artificial Sequence
  hVK3LP-[hCD40 EC]-(g4s)4-[SSShinge-P238S Fc]
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - DNA, 2425 bp, Artificial Sequence
  hVK3LP-[hCD40 EC]-(g4s)4-[SSShinge-P238S Fc]-NGS-[PON1 Q192K] DNA
atggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaataatctagaa

SEQ ID NO: 74 - protein, 804 aa, Artificial Sequence
  hVK3LP-[hCD40 EC]-(g4s)4-[SSShinge-P238S Fc]-NGS-[PON1 Q192K]
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 75 - DNA, 1341 bp, Artificial Sequence
  hVK3LP-[hCD40 K81T EC]-(g4s)4-[SSShinge-P238S Fc] DNA
atggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacacatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaataa

SEQ ID NO: 76 - protein, 446 aa, Artificial Sequence
  hVK3LP-[hCD40 K81T EC]-(g4s)4-[SSShinge-P238S Fc]
METPAQLLFLLLLWLPDTTGEPPTACREKQYLINSQCCSLCQPGQKLVSDCTEFTETECLPCGESEFLDTWNRETHCHQHTYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - DNA, 2418 bp, Artificial Sequence
  hVK3LP-[hCD40 K81T EC]-(g4s)4-[SSShinge-P238S Fc]-NGS-[PON1 Q192K] DNA
atggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacacatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaataa

SEQ ID NO: 78 - protein, 804 aa, Artificial Sequence
  hVK3LP-[hCD40 K81T EC]-(g4s)4-[SSShinge-P238S Fc]-NGS-[PON1 Q192K]
METPAQLLFLLLLWLPDTTGEPPTACREKQYLINSQCCSLCQPGQKLVSDCTEFTETECLPCGESEFLDTWNRETHCHQHTYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 79 - DNA, 1341 bp, Artificial Sequence
  hVK3LP-[hCD40 K81H-L121P EC]-(g4s)4-[SSShinge-P238S Fc] DNA
atggaaaccccagcgcagcttctcttcctcctgctactctggctcccaggtaccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcaccactactgcgaccccaacctagggcttcgggtccagcagaagggcacctcagaaacagacaccatctgcacctgtgaagaaggctggcactgtacgagtgaggcctgtgagagctgtgtcccgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaataa

SEQ ID NO: 80 - protein, 446 aa, Artificial Sequence
  hVK3LP-[hCD40 K81H-L121P EC]-(g4s)4-[SSShinge-P238S Fc]
METPAQLLFLLLLWLPGTTGEPPTACREKQYLINSQCCSLCQPGQKLVSDCTEFTETECLPCGESEFLDTWNRETHCHQHHYCDPNLGLRVQQKGTSETDTICTCEEGWHCTSEACESCVPHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - DNA, 2415 bp, Artificial Sequence
  hVK3LP-[hCD40 K81H-L121P EC]-(g4s)4-[SSShinge-P238S Fc]-NGS-[PON1 Q192K] DNA
atggaaaccccagcgcagcttctcttcctcctgctactctggctcccaggtaccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcaccactactgcgaccccaacctagggcttcgggtccagcagaagggcacctcagaaacagacaccatctgcacctgtgaagaaggctggcactgtacgagtgaggcctgtgagagctgtgtcccgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaa

SEQ ID NO: 82 - protein, 804 aa, Artificial Sequence
  hVK3LP-[hCD40 K81H-L121P EC]-(g4s)4-[SSShinge-P238S Fc]-NGS-[PON1 Q192K]
METPAQLLFLLLLWLPGTTGEPPTACREKQYLINSQCCSLCQPGQKLVSDCTEFTETECLPCGESEFLDTWNRETHCHQHHYCDPNLGLRVQQKGTSETDTICTCEEGWHCTSEACESCVPHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 83 - DNA, 1341 bp, Artificial Sequence
  hVK3LP-[hCD40 K81S EC]-(g4s)4-[SSShinge-P238S Fc] DNA
atggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcactcctactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaataa

SEQ ID NO: 84 - protein, 446 aa, Artificial Sequence
  hVK3LP-[hCD40 K81S EC]-(g4s)4-[SSShinge-P238S Fc]
METPAQLLFLLLLWLPDTTGEPPTACREKQYLINSQCCSLCQPGQKLVSDCTEFTETECLPCGESEFLDTWNRETHCHQHSYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - DNA, 2415 bp, Artificial Sequence
  hVK3LP-[hCD40 K81S EC]-(g4s)4-[SSShinge-P238S Fc]-NGS-[PON1 Q192K] DNA
atggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcactcctactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaa

SEQ ID NO: 86 - protein, 804 aa, Artificial Sequence
  hVK3LP-[hCD40 K81S EC]-(g4s)4-[SSShinge-P238S Fc]-NGS-[PON1 Q192K]
METPAQLLFLLLLWLPDTTGEPPTACREKQYLINSQCCSLCQPGQKLVSDCTEFTETECLPCGESEFLDTWNRETHCHQHSYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 87 - DNA, 1341 bp, Artificial Sequence
  hVK3LP-[hCD40 E64Y-K81T-P85Y EC]-(g4s)4-[SSShinge-P238S Fc] DNA
atggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggttacagcgaattcctagacacctggaacagagagacacactgccaccagcacacatactgcgactacaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaataa

SEQ ID NO: 88 - protein, 446 aa, Artificial Sequence
  hVK3LP-[hCD40 E64Y-K81T-P85Y EC]-(g4s)4-[SSShinge-P238S Fc]
METPAQLLFLLLLWLPDTTGEPPTACREKQYLINSQCCSLCQPGQKLVSDCTEFTETECLPCGYSEFLDTWNRETHCHQHTYCDYNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - DNA, 2415 bp, Artificial Sequence
  hVK3LP-[hCD40 E64Y-K81T-P85Y EC]-(g4s)4-[SSShinge-P238S Fc]-NGS-[PON1 Q192K] DNA
atggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggttacagcgaattcctagacacctggaacagagagacacactgccaccagcacacatactgcgactacaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaa

SEQ ID NO: 90 - protein, 804 aa, Artificial Sequence
  hVK3LP-[hCD40 E64Y-K81T-P85Y EC]-(g4s)4-[SSShinge-P238S Fc]-NGS-[PON1 Q192K]
METPAQLLFLLLLWLPDTTGEPPTACREKQYLINSQCCSLCQPGQKLVSDCTEFTETECLPCGYSEFLDTWNRETHCHQHTYCDYNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 91 - DNA, 816 bp, Artificial Sequence
  hVK3LP-[VL-VH anti-TNFalpha scFv] DNA
aagcttgccgccatggaaaccccagcgcagcttctcttcctcctgctactctggctcccggataccaccggtgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaaggaggtggtggatctggtggaggaggttcaggtggtggaggatctgggggtggaggtagtgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgtca

SEQ ID NO: 92 - protein, 268 aa, Artificial Sequence
  hVK3LP-[VL-VH anti-TNFalpha scFv]
METPAQLLFLLLLWLPDTTGDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGGGSGGGGSGGGGSGGGGSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSS

SEQ ID NO: 93 - DNA, 2605 bp, Artificial Sequence
  hVK3LP-[VL-VH anti-TNFalpha scFv]-[SSShinge-P238S-P331S Fc]-NGS-[PON1 Q192K] DNA
aagcttgccgccatggaaaccccagcgcagcttctcttcctcctgctactctggctcccggataccaccggtgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaaggaggtggtggatctggtggaggaggttcaggtggtggaggatctgggggtggaggtagtgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgtcagatctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaataatctagaa

SEQ ID NO: 94 - protein, 860 aa, Artificial Sequence
  hVK3LP-[VL-VH anti-TNFalpha scFv]-[SSShinge-P238S-P331S Fc]-NGS-[PON1 Q192K]
METPAQLLFLLLLWLPDTTGDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKGGGGSGGGGSGGGGSGGGGSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 95 - DNA, 816 bp, Artificial Sequence
  hVK3LP-[VH-VL anti-TNFalpha scFv] DNA
aagcttgccgccatggaaaccccagcgcagcttctcttcctcctgctactctggctcccggataccaccggtgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtggaggtggtggatctggtggaggaggttcaggtggtggaggatctgggggtggaggtagtgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaa

SEQ ID NO: 96 - protein, 268 aa, Artificial Sequence
  hVK3LP-[VH-VL anti-TNFalpha scFv]
METPAQLLFLLLLWLPDTTGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIK

SEQ ID NO: 97 - DNA, 2605 bp, Artificial Sequence
  hVK3LP-[VH-VL anti-TNFalpha scFv]-[SSShinge-P238S-P331S Fc]-NGS-[PON1 Q192K] DNA
aagcttgccgccatggaaaccccagcgcagcttctcttcctcctgctactctggctcccggataccaccggtgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtcaccgtctcgagtggaggtggtggatctggtggaggaggttcaggtggtggaggatctgggggtggaggtagtgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaaggtggaaatcaaagatctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaataatctagaa

SEQ ID NO: 98 - protein, 860 aa, Artificial Sequence
  hVK3LP-[VH-VL anti-TNFalpha scFv]-[SSShinge-P238S-P331S Fc]-NGS-[PON1 Q192K]
METPAQLLFLLLLWLPDTTGEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 99 - DNA, 819 bp, Artificial Sequence
  hVK3LP-[anti-hTGFbeta VH-VL scFv] DNA
aagcttgccgccatggaaaccccagcgcagcttctcttcctcctgctactctggctcccggataccaccggtgaagttcagctggtggagtctggcggtggcctggtgcagccagggggctcactccgtttgtcctgtgcagcttctggctacgcattcaccaactatctgatcgagtgggtccgtcaggccccgggtaagggcctcgagtgggttggtgttaacaatcctggatccggaggctccaactataacgagaagttcaaggggcgcgccactatcagtgcagacaattcgaaaaacacattatacctgcagatgaacagcctgcgtgctgaggacactgccgtctattattgtgctcgatccggaggcttctacttcgactactggggtcaaggaaccctggtcaccgtctcctcgggaggtggtggatctggtggaggaggttcaggtggtggaggatctgggggtggaggtagtgatatccagatgacccagtccccgagctccctgtccgcctctgtgggcgatagggtcaccatcacctgcagagccagtcagagcgtgctgtatagttcgaatcagaagaactacctggcctggtatcaacagaaaccaggaaaagctccgaaactactgatttactgggctagcactagagaatctggagtcccttctcgcttctctggatccggttctgggacggatttcactctgaccatcagcagtctgcagccagaagacttcgcaacttattactgtcaccagtatctgagctctgacacatttggacagggtaccaaggtggagatcaaa

SEQ ID NO: 100 - protein, 269 aa, Artificial Sequence
  hVK3LP-[anti-hTGFbeta VH-VL scFv]
METPAQLLFLLLLWLPDTTGEVQLVESGGGLVQPGGSLRLSCAASGYAFTNYLIEWVRQAPGKGLEWVGVNNPGSGGSNYNEKFKGRATISADNSKNTLYLQMNSLRAEDTAVYYCARSGGFYFDYWGQGTLVTVSSGGGGSGGGGSGGGGSGGGGSDIQMTQSPSSLSASVGDRVTITCRASQSVLYSSNQKNYLAWYQQKPGKAPKLLIYWASTRESGVPSRFSGSGSGTDFTLTISSLQPEDFATYYCHQYLSSDTFGQGTKVEIK

SEQ ID NO: 101 - DNA, 2608 bp, Artificial Sequence
  hVK3LP-[anti-hTGFbeta VH-VL scFv]-SSShinge-P238S-P331S Fc]-NGS-PON1 Q192K] DNA
aagcttgccgccatggaaaccccagcgcagcttctcttcctcctgctactctggctcccggataccaccggtgaagttcagctggtggagtctggcggtggcctggtgcagccagggggctcactccgtttgtcctgtgcagcttctggctacgcattcaccaactatctgatcgagtgggtccgtcaggccccgggtaagggcctcgagtgggttggtgttaacaatcctggatccggaggctccaactataacgagaagttcaaggggcgcgccactatcagtgcagacaattcgaaaaacacattatacctgcagatgaacagcctgcgtgctgaggacactgccgtctattattgtgctcgatccggaggcttctacttcgactactggggtcaaggaaccctggtcaccgtctcctcgggaggtggtggatctggtggaggaggttcaggtggtggaggatctgggggtggaggtagtgatatccagatgacccagtccccgagctccctgtccgcctctgtgggcgatagggtcaccatcacctgcagagccagtcagagcgtgctgtatagttcgaatcagaagaactacctggcctggtatcaacagaaaccaggaaaagctccgaaactactgatttactgggctagcactagagaatctggagtcccttctcgcttctctggatccggttctgggacggatttcactctgaccatcagcagtctgcagccagaagacttcgcaacttattactgtcaccagtatctgagctctgacacatttggacagggtaccaaggtggagatcaaagatctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaataatctagaa

SEQ ID NO: 102 - protein, 861 aa, Artificial Sequence
  hVK3LP-[anti-hTGFbeta VH-VL scFv]-SSShinge-P238S-P331S Fc]-NGS-PON1 Q192K]
METPAQLLFLLLLWLPDTTGEVQLVESGGGLVQPGGSLRLSCAASGYAFTNYLIEWVRQAPGKGLEWVGVNNPGSGGSNYNEKFKGRATISADNSKNTLYLQMNSLRAEDTAVYYCARSGGFYFDYWGQGTLVTVSSGGGGSGGGGSGGGGSGGGGSDIQMTQSPSSLSASVGDRVTITCRASQSVLYSSNQKNYLAWYQQKPGKAPKLLIYWASTRESGVPSRFSGSGSGTDFTLTISSLQPEDFATYYCHQYLSSDTFGQGTKVEIKDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 103 - DNA, 819 bp, Artificial Sequence
  hVK3LP-[anti-hTGFbeta VL-VH scFv] DNA
aagcttgccgccatggaaaccccagcgcagcttctcttcctcctgctactctggctcccggataccaccggtgatatccagatgacccagtccccgagctccctgtccgcctctgtgggcgatagggtcaccatcacctgcagagccagtcagagcgtgctgtatagttcgaatcagaagaactacctggcctggtatcaacagaaaccaggaaaagctccgaaactactgatttactgggctagcactagagaatctggagtcccttctcgcttctctggatccggttctgggacggatttcactctgaccatcagcagtctgcagccagaagacttcgcaacttattactgtcaccagtatctgagctctgacacatttggacagggtaccaaggtggagatcaaaggaggtggtggatctggtggaggaggttcaggtggtggaggatctgggggtggaggtagtgaagttcagctggtggagtctggcggtggcctggtgcagccagggggctcactccgtttgtcctgtgcagcttctggctacgcattcaccaactatctgatcgagtgggtccgtcaggccccgggtaagggcctcgagtgggttggtgttaacaatcctggatccggaggctccaactataacgagaagttcaaggggcgcgccactatcagtgcagacaattcgaaaaacacattatacctgcagatgaacagcctgcgtgctgaggacactgccgtctattattgtgctcgatccggaggcttctacttcgactactggggtcaaggaaccctggtcaccgtctcctcg

SEQ ID NO: 104 - protein, 269 aa, Artificial Sequence
  hVK3LP-[anti-hTGFbeta VL-VH scFv]
METPAQLLFLLLLWLPDTTGDIQMTQSPSSLSASVGDRVTITCRASQSVLYSSNQKNYLAWYQQKPGKAPKLLIYWASTRESGVPSRFSGSGSGTDFTLTISSLQPEDFATYYCHQYLSSDTFGQGTKVEIKGGGGSGGGGSGGGGSGGGGSEVQLVESGGGLVQPGGSLRLSCAASGYAFTNYLIEWVRQAPGKGLEWVGVNNPGSGGSNYNEKFKGRATISADNSKNTLYLQMNSLRAEDTAVYYCARSGGFYFDYWGQGTLVTVSS

SEQ ID NO: 105 - DNA, 2608 bp, Artificial Sequence
  hVK3LP-[anti-hTGFbeta VL-VH scFv]-SSShinge-P238S-P331S Fc]-NGS-PON1 Q192K] DNA
aagcttgccgccatggaaaccccagcgcagcttctcttcctcctgctactctggctcccggataccaccggtgatatccagatgacccagtccccgagctccctgtccgcctctgtgggcgatagggtcaccatcacctgcagagccagtcagagcgtgctgtatagttcgaatcagaagaactacctggcctggtatcaacagaaaccaggaaaagctccgaaactactgatttactgggctagcactagagaatctggagtcccttctcgcttctctggatccggttctgggacggatttcactctgaccatcagcagtctgcagccagaagacttcgcaacttattactgtcaccagtatctgagctctgacacatttggacagggtaccaaggtggagatcaaaggaggtggtggatctggtggaggaggttcaggtggtggaggatctgggggtggaggtagtgaagttcagctggtggagtctggcggtggcctggtgcagccagggggctcactccgtttgtcctgtgcagcttctggctacgcattcaccaactatctgatcgagtgggtccgtcaggccccgggtaagggcctcgagtgggttggtgttaacaatcctggatccggaggctccaactataacgagaagttcaaggggcgcgccactatcagtgcagacaattcgaaaaacacattatacctgcagatgaacagcctgcgtgctgaggacactgccgtctattattgtgctcgatccggaggcttctacttcgactactggggtcaaggaaccctggtcaccgtctcctcagatctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaataatctagaa

SEQ ID NO: 106 - protein, 861 aa, Artificial Sequence
  hVK3LP-[anti-hTGFbeta VL-VH scFv]-SSShinge-P238S-P331S Fc]-NGS-PON1 Q192K]
METPAQLLFLLLLWLPDTTGDIQMTQSPSSLSASVGDRVTITCRASQSVLYSSNQKNYLAWYQQKPGKAPKLLIYWASTRESGVPSRFSGSGSGTDFTLTISSLQPEDFATYYCHQYLSSDTFGQGTKVEIKGGGGSGGGGSGGGGSGGGGSEVQLVESGGGLVQPGGSLRLSCAASGYAFTNYLIEWVRQAPGKGLEWVGVNNPGSGGSNYNEKFKGRATISADNSKNTLYLQMNSLRAEDTAVYYCARSGGFYFDYWGQGTLVTVSSDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 107 - DNA, 363 bp, Homo sapiens
gataccaccggtgaggtgcagctggtggagtctgggggaggcttggtacagcccggcaggtccctgagactctcctgtgcggcctctggattcacctttgatgattatgccatgcactgggtccggcaagctccagggaagggcctggaatgggtctcagctatcacttggaatagtggtcacatagactatgcggactctgtggagggccgattcaccatctccagagacaacgccaagaactccctgtatctgcaaatgaacagtctgagagctgaggatacggccgtatattactgtgcgaaagtctcgtaccttagcaccgcgtcctcccttgactattggggccaaggtaccctggtc

SEQ ID NO: 108 - protein, 121 aa, Homo sapiens [sequence omitted]

SEQ ID NO: 109 - DNA, 321 bp, Homo sapiens
gataccaccggtgacatccagatgacccagtctccatcctccctgtctgcatctgtaggggacagagtcaccatcacttgtcgggcaagtcagggcatcagaaattacttagcctggtatcagcaaaaaccagggaaagcccctaagctcctgatctatgctgcatccactttgcaatcaggggtcccatctcggttcagtggcagtggatctgggacagatttcactctcaccatcagcagcctacagcctgaagatgttgcaacttattactgtcaaaggtataaccgtgcaccgtatacttttggccaggggaccaag

SEQ ID NO: 110 - protein, 107 aa, Homo sapiens [sequence omitted]

SEQ ID NO: 111 - DNA, 351 bp, Artificial Sequence
  Humanized anti-hTGFbeta VH DNA
gataccaccggtgaagttcagctggtggagtctggcggtggcctggtgcagccagggggctcactccgtttgtcctgtgcagcttctggctacgcattcaccaactatctgatcgagtgggtccgtcaggccccgggtaagggcctcgagtgggttggtgttaacaatcctggatccggaggctccaactataacgagaagttcaaggggcgcgccactatcagtgcagacaattcgaaaaacacattatacctgcagatgaacagcctgcgtgctgaggacactgccgtctattattgtgctcgatccggaggcttctacttcgactactggggtcaaggaaccctggtc

SEQ ID NO: 112 - protein, 117 aa, Artificial Sequence
  Humanized anti-hTGFbeta VH
EVQLVESGGGLVQPGGSLRLSCAASGYAFTNYLIEWVRQAPGKGLEWVGVNNPGSGGSNYNEKFKGRATISADNSKNTLYLQMNSLRAEDTAVYYCARSGGFYFDYWGQGTLVTVSS

SEQ ID NO: 113 - DNA, 336 bp, Artificial Sequence
  Humanized anti-hTGFbeta VL DNA
gataccaccggtgatatccagatgacccagtccccgagctccctgtccgcctctgtgggcgatagggtcaccatcacctgcagagccagtcagagcgtgctgtatagttcgaatcagaagaactacctggcctggtatcaacagaaaccaggaaaagctccgaaactactgatttactgggctagcactagagaatctggagtcccttctcgcttctctggatccggttctgggacggatttcactctgaccatcagcagtctgcagccagaagacttcgcaacttattactgtcaccagtatctgagctctgacacatttggacagggtaccaag

SEQ ID NO: 114 - protein, 112 aa, Artificial Sequence
  Humanized anti-hTGFbeta VL
DIQMTQSPSSLSASVGDRVTITCRASQSVLYSSNQKNYLAWYQQKPGKAPKLLIYWASTRESGVPSRFSGSGSGTDFTLTISSLQPEDFATYYCHQYLSSDTFGQGTKVEIK

SEQ ID NO: 115 - DNA, 696 bp, Artificial Sequence
  Human IgG1 variant Fc DNA (SCC hinge, P238S, P331S)
gagcccaaatcttctgacaaaactcacacatgtccaccgtgcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaa

SEQ ID NO: 116 - protein, 232 aa, Artificial Sequence
  Human IgG1 variant Fc (SCC hinge, P238S, P331S)
EPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - DNA, 696 bp, Artificial Sequence
  Human IgG1 variant Fc DNA (SCC hinge, P238S)
gagcccaaatcttctgacaaaactcacacatgtccaccgtgcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaa

SEQ ID NO: 118 - protein, 232 aa, Artificial Sequence
  Human IgG1 variant Fc (SCC hinge, P238S)
EPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 5 aa, Artificial Sequence
  (gly4ser) tandem repeat sequence
GGGGS

SEQ ID NO: 120 - protein, 260 aa, Homo sapiens
LKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 121 - DNA, 1842 bp, Artificial Sequence
  hVK3LP-[KDL-SCChinge-P238S-P331S Fc]-NGS-[PON1 Q192K] DNA
atggaaaccccagcgcagcttctcttcctcctgctactctggctcccagataccaccggtaaagatctcgagcccaaatcttctgacaaaactcacacatgtccaccgtgcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaa

SEQ ID NO: 122 - protein, 613 aa, Artificial Sequence
  hVK3LP-[KDL-SCChinge-P238S-P331S Fc]-NGS-[PON1 Q192K]
METPAQLLFLLLLWLPDTTGKDLEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 123 - DNA, 1065 bp, Artificial Sequence
  Human PON1 variant G3C9 DNA
atggctaaactgacagcgctcacactcttggggctgggattggcactcttcgatggacagaagtcttctttccaaacacgatttaatgttcaccgtgaagtaactccagtggaacttcctaactgtaatttagttaaaggggttgacaatggttctgaagacttggaaatactgcccaatggactggctttcatcagctccggattaaagtatcctggaataatgagctttgaccctgataagtctggaaagatacttctaatggacctgaatgaggaagacccagtagtgttggaactgggcattactggaaatacattggatatatcttcatttaaccctcatgggattagcacattcacagatgaagataacactgtgtacctactggtggtaaaccatccagactcctcgtccaccgtggaggtgtttaaatttcaagaagaagaaaaatcacttttgcatctgaaaaccatcagacacaagcttctgcctagtgtgaatgacattgtcgctgtgggacctgaacacttttatgccacaaatgatcactattttgctgacccttacttaaaatcctgggaaatgcatttgggattagcgtggtcatttgttacttattatagtcccaatgatgttcgagtagtggcagaaggatttgattttgctaacggaatcaacatctcaccagacggcaagtatgtctatatagctgagttgctggctcataagatccatgtgtatgaaaagcacgctaattggactttaactccattgaagtccctcgactttgacacccttgtggataacatctctgtggatcctgtgacaggggacctctgggtgggatgccatcccaacggaatgcgaatcttctactatgacccaaagaatcctcccggctcagaggtgcttcgaatccaggacattttatccgaagagcccaaagtgacagtggtttatgcagaaaatggcactgtgttacagggcagcacggtggccgctgtgtacaaagggaaactgctgattggcacagtgtttcacaaagctctttactgtgagctg

SEQ ID NO: 124 - protein, 355 aa, Artificial Sequence
  Human PON1 variant G3C9
MAKLTALTLLGLGLALFDGQKSSFQTRFNVHREVTPVELPNCNLVKGVDNGSEDLEILPNGLAFISSGLKYPGIMSFDPDKSGKILLMDLNEEDPVVLELGITGNTLDISSFNPHGISTFTDEDNTVYLLVVNHPDSSSTVEVFKFQEEEKSLLHLKTIRHKLLPSVNDIVAVGPEHFYATNDHYFADPYLKSWEMHLGLAWSFVTYYSPNDVRVVAEGFDFANGINISPDGKYVYIAELLAHKIHVYEKHANWTLTPLKSLDFDTLVDNISVDPVTGDLWVGCHPNGMRIFYYDPKNPPGSEVLRIQDILSEEPKVTVVYAENGTVLQGSTVAAVYKGKLLIGTVFHKALYCEL

SEQ ID NO: 125 - DNA, 1065 bp, Artificial Sequence
  PON1 variant M-IIG1 DNA
atggctaaactgacagcgctcacactcttggggctgggattggcactcttcgatggacagaagtcttctttccaaacacgatttaatgttcaccgtgaagtaactccagtggaacttcctaactgtaatttagttaaaggggttgacaatggttctgaagacattgaaatactgcccaatggactggctttcatcagctccggagttaagtatcctggaataatgagctttgaccctgataagtctggaaagatacttctaatggacctgaatgaggaagacccagtagtgttggaactgggcattactggaaatacattggatatatcttcatttaaccctgctgggattagcacattcacagatgaagataacactgtgtacctactggtggtaaaccgaccagactcctcgtccaccgtggaggtgtttaaatttcaagaagaagaaaaatcacttttgcatctgaaaaccatcagacacaagcttctgcctagtgtgaatgacattgtcgctgtgggacctgaacacttttatgccacaaatgatcactattttgctgacccttacttaaaatcctgggaaatgcatttgggattagcgtggtcatttgttacttattatagtcccaatgatgttcgagtagtggcagaaggatttgatatggctaacggaatcaacatctcaccagacggcaagtatgtctatatagctgagttgctggctcataagatccatgtgtatgaaaagcacgctaattggactttaactccattgaagtccctcgactttgacacccttgtggataacatctctgtggatcctgtgacaggggacctctgggtgggatgccatcccaacggaatgcgattattctactatgacccaaagaatcctcccggctcagaggtgcttcgaatccaggacattttatccgaagagcccaaagtgacagtggtttatgcagaaaatggcactgtgttacagggcagcagtgtggccgctgtgtacaaagggaaactgctgattggcacagtgtttcacaaagctctttactgtgagctg

SEQ ID NO: 126 - protein, 355 aa, Artificial Sequence
  PON1 variant M-IIG1
MAKLTALTLLGLGLALFDGQKSSFQTRFNVHREVTPVELPNCNLVKGVDNGSEDIEILPNGLAFISSGVKYPGIMSFDPDKSGKILLMDLNEEDPVVLELGITGNTLDISSFNPAGISTFTDEDNTVYLLVVNRPDSSSTVEVFKFQEEEKSLLHLKTIRHKLLPSVNDIVAVGPEHFYATNDHYFADPYLKSWEMHLGLAWSFVTYYSPNDVRVVAEGFDMANGINISPDGKYVYIAELLAHKIHVYEKHANWTLTPLKSLDFDTLVDNISVDPVTGDLWVGCHPNGMRLFYYDPKNPPGSEVLRIQDILSEEPKVTVVYAENGTVLQGSSVAAVYKGKLLIGTVFHKALYCEL

SEQ ID NO: 127 - DNA, 2713 bp, Artificial Sequence
  hVK3LP-[hDNase1 N74K-G105R-A114F]-(g4s)4-[SSShinge-P238S-P331S Fc]-NGS-[G3C9 PON1] DNA
aagcttgccgccatggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcaggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcgatggacagaagtcttctttccaaacacgatttaatgttcaccgtgaagtaactccagtggaacttcctaactgtaatttagttaaaggggttgacaatggttctgaagacttggaaatactgcccaatggactggctttcatcagctccggattaaagtatcctggaataatgagctttgaccctgataagtctggaaagatacttctaatggacctgaatgaggaagacccagtagtgttggaactgggcattactggaaatacattggatatatcttcatttaaccctcatgggattagcacattcacagatgaagataacactgtgtacctactggtggtaaaccatccagactcctcgtccaccgtggaggtgtttaaatttcaagaagaagaaaaatcacttttgcatctgaaaaccatcagacacaagcttctgcctagtgtgaatgacattgtcgctgtgggacctgaacacttttatgccacaaatgatcactattttgctgacccttacttaaaatcctgggaaatgcatttgggattagcgtggtcatttgttacttattatagtcccaatgatgttcgagtagtggcagaaggatttgattttgctaacggaatcaacatctcaccagacggcaagtatgtctatatagctgagttgctggctcataagatccatgtgtatgaaaagcacgctaattggactttaactccattgaagtccctcgactttgacacccttgtggataacatctctgtggatcctgtgacaggggacctctgggtgggatgccatcccaacggaatgcgaatcttctactatgacccaaagaatcctcccggctcagaggtgcttcgaatccaggacattttatccgaagagcccaaagtgacagtggtttatgcagaaaatggcactgtgttacagggcagcacggtggccgctgtgtacaaagggaaactgctgattggcacagtgtttcacaaagctctttactgtgagctgtaataatctagaa

SEQ ID NO: 128 - protein, 896 aa, Artificial Sequence
  hVK3LP-[hDNase1 N74K-G105R-A114F]-(g4s)4-[SSShinge-P238S-P331S Fc]-NGS-[G3C9 PON1]
METPAQLLFLLLLWLPDTTGLKIAAFNIQTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFDGQKSSFQTRFNVHREVTPVELPNCNLVKGVDNGSEDLEILPNGLAFISSGLKYPGIMSFDPDKSGKILLMDLNEEDPVVLELGITGNTLDISSFNPHGISTFTDEDNTVYLLVVNHPDSSSTVEVFKFQEEEKSLLHLKTIRHKLLPSVNDIVAVGPEHFYATNDHYFADPYLKSWEMHLGLAWSFVTYYSPNDVRVVAEGFDFANGINISPDGKYVYIAELLAHKIHVYEKHANWTLTPLKSLDFDTLVDNISVDPVTGDLWVGCHPNGMRIFYYDPKNPPGSEVLRIQDILSEEPKVTVVYAENGTVLQGSTVAAVYKGKLLIGTVFHKALYCEL

SEQ ID NO: 129 - DNA, 2688 bp, Artificial Sequence
  hVK3LP-[hDNase1 N74K-G105R-A114F]-(g4s)4-[SSShinge-P238S-P331S Fc]-NGS-[M-IIG1 PON1] DNA
atggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcaggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcgatggacagaagtcttctttccaaacacgatttaatgttcaccgtgaagtaactccagtggaacttcctaactgtaatttagttaaaggggttgacaatggttctgaagacattgaaatactgcccaatggactggctttcatcagctccggagttaagtatcctggaataatgagctttgaccctgataagtctggaaagatacttctaatggacctgaatgaggaagacccagtagtgttggaactgggcattactggaaatacattggatatatcttcatttaaccctgctgggattagcacattcacagatgaagataacactgtgtacctactggtggtaaaccgaccagactcctcgtccaccgtggaggtgtttaaatttcaagaagaagaaaaatcacttttgcatctgaaaaccatcagacacaagcttctgcctagtgtgaatgacattgtcgctgtgggacctgaacacttttatgccacaaatgatcactattttgctgacccttacttaaaatcctgggaaatgcatttgggattagcgtggtcatttgttacttattatagtcccaatgatgttcgagtagtggcagaaggatttgatatggctaacggaatcaacatctcaccagacggcaagtatgtctatatagctgagttgctggctcataagatccatgtgtatgaaaagcacgctaattggactttaactccattgaagtccctcgactttgacacccttgtggataacatctctgtggatcctgtgacaggggacctctgggtgggatgccatcccaacggaatgcgattattctactatgacccaaagaatcctcccggctcagaggtgcttcgaatccaggacattttatccgaagagcccaaagtgacagtggtttatgcagaaaatggcactgtgttacagggcagcagtgtggccgctgtgtacaaagggaaactgctgattggcacagtgtttcacaaagctctttactgtgagctg

SEQ ID NO: 130 - protein, 896 aa, Artificial Sequence
  hVK3LP-[hDNase1 N74K-G105R-A114F]-(g4s)4-[SSShinge-P238S-P331S Fc]-NGS-[M-IIG1 PON1]
METPAQLLFLLLLWLPDTTGLKIAAFNIQTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFDGQKSSFQTRFNVHREVTPVELPNCNLVKGVDNGSEDIEILPNGLAFISSGVKYPGIMSFDPDKSGKILLMDLNEEDPVVLELGITGNTLDISSFNPAGISTFTDEDNTVYLLVVNRPDSSSTVEVFKFQEEEKSLLHLKTIRHKLLPSVNDIVAVGPEHFYATNDHYFADPYLKSWEMHLGLAWSFVTYYSPNDVRVVAEGFDMANGINISPDGKYVYIAELLAHKIHVYEKHANWTLTPLKSLDFDTLVDNISVDPVTGDLWVGCHPNGMRLFYYDPKNPPGSEVLRIQDILSEEPKVTVVYAENGTVLQGSSVAAVYKGKLLIGTVFHKALYCEL

SEQ ID NO: 131 - DNA, 1839 bp, Artificial Sequence
  hVK3LP-[KDL-SCChinge-P238S-P331S Fc]-NGS-[G3C9 PON1] DNA
atggaaaccccagcgcagcttctcttcctcctgctactctggctcccagataccaccggtaaagatctcgagcccaaatcttctgacaaaactcacacatgtccaccgtgcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcgatggacagaagtcttctttccaaacacgatttaatgttcaccgtgaagtaactccagtggaacttcctaactgtaatttagttaaaggggttgacaatggttctgaagacttggaaatactgcccaatggactggctttcatcagctccggattaaagtatcctggaataatgagctttgaccctgataagtctggaaagatacttctaatggacctgaatgaggaagacccagtagtgttggaactgggcattactggaaatacattggatatatcttcatttaaccctcatgggattagcacattcacagatgaagataacactgtgtacctactggtggtaaaccatccagactcctcgtccaccgtggaggtgtttaaatttcaagaagaagaaaaatcacttttgcatctgaaaaccatcagacacaagcttctgcctagtgtgaatgacattgtcgctgtgggacctgaacacttttatgccacaaatgatcactattttgctgacccttacttaaaatcctgggaaatgcatttgggattagcgtggtcatttgttacttattatagtcccaatgatgttcgagtagtggcagaaggatttgattttgctaacggaatcaacatctcaccagacggcaagtatgtctatatagctgagttgctggctcataagatccatgtgtatgaaaagcacgctaattggactttaactccattgaagtccctcgactttgacacccttgtggataacatctctgtggatcctgtgacaggggacctctgggtgggatgccatcccaacggaatgcgaatcttctactatgacccaaagaatcctcccggctcagaggtgcttcgaatccaggacattttatccgaagagcccaaagtgacagtggtttatgcagaaaatggcactgtgttacagggcagcacggtggccgctgtgtacaaagggaaactgctgattggcacagtgtttcacaaagctctttactgtgagctg

SEQ ID NO: 132 - protein, 613 aa, Artificial Sequence
  hVK3LP-[KDL-SCChinge-P238S-P331S Fc]-NGS-[G3C9 PON1]
METPAQLLFLLLLWLPDTTGKDLEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFDGQKSSFQTRFNVHREVTPVELPNCNLVKGVDNGSEDLEILPNGLAFISSGLKYPGIMSFDPDKSGKILLMDLNEEDPVVLELGITGNTLDISSFNPHGISTFTDEDNTVYLLVVNHPDSSSTVEVFKFQEEEKSLLHLKTIRHKLLPSVNDIVAVGPEHFYATNDHYFADPYLKSWEMHLGLAWSFVTYYSPNDVRVVAEGFDFANGINISPDGKYVYIAELLAHKIHVYEKHANWTLTPLKSLDFDTLVDNISVDPVTGDLWVGCHPNGMRIFYYDPKNPPGSEVLRIQDILSEEPKVTVVYAENGTVLQGSTVAAVYKGKLLIGTVFHKALYCEL

SEQ ID NO: 133 - DNA, 1839 bp, Artificial Sequence
  hVK3LP-[KDL-SCChinge-P238S-P331S Fc]-NGS-[M-IIG1 PON1] DNA
atggaaaccccagcgcagcttctcttcctcctgctactctggctcccagataccaccggtaaagatctcgagcccaaatcttctgacaaaactcacacatgtccaccgtgcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcgatggacagaagtcttctttccaaacacgatttaatgttcaccgtgaagtaactccagtggaacttcctaactgtaatttagttaaaggggttgacaatggttctgaagacattgaaatactgcccaatggactggctttcatcagctccggagttaagtatcctggaataatgagctttgaccctgataagtctggaaagatacttctaatggacctgaatgaggaagacccagtagtgttggaactgggcattactggaaatacattggatatatcttcatttaaccctgctgggattagcacattcacagatgaagataacactgtgtacctactggtggtaaaccgaccagactcctcgtccaccgtggaggtgtttaaatttcaagaagaagaaaaatcacttttgcatctgaaaaccatcagacacaagcttctgcctagtgtgaatgacattgtcgctgtgggacctgaacacttttatgccacaaatgatcactattttgctgacccttacttaaaatcctgggaaatgcatttgggattagcgtggtcatttgttacttattatagtcccaatgatgttcgagtagtggcagaaggatttgatatggctaacggaatcaacatctcaccagacggcaagtatgtctatatagctgagttgctggctcataagatccatgtgtatgaaaagcacgctaattggactttaactccattgaagtccctcgactttgacacccttgtggataacatctctgtggatcctgtgacaggggacctctgggtgggatgccatcccaacggaatgcgattattctactatgacccaaagaatcctcccggctcagaggtgcttcgaatccaggacattttatccgaagagcccaaagtgacagtggtttatgcagaaaatggcactgtgttacagggcagcagtgtggccgctgtgtacaaagggaaactgctgattggcacagtgtttcacaaagctctttactgtgagctg

SEQ ID NO: 134 - protein, 613 aa, Artificial Sequence
  hVK3LP-[KDL-SCChinge-P238S-P331S Fc]-NGS-[M-IIG1 PON1]
METPAQLLFLLLLWLPDTTGKDLEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFDGQKSSFQTRFNVHREVTPVELPNCNLVKGVDNGSEDIEILPNGLAFISSGVKYPGIMSFDPDKSGKILLMDLNEEDPVVLELGITGNTLDISSFNPAGISTFTDEDNTVYLLVVNRPDSSSTVEVFKFQEEEKSLLHLKTIRHKLLPSVNDIVAVGPEHFYATNDHYFADPYLKSWEMHLGLAWSFVTYYSPNDVRVVAEGFDMANGINISPDGKYVYIAELLAHKIHVYEKHANWTLTPLKSLDFDTLVDNISVDPVTGDLWVGCHPNGMRLFYYDPKNPPGSEVLRIQDILSEEPKVTVVYAENGTVLQGSSVAAVYKGKLLIGTVFHKALYCEL

SEQ ID NO: 135 - DNA, 982 bp, Homo sapiens
atgtcacgggagctggccccactgctgcttctcctcctctccatccacagcgccctggccatgaggatctgctccttcaacgtcaggtcctttggggaaagcaagcaggaagacaagaatgccatggatgtcattgtgaaggtcatcaaacgctgtgacatcatactcgtgatggaaatcaaggacagcaacaacaggatctgccccatactgatggagaagctgaacagaaattcaaggagaggcataacgtacaactatgtgattagctctcggcttggaagaaacacatataaagaacaatatgcctttctctacaaggaaaagctggtgtctgtgaagaggagttatcactaccatgactatcaggatggagacgcagatgtgttttccagggagccctttgtggtctggttccaatctccccacactgctgtcaaagacttcgtgattatccccctgcacaccaccccagagacatccgttaaggagatcgatgagttggttgaggtctacacggacgtgaaacaccgctggaaggcggagaatttcattttcatgggtgacttcaatgccggctgcagctacgtccccaagaaggcctggaagaacatccgcttgaggactgaccccaggtttgtttggctgatcggggaccaagaggacaccacggtgaagaagagcaccaactgtgcatatgacaggattgtgcttagaggacaagaaatcgtcagttctgttgttcccaagtcaaacagtgtttttgacttccagaaagcttacaagctgactgaagaggaggccctggatgtcagcgaccactttccagttgaatttaaactacagtcttcaagggccttcaccaacagcaaaaaatctgtcactctaaggaagaaaacaaagaggaaacgctcctacccaactttcttgtacaaagttggcattataagaaagcattgcttatcaatttgttgcaacgaac

SEQ ID NO: 136 - protein, 305 aa, Homo sapiens
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKRKRS

SEQ ID NO: 137 - DNA, 1647 bp, Artificial Sequence
  hVK3LP-[hDNase1L3 NLS1mutAAA, NLS2delta]-(g4s)4-[SSShinge-P238S-P331S Fc] DNA
atggaaaccccagcgcagcttctcttcctcctgctactctggctcccagataccaccggtatgaggatctgctccttcaacgtcaggtcctttggggaaagcaagcaggaagacaagaatgccatggatgtcattgtgaaggtcatcaaacgctgtgacatcatactcgtgatggaaatcaaggacagcaacaacaggatctgccccatactgatggagaagctgaacagaaattcagcaagaggcataacgtacaactatgtgattagctctcggcttggagcagctacatataaagaacaatatgcctttctctacaaggaaaagctggtgtctgtgaagaggagttatcactaccatgactatcaggatggagacgcagatgtgttttccagggagccctttgtggtctggttccaatctccccacactgctgtcaaagacttcgtgattatccccctgcacaccaccccagagacatccgttaaggagatcgatgagttggttgaggtctacacggacgtgaaacaccgctggaaggcggagaatttcattttcatgggtgacttcaatgccggctgcagctacgtccccaagaaggcctggaagaacatccgcttgaggactgaccccaggtttgtttggctgatcggggaccaagaggacaccacggtgaagaagagcaccaactgtgcatatgacaggattgtgcttagaggacaagaaatcgtcagttctgttgttcccaagtcaaacagtgtttttgacttccagaaagcttacaagctgactgaagaggaggccctggatgtcagcgaccactttccagttgaatttaaactacagtcttcaagggccttcaccaactcagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaataa

SEQ ID NO: 138 - protein, 548 aa, Artificial Sequence
  hVK3LP-[hDNase1L3 NLS1mutAAA, NLS2delta]-(g4s)4-[SSShinge-P238S-P331S Fc]
METPAQLLFLLLLWLPDTTGMRICSFNVRSFGESKQEDKNAMDVIVKVIKRCDIILVMEIKDSNNRICPILMEKLNRNSARGITYNYVISSRLGAATYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - DNA, 1647 bp, Artificial Sequence
  hVK3LP-[hDNase1L3 NLS1mutSSS, NLS2delta]-(g4s)4-[SSShinge-P238S-P331S Fc] DNA
atggaaaccccagcgcagcttctcttcctcctgctactctggctcccagataccaccggtatgaggatctgctccttcaacgtcaggtcctttggggaaagcaagcaggaagacaagaatgccatggatgtcattgtgaaggtcatcaaacgctgtgacatcatactcgtgatggaaatcaaggacagcaacaacaggatctgccccatactgatggagaagctgaacagaaattcatcaagaggcataacgtacaactatgtgattagctctcggcttggaagttctacatataaagaacaatatgcctttctctacaaggaaaagctggtgtctgtgaagaggagttatcactaccatgactatcaggatggagacgcagatgtgttttccagggagccctttgtggtctggttccaatctccccacactgctgtcaaagacttcgtgattatccccctgcacaccaccccagagacatccgttaaggagatcgatgagttggttgaggtctacacggacgtgaaacaccgctggaaggcggagaatttcattttcatgggtgacttcaatgccggctgcagctacgtccccaagaaggcctggaagaacatccgcttgaggactgaccccaggtttgtttggctgatcggggaccaagaggacaccacggtgaagaagagcaccaactgtgcatatgacaggattgtgcttagaggacaagaaatcgtcagttctgttgttcccaagtcaaacagtgtttttgacttccagaaagcttacaagctgactgaagaggaggccctggatgtcagcgaccactttccagttgaatttaaactacagtcttcaagggccttcaccaactcagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaataa

SEQ ID NO: 140 - protein, 548 aa, Artificial Sequence
  hVK3LP-[hDNase1L3 NLS1mutSSS, NLS2delta]-(g4s)4-[SSShinge-P238S-P331S Fc]
METPAQLLFLLLLWLPDTTGMRICSFNVRSFGESKQEDKNAMDVIVKVIKRCDIILVMEIKDSNNRICPILMEKLNRNSSRGITYNYVISSRLGSSTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - DNA, 1842 bp, Artificial Sequence
  hVK3LP-[SCChinge-P238S-P331S Fc]-NGS-[PON1 Q192K] DNA
atggaaaccccagcgcagcttctcttcctcctgctactctggctcccagataccaccggtgagcccaaatcttctgacaaaactcacacatgtccaccgtgcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaataatctaga

SEQ ID NO: 142 - protein, 610 aa, Artificial Sequence
  hVK3LP-[SCChinge-P238S-P331S Fc]-NGS-[PON1 Q192K]
METPAQLLFLLLLWLPDTTGEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 143 - DNA, 1830 bp, Artificial Sequence
  hVK3LP-[SCChinge-P238S-P331S Fc]-NGS-[G3C9 PON1] DNA
atggaaaccccagcgcagcttctcttcctcctgctactctggctcccagataccaccggtgagcccaaatcttctgacaaaactcacacatgtccaccgtgcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcgatggacagaagtcttctttccaaacacgatttaatgttcaccgtgaagtaactccagtggaacttcctaactgtaatttagttaaaggggttgacaatggttctgaagacttggaaatactgcccaatggactggctttcatcagctccggattaaagtatcctggaataatgagctttgaccctgataagtctggaaagatacttctaatggacctgaatgaggaagacccagtagtgttggaactgggcattactggaaatacattggatatatcttcatttaaccctcatgggattagcacattcacagatgaagataacactgtgtacctactggtggtaaaccatccagactcctcgtccaccgtggaggtgtttaaatttcaagaagaagaaaaatcacttttgcatctgaaaaccatcagacacaagcttctgcctagtgtgaatgacattgtcgctgtgggacctgaacacttttatgccacaaatgatcactattttgctgacccttacttaaaatcctgggaaatgcatttgggattagcgtggtcatttgttacttattatagtcccaatgatgttcgagtagtggcagaaggatttgattttgctaacggaatcaacatctcaccagacggcaagtatgtctatatagctgagttgctggctcataagatccatgtgtatgaaaagcacgctaattggactttaactccattgaagtccctcgactttgacacccttgtggataacatctctgtggatcctgtgacaggggacctctgggtgggatgccatcccaacggaatgcgaatcttctactatgacccaaagaatcctcccggctcagaggtgcttcgaatccaggacattttatccgaagagcccaaagtgacagtggtttatgcagaaaatggcactgtgttacagggcagcacggtggccgctgtgtacaaagggaaactgctgattggcacagtgtttcacaaagctctttactgtgagctg

SEQ ID NO: 144 - protein, 610 aa, Artificial Sequence
  hVK3LP-[SCChinge-P238S-P331S Fc]-NGS-[G3C9 PON1]
METPAQLLFLLLLWLPDTTGEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFDGQKSSFQTRFNVHREVTPVELPNCNLVKGVDNGSEDLEILPNGLAFISSGLKYPGIMSFDPDKSGKILLMDLNEEDPVVLELGITGNTLDISSFNPHGISTFTDEDNTVYLLVVNHPDSSSTVEVFKFQEEEKSLLHLKTIRHKLLPSVNDIVAVGPEHFYATNDHYFADPYLKSWEMHLGLAWSFVTYYSPNDVRVVAEGFDFANGINISPDGKYVYIAELLAHKIHVYEKHANWTLTPLKSLDFDTLVDNISVDPVTGDLWVGCHPNGMRIFYYDPKNPPGSEVLRIQDILSEEPKVTVVYAENGTVLQGSTVAAVYKGKLLIGTVFHKALYCEL

SEQ ID NO: 145 - DNA, 1830 bp, Artificial Sequence
  hVK3LP-[SCChinge-P238S-P331S Fc]-NGS-[M-IIG1 PON1] DNA
atggaaaccccagcgcagcttctcttcctcctgctactctggctcccagataccaccggtgagcccaaatcttctgacaaaactcacacatgtccaccgtgcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcgatggacagaagtcttctttccaaacacgatttaatgttcaccgtgaagtaactccagtggaacttcctaactgtaatttagttaaaggggttgacaatggttctgaagacattgaaatactgcccaatggactggctttcatcagctccggagttaagtatcctggaataatgagctttgaccctgataagtctggaaagatacttctaatggacctgaatgaggaagacccagtagtgttggaactgggcattactggaaatacattggatatatcttcatttaaccctgctgggattagcacattcacagatgaagataacactgtgtacctactggtggtaaaccgaccagactcctcgtccaccgtggaggtgtttaaatttcaagaagaagaaaaatcacttttgcatctgaaaaccatcagacacaagcttctgcctagtgtgaatgacattgtcgctgtgggacctgaacacttttatgccacaaatgatcactattttgctgacccttacttaaaatcctgggaaatgcatttgggattagcgtggtcatttgttacttattatagtcccaatgatgttcgagtagtggcagaaggatttgatatggctaacggaatcaacatctcaccagacggcaagtatgtctatatagctgagttgctggctcataagatccatgtgtatgaaaagcacgctaattggactttaactccattgaagtccctcgactttgacacccttgtggataacatctctgtggatcctgtgacaggggacctctgggtgggatgccatcccaacggaatgcgattattctactatgacccaaagaatcctcccggctcagaggtgcttcgaatccaggacattttatccgaagagcccaaagtgacagtggtttatgcagaaaatggcactgtgttacagggcagcagtgtggccgctgtgtacaaagggaaactgctgattggcacagtgtttcacaaagctctttactgtgagctg

SEQ ID NO: 146 - protein, 610 aa, Artificial Sequence
  hVK3LP-[SCChinge-P238S-P331S Fc]-NGS-[M-IIG1 PON1]
METPAQLLFLLLLWLPDTTGEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFDGQKSSFQTRFNVHREVTPVELPNCNLVKGVDNGSEDIEILPNGLAFISSGVKYPGIMSFDPDKSGKILLMDLNEEDPVVLELGITGNTLDISSFNPAGISTFTDEDNTVYLLVVNRPDSSSTVEVFKFQEEEKSLLHLKTIRHKLLPSVNDIVAVGPEHFYATNDHYFADPYLKSWEMHLGLAWSFVTYYSPNDVRVVAEGFDMANGINISPDGKYVYIAELLAHKIHVYEKHANWTLTPLKSLDFDTLVDNISVDPVTGDLWVGCHPNGMRLFYYDPKNPPGSEVLRIQDILSEEPKVTVVYAENGTVLQGSSVAAVYKGKLLIGTVFHKALYCEL

SEQ ID NO: 147 - DNA, 2721 bp, Artificial Sequence
  hVK3LP-[hDNase1 N74K-G105R-A114F]-(g4s)6-[SSShinge-P238S-P331S Fc]-NGS-[PON1 Q192K] DNA
atggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcaggaacgacaccttcaaccgagagccattcattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctctccggaggaggtggctcaggtggtggaggatctggaggaggtgggagtggtggaggtggttctggaggaggtggtagtggaggtggaggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaa

SEQ ID NO: 148 - protein, 906 aa, Artificial Sequence
  hVK3LP-[hDNase1 N74K-G105R-A114F]-(g4s)6-[SSShinge-P238S-P331S Fc]-NGS-[PON1 Q192K]
METPAQLLFLLLLWLPDTTGLKIAAFNIQTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKDLSGGGGSGGGGSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 149 - protein, 528 aa, Artificial Sequence
  [hDNase1 N74X-G105X-A114X]-[gs linker]-[SSShinge Fc]
  MISC_FEATURE (74)..(74): Xaa is Lys, Arg, or His
  MISC_FEATURE (105)..(105): Xaa is Lys, Arg, or His
  MISC_FEATURE (114)..(114): Xaa is Phe, Glu, Met, Arg, or Tyr
  MISC_FEATURE (261)..(263): Xaa is any naturally occurring non-cysteine amino acid
  MISC_FEATURE (264)..(283): Xaa is Gly or Ser
  MISC_FEATURE (284)..(293): Xaa is Gly or Ser or is absent
  MISC_FEATURE (294)..(296): Xaa is any naturally occurring non-cysteine amino acid
  MISC_FEATURE (315)..(316): Xaa is Leu or Ala
  MISC_FEATURE (319)..(319): Xaa is Pro or Ser
  MISC_FEATURE (333)..(333): Xaa is Met or Tyr
  MISC_FEATURE (335)..(335): Xaa is Ser or Thr
  MISC_FEATURE (337)..(337): Xaa is Thr or Glu
  MISC_FEATURE (346)..(346): Xaa is Asp or Ala
  MISC_FEATURE (378)..(378): Xaa is Asn, Ala, Gln, or Gly
  MISC_FEATURE (410)..(410): Xaa is Pro, Ala, Gly, or Ser
  MISC_FEATURE (412)..(412): Xaa is Pro, Ser, or Ala
  MISC_FEATURE (509)..(509): Xaa is Met or Leu
  MISC_FEATURE (515)..(515): Xaa is Asn or Ser
  MISC_FEATURE (528)..(528): Xaa is Lys or is absent
LKIAAFNIQTFGETKMSNATLVSYIVQILSRYDIALVQEVRDSHLTAVGKLLDNLNQDAPDTYHYVVSEPLGRXSYKERYLFVYRPDQVSAVDSYYYDDGCEPCXNDTFNREPXIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKXXXXXXXXXXXXXXXXXXXXXXXXXXXXXXXXXXXXEPKSSDKTHTSPPSPAPEXXGGXSVFLFPPKPKDTLXIXRXPEVTCVVVXVSHEDPEVKFNWYVDGVEVHNAKTKPREEQYXSTYRVVSVLTVLHQDWLNGKEYKCKVSNKALXAXIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVXHEALHXHYTQKSLSLSPGX

SEQ ID NO: 150 - protein, 528 aa, Artificial Sequence
  [hDNase1 N74K-G105R-A114F]-[gs linker]-[SSShinge Fc]
  MISC_FEATURE (261)..(263): Xaa is any naturally occurring non-cysteine amino acid
  MISC_FEATURE (264)..(283): Xaa is Gly or Ser
  MISC_FEATURE (284)..(293): Xaa is Gly or Ser or is absent
  MISC_FEATURE (294)..(296): Xaa is any naturally occurring non-cysteine amino acid
  MISC_FEATURE (315)..(316): Xaa is Leu or Ala
  MISC_FEATURE (319)..(319): Xaa is Pro or Ser
  MISC_FEATURE (333)..(333): Xaa is Met or Tyr
  MISC_FEATURE (335)..(335): Xaa is Ser or Thr
  MISC_FEATURE (337)..(337): Xaa is Thr or Glu
  MISC_FEATURE (346)..(346): Xaa is Asp or Ala
  MISC_FEATURE (378)..(378): Xaa is Asn, Ala, Gln, or Gly
  MISC_FEATURE (410)..(410): Xaa is Pro, Ala, Gly, or Ser
  MISC_FEATURE (412)..(412): Xaa is Pro, Ser, or Ala
  MISC_FEATURE (509)..(509): Xaa is Met or Leu
  MISC_FEATURE (515)..(515): Xaa is Asn or Ser
  MISC_FEATURE (528)..(528): Xaa is Lys or is absent
LKIAAFNIQTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCRNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKXXXXXXXXXXXXXXXXXXXXXXXXXXXXXXXXXXXXEPKSSDKTHTSPPSPAPEXXGGXSVFLFPPKPKDTLXIXRXPEVTCVVVXVSHEDPEVKFNWYVDGVEVHNAKTKPREEQYXSTYRVVSVLTVLHQDWLNGKEYKCKVSNKALXAXIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVXHEALHXHYTQKSLSLSPGX

SEQ ID NO: 151 - DNA, 840 bp, Artificial Sequence
  hVK3LP-[hDNase1 N74K-G105R] DNA
atggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcaggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaa

SEQ ID NO: 152 - protein, 280 aa, Artificial Sequence
  hVK3LP-[hDNase1 N74K-G105R]
METPAQLLFLLLLWLPDTTGLKIAAFNIQTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCRNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 153 - protein, 528 aa, Artificial Sequence
  [hDNase1 N74X-G105X]-[gs linker]-[SSShinge Fc]
  MISC_FEATURE (74)..(74): Xaa is Lys, Arg, or His
  MISC_FEATURE (105)..(105): Xaa is Lys, Arg, or His
  MISC_FEATURE (261)..(263): Xaa is any naturally occurring non-cysteine amino acid
  MISC_FEATURE (264)..(283): Xaa is Gly or Ser
  MISC_FEATURE (284)..(293): Xaa is Gly or Ser or is absent
  MISC_FEATURE (294)..(296): Xaa is any naturally occurring non-cysteine amino acid
  MISC_FEATURE (315)..(316): Xaa is Leu or Ala
  MISC_FEATURE (319)..(319): Xaa is Pro or Ser
  MISC_FEATURE (333)..(333): Xaa is Met or Tyr
  MISC_FEATURE (335)..(335): Xaa is Ser or Thr
  MISC_FEATURE (337)..(337): Xaa is Thr or Glu
  MISC_FEATURE (346)..(346): Xaa is Asp or Ala
  MISC_FEATURE (378)..(378): Xaa is Asn, Ala, Gln, or Gly
  MISC_FEATURE (410)..(410): Xaa is Pro, Ala, Gly, or Ser
  MISC_FEATURE (412)..(412): Xaa is Pro, Ser, or Ala
  MISC_FEATURE (509)..(509): Xaa is Met or Leu
  MISC_FEATURE (515)..(515): Xaa is Asn or Ser
  MISC_FEATURE (528)..(528): Xaa is Lys or is absent
LKIAAFNIQTFGETKMSNATLVSYIVQILSRYDIALVQEVRDSHLTAVGKLLDNLNQDAPDTYHYVVSEPLGRXSYKERYLFVYRPDQVSAVDSYYYDDGCEPCXNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKXXXXXXXXXXXXXXXXXXXXXXXXXXXXXXXXXXXXEPKSSDKTHTSPPSPAPEXXGGXSVFLFPPKPKDTLXIXRXPEVTCVVVXVSHEDPEVKFNWYVDGVEVHNAKTKPREEQYXSTYRVVSVLTVLHQDWLNGKEYKCKVSNKALXAXIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVXHEALHXHYTQKSLSLSPGX

SEQ ID NO: 154 - protein, 528 aa, Artificial Sequence
  [hDNase1 N74K-G105R]-[gs linker]-[SSShinge Fc]
  MISC_FEATURE (261)..(263): Xaa is any naturally occurring non-cysteine amino acid
  MISC_FEATURE (264)..(283): Xaa is Gly or Ser
  MISC_FEATURE (284)..(293): Xaa is Gly or Ser or is absent
  MISC_FEATURE (294)..(296): Xaa is any naturally occurring non-cysteine amino acid
  MISC_FEATURE (315)..(316): Xaa is Leu or Ala
  MISC_FEATURE (319)..(319): Xaa is Pro or Ser
  MISC_FEATURE (333)..(333): Xaa is Met or Tyr
  MISC_FEATURE (335)..(335): Xaa is Ser or Thr
  MISC_FEATURE (337)..(337): Xaa is Thr or Glu
  MISC_FEATURE (346)..(346): Xaa is Asp or Ala
  MISC_FEATURE (378)..(378): Xaa is Asn, Ala, Gln, or Gly
  MISC_FEATURE (410)..(410): Xaa is Pro, Ala, Gly, or Ser
  MISC_FEATURE (412)..(412): Xaa is Pro, Ser, or Ala
  MISC_FEATURE (509)..(509): Xaa is Met or Leu
  MISC_FEATURE (515)..(515): Xaa is Asn or Ser
  MISC_FEATURE (528)..(528): Xaa is Lys or is absent
LKIAAFNIQTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCRNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKXXXXXXXXXXXXXXXXXXXXXXXXXXXXXXXXXXXXEPKSSDKTHTSPPSPAPEXXGGXSVFLFPPKPKDTLXIXRXPEVTCVVVXVSHEDPEVKFNWYVDGVEVHNAKTKPREEQYXSTYRVVSVLTVLHQDWLNGKEYKCKVSNKALXAXIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVXHEALHXHYTQKSLSLSPGX

SEQ ID NO: 155 - protein, 538 aa, Artificial Sequence
  hVK3LP-[hDNase1 N96K-G127R]-(g4s)4-[SSShinge-P238S-P331S Fc]
METPAQLLFLLLLWLPDTTGLKIAAFNIQTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCRNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 548 aa, Artificial Sequence
  hVK3LP-[hDNase1 N96K-G127R]-(g4s)6-[SSShinge-P238S-P331S Fc]
METPAQLLFLLLLWLPDTTGLKIAAFNIQTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCRNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKDLSGGGGSGGGGSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - DNA, 2713 bp, Artificial Sequence
  hVK3LP-[hDNase1 N74K-G105R]-(g4s)4-[SSShinge-P238S-P331S Fc]-NGS-[PON1 Q192K] DNA
aagcttgccgccatggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcaggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaataatctagaa

SEQ ID NO: 158 - protein, 896 aa, Artificial Sequence
  hVK3LP-[hDNase1 N74K-G105R]-(g4s)4-[SSShinge-P238S-P331S Fc]-NGS-[PON1 Q192K]
METPAQLLFLLLLWLPDTTGLKIAAFNIQTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCRNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 159 - DNA, 2743 bp, Artificial Sequence
  hVK3LP-[hDNase1 N74K-G105R]-(g4s)6-[SSShinge-P238S-P331S Fc]-NGS-[PON1 Q192K] DNA
aagcttgccgccatggaaaccccagcgcagcttctcttcctcctgctactctggctcccagataccaccggtctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaagagctataaggagcgctacctgttcgtgtacaggcctgaccaggtgtctgcggtggacagctactactacgatgatggctgcgagccctgcaggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaaagatctctccggaggaggtggctcaggtggtggaggatctggaggaggtgggagtggtggaggtggttctggaggaggtggtagtggaggtggaggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaataatctagaa

SEQ ID NO: 160 - protein, 906 aa, Artificial Sequence
  hVK3LP-[hDNase1 N74K-G105R]-(g4s)6-[SSShinge-P238S-P331S Fc]-NGS-[PON1 Q192K]
METPAQLLFLLLLWLPDTTGLKIAAFNIQTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCRNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKDLSGGGGSGGGGSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 161 - DNA, 2721 bp, Artificial Sequence
  hVK3LP-[hDNase1L3 R80A-R95A-N96A NLS2delta]-(g4s)4-[SSShinge-P238S-P331S Fc]-NGS-[PON1 Q192K] DNA
atggaaaccccagcgcagcttctcttcctcctgctactctggctcccagataccaccggtatgaggatctgctccttcaacgtcaggtcctttggggaaagcaagcaggaagacaagaatgccatggatgtcattgtgaaggtcatcaaacgctgtgacatcatactcgtgatggaaatcaaggacagcaacaacaggatctgccccatactgatggagaagctgaacagaaattcagcaagaggcataacgtacaactatgtgattagctctcggcttggagcagctacatataaagaacaatatgcctttctctacaaggaaaagctggtgtctgtgaagaggagttatcactaccatgactatcaggatggagacgcagatgtgttttccagggagccctttgtggtctggttccaatctccccacactgctgtcaaagacttcgtgattatccccctgcacaccaccccagagacatccgttaaggagatcgatgagttggttgaggtctacacggacgtgaaacaccgctggaaggcggagaatttcattttcatgggtgacttcaatgccggctgcagctacgtccccaagaaggcctggaagaacatccgcttgaggactgaccccaggtttgtttggctgatcggggaccaagaggacaccacggtgaagaagagcaccaactgtgcatatgacaggattgtgcttagaggacaagaaatcgtcagttctgttgttcccaagtcaaacagtgtttttgacttccagaaagcttacaagctgactgaagaggaggccctggatgtcagcgaccactttccagttgaatttaaactacagtcttcaagggccttcaccaactcagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaa

SEQ ID NO: 162 - protein, 906 aa, Artificial Sequence
  hVK3LP-[hDNase1L3 R80A-R95A-N96A NLS2delta]-(g4s)4-[SSShinge-P238S-P331S Fc]-NGS-[PON1 Q192K]
METPAQLLFLLLLWLPDTTGMRICSFNVRSFGESKQEDKNAMDVIVKVIKRCDIILVMEIKDSNNRICPILMEKLNRNSARGITYNYVISSRLGAATYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

SEQ ID NO: 163 - DNA, 2721 bp, Artificial Sequence
  hVK3LP-[hDNase1L3 R80S-R95S-N96S NLS2delta]-(g4s)4-[SSShinge-P238S-P331S Fc]-NGS-[PON1 Q192K] DNA
atggaaaccccagcgcagcttctcttcctcctgctactctggctcccagataccaccggtatgaggatctgctccttcaacgtcaggtcctttggggaaagcaagcaggaagacaagaatgccatggatgtcattgtgaaggtcatcaaacgctgtgacatcatactcgtgatggaaatcaaggacagcaacaacaggatctgccccatactgatggagaagctgaacagaaattcatcaagaggcataacgtacaactatgtgattagctctcggcttggaagttctacatataaagaacaatatgcctttctctacaaggaaaagctggtgtctgtgaagaggagttatcactaccatgactatcaggatggagacgcagatgtgttttccagggagccctttgtggtctggttccaatctccccacactgctgtcaaagacttcgtgattatccccctgcacaccaccccagagacatccgttaaggagatcgatgagttggttgaggtctacacggacgtgaaacaccgctggaaggcggagaatttcattttcatgggtgacttcaatgccggctgcagctacgtccccaagaaggcctggaagaacatccgcttgaggactgaccccaggtttgtttggctgatcggggaccaagaggacaccacggtgaagaagagcaccaactgtgcatatgacaggattgtgcttagaggacaagaaatcgtcagttctgttgttcccaagtcaaacagtgtttttgacttccagaaagcttacaagctgactgaagaggaggccctggatgtcagcgaccactttccagttgaatttaaactacagtcttcaagggccttcaccaactcagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcctcttcaggaaccaccagtcttcttaccaaacacgacttaatgctctccgagaggtacaacccgtagaacttcctaactgtaatttagttaaaggaatcgaaactggctctgaagacttggagatactgcctaatggactggctttcattagctctggattaaagtatcctggaataaagagcttcaaccccaacagtcctggaaaaatacttctgatggacctgaatgaagaagatccaacagtgttggaattggggatcactggaagtaaatttgatgtatcttcatttaaccctcatgggattagcacattcacagatgaagataatgccatgtacctcctggtggtgaaccatccagatgccaagtccacagtggagttgtttaaatttcaagaagaagaaaaatcgcttttgcatctaaaaaccatcagacataaacttctgcctaatttgaatgatattgttgctgtgggacctgagcacttttatggcacaaatgatcactattttcttgacccctacttaaaatcctgggagatgtatttgggtttagcgtggtcgtatgttgtctactatagtccaagtgaagttcgagtggtggcagaaggatttgattttgctaatggaatcaacatttcacccgatggcaagtatgtctatatagctgagttgctggctcataagattcatgtgtatgaaaagcatgctaattggactttaactccattgaagtcccttgactttaataccctcgtggataacatatctgtggatcctgagacaggagacctttgggttggatgccatcccaatggcatgaaaatcttcttctatgactcagagaatcctcctgcatcagaggtgcttcgaatccagaacattctaacagaagaacctaaagtgacacaggtttatgcagaaaatggcacagtgttgcaaggcagtacagttgcctctgtgtacaaagggaaactgctgattggcacagtgtttcacaaagctctttactgtgagctctaa

SEQ ID NO: 164 - protein, 906 aa, Artificial Sequence
  hVK3LP-[hDNase1L3 R80S-R95S-N96S NLS2delta]-(g4s)4-[SSShinge-P238S-P331S Fc]-NGS-[PON1 Q192K]
METPAQLLFLLLLWLPDTTGMRICSFNVRSFGESKQEDKNAMDVIVKVIKRCDIILVMEIKDSNNRICPILMEKLNRNSSRGITYNYVISSRLGSSTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDILFRNHQSSYQTRLNALREVQPVELPNCNLVKGIETGSEDLEILPNGLAFISSGLKYPGIKSFNPNSPGKILLMDLNEEDPTVLELGITGSKFDVSSFNPHGISTFTDEDNAMYLLVVNHPDAKSTVELFKFQEEEKSLLHLKTIRHKLLPNLNDIVAVGPEHFYGTNDHYFLDPYLKSWEMYLGLAWSYVVYYSPSEVRVVAEGFDFANGINISPDGKYVYIAELLAHKIHVYEKHANWTLTPLKSLDFNTLVDNISVDPETGDLWVGCHPNGMKIFFYDSENPPASEVLRIQNILTEEPKVTQVYAENGTVLQGSTVASVYKGKLLIGTVFHKALYCEL

(19) United States
(12) Patent Application Publication
(10) Pub. No.: US 2024/0101982 A1
(43) Pub. Date: Mar. 28, 2024

(54) PARAOXONASE FUSION POLYPEPTIDES AND RELATED COMPOSITIONS AND METHODS

(71) Applicant: Theripion, Inc., Shoreline, WA (US)

(72) Inventors: Jeffrey A. Ledbetter, Shoreline, WA (US); Martha S. Hayden-Ledbetter, Shoreline, WA (US)

(21) Appl. No.: 18/546,413

(22) PCT Filed: Feb. 17, 2022
(86) PCT No.: PCT/US2022/016723

§ 371 Date: Aug. 14, 2023

Related U.S. Application Data
(60) Provisional application No. 63/151,236, filed on Feb. 19, 2021.
(60) Provisional application No. 63/151,272, filed on Feb. 19, 2021.

(51) Int. Cl. C12N 9/16; A61P 11/00; C07K 16/24; C12N 9/22

(52) U.S. Cl. CPC C12N 9/16; A61P 11/00; C07K 16/241; C12N 9/22; C12Y 301/08001; C12Y 301/21001; A61K 38/00; C07K 2317/622; C07K 2319/01; C07K 2319/30

(57) ABSTRACT

Compositions and methods relating to paraoxonase fusion polypeptides are disclosed. In some aspects, the fusions are bispecific molecules that include a first biologically active polypeptide linked amino-terminal to a biologically active paraoxonase (e.g., human PON1 or a variant thereof), wherein the first biologically active polypeptide is a DNase, an RNase, a SOD1, a CTLA-4 extracellular domain, a CD40 extracellular domain, or a polypeptide that specifically binds and neutralizes an inflammatory cytokine. Bispecific fusions may further include a second biologically active polypeptide (e.g., a dimerizing domain or a domain that specifically binds to the neonatal Fc receptor (FcRn)) linked carboxyl-terminal to the first biologically active polypeptide and amino-terminal to the paraoxonase. In other aspects, a fusion polypeptide includes a biologically active paraoxonase linked carboxyl-terminal or amino-terminal to a dimerizing or FcRn-binding domain. In certain variations, a dimerizing or FcRn-binding domain is an immunoglobulin Fc region. Also disclosed are dimeric proteins comprising first and second paraoxonase fusion polypeptides as disclosed herein. The fusion polypeptides and dimeric proteins are useful in methods for therapy.

### CROSS-REFERENCE TO RELATED APPLICATIONS

This application claims the benefit of U.S. Provisional Application Nos. 63/151,236 and 63/151,272, filed Feb. 19, 2021. Each of the foregoing applications is incorporated by reference herein in its entirety.

### REFERENCE TO SEQUENCE LISTING

The instant application contains a Sequence Listing which has been submitted in ASCII format via EFS-Web and is hereby incorporated by reference in its entirety. Said ASCII Copy, created on Feb. 16, 2022, is named “TRP_0410PC_20220216_Seq_Listing_ST25” and is 552,734 bytes in size.

### BACKGROUND OF THE INVENTION

Lung Disease and Fibrosis

Air pollution, primarily caused by atmospheric ozone levels, is a global health problem that is projected to increase in the future (Zhang et al., Front. Immunol. 10:2518, 2019). Ozone is a strong oxidant and tropospheric (ground level) ozone causes lung inflammation and damage. Ozone pollution is also linked to vascular disease and neurological disease. (Zhang et al., supra).

Lung disease is already a significant health and economic burden world-wide. Lung diseases are the third leading cause of death in the United States. Including chronic obstructive pulmonary disease (COPD) and asthma, the cost is projected to be over 80 billion dollars per year by 2020 (Foster el al., J. COPD 3:2011-2018, 2006; Nurmagambetov el al., Annals of Am. Thoracic Soc., Center for Disease Control and Prevention, 2017). Current therapies include nebulized steroids, anticholinergics, and bronchodilators for COPD and leukotriene modulators and long acting beta agonists for asthma. Humanized antibodies, given by subcutaneous or intravenous injection, are approved as add-on therapy for hard-to-control asthma patients; these antibodies deplete IgE (Omalizumab), inhibit IL-5 (Mepolizumab and Reslizumab), or deplete eosinophils (Benralizumab). There is a need for new targets and approaches for improved therapy of lung diseases.

Lung inflammation is associated with infiltration of neutrophils, eosinophils, macrophages, and lymphocytes into the lung lumen and tissues. Extracellular DNA derived from neutrophil extracellular traps (NETS) is present in the lungs in patients with asthma, cystic fibrosis, and COPD (Liu et al., Chinese Med. Journal 130:730-736, 2017; Twaddell et al., CHEST 156:774-782, 2019; Uddin et al., Front. Immunol. 10:47, 2019; Yamashita et al., J. Immunol. 191:949-960, 2013). In addition, eosinophils contribute to extracellular traps in patients with asthma (Dworski et al., J. Allergy Clinical Immunol. 127:1260-1266, 2011). The traps thicken lung mucus and contain inflammatory mediators including myeloperoxidase and neutrophil elastase that promote tissue damage (Uddin et al., supra). NET-associated myeloperoxidase and elastase are increased in the sputum and bronchioalveolar lavage of cystic fibrosis patients and correlate with lung disease severity (see generally Kahn et al., Genes 10:183, 2019). DNase1 can digest NETS and reduce the viscosity of mucus, allowing improved lung clearance (id.).

Extracellular DNA that can be digested with DNase1 was found to be essential for silica-induced lung fibrosis (see Benmerzoug et al., Nature Comm. 9:5226, 2018). Silica induces cell death and release of self-DNA in the alveolar space and induces the stimulator of interferon genes (STING) inflammatory pathway leading to fibrosis (id.).

P. aeruginosa Infections

P. aeruginosa is a ubiquitous Gram-negative rod that causes severe opportunistic infections in immunocompromised individuals, burn patients, and patients with lung diseases such as COPD and cystic fibrosis (CF) (see Mulcahy et al., Microb. Ecol. 68:1-12, 2014). It is particularly troublesome on medical surfaces such as ventilators and is among the most common nosocomial pathogens and a frequently isolated pathogen from patients with ventilator-associated pneumonia. Because it is a major pathogen in adults with cystic fibrosis (CF) and because it is associated with increased clinical deterioration, eradication of P. aeruginosa would have important implications for CF therapies and other P. aeruginosa infections. (See. e.g., Martinez-Solano et al., Chronic Infect. Dis. 47:1526-1533, 2008.; Meyer-Hamblett et al., Clin Inf. Dis. 59:624-631, 2014; Gallego et al., BMC Pulmonary Med. 14:103, 2014.)

Pulmonary failure, a consequence of persistent bacterial lung infections particularly with P. aeruginosa, accounts for 80% of deaths in CF patients. Infection with P. aeruginosa in CF increases the risk of early death by almost three-fold (see McColley et al., Pediatric Pulmonology 52:909-915, 2017). Despite intense research being focused on easing bacterial lung infections, there is still a need for additional approaches to effective treatment in the CF lung.

Quorum sensing (QS) is an important mechanism that assists in P. aeruginosa persistence in the CF lung and contributes significantly to its pathogenesis. QS is mediated by small molecules produced by the bacteria that allow the coordination of communitywide activities, particularly acyl homoserine lactones (or AHLs). Acyl homoserine lactones share a basic structure, consisting of a homoserine lactone ring (HSL) and an acyl chain which can vary in length and degree of saturation. The variant side chains give specificity to the QS mechanism, enabling bacteria to recognize and respond to their specific AHLs. Almost a quarter of the expressed P. aeruginosa proteome is controlled by QS. In P. aeruginosa, QS plays an essential role in the expression and regulation of numerous virulence factors. QS also plays an important role in the formation of biofilms. Biofilms essentially envelop the bacterial community in a matrix of polysaccharides, extracellular DNA, and proteins, protecting it from the host's immune responses and external antimicrobial agents like antibiotics, facilitating survival of the P. aeruginosa. (Mulcahy et al., supra.)

### Paraoxonase 1 (PON1)

Paraoxonase 1 (PON1) is an antioxidant enzyme that inhibits oxidation of low-density lipoprotein (LDL) and preserves its function (Aviram et al., J. Clin. Invest. 101:1581-1590, 1998). PON1 has multiple substrates, including organophosphates and acyl homoserine lactones, the quorum sensing molecules made by Pseudomonas aeruginosa. PON1 activity is important in protection from arteriosclerosis and ischemic stroke (Litvinov et al., N. Am. J. Med. Sci. 4:523-532, 2012). In addition, PON1 activity is reduced in patients with intestinal inflammation including those with irritable bowel syndrome (IBS) or inflammatory bowel disease (IBD) such as Crohn's disease (Oran et al., J. Pak. Med. Assoc. 64:820-822, 2014; Szczelkik et al., Molecules 23:2603, 2018; Rothem et al., Free Rad. Biol. Med. 43:730-739, 2007). Studies in a mouse model for colitis have demonstrated that treatment with N-acetyl cysteine stimulates upregulation of PON1, and that increased PON1 activity is associated with amelioration of colon damage (You et al., Dig. Dis. Sci. 54:1643-1650, 2009).

PON1 is also deficient in multiple lung diseases. There is strong evidence that PON1 is important in protection from lung inflammation, and that low PON1 activity is associated with lung diseases. Oxidative stress is an important factor in the pathogenesis of asthma (see Sahiner et al., WAO Journal 4:151-158, 2011), and the antioxidant activity of PON1 is impaired in asthma patients. In children with asthma, PON1 levels were significantly lower than controls (p<0.001) (Emin et al., Allergol. Immunopathol. (Madr) 43:346-352, 2014). Additional studies have reported that PON1 activity is significantly lower in asthma patients (p<0.024) (Sarioglu et al., Iran J. Asthma Immunol. 14:60-66, 2015). Another study showed that PON1 activity is lower in asthmatic patients during active disease, but PON1 activity increases during disease remission (Tolgyesi et al., Internal. Immunol. 21:967-975, 2009). This study also used gene profiling in an experimental mouse asthma model to identify PON1 downregulation as a potential target for monitoring disease activity (see id.). In addition, a mouse asthma model showed that PON1 overexpression reduced airway inflammation and remodeling, and reduced inflammatory cytokines (Chen et al., J. Cell Biochem. 119:793-805, 2018).

Compared to controls, patients with COPD were found to have significantly lower PON1 activity and increased oxidative stress as evidenced by lower levels of reduced thiol groups (Rumora et al., J. COPD 11:539-545, 2014). Another study found a polymorphism at −108 C>T in the PON1 promoter was strongly associated with COPD (Rajkovic et al., J. Clin. Path. 71:963-970, 2018). Both the −108 TT genotype and T allele were strongly associated (p<0.001), and the −108T allele could be a factor in the reduced expression of PON1 in COPD patients (id.).

PON1 activity was reported to be reduced in patients with sarcoidosis and interstitial lung disease, particularly during active disease (Ivanisevic et al., Eur. J. Clin. Invest. 46:418-424, 2016; Uzun et al., Curr. Med. Res. Opin. 24:1651-1657, 2008). Another study found lower PON1 during hypoxia (Okur et al., Sleep Breath. 17:365-371, 2013). PON1 activity was absent in patients with lung disease who had been exposed to sulfa mustard gas, even though the exposure was decades ago (Golmanesh et al., Immunopharmacol. and Immunotoxicol. 35:419-425, 2013).

PON1 and myeloperoxidase were reported to exert reciprocal control, in that an excess of PON1 inhibits myeloperoxidase, whereas an excess of myeloperoxidase inhibits PON1 (Huang et al., J. Clin. Invest. 123:3815-3828, 2013). Huang et al. found that both PON1 and myeloperoxidase were tightly bound to apoA-I. This provides one mechanism for the decreased PON1 activity under conditions of inflammation, where neutrophils and eosinophils produce an excess of myeloperoxidase. However, under normal conditions, PON1 is in excess and represses the activity of myeloperoxidase. (See id.)

Recombinant PON1 (rPON1) was reported to inhibit PMA-induced differentiation of monocytic cell line THP-1 (Rosenblat et al., Atherosclerosis 219:49-56, 2011). Mice injected IP with rPON1 had reduced differentiation of monocytes to macrophages and reduced expression of CD11b and CD36. The total cellular peroxides of peritoneal macrophages decreased by 18%. (Id.)

PON1 therapy has been studied in experimental models. One study showed that recombinant human PON1 injection was able to protect guinea pigs from sarin and soman inhalation toxicity (Valiyaveettil et al., Biochem. Pharmacol. 81:800-809, 2011; Valiyaveettil et al., Toxicol. Letters 202:203-208, 2011). Other studies showed therapy with PON1 protected from organophosphate poisoning in mice (Bajaj et al., Appl. Biochem. Biotechnol. 180:165, 2016; Stevens et al., Proc. Natl. Acad. Sci. USA 105:12780-12784, 2008). These studies used recombinant PON1 purified from bacterial inclusion bodies or made in T. ni larvae with a baculovirus vector. Both groups also expressed PON1 192K, a variant derived from the rabbit sequence, that helps stabilize and increase PON1 enzymatic activity. (See Bajaj et al., supra; Stevens et al., supra.)

Expression of PON1 in bacteria has also been used to select mutants that have enhanced enzymatic activity towards organophosphates and nerve agents (see Aharoni et al., Proc. Natl. Acad. Sci. USA 101:482-487, 2004; Goldsmith et al., Chemistry and Biology 19:456, 2012). Engineering of PON1 was done by directed evolution to find a variant with improved, soluble expression in bacteria. One evolved PON1 was crystallized and the PON1 structure described (see Harel et al., Nature Struct. Mol. Biol. 11:412-419, 2004). Another PON1 variant widely used is G3C9, a hybrid PON1 formed from shuffling mouse, rat, rabbit, and human PON1 sequences (see Aharoni et al., Proc. Natl. Acad. Sci. USA 101:482487, 2004). Other PON1 variants with increased organophosphatase activity but reduced esterase activity have been described (see Aharoni et al., supra). Further evolution of G3C9 to increase activity towards G-type nerve agents tabun, sarin, soman, and cyclosarin has also been reported (Gupta et al., Nat. Chem. Biol. 7:120-125, 2011. Goldsmith et al., Chemistry and Biology 19:456-466, 2012). When compared to human PON1, G3C9 has amino acid differences at 55 positions, and the further evolved PON1 variants have even more amino acid differences (Goldsmith et al., supra).

PON1 and the PON1 variant G3C9 have been tested in mouse models of experimental colitis (Yamashita et al., J. Immunol. 191:949-960, 2013). PON1 therapy was effective in TNBS-induced colitis and was equivalent to anti-TNFα therapy, currently the most effective clinically used therapeutic agent in man. However, in a chronic colitis model with CD4+CD45RB^{high} cell transfer, PON1 was not effective but G3C9 showed strong efficacy. Efficacy of G3C9 was equivalent to anti-TNFα in the chronic colitis model. (Id.) In another study, PON1 activity was found to correlate with severity of Crohn's disease (Sczceklik et al., Molecules 23:2603, 2018).

Another approach towards therapy with PON1 has been to inject HDL-like particles, formed from reconstituted apo A-I plus PON1, a therapy termed BL-3050 (Gaidukov et al., BMC Clinical Pharmacol. 9:18, 2009). In these studies, both G3C9 expressed in bacteria and BL-3050 complexes were protective in a mouse model of organophosphate poisoning (id.).

PON1 has been expressed as a fusion protein attached to the C-terminus of a mAb heavy chain specific for insulin receptor (see, e.g., Boado el al., Mol. Pharm. 5:1037, 2008). Expression of this hybrid heavy chain with an insulin receptor-specific light chain resulted in secretion of a bifunctional antibody that bound insulin receptor and had PON1 enzyme activity (id.). This hybrid molecule was expressed in mammalian cells (COS and CHO) (see id.; Boado et al., Biotechnol. and Bioengineering, 108:186, 2011). The hybrid molecule was also tested in nonhuman primates and was found to penetrate the blood/brain barrier; however, the molecule was rapidly cleared from the peripheral blood into the peripheral tissues, primarily the liver (Boado et al. (2011), supra). There are no reports of clinical testing of this molecule. Expression levels of the fusion protein in CHO cells were low (5-10 mg/liter) even after multiple transfections and subcloning (see id.).

PON1 has also been fused with a membrane transduction domain, termed PEP-1-PON1 (Kim et al., Biomaterials 64:45-56, 2015). This molecule inhibited the inflammatory response of a microglial cell line and protected against dopaminergic cell death in vivo in a mouse model of Parkinson's disease (id.).

Paraoxonase enzymes are able to hydrolyse QS molecules that are acyl homoserine lactones (AHL), including C12HSL (N-3-oxo-dodecanoyl-L-Homoresine Lactone), the primary QS molecule from P. aeruginosa. Studies of human PON1 indicate that it has pleiotropic enzyme activities, hydrolyzing a variety of substrates, including lactones (Billecke et al., Drug Metabolism and Disposition 28:1335-1342, 2000; Khersonsky and Tawfik. Biochemistry 44:6371-6382, 2005; Gaidukov and Tawfik, J. Lipid Res. 48:1637-1646, 2007; Bajaj et al., Protein Science 22:1799-1807, 2013). One study in Drosophila melanogaster demonstrated protection from Pseudomonas aeruginosa lethality by expression of a human PON1 transgene capable of hydrolyzing a critical quorum sensing molecule N-3-oxodecanoyl homoserine lactone (30C12-HSL) (Stoltz et al., J. Clin. Invest. 118:3123-3131, 2008).

### Dnase1

DNase1 is a calcium and magnesium-dependent endonuclease coded by the human DNASE1 gene. DNase1 cleaves single-stranded and double-stranded DNA and chromatin. Cleavage of chromatin occurs in hypersensitive sites where chromatin is open and accessible, and is used in genomics to identify regions likely to contain active genes (Boyle et al., Cell 132:311-322, 2008). DNase1 helps degrade DNA during apoptosis (Samejima et al., Nat. Rev. Mol. Cell Biol. 6:677-688, 2008) and also degrades neutrophil extracellular traps (NETS) (Hakkim et al., Proc. Natl. Acad. Sci. USA 107:9813, 2010). DNase1 can digest biofilm DNA, resulting in a reduction in biofilm biomass, and increase antibiotic mediated biofilm killing (Tetz et al., Antimicrob. Agents Chemother. 53:1204-1209, 2009).

Accumulation of extracellular DNA from dead and dying cells was identified as a driver of disease in patients with systemic lupus erythematosus (SLE) long ago. DNase1 was administered to SLE patients at the Rockefeller starting in 1959. However, bovine DNase1 was used, and therapy was limited by its immunogenicity (see Valle et al., Autoimmunity Rev. 7:359, 2008). Decreased levels of DNase1 and DNase1 mutations with reduced activity have been reported in patients with SLE (see Valle et al., supra). Reduced DNase1 activity in urine was found in patients with lupus nephritis, and reduced DNase1 activity was a biomarker of disease progression (Pedersen et al., J. Pathol. Clin. Res. 4:193, 2018). In SLE patients, defective clearance of apoptotic debris and accumulation of chromatin contributes to breaking of tolerance and development of autoimmune disease.

Activated neutrophils extrude DNA bound with cytoplasmic and granule proteins, called neutrophil extracellular traps (NETs), to combat infectious agents, a process also known as “NETosis” (see Brinkmann, J. Innate Immun. 10:422-431, 2018). However, NETs are not targeted and often cause collateral tissue damage and inflammation if not effectively cleared. NETs have been implicated in driving disease progression in multiple incurable and serious diseases. Evidence for the importance of NETs in psoriasis, cystic fibrosis, SLE, RA, type I diabetes, sepsis, small vessel vasculitis, IBD, type II diabetes, and obesity was recently reviewed (Mutua and Gershwin, Clin. Rev. Allergy Immunol., 2020, DOI 10.1007/s12016-020-08804-7). See also Michailidou, Front. Immunol. 11:619705, 2020 (reviewing involvement of neutrophils and NETs in vasculitis). NETs also play an important role in cardiovascular disease and thrombosis, and in cancer where NETs promote tumor progression and metastasis (Brinkmann, supra). In addition to their importance in cystic fibrosis, NETs also play a role in the clinicopathology of other respiratory diseases such as asthma and chronic obstructive pulmonary disease (COPD) (see Liu et al., Chinese Med. Journal 130:730-736, 2017; Twaddell et al., CHEST 156:774-782, 2019; Uddin et al., Front. Immunol. 10:47, 2019). Recently, NETs were found to contribute to immunothrombosis in COVID-19 acute respiratory distress syndrome (Middleton el al., Blood 136:1169, 2020; Zuo et al., JCI Insight 5:e138999, 2020; Barnes et al., J. Exp. Med. 217:e20200652, 2020).

DNase1 is capable of digesting NETs and works together with another extracellular DNase called DNase1 L3 to clear NETs. Mice deficient in both DNase1 and DNase1L3 were unable to clear NETs and rapidly died of massive intravascular clot formation and organ damage after neutrophil activation (Brinkmann, supra). The contribution of NETs to thrombosis has already led to a clinical trial of wild-type DNase1 (Pulmozyme@) taken by inhalation therapy in COVID-19 patients (NCT04359654).

Recombinant wild-type human DNase1 (Pulmozyme@) was produced by Genentech and first approved for cystic fibrosis (CF) to digest DNA in the lungs and reduce congestion. High levels of DNA derived primarily from NETs are pathogenic in CF lung (Law et al., J. Inflammation, 14:1-8, 2017). Bacterial DNA from Pseudomonas aeruginosa biofilm formation is also present in many CF patients and may also be digested by DNase1 (Whitchurch el al., Science 295:1487, 2002).

Pulmozyme is delivered directly to the lungs by nebulization and is taken daily. Pulmozyme has been studied in patients with lung diseases other than CF, but has not been approved for alternative uses (Torbic et al., J. Pharmacy Practice 29:480, 2016). Pulmozyme was tested in a mouse lupus model (Verthelyi et al., Lupus 7:223, 1998) and in a phase I clinical trial in SLE patients (Davis et al., Lupus 8:68-76, 1999) without a decrease in disease measurements. Systemic administration of pulmozyme therapy may be limited because the enzyme is inhibited by globular actin that is abundant in blood and lungs (Ulmer et al., Proc. Natl. Acad. Sci. USA 93: 8225, 1996) and because of its short half-life.

Efforts have been made to enhance DNase1 for CF and SLE patients. A site-directed mutagenesis study of the DNA-binding interface identified 27 amino acid positions critical for enzyme activity and another 13 positions peripherally involved in DNA interactions with minimal impact on enzyme activity (see Pan et al., Prot. Sci. 7:628-636, 1998). This group also showed that increasing the local electrostatic attraction toward DNA by adding specific additional positively charged residues not only improved binding affinity to DNA but also improved functional activity and eliminated the inhibition by salt (Pan et al., Biochemistry 36:6624-6632, 1997; Pan and Lazarus, J. Biol. Chem. 273:11701-11708, 1998). Consistent with this observation, alanine replacement of two arginines (Arg-41 and Arg-111) that make critical contacts with DNA greatly reduced the DNA cleavage activity of human DNase1 (Pan et al., 1998, supra). Furthermore, the introduction of positively or negatively charged residues at sites distal to the DNA interface did not alter specific activity (Ulmer et al., Proc. Natl. Acad. Sci. USA 93:8225-8229, 1996).

The hyperactive DNase1 variants showed significant enhancement (up to 10,000-fold) under conditions of low DNA concentration and short DNA length (Pan and Lazarus, supra). Under optimal conditions for wild-type DNase1, however, the best variant showed activity only 7-fold higher and contained a single amino acid change (N74K). The N74K hyperactive variant showed reduced dependence on calcium (Pan and Lazarus, Prot. Sci. 8:1780-1788, 1999).

Ulmer et. al. (supra) and Pan et al. (1998, supra) showed that phenylalanine replacement of Ala-114 (A114F) eliminated inhibition of DNase1 by G-actin.

Actin-resistant and hyperactive DNase1 mutant enzymes are further described in U.S. Pat. Nos. 6,348,343 and 6,391,607.

DNASE1 is polymorphic, and alleles that encode for enzyme with reduced activity are associated with increased risk for autoimmune disease. However, a naturally occurring allele, G105R, was found to encode a DNase1 variant with a three-fold increase in activity (Yasuda et al., Int. J. Biochem. Cell. Biol. 42:1216-1225, 2010). This is a minor allele found in African populations and could confer resistance to autoimmune disease (id.).

There is great potential for DNase1 therapy using actin/salt-resistant and hyperactive DNase1. However, it was not possible to obtain stable CHO cells that constitutively express an enhanced DNase1 despite screening of thousands of clones (see Lam et al., Biotech. Progress 32: 523-533, 2017). This was thought to be due to toxicity of the enzyme during high expression in CHO cells. Lam et al. (supra) describe an inducible expression system in CHO cells as one potential solution to the problem of manufacturing an enhanced DNase1 enzyme.

Dwyer et al. (J. Biol. Chem. 274:9738-43, 1999) describe DNase1-Fc fusion proteins with impaired enzyme activity due to dimerization by the Fc domain. Dwyer et al. added short linkers (up to 7 aa) between the DNase1 and the Fc domain without improving activity (see id.). U.S. Pat. No. 8,841,416 to Ledbetter et al. describe a DNase1-Fc fusion with a linker of 21 amino acids and having activity at least equivalent to recombinant DNase1 control.

Although wild-type DNase1 (Pulmozyme) therapy is FDA approved in CF, there is not a CF mouse model where DNase1 is active. Pulmozyme was approved for CF therapy without testing for activity in a relevant in vivo disease model (Greene, Hum. Exp. Toxicol., May:13 Suppl 1:S1-42, 1994). More recently, wild-type DNase1 therapy has been studied in several in vivo models with encouraging results. DNase1 was found to significantly reduce airway resistance but did not reduce inflammation in an acute asthma model (da Cunha et al., Exp. Lung Res. 42:66, 2016). In a silica-induced lung inflammation model, DNase1 therapy prevented DNA-mediated STING activation and blocked the downstream type I IFN response (Benmerzoug et al., Nat. Comm. 9:5226, 2018). In a model of acute lung injury, DNase1 therapy protected mice from lung edema and lung vascular permeability. DNase1 therapy also reduced NET formation and platelet sequestration in the lung (Caudrillier et al., J. Clin. Invest. 122:2661, 2012).

Studies in mouse tumor models have shown that NETs promote metastasis and that injection of DNase1 or DNase1-coated nanoparticles reduced tumor metastasis (Cools-Lartigue et al., J. Clin. Invest. 123:3446, 2013; Park et al., Sci. Translational Med. 8:361ral38, 2016). Another study showed that a combination of DNase1 and proteases significantly inhibited growth of human colon cancer cells in a nude mouse model (Trejo-Becirril et al., Integrative Cancer Therapies 15:NP35-NP43, 2016). In cancer patients, increased concentration of citrullinated histone H3, a marker of neutrophil NET formation, strongly predicted a poor clinical outcome (Thalin et al., PLoS ONE 13:e0191231, 2018).

In a murine DSS colitis model (Babicova et al., Folia Biolica (Praha) 64:10, 2018) DNase1 therapy caused a reduction in TNFα and myeloperoxidase in the colon. Another study (Li et al., J. Crohns Colitis 2020, 14:240-253, 2020) found that DNase1 therapy decreased cytokine production and attenuated accelerated thrombus formation and platelet activation after DSS-induced colitis.

DNase1 therapy in a hind-limb ischemia reperfusion model caused increased perfusion, decreased infiltrating inflammatory cells, and reduced a local marker of thrombosis (Albadawi et al., J. Vasc. Surg. 64:484, 2016). In a rat model of ischemia-reperfusion-induced acute kidney injury, DNase1 treatment showed significant renoprotective effects. Exogenous administration of DNase1 ameliorated both functional and histologic hallmarks of acute injury in kidneys of ischemic rats (Peer et al., Am. J. Nephrol. 43:195, 2016).

DNase1 treatment prevented organ damage and protected from death when given 4 or 6 hours after injury in a cecal ligation and puncture sepsis model in mice (Mai et al., Shock 44:166, 2015). Extracellular DNA from NETs has been recognized as a scaffold for thrombus formation and infusion of DNase1 prevented thrombosis in a model of inferior vena cava stenosis (Brill et al., J. Thrombosis Haemostasis 10:136, 2012).

### RNase

RNase 1 is a member of the RNase A superfamily consisting of RNases 1 to 8 in humans. RNase A was first isolated from bovine pancreas where it functions primarily as a digestive enzyme. In humans, RNase 1 digests single stranded and double stranded RNA and RNA/DNA hybrids. RNase 1 is produced by multiple cell types in humans, and is thought to participate primarily in vascular homeostasis. RNase 1 plays a major role in polynucleotide digestion to remove inflammatory extracellular RNA. In addition, RNase 1 has anti-viral activity.

Therapy with RNase has been studied in multiple disease models. For example, growth of the human lung tumor line A549 in athymic mice was inhibited by PEG-RNase1 (Rutkoski et al., Translational Oncology 6:392-397, 2013). RNase therapy also showed anti-tumor activity in a syngeneic mouse tumor model with Lewis Lung carcinoma and altered microRNA profiles (Mironova et al., Oncolarget. 8:78796-78810, 2017). RNase1 was reported to bind to a tumor-associated antigen, Globo H, expressed on the surface of breast adenocarcinoma cells (Eller et al., ACS Central Sci. 1:181-190, 2015).

In addition, RNase therapy has been found effective in animal models of heart transplantation (Kleinert et al., J. Am. Heart Assn. doi: 10.1021, 2016), plaque formation (Simsekyilmaz et al., Circulation 129: 598-606, 2014), and myocardial infarction (Steiger et al., JAMA 6:e004541, 2017). RNase1 therapy also reduced cerebral edema and infarction size in an acute stroke model (Walberer et al., Curr. Neovas. Res. 6:12-19, 2009).

RNase therapy was found to prevent postoperative cognitive decline in aged mice (Chen et al., PLoS One 10:e0134307, 2015).

RNase therapy has also been studied in a mouse model of systemic lupus erythematosus (SLE) (Sun et al., J. Immunol. 190:2536-2543, 2013). Overexpression of TLR7, an RNA sensor, causes a lupus-like disease with autoantibodies, kidney disease, and early mortality. Crossing these mice with mice that overexpress RNase A as a transgene resulted in progeny with increased survival, reduced lymphocyte activation, reduced kidney deposits of IgG and C3, and reduced hepatic inflammation and necrosis. (Id.)

RNase-Ig in which human RNase1 is fused to a mutated human IgG1 Fc domain comprising P238S and P331S mutations (see U.S. Pat. No. 8,937,157) has been tested in phase I and phase 11 clinical trials in patients with systemic lupus erythematosus (SLE) and Sjogren's syndrome.

### Superoxide Dismutase (SOD)

Superoxide dismutase (SOD) is an antioxidant enzyme that converts oxygen free radicals to H₂O₂ and O₂. There are three human SOD enzymes. SOD1, also called Cu/Zn SOD, is a cytoplasmic enzyme that is a homodimer stabilized by an intrachain disulfide bond and by metal binding (Doucette et al., J. Biol. Chem. 279:54558-54566, 2004). SOD2 is a tetramer that is localized in the mitochondria and uses manganese in its active site. SOD3 is a tetramer that is secreted and contains copper and zinc. For a review of the roles of SODs, glutathione peroxidase, and catalase in overall protection from oxidative damage, see Ighodaro and Akinolyc, Alexandria J. Med. 54:287-293, 2018.

SOD3 is the major extracellular antioxidant enzyme in the lungs, where it protects the extracellular matrix during lung injury. Genetic variants of SOD3 are associated with decreased lung function in adults and rapid progression of COPD (see Ganguly et al., Physiol. Genomics 37:260-267, 2009 for review).

SOD1 has been fused with a cell membrane transduction domain in order to deliver SOD1 to the cell cytoplasm. For example, Eum et al. (Free Rad. Biol. Med. 37:1656-1669, 2004) made a fusion of a 21 amino acid peptide called PEP-1 with SOD, and expressed the fusion protein in bacteria. They showed that the PEP-I-SOD molecule delivered SOD through the cell membrane and was effective after IP injection in vivo in preventing neuronal death following ischemic insult. SOD1 and variant forms of SOD1 have also been fused to an enhanced green fluorescent protein (EGFP) tag to create a fusion protein which can be detected in fluorescence based binding and chaperone assays (Ganesan et al., Cell Death and Differentiation 15:312-321, 2008).

### Cytotoxic T Lymphocyte-Associated Molecule-4 (CTLA-4)

Cytotoxic T lymphocyte-associated molecule-4 (CTLA-4) is an immunoregulatory membrane receptor resulting in the downregulation of T-cell responses by inhibiting the costimulatory interactions of CD28-B7 (Carreno et al., J. Immunol. 165:1352-1356, 2000). The extracellular domain of CTLA-4 fused with a human IgG1 mutant Fc domain (abatacept, Orencia™) is approved by the FDA for therapy of RA because of its ability to suppress activation of the CD28 receptor on T cells (see Linsley et al., J. Exp. Med. 174:561-569, 1991; Emma Hitt, FDA Approves Subcutaneous Abatacept for RA, Medscape Medical News, Aug. 2, 2011). Abatacept inhibits T cell production of inflammatory cytokines and inhibits B cell class switching and IgG antibody responses.

Abatacept treatment has been shown to prevent dermal fibrosis and induce regression of established inflammation-driven fibrosis (Ponsoye et al., Ann. Rheum. Dis. 75:2142-2149, 2016). In addition, Abatacept significantly reduced fibrogenic marker levels, T-cell proliferation, and M1/M2 macrophage infiltration in lesioned lungs of Fra-2 mice, indicating it might be useful in treatment of lung fibrosis in humans (Bolete et al., Arthritis Research and Therapy 20:197, 2018). According to the Pulmonary Fibrosis Foundation, Abatacept is currently being tested in a phase 2 clinical trial in interstitial fibrosis patients (trial #NCT03215927), and is also being tested in a phase 3 clinical trial for treatment of relapsing non-severe granulomatosis with polyangiitis (Wegener's granulomatosis), (trial #NCT02108860). Costimulation inhibitors are being evaluated as promising candidates for suppressing the damage associated with lung inflammation and repair processes.

Cd40-Cd154

CD40 is a member of the TNFα receptor superfamily and is expressed on lymphocytes, hematopoeitic and structural cells, including fibroblasts, epithelial cells and endothelial cells (Sime and O'Reilly, Clin. Immunol. 99:308-319, 2001). The ligand for CD40, (CD40L or CD154), is expressed on activated T lymphocytes, activated platelets, mast cells and eosinophils. CD154 expressed by activated T cells provides an essential signal to CD40 on B cells that is needed for class switching and IgG production. However, CD40 expressed on the surface of structural cells such as endothelial cells can be stimulated by binding CD154, resulting in the production of inflammatory cytokines (id.). Activated platelets express CD154 on their surface and are also the primary source of soluble CD154 in the blood.

The CD40-CD154 signaling pathway has been implicated in lung injury and disease processes including fibrosis (Kaufman et al., J. Immunol. 172:1862-1871, 2004). The interaction between CD40 receptor and CD154 can trigger cellular activation, including production of proinflammatory cytokines, expression of cell adhesion molecules, and induction of cyclooxygenase 2 (COX-2), and prostaglandin E2 (PGE2) (Zhang, J. Immunol. 160:1053-1057, 1998). These events can in turn tip the balance between Type 1 and Type 2 cytokine responses in favor of Type 2 fibrogenic responses. Suppression of the CD40-CD154 pathway has been shown to have therapeutic benefit in animal lung disease models. For example, a monoclonal anti-murine CD40L antibody, MR1, was effective in protection of hyperoxic lung injury and radiation induced lung injury and fibrosis in mouse models (Adawi et al., Clin. Immunol. Immunopathol. 89:222-230, 1998; Adawi et al., Am. J. Pathol. 152:651-657, 1998). In addition, molecules which suppress the CD40-CD40L pathway such as the CXXC5 zinc finger protein, involved in epigenetic and transcriptional regulation, have been shown to ameliorate bleomycin induced lung fibrosis in mice (Cheng et al., BioMed. Research Intl. Volume 2020, Article ID 7840652, 2020; Xiong et al., J. Cell. Mol. Med. 23:740-749, 2018), indicating an important role for the CD40-CD40L pathway in disease progression.

### Tumor Necrosis Factor α (TNFα)

The tumor necrosis factor α (TNFα) cytokine is a pleotropic pro-inflammatory mediator that is produced by a variety of cell types including macrophages and monocytes. Macrophages are the major source of TNFα in the lung; however, epithelial cells, eosinophils, and mast cells also may release TNFα upon activation (Malaviya et al., Pharmacol. Ther. 180:90-98, 2017; Aggarwal et al., Blood 119:651-665, 2012). Increased levels of TNFα have been linked to several pulmonary inflammatory diseases including asthma, chronic obstructive pulmonary disease (COPD), acute lung injury (ALI)/acute respiratory distress syndrome (ARDS), sarcoidosis, and interstitial pulmonary fibrosis (IPF) (Malaviya et al., supra).

In addition to lung pathology, TNFα is increased and implicated in inflammatory bowel disease such as Crohn's disease (CD) and ulcerative colitis (UC). Monoclonal antibody therapy directed against tumor necrosis factor-alpha (anti-TNF) has revolutionized the care of patients with inflammatory bowel disease (IBD) (Pouillon et al., Expert Opinion on Biological Therapy 16:1277-1290, 2016; Peyrin-Biroulet et al., The Lancet 372:67-81, 2008).

### Transforming Growth Factor-β (TGF-β)

The TGF-β cytokine plays an important role in immune responses and regulation (Travis and Sheppard, Annu. Rev. Immunol. 32:51-82, 2014). In mammals, three major isoforms of TGF-β have been identified, TGF-β1, TGF-β2, and TGF-β3 (Fernandez and Eickelberg, Proc. Amer. Thorac. Soc. 9:111-116, 2012). Many cell types express TGF-β, but it is expressed in a non-active form complexed to other molecules and requires activation to exert its functional effects. Latent TGF-β is activated upon cleavage from a preprotein form into a cytokine with pleiotropic activities, including chemotaxis, proliferation, and stimulation of cytokines associated with inflammation. TGF-β regulates cell proliferation, differentiation, migration, adhesion, survival, epithelial-mesenchymal transition (EMT), and collagen and extracellular matrix synthesis, and is essential for angiogenesis, wound healing and immune regulation, but also can drive cancer, metastasis, diabetes, and fibrosis disease progression (Varga and Pasche, Curr. Opin. Rheum. 20:720-728, 2008). TGF-β has also been shown to be critical in regulating adaptive T cell responses and recent studies implicate TGF-β as an important mediator of fibrosis in different organs, including the lung. Clinical trials using several antibodies which target TGF-β isoforms, such as fresolimumab (GC-1008), have been completed or are underway for cancer therapy (melanoma, glioma, breast cancer, and non-small lung cancer) and fibrosis. In addition, antibodies which target integrins such as αVβ6, involved or important in TGF-β activation, are also being used in phase 2 clinical trials to determine their efficacy for idiopathic pulmonary fibrosis (IPF) (Fernandez and Eickelberg, supra).

### Expression of Enhanced DNase1 in Mammalian Cells

Wild-type DNase1, such as the FDA approved drug Pulmozyme*, is inhibited by G-actin in the lungs and plasma. An enhanced DNase1 was engineered by Genentech, creating DNase1 resistant to inhibition by actin and with increased enzyme activity (Pan and Lazarus, Prot. Sci. 8:1780-8, 1999). This enhanced DNase1, however, was toxic in mammalian cells, and no stable CHO cells could be established despite screening thousands of clones. Inducible expression in CHO cells was one potential solution that was reported (Lam et al., Biotech. Progress 32: 523-33, 2017).

### SUMMARY OF THE INVENTION

In one aspect, the present invention provides a fusion polypeptide comprising, from an amino terminal position to a carboxyl terminal position, T-L1-X-L2-P, wherein T is a first biologically active polypeptide selected from a DNase, an RNase, a superoxide dismutase 1 (SOD1), a cytotoxic T-lymphocyte associated molecule-4 (CTLA-4) extracellular domain, a CD40 extracellular domain, and a polypeptide that specifically binds and neutralizes tumor necrosis factor α (TNFα) or transforming growth factor-β(TGF-β); L1 is a first polypeptide linker, wherein L1 is optionally present; X is a immunoglobulin heavy chain constant region, wherein the immunoglobulin heavy chain constant region is capable of forming dimers and specifically binding the neonatal Fc receptor (FcRn); L2 is a second polypeptide linker, wherein L2 is optionally present; and P is a biologically active paraoxonase, wherein the paraoxonase has at least 80% or at least 90% identity with the amino acid sequence shown in residues 16-355 or 26-355 of SEQ ID NO:6 and does not contain an amino terminal leader sequence corresponding to residues 1-15 of SEQ ID NO:6. In some embodiments, the biologically active paraoxonase has at least 95% identity with the amino acid sequence shown in (i) residues 16-355 or 26-355 of SEQ ID NO:6, (ii) residues 16-355 or 26-355 of SEQ ID NO:124, or (iii) residues 16-355 or 26-355 of SEQ ID NO:126.

In another aspect, the present invention provides a fusion polypeptide comprising, from an amino terminal position to a carboxyl terminal position, T-L1-X-L2-P, wherein T is a first biologically active polypeptide selected from a DNase, an RNase, a superoxide dismutase 1 (SOD1), a cytotoxic T-lymphocyte associated molecule-4 (CTLA-4) extracellular domain, a CD40 extracellular domain, and a polypeptide that specifically binds and neutralizes tumor necrosis factor α (TNFα) or transforming growth factor-β(TGF-β); L1 is a first polypeptide linker, wherein L1 is optionally present; X is a second biologically active polypeptide, wherein X is optionally present; L2 is a second polypeptide linker, wherein L2 is optionally present; and P is a biologically active paraoxonase, wherein the paraoxonase has at least 80% or at least 90% identity with the amino acid sequence shown in residues 16-355 or 26-355 of SEQ ID NO:6 and does not contain an amino terminal leader sequence corresponding to residues 1-15 of SEQ ID NO:6. In some variations, the second biologically active polypeptide is present and is selected from a dimerizing domain and a domain that specifically binds to the neonatal Fc receptor (FcRn). In some embodiments, the biologically active paraoxonase has at least 95% identity with the amino acid sequence shown in (i) residues 16-355 or 26-355 of SEQ ID NO:6, (ii) residues 16-355 or 26-355 of SEQ ID NO:124, or (iii) residues 16-355 or 26-355 of SEQ ID NO:126.

In certain embodiments of a fusion polypeptide as above comprising a DNase as the first biologically active polypeptide and in which L1 is present, the DNase has at least 90% or at least 95% identity with the amino acid sequence shown in (i) residues 21-280 of SEQ ID NO:18, (ii) residues 21-280 of SEQ ID NO:152, or (iii) residues 21-290 of SEQ ID NO:136. In some embodiments wherein the DNase has at least 90% or at least 95% identity with the amino acid sequence shown in residues 21-280 of SEQ ID NO:18 or residues 21-280 of SEQ ID NO:152, the DNase contains at least one amino acid substitution at a position corresponding to an amino acid of human wild-type DNase1 (SEQ ID NO:120) selected from N74, G105, and A114, wherein (1) the amino acid substitution at a position corresponding to N74 of human DNase1 (N74 substitution), if present, increases DNA binding relative to human DNase1, (2) the amino acid substitution at a position corresponding to G105 of human DNase1 (G105 substitution), if present, increases DNA binding relative to human DNase1, and (3) the amino acid substitution at a position corresponding to A114 of human DNase1 (A114 substitution), if present, decreases G-actin-induced inhibition of endonuclease activity relative to human DNase1. In some variations, the fusion polypeptide contains both the N74 and G105 substitutions, both the G105 and A114 substitutions, or each of the N74, G105, and A114 substitutions. Particularly suitable amino acid substitutions at these positions are lysine at the position corresponding to N74 of human DNase1, arginine at the position corresponding to G105 of human DNase1, and/or phenylalanine at the position corresponding to A114 of human DNase1. In some embodiments wherein the fusion polypeptide contains both the N74 and G105 substitutions, the fusion polypeptide does not contain the A114 substitution. In some embodiments wherein the DNase has at least 90% or at least 95% identity with amino acid residues 21-290 of SEQ ID NO:136, each of the amino acids at positions corresponding to R80, R95, and N96 of SEQ ID NO:136 is alanine or serine. In more specific variations, the DNase has the amino acid sequence shown in (i) residues 21-280 of SEQ ID NO:18, (ii) residues 21-280 of SEQ ID NO:20, (iii) residues 21-280 of SEQ ID NO:152; (iv) residues 21-290 of SEQ ID NO:138, or (v) residues 21-290 of SEQ ID NO:140. In some embodiments of a fusion polypeptide comprising a DNase as above, L1 comprises at least 15 amino acid residues or at least 26 amino acid residues (e.g., an L1 linker consisting of from 26 to 60 amino acid residues or from 26 to 36 amino acid residues). In particular variations, L1 comprises three or more (e.g., four or more) tandem repeats of the amino acid sequence of SEQ ID NO:119; in some such embodiments, L1 has the amino acid sequence shown in SEQ ID NO:12 or SEQ ID NO:14.

In some embodiments of a fusion polypeptide as above comprising an RNase as the first biologically active polypeptide, the RNase has at least 90% or at least 95% identity with the amino acid sequence shown in residues 29-156 of SEQ ID NO:22. In more specific variations, the RNase has the amino acid sequence shown in residues 29-156 of SEQ ID NO:22. In certain embodiments, L1 is present and comprises at least two amino acid residues (e.g., an L1 linker consisting of from 10 to 36 amino acid residues). In particular variations, L1 comprises two or more tandem repeats of the amino acid sequence of SEQ ID NO:119; in some such embodiments, L1 has the amino acid sequence shown in SEQ ID NO:10 or SEQ ID NO:12.

In some embodiments of a fusion polypeptide as above comprising a SOD1 as the first biologically active polypeptide, the SOD1 has at least 90% or at least 95% identity with the amino acid sequence shown in residues 2-154 of SEQ ID NO:32. In some such embodiments, the SOD1 contains at least one of the following amino acid substitutions relative to human SOD1 (SEQ ID NO:32): alanine at the position corresponding to C7 of human SOD1 (C7 substitution), and serine at the position corresponding to C112 of human SOD1 (C112 substitution). In certain variations, the fusion polypeptide contains both the C7 and C112 substitutions. In more specific variations, the SOD1 has the amino acid sequence shown in residues 23-175 of SEQ ID NO:54. In certain embodiments, L1 is present and comprises at least two amino acid residues (e.g., an L1 linker consisting of from 10 to 36 amino acid residues). In particular variations, L1 comprises two or more tandem repeats of the amino acid sequence of SEQ ID NO:119; in some such embodiments, L1 has the amino acid sequence shown in SEQ ID NO:10 or SEQ ID NO:12.

In some embodiments of a fusion polypeptide as above comprising a CTLA-4 extracellular domain as the first biologically active polypeptide, the CTLA-4 extracellular domain has at least 90% or at least 95% identity with the amino acid sequence shown in residues 21-144 of SEQ ID NO:66. In more specific variations, the CTLA-4 extracellular domain has the amino acid sequence shown in residues 21-144 of SEQ ID NO:66.

In some embodiments of a fusion polypeptide as above comprising a CD40 extracellular domain as the first biologically active polypeptide, the CD40 extracellular domain has at least 90% or at least 95% identity with the amino acid sequence shown in (i) residues 21-188 of SEQ ID NO:74, (ii) residues 21-188 of SEQ ID NO:78, (iii) residues 21-188 of SEQ ID NO:82, (iv) residues 21-188 of SEQ ID NO:86, or (v) residues 21-188 of SEQ ID NO:90. In some such embodiments, the CD40 extracellular domain contains at least one amino acid substitution at a position corresponding to an amino acid of human CD40 (SEQ ID NO:68) selected from E64, K81, P85, and L121, wherein the at least one amino acid substitution increases CD40 ligand binding relative to human CD40. Particularly suitable amino acid substitutions at these positions are tyrosine at the position corresponding to E64 of human CD40, threonine, histidine, or serine at the position corresponding to K81 of human CD40, tyrosine at the position corresponding to P85 of human CD40, and/or proline at the position corresponding to L121 of human CD40. In some variations, the amino acid at the position corresponding to K81 of human CD40 is selected from threonine, histidine, and serine; the amino acid at the position corresponding to K81 of human CD40 is histidine and the amino acid at the position corresponding to L121 of human CD40 is proline; or the amino acid at the position corresponding to E64 of human CD40 is tyrosine, the amino acid at the position corresponding to K81 of human CD40 is threonine, and the amino acid at the position corresponding to P85 of human CD40 is tyrosine. In more specific variations, the CD40 extracellular domain has the amino acid sequence shown in (i) residues 21-188 of SEQ ID NO:74, (ii) residues 21-188 of SEQ ID NO:78, (iii) residues 21-188 of SEQ ID NO:82, (iv) residues 21-188 of SEQ ID NO:86, or (v) residues 21-188 of SEQ ID NO:90.

In certain embodiments of a fusion polypeptide as above, the first biologically active polypeptide is the polypeptide that specifically binds and neutralizes TNFα or TGF-β. In some such embodiments, the first biologically active polypeptide is a single-chain antibody such as, e.g., a single-chain Fv (scFv) or a single-domain antibody (sdAb).

In some embodiments of a fusion polypeptide as above comprising a single-chain antibody that specifically binds and neutralizes TNFα, the single-chain antibody comprises a VH domain comprising complementarity determining regions (CDRs) CDR-H1_{TNFα}, CDR-H2_{TNFα}, and CDR-H3_{TNFα}, wherein the set of VH CDRs has three or fewer amino acid substitutions relative to a set of reference CDRs CDR-H1, CDR-H2, and CDR-H3 of the VH domain having the amino acid sequence shown in SEQ ID NO:108 (e.g., a set of VH CDRs having zero amino acid substitutions relative to CDR-H1, CDR-H2, and CDR-H3 of SEQ ID NO:108, whereby CDR-H1_{TNFα}, CDR-H2_{TNFα}, and CDR-H3_{TNFα} are, respectively, a CDR-H1, a CDR-H2, and a CDR-H3 of SEQ ID NO:108). Each VH CDR may be defined, for example, according to the Chothia definition, the Kabat definition, the AbM definition, the IMGT database definition, or the contact definition of CDR. In some variations, each VH CDR is defined according the IMGT database definition of CDR, whereby the reference CDRs CDR-H1, CDR-H2, and CDR-H3 of SEQ ID NO:108 correspond to residues 26-33, 50-59, and 97-110 of SEQ ID NO:108, respectively; in some such embodiments, CDR-H1_{TNFα}, CDR-H2_{TNFα}, and CDR-H3_{TNFα} are the VH CDRs of SEQ ID NO:108 according to the IMGT database definition, whereby CDR-H1_{TNFα} a has the amino acid sequence shown in residues 26-33 of SEQ ID NO:108, CDR-H2_{TNFα} has the amino acid sequence shown in residues 50-59 of SEQ ID NO:108, and CDR-H3_{TNFα} has the amino acid sequence shown in residues 97-110 of SEQ ID NO:108.

In other, non-mutually exclusive embodiments of a fusion polypeptide as above comprising a single-chain antibody that specifically binds and neutralizes TNFα, the single-chain antibody comprises a VL domain comprising complementarity determining regions (CDRs) CDR-L1_{TNFα}, CDR-L2_{TNFα}, and CDR-L3_{TNFα}, wherein the set of VL CDRs has three or fewer amino acid substitutions relative to a set of reference CDRs CDR-L1, CDR-L2, and CDR-L3 of the VL domain having the amino acid sequence shown in SEQ ID NO: 110 (e.g., a set of VL CDRs having zero amino acid substitutions relative to CDR-L1, CDR-L2, and CDR-L3 of SEQ ID NO:110, whereby CDR-L1_{TNFα}, CDR-L2_{TNFα}, and CDR-L3_{TNFα} are, respectively, a CDR-L1, a CDR-L2, and a CDR-L3 of SEQ ID NO:110). Each VL CDR may be defined, for example, according to the Chothia definition, the Kabat definition, the AbM definition, the IMGT database definition, or the contact definition of CDR. In some variations, each VL CDR is defined according to the IMGT database definition of CDR, wherein the reference CDRs CDR-L1, CDR-L2, and CDR-L3 of SEQ ID NO:110 correspond to residues 27-32, 50-52, and 89-97 of SEQ ID NO:110, respectively; in some such embodiments, CDR-L1_{TNFα}, CDR-L2_{TNFα}, and CDR-L3_{TNFα} are VL CDRs of SEQ ID NO:110 according to the IMGT database definition, whereby CDR-L1_{TNFα} has the amino acid sequence shown in residues 27-32 of SEQ ID NO:110; CDR-L2_{TNFα} has the amino acid sequence shown in residues 50-52 of SEQ ID NO:110; and CDR-L3_{TNFα} has the amino acid sequence shown in residues 89-97 of SEQ ID NO:110.

In some embodiments of a fusion polypeptide as above comprising a single-chain antibody that specifically binds and neutralizes TNFα, the single-chain antibody comprises a VH domain having at least 90% or at least 95% identity with the amino acid sequence shown in SEQ ID NO:108, and/or the single-chain antibody comprises a VL domain having at least 90% or at least 95% identity with the amino acid sequence shown in SEQ ID NO:110. In more specific variations, the single-chain antibody comprises a VH domain having the amino acid sequence shown in SEQ ID NO:108, and/or the single-chain antibody comprises a VL domain having the amino acid sequence shown in SEQ ID NO:110.

In some embodiments of a fusion polypeptide as above comprising a single-chain antibody that specifically binds and neutralizes TGF-β, the single-chain antibody comprises a VH domain comprising complementarity determining regions (CDRs) CDR-H1_{TGF-β}, CDR-H2_{TGF-β}, AND CDR-H3_{TGF-β}, WHEREIN THE SET OF VH CDRS HAS THREE OR FEWER amino acid substitutions relative to a set of reference CDRs CDR-H1, CDR-H2, and CDR-H3 of the VH domain having the amino acid sequence shown in SEQ ID NO: 112 (e.g., a set of VH CDRs having zero amino acid substitutions relative to CDR-H1, CDR-H2, and CDR-H3 of SEQ ID NO:112, whereby CDR-H1_{TGF-β}, CDR-H2_{TGF-β}, and CDR-H3_{TGF-β} are, respectively, a CDR-H1, a CDR-H2, and a CDR-H3 of SEQ ID NO:112). Each VH CDR may be defined, for example, according to the Chothia definition, the Kabat definition, the AbM definition, the IMGT database definition, or the contact definition of CDR. In some variations, each VH CDR is defined according the Kabat definition of CDR, whereby the reference CDRs CDR-H1, CDR-H2, and CDR-H3 of SEQ ID NO: 112 correspond to residues 31-35, 50-66, and 99-106 of SEQ ID NO: 112, respectively, in some such embodiments, CDR-H1_{TGF-β}, CDR-H2_{TGF-β}, and CDR-H3_{TGF-β} are the VH CDRs of SEQ ID NO:112 according to the Kabat definition, whereby CDR-H1_{TGF-β} has the amino acid sequence shown in residues 31-35 of SEQ ID NO:112, CDR-H2_{TGF-β} has the amino acid sequence shown in residues 50-66 of SEQ ID NO:112, and CDR-H3_{TGF-β} has the amino acid sequence shown in residues 99-106 of SEQ ID NO: 112.

In other, non-mutually exclusive embodiments of a fusion polypeptide as above comprising a single-chain antibody that specifically binds and neutralizes TGF-β, the single-chain antibody comprises a VL domain comprising complementarity determining regions (CDRs) CDR-L1_{TGF-β}, CDR-L2_{TGF-β}, and CDR-L3_{TGF-β}, wherein the set of VL CDRs has three or fewer amino acid substitutions relative to a set of reference CDRs CDR-L1, CDR-L2, and CDR-L3 of the VL domain having the amino acid sequence shown in SEQ ID NO:114 (e.g., a set of VL CDRs having zero amino acid substitutions relative to CDR-L1, CDR-L2, and CDR-L3 of SEQ ID NO:114, whereby CDR-L2_{TGF-β}, CDR-L2_{TGF-β}, and CDR-L3_{TGF-β} are, respectively, a CDR-L1, a CDR-L2, and a CDR-L3 of SEQ ID NO:114). Each VL CDR may be defined, for example, according to the Chothia definition, the Kabat definition, the AbM definition, the IMGT database definition, or the contact definition of CDR. In some variations, each VL CDR is defined according to the Kabat definition of CDR, wherein the reference CDRs CDR-L1, CDR-L2, and CDR-L3 of SEQ ID NO: 114 correspond to residues 24-40, 56-62, and 95-102 of SEQ ID NO: 114, respectively; in some such embodiments, CDR-L2_{TGF-β}, CDR-L2_{TGF-β}, and CDR-L3_{TGF-β} are VL CDRs of SEQ ID NO: 14 according to the Kabat definition, whereby CDR-L1_{TGF-β} has the amino acid sequence shown in residues 24-40 of SEQ ID NO:114; CDR-L2_{TGF-β} has the amino acid sequence shown in residues 56-62 of SEQ ID NO:114; and CDR-L3_{TGF-β} has the amino acid sequence shown in residues 95-102 of SEQ ID NO:114.

In some embodiments of a fusion polypeptide as above comprising a single-chain antibody that specifically binds and neutralizes TGF-β, the single-chain antibody comprises a VH domain having at least 90% or at least 95% identity with the amino acid sequence shown in SEQ ID NO:112, and/or the single-chain antibody comprises a VL domain having at least 90% or at least 95% identity with the amino acid sequence shown in SEQ ID NO:114. In more specific variations, the single-chain antibody comprises a VH domain having the amino acid sequence shown in SEQ ID NO:112, and/or the single-chain antibody comprises a VL domain having the amino acid sequence shown in SEQ ID NO:114.

In some embodiments of a fusion polypeptide as above comprising a single-chain antibody that specifically binds and neutralizes TNFα or TGF-β, the single-chain antibody has at least 90% or at least 95% identity with the amino acid sequence shown in (i) residues 21-268 of SEQ ID NO:92, (ii) residues 21-268 of SEQ ID NO:96, (iii) residues 21-269 of SEQ ID NO:100, or (iv) residues 21-269 of SEQ ID NO:104. In more specific variations, the single-chain antibody has the amino acid sequence shown in (i) residues 21-268 of SEQ ID NO:92, (ii) residues 21-268 of SEQ ID NO:96, (iii) residues 21-269 of SEQ ID NO:100, or (iv) residues 21-269 of SEQ ID NO:104.

In another aspect, the present invention provides a fusion polypeptide comprising, from an amino terminal position to a carboxyl terminal position, X-L2-P, wherein X is an immunoglobulin heavy chain constant region, wherein the immunoglobulin heavy chain constant region is capable of forming dimers and specifically binding the neonatal Fc receptor (FcRn); L2 is a polypeptide linker, wherein L2 is optionally present; and P is a biologically active paraoxonase, wherein the paraoxonase has at least 80% or at least 90% identity with the amino acid sequence shown in residues 16-355 or 26-355 of SEQ ID NO:6 and does not contain an amino terminal leader sequence corresponding to residues 1-15 of SEQ ID NO:6, wherein the fusion polypeptide does not comprise a biologically active polypeptide N-terminal to the immunoglobulin heavy chain constant region. In some embodiments, the biologically active paraoxonase has at least 95% identity with the amino acid sequence shown in (i) residues 16-355 or 26-355 of SEQ ID NO:6, (ii) residues 16-355 or 26-355 of SEQ ID NO:124, or (iii) residues 16-355 or 26-355 of SEQ ID NO:126.

In some embodiments of a fusion polypeptide as above comprising the formula T-L1-X-L2-P or X-L2-P, the amino acid at a position corresponding to Q192 of the human paraoxonase 1 Q192 isoform (hPON1-Q192; SEQ ID NO:4) is lysine or arginine. In other, non-mutually exclusive variations, the amino acid at the position corresponding to H115 of hPON1-Q192 is tryptophan. In specific variations, the paraoxonase has an amino acid sequence selected from (i) residues n-355 of SEQ ID NO:6, (ii) residues n-355 of SEQ ID NO:124, and (iii) residues n-355 of SEQ ID NO:126, wherein n is an integer from 16 to 26, inclusive.

In certain embodiments of a fusion polypeptide as above comprising the formula T-L1-X-L2-P or X-L2-P, L2 is present and comprises at least eight amino acid residues. In some such embodiments, L2 consists of from 12 to 25 amino acid residues. A particularly suitable L2 linker has the amino acid sequence shown in SEQ ID NO:56.

In some embodiments of a fusion polypeptide as above having the formula T-L1-X-L2-P or X-L2-P and wherein X is an immunoglobulin heavy chain constant region, the immunoglobulin heavy chain constant region is an immunoglobulin Fc region. In some such embodiments, the Fc region is a human Fc region such as, e.g., a human Fc variant comprising one or more (e.g., from one to 10) amino acid substitutions relative to the wild-type human sequence. Particularly suitable Fc regions include human γ1 and γ4 Fc regions. In some variations, the Fc region is a human γ1 Fc variant in which Eu residue C220 is replaced by serine; in some such embodiments Eu residues C226 and C229 are each replaced by serine, and/or Eu residue P238 is replaced by serine. In further variations comprising an Fc region as above, the Fc region is a human γl Fc variant in which Eu residue P331 is replaced by serine. In some embodiments, the Fc region has at least 90% or at least 95% identity with the amino acid sequence shown in (i) residues 1-232 or 1-231 of SEQ ID NO:26, (ii) residues 1-232 or 1-231 of SEQ ID NO:28, (iii) residues 159-390 or 159-389 of SEQ ID NO:42, (iv) residues 1-232 or 1-231 of SEQ ID NO: 116, or (v) residues 1-232 or 1-231 of SEQ ID NO:118. In more specific variations, the Fc region has the amino acid sequence shown in (i) residues 1-232 or 1-231 of SEQ ID NO:26, (ii) residues 1-232 or 1-231 of SEQ ID NO:28, (iii) residues 159-390 or 159-389 of SEQ ID NO:42, (iv) residues 1-232 or 1-231 of SEQ ID NO:116, or (v) residues 1-232 or 1-231 of SEQ ID NO:118.

In some embodiments of a fusion polypeptide as above having the formula T-L1-X-L2-P or X-L2-P and wherein X is an immunoglobulin heavy chain constant region, the immunoglobulin heavy chain constant region comprises an amino acid sequence having at least 90% or at least 95% identity with the amino acid sequence shown in (i) residues 16-232 or 16-231 of SEQ ID NO:26, (ii) residues 16-232 or 16-231 of SEQ ID NO: 116, or (iii) residues 16-232 or 16-231 of SEQ ID NO: 118. In more specific variations, the immunoglobulin heavy chain constant region comprises the amino acid sequence shown in (i) residues 16-232 or 16-231 of SEQ ID NO:26, (ii) residues 16-232 or 16-231 of SEQ ID NO:116, or (iii) residues 16-232 or 16-231 of SEQ ID NO:118.

In some embodiments of a fusion polypeptide as above having the formula T-L1-X-L2-P and wherein the first biologically active polypeptide is the DNase, the fusion polypeptide comprises an amino acid sequence having at least 90% or at least 95% identity with the amino acid sequence shown in (i) residues 21-896 of SEQ ID NO:2, (ii) residues 21-896 of SEQ ID NO:128, (iii) residues 21-896 of SEQ ID NO:130, (iv) residues 21-906 of SEQ ID NO:148, (v) residues 21-896 of SEQ ID NO:158, (vi) residues 21-906 of SEQ ID NO:160, (vii) residues 21-906 of SEQ ID NO:162, or (viii) residues 21-906 of SEQ ID NO:164; in some such embodiments, the fusion polypeptide comprises the amino acid sequence shown in (i) residues 21-896 of SEQ ID NO:2, (ii) residues 21-896 of SEQ ID NO:128, (iii) residues 21-896 of SEQ ID NO:130, (iv) residues 21-906 of SEQ ID NO:148, (v) residues 21-896 of SEQ ID NO:158, (vi) residues 21-906 of SEQ ID NO:160, (vii) residues 21-906 of SEQ ID NO:162, or (viii) residues 21-906 of SEQ ID NO:164.

In some embodiments of a fusion polypeptide as above having the formula T-L1-X-L2-P and wherein the first biologically active polypeptide is the DNase, the polypeptide region corresponding to T-L1-X (“T-L1-X region”) comprises the amino acid sequence shown in SEQ ID NO:149, SEQ ID NO:150, SEQ ID NO:153, or SEQ ID NO:154. In certain variations of a T-L1-X region comprising the amino acid sequence shown in SEQ ID NO:149, SEQ ID NO:150, SEQ ID NO:153, or SEQ ID NO:154, (i) the amino acid at each of positions 315 and 316 is alanine; (ii) the amino acid at position 319 is serine; (iii) the amino acid at position 378 is alanine, glutamine, or glycine, and the amino acid at position is optionally alanine; (iv) the amino acid at position 410 is alanine, glycine, or serine; (v) the amino acid at position 412 is serine or alanine; (vi) the amino acid at position 333 is tyrosine, the amino acid at position 335 is threonine, and the amino acid at position 337 is glutamate; and/or (vii) the amino acid at position 509 is leucine, and the amino acid at position 515 is serine. In other, non-mutually exclusive variations of a T-L1-X region comprising the amino acid sequence shown in SEQ ID NO:149, SEQ ID NO:150, SEQ ID NO:153, or SEQ ID NO:154, the amino acid sequence corresponding to positions 261-296 includes a Gly-Ser tandem repeat sequence having a formula selected from the group consisting of (i) [Gly-Gly-Gly-Gly-Ser]ₙ, wherein n is an integer from 4 to 7, (ii) [Gly-Gly-Gly-Ser]ₙ, wherein n is an integer from 5 to 9, and (iii) [Gly-Gly-Ser]ₙ, wherein n is an integer from 6 to 12; in some such embodiments, the amino acid sequence corresponding to positions 261-296 includes a Gly-Ser tandem repeat sequence having a formula selected from the group consisting of (i) [Gly-Gly-Gly-Gly-Ser]ₙ, wherein n is an integer from 4 to 6, (ii) [Gly-Gly-Gly-Ser]ₙ, wherein n is an integer from 5 to 7, and (iii) [Gly-Gly-Ser]ₙ, wherein n is an integer from 6 to 10. In yet other, non-mutually exclusive variations of a T-L1-X region comprising the amino acid sequence shown in SEQ ID NO:149, SEQ ID NO:150, SEQ ID NO:153, or SEQ ID NO:154, the amino acid at position 261 is aspartate, the amino acid at position 262 is leucine, the amino acid at position 263 is serine, the amino acid at position is threonine, the amino acid at position is 295 is glycine, and/or the amino acid at position 296 is leucine. In other, non-mutually exclusive variations of a T-L1-X region comprising the amino acid sequence shown in SEQ ID NO:149 or SEQ ID NO:153, the amino acid at each of positions 74 and 105 is independently lysine or arginine. In other, non-mutually exclusive variations of a T-L1-X region comprising the amino acid sequence shown in SEQ ID NO:149, the amino acid at position 114 is phenylalanine.

In some embodiments of a fusion polypeptide as above having the formula T-L1-X-L2-P and wherein the first biologically active polypeptide is the RNase, the fusion polypeptide comprises an amino acid sequence having at least 90% or at least 95% identity with the amino acid sequence shown in (i) residues 21-764 of SEQ ID NO:40 or (ii) residues 21-740 of SEQ ID NO:48; in some such embodiments, the fusion polypeptide comprises the amino acid sequence shown in (i) residues 21-764 of SEQ ID NO:40 or (ii) residues 21-740 of SEQ ID NO:48.

In some embodiments of a fusion polypeptide as above having the formula T-L1-X-L2-P and wherein the first biologically active polypeptide is the SOD1, the fusion polypeptide comprises an amino acid sequence having at least 90% or at least 95% identity with the amino acid sequence shown in (i) residues 23-781 of SEQ ID NO:50, (ii) residues 23-781 of SEQ ID NO:52, or (iii) residues 23-791 of SEQ ID NO:54; in some such embodiments, the fusion polypeptide comprises the amino acid sequence shown in (i) residues 23-781 of SEQ ID NO:50, (ii) residues 23-781 of SEQ ID NO:52, or (iii) residues 23-791 of SEQ ID NO:54.

In some embodiments of a fusion polypeptide as above having the formula T-L1-X-L2-P and wherein the first biologically active polypeptide is the CTLA-4 extracellular domain, the fusion polypeptide comprises an amino acid sequence having at least 90% or at least 95% identity with the amino acid sequence shown in residues 21-736 of SEQ ID NO:66; in some such embodiments, the fusion polypeptide comprises the amino acid sequence shown in residues 21-736 of SEQ ID NO:66.

In some embodiments of a fusion polypeptide as above having the formula T-L1-X-L2-P and wherein the first biologically active polypeptide is the CD40 extracellular domain, the fusion polypeptide comprises an amino acid sequence having at least 90% or at least 95% identity with the amino acid sequence shown in (i) residues 21-804 of SEQ ID NO:74, (ii) residues 21-804 of SEQ ID NO:78, (iii) residues 21-804 of SEQ ID NO:82, (iv) residues 21-804 of SEQ ID NO:86, or (v) residues 21-804 of SEQ ID NO:90; in some such embodiments, the fusion polypeptide comprises the amino acid sequence shown in (i) residues 21-804 of SEQ ID NO:74, (ii) residues 21-804 of SEQ ID NO:78, (iii) residues 21-804 of SEQ ID NO:82, (iv) residues 21-804 of SEQ ID NO:86, or (v) residues 21-804 of SEQ ID NO:90.

In some embodiments of a fusion polypeptide as above having the formula T-L1-X-L2-P and wherein the first biologically active polypeptide is a single-chain antibody that specifically binds and neutralizes TNFα, the fusion polypeptide comprises an amino acid sequence having at least 90% or at least 95% identity with the amino acid sequence shown in (i) residues 21-860 of SEQ ID NO:94, or (ii) residues 21-860 of SEQ ID NO:98; in some such embodiments, the fusion polypeptide comprises the amino acid sequence shown in (i) residues 21-860 of SEQ ID NO:94, or (ii) residues 21-860 of SEQ ID NO:98.

In some embodiments of a fusion polypeptide as above having the formula T-L1-X-L2-P and wherein the first biologically active polypeptide is a single-chain antibody that specifically binds and neutralizes TGF-β, the fusion polypeptide comprises an amino acid sequence having at least 90% or at least 95% identity with the amino acid sequence shown in (i) residues 21-861 of SEQ ID NO:102, or (ii) residues 21-861 of SEQ ID NO:106; in some such embodiments, the fusion polypeptide comprises the amino acid sequence shown in (i) residues 21-861 of SEQ ID NO:102, or (ii) residues 21-861 of SEQ ID NO:106.

In some embodiments of a fusion polypeptide as above having the formula X-L2-P, the fusion polypeptide comprises an amino acid sequence having at least 90% or at least 95% identity with the amino acid sequence shown in (i) residues 21-613 of SEQ ID NO:122, (ii) residues 21-613 of SEQ ID NO:132, (iii) residues 21-613 of SEQ ID NO:134, (iv) residues 21-610 of SEQ ID NO:142, (v) residues 21-610 of SEQ ID NO:144, or (vi) residues 21-610 of SEQ ID NO:146; in some such embodiments, the fusion polypeptide comprises the amino acid sequence shown in (i) residues 21-613 of SEQ ID NO:122, (ii) residues 21-613 of SEQ ID NO:132, (iii) residues 21-613 of SEQ ID NO:134, (iv) residues 21-610 of SEQ ID NO:142, (v) residues 21-610 of SEQ ID NO:144, or (vi) residues 21-610 of SEQ ID NO:146.

In another aspect, the present invention provides a dimeric protein comprising a first fusion polypeptide and a second fusion polypeptide, wherein each of the first and second fusion polypeptides is (i) a fusion polypeptide as above having the formula T-L1-X-L2-P and wherein the X is a dimerizing domain (e.g., an immunoglobulin heavy chain constant region such as, for example, an immunoglobulin Fc region), or (ii) a fusion polypeptide as above having the formula X-L2-P.

In another aspect, the present invention provides a polynucleotide encoding a fusion polypeptide as described above.

In still another aspect, the present invention provides an expression cassette comprising a DNA segment encoding a fusion polypeptide as described above and which is operably linked to a promoter. In particular variations, the encoded fusion polypeptide is a fusion polypeptide as above having the formula T-L1-X-L2-P and wherein the first biologically active polypeptide is the DNase (i) having at least 90% or at least 95% identity with amino acid residues 21-280 of SEQ ID NO:18 or residues 21-280 of SEQ ID NO:152 and (ii) comprising at least one of the N74, G105, and A114 substitutions (e.g., a DNase having two or all three substitutions). Also provided is a cultured cell into which has been introduced an expression cassette as described above, wherein the cell expresses the DNA segment. In a related aspect, the present invention provides a stable cell line comprising, within its genomic DNA, an expression cassette as described above, wherein the stable cell line constitutively expresses the DNA segment. In some embodiments, the stable cell line is a Chinese hamster ovary (CHO) cell line.

In another aspect, the present invention provides a vector comprising an expression cassette as described above.

In another aspect, the present invention provides a method of making a fusion polypeptide. The method generally includes (i) culturing a cell into which has been introduced an expression cassette as described above, wherein the cell expresses the DNA segment and the encoded fusion polypeptide is produced, and (ii) recovering the fusion polypeptide. In some variations, the cultured cell is a stable cell line as described above.

In yet another aspect, the present invention provides a method of making a dimeric protein. The method generally includes (i) culturing a cell into which has been introduced an expression cassette as described above, wherein the cell expresses the DNA segment and the encoded fusion polypeptide is produced as a dimeric protein, and (ii) recovering the dimeric protein. In some variations, the cultured cell is a stable cell line as described above.

In another aspect, the present invention provides a composition comprising a fusion polypeptide as described above and a pharmaceutically acceptable carrier.

In another aspect, the present invention provides a composition comprising a dimeric protein as described above and a pharmaceutically acceptable carrier.

In some embodiments of a composition as described above, the composition is formulated for delivery to the lung by nebulization.

In still another aspect, the present invention provides a method for treating an inflammatory disease. The method generally includes administering to a subject having the inflammatory disease an effective amount of a fusion polypeptide or dimeric protein as described above. In some embodiments, the inflammatory disease is an inflammatory lung disease such as, for example, chronic obstructive pulmonary disease (COPD), asthma, cystic fibrosis (CF), bronchiectasis, hypoxia, acute respiratory distress syndrome (ARDS) (e.g., COVID-19-associated ARDS), and interstitial lung disease (e.g., idiopathic pulmonary fibrosis (IPF) or sarcoidosis); in some variations, the inflammatory lung disease is characterized by Pseudonionas aeruginosa infection. In other embodiments, the inflammatory disease is selected from an inflammatory bowel disease (IBD) (e.g., Crohn's disease or ulcerative colitis), systemic lupus erythematosus (SLE) (e.g., SLE with lupus nephritis), type 1 diabetes, and type 2 diabetes. In still other embodiments, the inflammatory disease is an inflammatory skin disease such as, for example, psoriasis or atopic dermatitis.

In another aspect, the present invention provides a method for treating an autoimmune disease. The method generally includes administering to a subject having the autoimmune disease an effective amount of a fusion polypeptide or dimeric protein as described above. In some embodiments, the autoimmune disease is selected from systemic lupus erythematosus (SLE) (e.g., SLE with lupus nephritis), Sjogren's syndrome, rheumatoid arthritis, psoriasis, psoriatic arthritis, antiphospholipid syndrome, type 1 diabetes, vasculitis, and systemic sclerosis.

In another aspect, the present invention provides a method for treating biofilm formation by a gram-negative bacteria. The method generally includes administering to a subject having the biofilm formation an effective amount of a fusion polypeptide or dimeric protein as described above. In some embodiments, the gram-negative bacteria is Pseudomonas aeruginosa.

In another aspect, the present invention provides a method for treating exposure to sulfur mustard gas or an organophosphate. The method generally includes administering to a subject exposed to the sulfur mustard gas or to the organophosphate an effective amount of a fusion polypeptide or dimeric protein as described above. In some embodiments, the organophosphate is an insecticide selected from parathion, malathion, chlorpyrifos, diazinon, dichlorvos, phosmet, fenitrothion, terbufos, tetrachlorvinphos, azamethiphos, and azinphos-methyl. In some embodiments the organophosphate is a nerve agent selected from tabun, sarin, soman, and cyclosarin.

In yet another aspect, the present invention provides a method for treating a neurological disease. The method generally includes administering to a subject having the neurological disease an effective amount of a fusion polypeptide or dimeric protein as described above. In some embodiments, the neurological disease is selected from Parkinson's disease and Alzheimer's disease. In some embodiments, the neurological disease is a disease characterized by dementia such as, for example, Alzheimer's disease.

In another aspect, the present invention provides a method for treating a cardiovascular disease. The method generally includes administering to a subject having the cardiovascular disease an effective amount of a fusion polypeptide or dimeric protein as described above. In some embodiments, the cardiovascular disease is a disease characterized by atherosclerosis such as, e.g., coronary heart disease or ischemic stroke. In some variations, the coronary heart disease is characterized by acute coronary syndrome.

In another aspect, the present invention provides a method for treating a chronic liver disease. The method generally includes administering to a subject having the chronic liver disease an effective amount of a fusion polypeptide or dimeric protein as describe above. In some embodiments, the chronic liver disease is selected from nonalcoholic fatty liver disease (NAFLD), alcohol-associated liver disease (ALD), portal hypertension, or a complication following liver transplantation. In some variations, the nonalcoholic fatty liver disease is nonalcoholic steatohepatitis (NASH).

In another aspect, the present invention provides a method for treating a fibrotic disease. The method generally includes administering to a subject having the fibrotic disease an effective amount of a fusion polypeptide or dimeric protein as described above. In some embodiments, the fibrotic disease is selected from the group consisting of systemic sclerosis, systemic lupus erythematosus (SLE), an inflammatory lung disease, a chronic liver disease, and a chronic kidney disease (e.g., lupus nephritis, IgA nephropathy, or membranous glomerulonephritis).

In still another aspect, the present invention provides a method for treating a disease or disorder characterized by NETosis. In some embodiments, the method generally includes administering to a subject having the disease or disorder characterized by NETosis an effective amount of a fusion polypeptide having the formula T-L1-X-L2-P as described above, or a dimeric protein formed by dimerization of the fusion polypeptide, wherein the fusion polypeptide or dimeric protein comprises the DNase as the first biologically active polypeptide.

In other embodiments of a method for treating a disease or disorder characterized by NETosis, the method is a combination therapy that generally includes administering to a subject having the disease or disorder characterized by NETosis (a) an effective amount of a fusion polypeptide having the formula X-L2-P as described above, or a dimeric protein formed by dimerization of the fusion polypeptide, and (b) an effective amount of a biologically active DNase. In certain embodiments of the combination therapy method, the DNase has at least 90% or at least 95% identity with the amino acid sequence shown in (i) residues 21-280 of SEQ ID NO:18, (ii) residues 21-280 of SEQ ID NO:152, or (iii) residues 21-290 of SEQ ID NO:136. In some embodiments wherein the DNase has at least 90% or at least 95% identity with the amino acid sequence shown in residues 21-280 of SEQ ID NO:18 or residues 21-280 of SEQ ID NO:152, the DNase contains at least one amino acid substitution at a position corresponding to an amino acid of human wild-type DNase1 (SEQ ID NO:120) selected from N74, G105, and A114, wherein (1) the amino acid substitution at a position corresponding to N74 of human DNase1 (N74 substitution), if present, increases DNA binding relative to human DNase1, (2) the amino acid substitution at a position corresponding to G105 of human DNase1 (G105 substitution), if present, increases DNA binding relative to human DNase1, and (3) the amino acid substitution at a position corresponding to A114 of human DNase1 (A114 substitution), if present, decreases G-actin-induced inhibition of endonuclease activity relative to human DNase1. In some variations, the DNase contains both the N74 and G105 substitutions, or each of the N74, G105, and A114 substitutions. Particularly suitable amino acid substitutions at these positions are lysine at the position corresponding to N74 of human DNase1, arginine at the position corresponding to G105 of human DNase1, and/or phenylalanine at the position corresponding to A114 of human DNase1. In some embodiments wherein the DNase has at least 90% or at least 95% identity with amino acid sequence shown in residues 21-290 of SEQ ID NO:136, each of the amino acids at positions corresponding to R80, R95, and N96 of SEQ ID NO:136 is alanine or serine. In more specific variations, the DNase has the amino acid sequence shown in (i) residues 21-280 of SEQ ID NO:18, (ii) residues 21-280 of SEQ ID NO:20, (iii) residues 21-280 of SEQ ID NO:152, (iv) residues 21-290 of SEQ ID NO:138, or (v) residues 21-290 of SEQ ID NO:140.

In certain embodiments of a combination therapy method for treating a disease or disorder characterized by NETosis as above, the DNase is contained within a DNase fusion polypeptide comprising, from an amino terminal position to a carboxyl terminal position, D-L1-X_{d}, wherein D is the DNase, L1 is a polypeptide linker (e.g., a linker comprising at least 15 amino acid residues or at least 26 amino acid residues), and X_{d} is an immunoglobulin Fc region. The immunoglobulin Fc region may be an Fc region as described above for a fusion polypeptide having the formula T-L1-X-L2-P or X-L2-P. In some variations, the DNase fusion polypeptide comprises an amino acid sequence having at least 90% or at least 95% identity with the amino acid sequence shown in (i) residues 21-538 or 21-537 of SEQ ID NO:60, (ii) residues 21-548 or 21-547 of SEQ ID NO:62, (iii) residues 21-538 or 21-537 of SEQ ID NO:155, (iv) residues 21-548 or 21-547 of SEQ ID NO:156, (v) residues 21-548 or 21-547 of SEQ ID NO:138, or (vi) residues 21-548 or 21-547 of SEQ ID NO:140; in some such embodiments, the DNase fusion polypeptide comprises the amino acid sequence shown in (i) residues 21-538 or 21-537 of SEQ ID NO:60, (ii) residues 21-548 or 21-547 of SEQ ID NO:62, (iii) residues 21-538 or 21-537 of SEQ ID NO:155, (iv) residues 21-548 or 21-547 of SEQ ID NO:156, (v) residues 21-548 or 21-547 of SEQ ID NO:138, or (vi) residues 21-548 or 21-547 of SEQ ID NO:140. In certain variations, the DNase comprises the amino acid sequence shown in SEQ ID NO:149, SEQ ID NO:150, SEQ ID NO:153, or SEQ ID NO:154.

In some embodiments of a method for treating a disease or disorder characterized by NETosis, the disease or disorder is an inflammatory lung disease such as, for example, chronic obstructive pulmonary disease (COPD), asthma, cystic fibrosis (CF), bronchiectasis, hypoxia, acute respiratory distress syndrome (ARDS) (e.g., COVID-19-associated ARDS), or interstitial lung disease (e.g., idiopathic pulmonary fibrosis (IPF) or sarcoidosis). In other embodiments, the disease or disorder is an inflammatory skin disease such as, for example, psoriasis or atopic dermatitis. In other embodiments, the disease or disorder is an autoimmune disease such as, e.g., systemic lupus erythematosus (SLE) (e.g., SLE with lupus nephritis), rheumatoid arthritis (RA), psoriasis, antiphospholipid syndrome, type 1 diabetes mellitus, vasculitis, or systemic sclerosis. In other embodiments, the disease or disorder is an autoinflammatory disease such as, for example, an inflammatory bowel disease (IBD) (e.g., Crohn's disease or ulcerative colitis) or gout. In other embodiments, the disease or disorder is a neurological disease or disorder such as, for example, a chronic neurodegenerative disease (e.g., Alzheimer's disease or multiple sclerosis), a central nervous system infection (e.g., meningitis, encephalitis, or cerebral malaria), or ischemic stroke. In other embodiments, the disease or disorder is a metabolic disease such as, e.g., type 2 diabetes or obesity. In other embodiments, the disease or disorder is a cardiovascular disease such as, for example, a cardiovascular disease characterized by atherosclerosis (e.g., coronary heart disease or ischemic stroke). In still other embodiments, the disease or disorder is selected from thrombosis, sepsis, and ischemia reperfusion. In other embodiments, the disease or disorder is a chronic liver disease such as, e.g., nonalcoholic steatohepatitis (NASH). In yet other embodiments, the disease or disorder is a fibrotic disease such as, e.g., systemic sclerosis, systemic lupus erythematosus (SLE), an inflammatory lung disease, a chronic liver disease, or a chronic kidney disease (e.g., lupus nephritis, IgA nephropathy, or membranous glomerulonephritis).

In yet other embodiments of a method for treating a disease or disorder characterized by NETosis, the disease or disorder is a cancer. In certain variations, the cancer treatment is a combination therapy. In some combination therapy embodiments, the combination therapy includes an immunomodulatory therapy comprising an anti-PD-1/PD-L1 therapy, an anti-CTLA-4 therapy, a CAR T cell therapy, or a combination thereof (e.g., both an anti-PD-1/PD-L1 therapy and an anti-CTLA-4 therapy). In other combination therapy embodiments, the combination therapy includes radiation therapy or chemotherapy. In some combination therapy embodiments, the combination therapy includes a targeted therapy: in some such embodiments, the targeted therapy includes (i) a therapeutic monoclonal antibody targeting a specific cell-surface or extracellular antigen (e.g., VEGF, EGFR, CTLA-4, PD-1, or PD-L1) or (ii) a small molecule targeting an intracellular protein such as, for example, an intracellular enzyme (e.g., a proteasome, a tyrosine kinase, a cyclin-dependent kinase, serine/threonine-protein kinase B-Raf (BRAF), or a MEK kinase).

In another aspect, the present invention provides a method for reducing lipid oxidation in a subject. The method generally includes administering to the subject an effective amount of a fusion polypeptide or dimeric protein as described above. In some embodiments, the method is a combination therapy that further reduces NETosis in the subject and that generally includes administering (a) an effective amount of a fusion polypeptide having the formula X-L2-P as described above, or a dimeric protein formed by dimerization of the fusion polypeptide, and (b) an effective amount of a biologically active DNase (e.g., a DNase fusion polypeptide having the formula D-L1-Xa as described above).

In another aspect, the present invention provides a method for protecting a subject from aging. The method generally includes administering to the subject an effective amount of a fusion polypeptide or dimeric protein as described above. In some embodiments, the method is a combination therapy that generally includes administering (a) an effective amount of a fusion polypeptide having the formula X-L2-P as described above, or a dimeric protein formed by dimerization of the fusion polypeptide, and (b) an effective amount of a biologically active DNase (e.g., a DNase fusion polypeptide having the formula D-L1-Xa as described above).

These and other aspects of the invention will become evident upon reference to the following detailed description of the invention.

### Definitions

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

The terms “a,” “an,” and “the” include plural referents, unless the context clearly indicates otherwise.

A “polypeptide” is a polymer of amino acid residues joined by peptide bonds, whether produced naturally or synthetically. Polypeptides of less than about 50 amino acid residues may also be referred to as “peptides.”

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The terms “amino-terminal” (or “N-terminal”) and “carboxyl-terminal” (or “C-terminal”) are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The terms “polynucleotide” and “nucleic acid” are used synonymously herein and refer to a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will in general not exceed 20 nt in length.

A “segment” is a portion of a larger molecule (e.g., polynucleotide or polypeptide) having specified attributes. For example, a DNA segment encoding a specified polypeptide is a portion of a longer DNA molecule, such as a plasmid or plasmid fragment that, when read from the 5′ to the 3′ direction, encodes the sequence of amino acids of the specified polypeptide. Also, for example, in the context of a fusion polypeptide as described herein, different components of the fusion polypeptide (e.g., a DNase, an RNase, a superoxide dismutase (SOD), linker(s), an immunoglobulin Fc region, a paraoxonase) may each be referred to as a polypeptide segment.

The term “biologically active,” when used in reference to a polypeptide segment of a fusion molecule as described herein, means a polypeptide that causes a measurable or detectable physiological, biochemical, or molecular effect in a biological system. Biological activities include, for example, enzymatic activity, antigen-binding, binding to a cell-surface receptor, dimerization, activation of a signaling pathway in a eukaryotic cell, induction of cell proliferation, induction of cell differentiation, and the like. When used in specific reference to a polypeptide segment that is a DNase, RNase, paraoxonase (PON), or superoxide dismutase (SOD), “biologically active” means that the polypeptide exhibits the same type of enzymatic activity as a corresponding, naturally occurring enzyme (e.g., the same type of enzymatic activity as a full-length, wild-type human DNase1/DNase1 L3, RNase1, PON1, or SOD1, respectively), allowing for differences in degree of activity, enzyme kinetics, and the like. An immunoglobulin Fc region, as referenced herein, is understood to be “biologically active” at least by virtue of its dimerizing and FcRn-binding activities.

Unless the context clearly indicates otherwise, reference herein to “paraoxonase” (e.g., “paraoxonase 1” or “PON1”), “DNase” (e.g., “DNase1” or “DNase1 L3”), “RNase” (e.g., “RNase 1”), “superoxide dismutase” (e.g., “superoxide dismutase 1” or “SOD1”), “CTLA-4 extracellular domain,” and “CD40 extracellular domain” is understood to include naturally occurring polypeptides of any of the foregoing, as well as functional variants and functional fragments thereof.

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The term “enhanced DNase1” as used herein denotes a hyperactive and/or actin-resistant variant of a naturally occurring DNase1 (e.g., human DNase1), the variant having (a) at least one amino acid substitution that decreases G-actin-induced inhibition of endonuclease activity relative to the naturally occurring DNase1, and/or (b) at least one amino acid substitution that increases DNA binding relative to the naturally occurring DNase1. In some embodiments, the enhanced DNase1 comprises the at least one amino acid substitution that decreases G-actin-induced inhibition of endonuclease activity. In some embodiments, the enhanced DNase1 comprises both the at least one amino acid substitution that decreases G-actin-induced inhibition of endonuclease activity and the at least one amino acid substitution that increases DNA binding. In some preferred embodiments, an enhanced DNase1 is a hyperactive variant of wild-type human DNase1 (mature amino acid sequence shown in SEQ ID NO:120).

The term “linker” or “polypeptide linker” is used herein to indicate two or more amino acids joined by peptide bond(s) and linking two discrete, separate polypeptide regions. The linker is typically designed to allow the separate polypeptide regions (such as, e.g., a DNase or paraoxonase polypeptide linked to an Fc region) to perform their separate functions. The linker can be a portion of a native sequence, a variant thereof, or a synthetic sequence. Linkers are also referred to herein using the abbreviation “L.” The use of a numerical identifier (e.g., “1” or “2”) with “L” is used herein to differentiate among linkers joining different fusion components: “L1” refers to a linker joining the C-terminus of a first biologically active polypeptide that is not a paraoxonase (e.g., a DNase, an RNase, or a SOD1) to the N-terminus of another polypeptide segment such, e.g., an immunoglobulin Fc region, and “L2” refers to a linker joining the N-terminus of a biologically active paraoxonase (e.g., a PON1) to the C-terminus of another polypeptide segment such as, e.g., an immunoglobulin Fc region. In the context of a polypeptide chain containing both L1 and L2 linkers, the linkers may be the same or different with respect to amino acid sequence. In some variations in which the carboxyl-terminus of the first biologically active polypeptide is linked directly to the amino-terminus of a biologically active paraoxonase via a single polypeptide linker (i.e., with no intervening biologically active polypeptide), such polypeptide linker may be referred to as either L1 or L2.

The term “expression cassette” is used to denote a DNA construct that comprises a segment encoding a polypeptide of interest operably linked to additional segments that provide for its transcription in an appropriate host cell. Such additional sequences include a promoter and, typically, a transcription terminator, and may also include one or more selectable markers, an enhancer, a polyadenylation signal, etc.

The term “vector” is used to denote a polynucleotide produced by recombinant DNA techniques for delivering genetic material into a cell, where it can be replicated. As is well-known in the art, it may refer, e.g., to a plasmid, a cosmid, a viral vector, an artificial chromosome, a cloning vector, or an expression vector. The term “expression vector” is used to denote a vector comprising an expression cassette.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “secretory signal sequence” is a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

“Operably linked” means that two or more entities are joined together such that they function in concert for their intended purposes. When referring to DNA segments, the phrase indicates, for example, that coding sequences are joined in the correct reading frame, and transcription initiates in the promoter and proceeds through the coding segment(s) to the terminator. When referring to polypeptides, “operably linked” includes both covalently (e.g., by disulfide bonding) and non-covalently (e.g., by hydrogen bonding, hydrophobic interactions, or salt-bridge interactions) linked sequences, wherein the desired function(s) of the sequences are retained.

The term “recombinant” when used with reference, e.g., to a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under-expressed or not expressed at all. By the term “recombinant nucleic acid” herein is meant nucleic acid, originally formed in vitro, in general, by the manipulation of nucleic acid using, e.g., polymerases and endonucleases, in a form not normally found in nature. In this manner, operable linkage of different sequences is achieved. Thus an isolated nucleic acid, in a linear form, or an expression vector formed in vitro by ligating DNA molecules that are not normally joined, are both considered recombinant for the purposes disclosed herein. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes disclosed herein. Similarly, a “recombinant protein” is a protein made using recombinant techniques, i.e., through the expression of a recombinant nucleic acid as depicted above.

The term “heterologous,” when used with reference to portions of a nucleic acid, indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, “heterologous,” when used in reference to portions of a protein, indicates that the protein comprises two or more subsequences that are not found in the same relationship to each other in nature (e.g., two or more segments of a fusion polypeptide).

As used herein, the term “immunoglobulin” refers to a protein consisting of one or more polypeptides substantially encoded by immunoglobulin gene(s). One form of immunoglobulin constitutes the basic structural unit of an intact, native antibody. This form is a tetramer and consists of two identical pairs of immunoglobulin chains, each pair having one light and one heavy chain. In each pair, the light and heavy chain variable regions are together responsible for binding to an antigen, and the constant regions are responsible for the antibody effector functions. Immunoglobulins typically function as antibodies in a vertebrate organism. Five classes of immunoglobulin protein (IgG, IgA, IgM, IgD, and IgE) have been identified in higher vertebrates. IgG comprises the major class; it normally exists as the second most abundant protein found in plasma. In humans, IgG consists of four subclasses, designated IgG1, IgG2, IgG3, and IgG4. The heavy chain constant regions of the IgG class are identified with the Greek symbol γ. For example, immunoglobulins of the IgG1 subclass contain a γ1 heavy chain constant region. Each immunoglobulin heavy chain possesses a constant region that consists of constant region protein domains (CHI, hinge, CH2, and CH3; IgG3 also contains a CH4 domain) that are essentially invariant for a given subclass in a species. DNA sequences encoding human and non-human immunoglobulin chains are known in the art. (See, e.g., Ellison et al., DNA 1:11-18, 1981; Ellison et al., Nucleic Acids Res. 10:4071-4079, 1982; Kenten et al., Proc. Nal. Acad. Sci. USA 79:6661-6665, 1982; Seno et al., Nuc. Acids Res. 11:719-726, 1983; Riechmann et al., Nature 332:323-327, 1988; Amster et al., Nuc. Acids Res. 8:2055-2065, 1980; Rusconi and Kohler, Nature 314:330-334, 1985; Boss et al., Nuc. Acids Res. 12:3791-3806, 1984; Bothwell et al., Nature 298:380-382, 1982; van der Loo et al., Immunogenetics 42:333-341, 1995; Karlin et al., J. Mol. Evol. 22:195-208, 1985; Kindsvogel et al., DNA 1:335-343, 1982; Breiner et al., Gene 18:165-174, 1982; Kondo et al., Eur. J. Immunol. 23:245-249, 1993; and GenBank Accession No. J00228.) For a review of immunoglobulin structure and function see Putnam, The Plasma Proteins, Vol V, Academic Press, Inc., 49-140, 1987; and Padlan, Mol. Immunol. 31:169-217, 1994. The term “immunoglobulin” is used herein for its common meaning, denoting an intact antibody, its component chains, or fragments of chains, depending on the context.

The term “antibody,” as used herein, refers to an immunoglobulin molecule, or a fragment and/or engineered variant thereof, which has the ability to specifically bind to an antigen. The term “antibody” includes intact monoclonal antibodies and antigen-binding antibody fragments such as, e.g., F(ab′)2 and Fab fragments. Genetically engineered intact antibodies and fragments, such as chimeric antibodies, humanized antibodies, single-chain Fv fragments, single-chain antibodies, diabodies, minibodies, and the like are also included. Thus, the term “antibody” is used expansively to include any protein that comprises an antigen-binding site and is capable of binding to its antigen.

The term “genetically engineered antibodies” means antibodies wherein the amino acid sequence has been varied from that of a native (i.e., naturally occurring) antibody. Because of the relevance of recombinant DNA techniques in the generation of antibodies, one need not be confined to the sequences of amino acids found in natural antibodies; antibodies can be redesigned to obtain desired characteristics. The possible variations are many and range from the changing of just one or a few amino acids to the complete redesign of, for example, the variable or constant region. Changes in the constant region will, in general, be made in order to improve or alter characteristics, such as complement fixation, interaction with cells and other effector functions. Typically, changes in the variable region will be made in order to improve the antigen binding characteristics, improve variable region stability, or reduce the risk of immunogenicity.

An “antigen-binding site” is that portion of an antibody that is sufficient to bind to its antigen. The minimum such region is typically an immunoglobulin variable domain or fragment thereof, or a genetically engineered variant of an immunoglobulin variable domain or fragment thereof. Single-domain binding sites can be generated from camelid antibodies (see Muyldermans and Lauwereys, J. Mol. Recog. 12:131-140, 1999; Nguyen et al., EMBO J. 19:921-930, 2000) or from VH domains of other species to produce single-domain antibodies (“dAbs”; see Ward et al., Nature 341:544-546, 1989; U.S. Pat. No. 6,248,516 to Winter et al.). In certain variations, an antigen-binding site is a polypeptide region having only 2 complementarity determining regions (CDRs) of a naturally or non-naturally (e.g., mutagenized) occurring heavy chain variable domain or light chain variable domain, or combination thereof (see, e.g., Pessi et al., Nature 362:367-369, 1993; Qiu et al., Nature Biotechnol. 25:921-929, 2007).

Full-length immunoglobulin “light chains” (about 25 Kd or 214 amino acids) are encoded by a variable region gene at the N-terminus (encoding about 110 amino acids) and by a kappa or lambda constant region gene at the C-terminus. Full-length immunoglobulin “heavy chains” (about 50 Kd or 446 amino acids) are encoded by a variable region gene (encoding about 116 amino acids) and a gamma, mu, alpha, delta, or epsilon constant region gene (encoding about 330 amino acids), the latter defining the antibody's isotype as IgG, IgM, IgA, IgD, or IgE, respectively. Within light and heavy chains, the variable and constant regions are joined by a “J” region of about 12 or more amino acids, with the heavy chain also including a “D” region of about 10 more amino acids. (See generally Fundamental Immunology (Paul, ed., Raven Press, N.Y., 2nd ed. 1989), Ch. 7).

An immunoglobulin light or heavy chain variable region consists of a framework region interrupted by three hypervariable regions. The term “hypervariable region,” also referred to herein as “complementarity determining region” (“CDR”), refers to the amino acid residues of an antibody that are responsible for antigen binding. A CDR may be defined according to any of several known methods of analysis. Examples of such methods include, e.g., the Kabat definition, the Chothia definition, the AbM definition, the IMGT database definition, and the contact definition. The Kabat definition is a standard for numbering the residues in an antibody and is typically used to identify CDR regions (see. e.g., Johnson & Wu, Nucleic Acids Res. 28:214-8, 2000). The Chothia definition is similar to the Kabat definition, but the Chothia definition takes into account positions of certain structural loop regions (see, e.g., Chothia et al., J. Mol. Biol. 196:901-17, 1986; Chothia et al., Nature 342:877-83, 1989). The AbM definition uses an integrated suite of computer programs produced by Oxford Molecular Group that model antibody structure (see, e.g., Martin et al., 1989, Proc. Natl. Acad. Sci. USA 86:9268-9272, 1989; “AbM™. A Computer Program for Modeling Variable Regions of Antibodies,” Oxford, UK; Oxford Molecular, Ltd.). The AbM definition models the tertiary structure of an antibody from primary sequence using a combination of knowledge databases and ab initio methods, such as those described by Samudrala et al., “Ab Initio Protein Structure Prediction Using a Combined Hierarchical Approach,” in PROTEINS. Structure. Function and Genetics Suppl. 3:194-198, 1999. The IMGT database definition takes into account and combines the definition of the framework (FR) and complementarity determining regions (CDRs), structural data from X-ray deffraction studies, and characterization of hypervariable loops to number the variable regions/structures (see LeFranc, Immunol. Today 18:509, 1997). The contact definition is based on an analysis of the available complex crystal structures (see. e.g., MacCallum et al., J. Mol. Biol. 5:732-45, 1996). CDRs L1, L2, and L3 of a VL domain are also referred to herein, respectively, as CDR-L1. CDR-L2, and CDR-L3; CDRs H1, H2, and H3 of a VH domain are also referred to herein, respectively, as CDR-H1, CDR-H2, and CDR-H3.

“Framework region” are those variable domain residues other than the hypervariable region residues (e.g., other than the residues of CDRs as defined by the Kabat definition, the Chothia definition, the AbM definition, the IMGT database definition, or the contact definition of CDR). The sequences of the framework regions of different light or heavy chains are relatively conserved within a species. Thus, a “human framework region” is a framework region that is substantially identical (about 85% or more, usually 90-95% or more) to the framework region of a naturally occurring human immunoglobulin. The framework region of an antibody, that is the combined framework regions of the constituent light and heavy chains, serves to position and align the CDRs.

An “immunoglobulin hinge” is that portion of an immunoglobulin heavy chain connecting the CHI and CH2 domains. The hinge region of human γ1 corresponds approximately to Eu residues 216-230.

The terms “Fc fragment,” “Fc region,” or “Fc domain,” as used herein, are synonymous and refer to the portion of an immunoglobulin that is responsible for binding to antibody receptors on cells and the C1q component of complement (in the absence of any amino acid changes, relative to the naturally occurring sequence, to remove such binding activity). Fc stands for “fragment crystalline,” the fragment of an antibody that will readily form a protein crystal. Distinct protein fragments, which were originally described by proteolytic digestion, can define the overall general structure of an immunoglobulin protein. As originally defined in the literature, the Fc fragment consists of the disulfide-linked heavy chain hinge regions, CH2, and CH3 domains. As used herein, the term also refers to a single chain consisting of CH3, CH2, and at least a portion of the hinge sufficient to form a disulfide-linked dimer with a second such chain. As used herein, the term Fc region further includes variants of naturally occurring hinge-CH2-CH3 sequences, wherein the variants are capable of forming dimers at least through dimerization of the CH3 domain and including such variants that have increased or decreased Fc receptor-binding or complement-binding activity while retaining at least sufficient binding to the neonatal Fc receptor (FcRn) to confer improved half-life to a fusion partner in vivo (relative to the fusion partner in the absence of the Fc region). The abbreviated term “Fc” may also be used herein to denote “Fc region” when referring to a fusion polypeptide by its general amino- to carboxyl-terminal structure (e.g., “DNase1-L1-Fc-L2-PON1”).

As used herein, the term “single-chain antibody” refers to an antibody having an antigen-binding site contained within a single polypeptide chain (e.g., the variable regions from both heavy and light chains within a single polypeptide chain). The term “single-chain Fv” refers to a single-chain antibody that comprises the variable regions from both heavy and light chains but lacks constant regions. In general, a single-chain Fv further comprises a polypeptide linker between the VH and VL domains, which enables it to form the desired structure that allows for antigen binding. Single-chain antibodies are discussed in detail by, for example, Pluckthun in The Pharmacology of Monoclonal Antibodies, vol. 113 (Rosenburg and Moore eds., Springer-Verlag, New York, 1994), pp. 269-315. (See also International PCT Publication No. WO 88/01649; U.S. Pat. Nos. 4,946,778 and 5,260,203; Bird et al., Science 242:423-426, 1988.) Single-chain antibodies can also be bi-specific and/or humanized.

The term “alternative scaffold protein” refers to a non-antibody protein in which one or more regions may be diversified to produce one or more binding domains that specifically bind to a target molecule (e.g., an inflammatory cytokine such as TNFα or TGF-β). In some embodiments, the binding domain binds the target molecule with specificity and affinity similar to that of an antibody. Exemplary alternative scaffolds include those derived from fibronectin (e.g., Adnectins™), the β-sandwich (e.g., iMab), lipocalin (e.g., Anticalins@), EETI-II/AGRP, BPTI/LACI-D1/ITI-D2 (e.g., Kunitz domains), protein A (e.g., Affibody@), ankyrin repeats (e.g., DARPins), gamma-B-crystallin/ubiquitin (e.g., Affilins), CTLD₃ (e.g., Tetranectins), Fynomers, and Avimers. Additional information on alternative scaffolds is provided in Binz et al., Nat. Biotechnol. 23:1257-1268, 2005; Skerra, Current Opin. in Biotech. 18:295-304, 2007; and Silacci et al., J. Biol. Chem. 289:14392-14398, 2014.

The term “antiTNFα” or “antiTGFβ,” as used herein when referring to a fusion polypeptide by its general amino- to carboxyl-terminal structure (e.g., “antiTNFα-L1-Fc-L2-PON1” or “antiTGFβ-L1-Fc-L2-PON1”), refers to a polypeptide (e.g., single-chain antibody) that specifically binds and neutralizes TNFα or TGFβ, respectively.

“Dimerizing domain,” as used herein, refers to a polypeptide having affinity for a second polypeptide, such that the two polypeptides associate under physiological conditions to form a dimer. Typically, the second polypeptide is the same polypeptide, although in some variations the second polypeptide is different. The polypeptides may interact with each other through covalent and/or non-covalent association(s). Examples of dimerizing domains include an Fc region; a hinge region; a CH3 domain; a CH4 domain; a CH1 or CL domain; a leucine zipper domain (e.g., a jun/fos leucine zipper domain, see, e.g., Kostelney et al., J. Immunol., 148:1547-1553, 1992; or a yeast GCN4 leucine zipper domain); an isoleucine zipper domain; a dimerizing region of a dimerizing cell-surface receptor (e.g., interleukin-8 receptor (IL-8R); or an integrin heterodimer such as LFA-1 or GPIIIb/IIIa); a dimerizing region of a secreted, dimerizing ligand (e.g., nerve growth factor (NGF), neurotrophin-3 (NT-3), interleukin-8 (IL-8), vascular endothelial growth factor (VEGF), or brain-derived neurotrophic factor (BDNF); see, e.g., Arakawa et al., J. Biol. Chem. 269:27833-27839, 1994, and Radziejewski et al., Biochem. 32:1350, 1993); and a polypeptide comprising at least one cysteine residue (e.g., from about one, two, or three to about ten cysteine residues) such that disulfide bond(s) can form between the polypeptide and a second polypeptide comprising at least one cysteine residue (hereinafter “a synthetic hinge”). A preferred dimerizing domain in accordance with the present invention is an Fc region.

The term “dimer” or “dimeric protein” as used herein, refers to a multimer of two (“first” and “second”) fusion polypeptides as disclosed herein linked together via a dimerizing domain. Unless the context clearly indicates otherwise, a “dimer” or “dimeric protein” includes reference to dimerized first and second fusion polypeptides in the context of higher order multimers that may be created by inclusion of an additional dimerizing domain in a first or second fusion polypeptide (e.g., a first fusion polypeptide comprising an immunoglobulin light chain and a second fusion polypeptide comprising an immunoglobulin heavy chain can heterodimerize via the interaction between the CH1 and CL domains, and two such heterodimers may further dimerize via the Fc region of the immunoglobulin heavy chain, thereby forming a tetramer).

The term “domain that specifically binds to the neonatal Fc receptor (FcRn)” or “FcRn-binding domain,” as used herein, means a polypeptide that (i) binds to FcRn with a high affinity at pH 5.8, typically with a binding affinity (Kₐ) of 10⁶ M⁻¹ or greater (e.g., 10⁷ M⁻¹ or greater, 10⁸ M⁻¹ or greater, or 109 M⁻¹ or greater), and (ii) does not have affinity for FcRn at physiological pH (e.g., pH 7.4). The binding affinity of a polypeptide for FcRn can be readily determined by one of ordinary skill in the art, for example, by Scatchard analysis (Scatchard, Ann. NY Acad. Sci. 51:660, 1949). Typically, a FcRn-binding domain does not significantly cross-react with polypeptides related to FcRn. A polypeptide does not significantly cross-react with a polypeptide related to FcRn if, for example, it detects FcRn, but not presently known FcRn-related polypeptides, using a molecular binding assay such as, e.g., a multiwell plate assay (e.g., ELISA), a filter assay, or a surface plasmon resonance assay. Examples of known related polypeptides include known members of the major histocompatibility complex (MHC) class I protein family. An FcRn-binding domain is not mutually exclusive of a dimerizing domain, i.e., a dimerizing domain can also be an FcRn-binding domain. Examples of FcRn-binding domains that are also dimerizing domains include Fc regions that retain FcRn-binding activity.

Fusion polypeptides of the present disclosure may be referred to herein by formulae such as, for example, “DNase1-L1-Fc,” “DNase1 L3-L1-Fc,” “DNase1-L1-Fc-L2-PON1,” “DNase1L3-L1-Fc-L2-PON1,” “RNase1-L1-Fc-L2-PON1,” “SOD1-L1-Fc-L2-PON1,” “anti-TNFα-L1-Fc-L2-PON1,” or “anti-TGFβ-L1-Fc-L2-PON1.” In each such case, unless the context clearly dictates otherwise, a term referring to a particular segment of a fusion polypeptide (e.g., “DNase1,” “DNase1 L3,” “PON1,” “RNase1,” “SOD1,” “L1” or “L2” (for first or second polypeptide linkers, respectively), “Fc” (for “Fc region”), etc.) is understood to have the meaning ascribed to such term herein and is inclusive of the various embodiments as described herein.

The term “disease or disorder characterized by NETosis,” as used herein, means a disease or disorder in which NETosis—the process wherein activated neutrophils extrude DNA bound with cytoplasmic and granule proteins, called neutrophil extracellular traps (NETs)—plays at least some part of the clinicopathology.

The term “effective amount,” in the context of treatment of a disease by administration of a soluble fusion polypeptide or dimeric protein to a subject as described herein, refers to an amount of such molecule that is sufficient to inhibit the occurrence or ameliorate one or more symptoms of the disease. For example, in the specific context of treatment of an inflammatory lung disease by administration of a fusion protein to a subject as described herein, the term “effective amount” refers to an amount of such molecule that is sufficient to modulate an inflammatory response in the subject so as to inhibit the occurrence or ameliorate one or more symptoms of the inflammatory lung disease. An effective amount of an agent is administered according to the methods of the present invention in an “effective regime.” The term “effective regime” refers to a combination of amount of the agent being administered and dosage frequency adequate to accomplish treatment or prevention of the disease.

The term “patient” or “subject,” in the context of treating a disease or disorder as described herein, includes mammals such as, for example, humans and other primates. The term also includes domesticated animals such as, e.g., cows, hogs, sheep, horses, dogs, and cats.

The term “combination therapy” refers to a therapeutic regimen that involves the provision of at least two distinct therapies to achieve an indicated therapeutic effect. For example, a combination therapy may involve the administration of two or more chemically distinct active ingredients, or agents, for example, a soluble PON1 fusion polypeptide or dimeric protein according to the present invention and another agent such as, e.g., another anti-inflammatory or immunomodulatory agent. Alternatively, a combination therapy may involve the administration of a soluble PON1 fusion polypeptide or dimeric protein according to the present invention, alone or in conjunction with another agent, as well as the delivery of another therapy (e.g., radiation therapy). The distinct therapies constituting a combination therapy may be delivered, e.g., as simultaneous, overlapping, or sequential dosing regimens. In the context of the administration of two or more chemically distinct agents, it is understood that the active ingredients may be administered as part of the same composition or as different compositions. When administered as separate compositions, the compositions comprising the different active ingredients may be administered at the same or different times, by the same or different routes, using the same or different dosing regimens, all as the particular context requires and as determined by the attending physician.

The term “targeted therapy,” in the context of treating cancer, refers to a type of treatment that uses a therapeutic agent to identify and attack a specific type of cancer cell, typically with less harm to normal cells. In some embodiments, a targeted therapy blocks the action of an enzyme or other molecule involved in the growth and spread of cancer cells. In other embodiments, a targeted therapy either helps the immune system to attack cancer cells or delivers a toxic substance directly to cancer cells. In certain variations, a targeted therapy uses a small molecule drug or a monoclonal antibody as a therapeutic agent.

The phrase “protect from aging,” as used herein, refers to inhibition or mitigation of any of broad aspects of aging, including, for example, age-related changes in systemic inflammation or disease risk, as indicated by accepted biomarkers. Protection from aging may also include treatment of an age-related disease where the disease is present in a subject, such as, for example, a chronic inflammatory, autoimmune, neurodegenerative, cardiovascular, or fibrotic disease.

Two amino acid sequences have “100% amino acid sequence identity” if the amino acid residues of the two amino acid sequences are the same when aligned for maximal correspondence. Sequence comparisons can be performed using standard software programs such as those included in the LASERGENE bioinformatics computing suite, which is produced by DNASTAR (Madison. Wisconsin). Other methods for comparing amino acid sequences by determining optimal alignment are well-known to those of skill in the art. (See, e.g., Peruski and Peruski, The Internet and the New Biology: Tools for Genomic and Molecular Research (ASM Press, Inc. 1997). Wu et al. (eds.), “Information Superhighway and Computer Databases of Nucleic Acids and Proteins,” in Methods in Gene Biotechnology 123-151 (CRC Press, Inc. 1997); Bishop (ed.), Guide to Human Genome Computing (2nd ed., Academic Press, Inc. 1998).) Two amino acid sequences are considered to have “substantial sequence identity” if the two sequences have at least 80%, at least 90%, or at least 95% sequence identity relative to each other.

Percent sequence identity is determined by conventional methods. (See, e.g., Altschul et al., Bull. Math. Bio. 48:603, 1986, and Henikoff and Henikoff, Proc. Natl. Acad. Sci. USA 89:10915, 1992.) For example, two amino acid sequences can be aligned to optimize the alignment scores using a gap opening penalty of 10, a gap extension penalty of 1, and the “BLOSUM62” scoring matrix of Henikoff and Henikoff, supra. The percent identity is then calculated as: ([Total number of identical matches]/[length of the longer sequence plus the number of gaps introduced into the longer sequence in order to align the two sequences])(100).

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and a second amino acid sequence. The FASTA algorithm is described by Pearson and Lipman, Proc. Nat′l Acad. Sci. USA 85:2444, 1988, and by Pearson, Meth. Enzymol. 183:63, 1990. Briefly, FastA first characterizes sequence similarity by identifying regions shared by the query sequence (e.g., residues 16-355 or 26-355 of SEQ ID NO:6) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444, 1970; Sellers, SIAM J. Appl. Math. 26:787, 1974), which allows for amino acid insertions and deletions. Illustrative parameters for FASTA analysis are: ktup=1, gap opening penalty=10, gap extension penalty=1, and substitution matrix=blosum62. These parameters can be introduced into a FASTA program by modifying the scoring matrix file (“SMATRIX”), as explained in Appendix 2 of Pearson, Meth. Enzymol. 183:63, 1990.

The term “corresponding to,” when applied to positions of amino acid residues in a reference sequence to describe positions within a subject sequence, means corresponding positions in the subject sequence when the reference and subject sequences are optimally aligned.

When such a value is expressed as “about” X or “approximately” X, the stated value of X will be understood to be accurate to ±10%.

Where aspects or embodiments of the present invention are described in terms of a Markush group or other grouping of alternatives, the present invention encompasses not only the entire group listed as a whole, but each member of the group individually and all possible subgroups of the main group. The present invention also envisages the explicit exclusion of one or more of any of the group members as embodiments.

### BRIEF DESCRIPTION OF THE DRAWINGS

FIG. 1 shows a Western blot of culture supernatants (serum free) from transiently transfected HEK293 cells expressing DNase1-Fc and DNase1-Fc-PON1 fusion proteins. Transfections and Western blot analysis were performed as described in Example 1, infra. From left to right: Lane 1—LICOR Chameleon™ molecular weight markers; Lane 2, Lane 3, Lane 4—transfection supernatants from three different individual clones (#1, #2, #4) of hDNase™-(g4s)4-Fc-PON1-Q192K; Lane 5—THER4-RNase (control); Lane 6—LICOR Chameleon MW Markers; Lane 7—hDNase™-(g4s)4-Fc #4; Lane 8—hDNase™-(g4s)6-Fc #2; Lane 9—THER4 (control); Lane 10—mock transfection.

FIG. 2 shows reducing SDS-PAGE analysis of DNase1-Fc and DNase1-Fc-PON1 fusion proteins purified from CHO clone spent culture supernatants. Stable CHO cell generation. Protein A purification of fusion proteins, and SDS-PAGE were performed as described in Example 1, infra. From left to right: Lane 1—THER4PON1-K (control); Lane 2—hDNase™-(g4s)4-Fc-PON1-K; Lane 3—hDNase™-(g4s)4-Fc; Lane 4—hDNase™-(g4s)6-Fc; Lane 5—L1-COR Chameleon® MW markers; Lane 6—BioRad Precision Plus Kaleidoscope MW markers.

FIGS. 3A and 3B show raw data from a modified SYTOX™ Green fluorescence assay assessing nuclease activity of DNase1 fusion proteins at two different substrate concentrations, 220 μM or 66 μM salmon sperm DNA (see Example 2, infra).

FIGS. 4A and 4B show Michaelis-Menten plots of data from two different DNase activity experiments using a modified SYTOX™ Green fluorescence assay (see Example 2, infra).

FIGS. 5A and 5B show results from a SYTOX™ Green fluorescence assay comparing relative actin-induced inhibition of DNase activity of recombinant human DNase1 and a DNase1-Fc fusion protein (see Example 2, infra).

FIGS. 6A and 6B show Michaelis-Menten and Lineweaver-Burk plots, respectively, for a representative experiment measuring the arylesterase activity of a DNase1-Fc-PON1 fusion protein (see Example 3, infra).

FIG. 7 shows the results of a fluorometric assay measuring the organophosphatase activity of a DNase1-Fc-PON1 fusion protein and an organophosphatase positive control enzyme (see Example 3, infra).

FIGS. 8 and 9 show Western blot analysis of PON1 fusion proteins immunoprecipitated from HEK293 transfection supernatants. Transfections, Protein A immunoprecipitation, and Western blot analysis were performed as described in Example 12, infra.

FIG. 10 shows reducing SDS-PAGE analysis of PON1 fusion proteins immunoprecipitated from HEK293 transfection supernatants. Transfections, Protein A immunoprecipitation, and SDS-PAGE were performed as described in Example 12, infra.

FIGS. 11-13 show arylesterase activity (FIG. 11), organophosphatase activity (FIG. 12), and lactonase activity (FIG. 13) of PON1 fusion proteins assayed directly from HEK293 culture supernatants (see Example 13, infra).

FIG. 14 shows high affinity binding of immobilized CD40 ligand to CD40-Fc-PON1 fusion protein present in HEK293 transfection supernatant (see Example 14, infra).

FIG. 15 shows high affinity binding of immobilized TNFα to anti-TNF-α scFv-Fc-PON1 fusion protein present in HEK293 transfection supernatant (see Example 14, infra).

### DESCRIPTION OF THE INVENTION

### I. Overview

The present invention provides compositions and methods relating to paraoxonase fusion polypeptides. In certain aspects, a fusion polypeptide comprises a first biologically active polypeptide linked amino-terminal to a biologically active paraoxonase, wherein the first biologically active polypeptide is selected from a DNase, an RNase, a superoxide dismutase 1 (SOD1), a cytotoxic T-lymphocyte associated molecule-4 (CTLA-4) extracellular domain, a CD40 extracellular domain, and a polypeptide that specifically binds and neutralizes an inflammatory cytokine (e.g., a polypeptide that specifically binds and neutralizes tumor necrosis factor α (TNFα) or transforming growth factor-β(TGFβ)). In some aspects, the fusion polypeptide further includes a second biologically active polypeptide linked carboxyl-terminal to the first biologically active polypeptide and amino-terminal to the paraoxonase. The second biologically active polypeptide may be, for example, a dimerizing domain or a domain that specifically binds to the neonatal Fc receptor (FcRn). A particularly suitable second biologically active polypeptide is a dimerizing and FcRn-binding immunoglobulin heavy chain constant region such as, e.g., an immunoglobulin Fc region.

In other aspects, a fusion polypeptide comprises a biologically active paraoxonase linked carboxyl-terminal to a dimerizing domain or domain that specifically binds to FcRn (for example, a dimerizing and FcRn-binding immunoglobulin heavy chain constant region such as, e.g., an immunoglobulin Fc region), wherein the fusion polypeptide does not comprise a biologically active polypeptide amino-terminal to the dimerizing or FcRn-binding domain.

In other aspects, a fusion polypeptide comprises a biologically active paraoxonase linked amino-terminal to a dimerizing domain or domain that specifically binds to FcRn (for example, a dimerizing and FcRn-binding immunoglobulin heavy chain constant region such as, e.g., an immunoglobulin Fc region).

A preferred paraoxonase in fusion polypeptides of the present disclosure is a paraoxonase 1 (PON1) polypeptide. Particularly suitable PON1 polypeptides lack an amino-terminal leader sequence corresponding to the naturally occurring, non-cleaved leader sequence of PON1 (generally corresponding to residues 1-15 of human PON1 (SEQ ID NO:4)). The association of PON1 with high density lipoprotein (HDL) in serum is primarily dependent on the direct binding of the hydrophobic, non-cleaved leader to phospholipids (see Sorenson et al., Arterioscler. Thromb. Vase. Biol. 19:2214-2225, 1999). The removal of this leader sequence removes PON1 from HDL binding and facilitates the retargeting of PON1 to other sites for therapeutic benefit in accordance with the present disclosure. For example, in the context of a fusion polypeptide comprising a DNase or RNase as described herein, retargeting PON1 to DNA or RNA directs the molecule to inflammatory NETs and sites of cellular death. In addition to targeting DNA or RNA at these sites, DNase-PON1 or RNase-PON1 fusion molecules as described herein can help to direct a PON1 enzyme to myeloperoxidase, another important inflammatory NET target. Also, a fusion polypeptide comprising a superoxide dismutase 1 (SOD1) as described herein provides a molecule in which PON1 can digest the hydrogen peroxide (H₂O₂) produced by the action of SOD1 on superoxide, thereby providing, e.g., an improved antioxidant for lung therapy. Further, fusion molecules comprising other biologically active polypeptides in accordance with the present invention (e.g., a CTLA-4 extracellular domain, a CD40 extracellular domain, or a polypetide that specifically binds and neutralizes an inflammatory cytokine) can target PON1 to other sites of proinflammatory and/or immune cell activation for therapeutic benefit such as described herein.

Paraoxonase fusion molecules of the present invention may be used for the treatment of various diseases or disorders through its antioxidant, anti-inflammatory, atheroprotective, and/or neuroprotective properties, including, e.g., treatment of an autoimmune or inflammatory disease. For example, studies support use of a paraoxonase for treatment of autoimmune disease such as systemic lupus erythematosus (SLE). The autoantibody titer in many patients with systemic lupus erythematosus (SLE) is correlated with loss of activity of PON1 (see Batukla et al., Ann. NY Acad. Sci. 1108:137-146, 2007), and SLE-disease activity assessed by SLEDAI and SLE disease related organ damage assessed by SLICC/ACR damage index are negatively correlated with PON1 activity (see Ahmed et al., EXCLI Journal 12:719-732, 2013). Other studies support use of a paraoxonase for treatment of inflammatory disease such as inflammatory lung diseases. Studies strongly suggest, for example, that PON1 is important in protection from lung inflammation, and further show that low PON1 activity is associated with lung diseases such as, e.g., asthma, chronic obstructive pulmonary disease (COPD), and interstitial lung disease (e.g., idiopathic pulmonary fibrosis (IPF) or sarcoidosis) (see, e.g., Sahiner et al., WAO Journal 4:151-158, 2011; Emin et al., Allergol. Immunopathol. (Madr) 43:346-352, 2014; Sarioglu et al., Iran J. Asthma Immunol. 14:60-66, 2015; Tolgyesi et al., Internat. Immunol. 21:967-975, 2009; Chen et al., J. Cell Biochem. 119:793-805, 2018; Rumora et al., J. COPD 11:539-545, 2014; Rajkovic et al., J. Clin. Path. 71:963-970, 2018; Ivanisevic et al., Eur. J. Clin. Invest. 46:418-424, 2016; Uzun et al., Curr. Med. Res. Opin. 24:1651-1657, 2008; Okur et al., Sleep Breath. 17:365-371, 2013; Golmanesh et al., Immunopharmacol. And Immunotoxicol. 35:419-425, 2013). Another study showed that low PON1 activity is correlated with the severity of Crohn's disease (Sczceklik et al., Molecules 23:2603, 2018). In addition, recombinant PON1 therapy has shown efficacy in animal models of organophosphate poisoning and colitis (see, e.g., Valiyaveettil et al., Biochem. Pharmacol. 81:800-809, 2011; Valiyaveettil et al., Toxicol. Letters 202:203-208, 2011; Bajaj et al., Appl. Biochem. Biotechnol. 180:165, 2016; Stevens et al., Proc. Natl. Acad. Sci. USA 105:12780-12784, 2008; Yamashita et al., J. Immunol. 191:949-960, 2013).

Paraoxonase fusion molecules as described herein may also be used, e.g., for treatment of a neurological disease. For example, PON1 is protective in the brain because of its antioxidant properties. A neuroprotective role of PON1 is supported by studies showing that PON1 activity is decreased in patients with Alzheimer's disease and other dementias (see, e.g., Menini et al., Redox Rep. 19:49-58, 2014). In addition, a study using a PON1 fusion containing a protein transduction domain to transduce PON1 into cells and tissues showed that PON1 transduction protected microglial cells in vitro from oxidative stress-induced inflammatory responses and protected against dopaminergic neuronal cell death in a Parkinson's disease model (see Kim et al., Biomaterials 64:45-56, 2015).

Bispecific fusions further comprising a DNase provide additional therapeutic benefit for the treatment of diseases amenable to PON-mediated therapy, including, for example, in treatment of diseases or disorders characterized by NETosis. NETs, which cause tissue damage by themselves or by increasing the pro-inflammatory response (see Mutua and Gershwin, Clin. Rev. Allergy Immunol., 2020), are implicated in a variety of serious diseases and conditions. For example, NETs can play a role in enhancement of inflammation seen in autoimmune disease, including, e.g., systemic lupus erythematosus (SLE) (e.g., SLE with lupus nephritis), rheumatoid arthritis, and psoriasis, and are also associated with autoinflammatory diseases such as, for example, inflammatory bowel disease (IBD) (see id.). NETs also play a role in sepsis, metabolic diseases (e.g., type 2 diabetes and obesity), neurological diseases (e.g., Alzheimer's disease, multiple sclerosis, meningitis, cerebral malaria, and ischemic stroke), infectious disease, cardiovascular disease and thrombosis, tumor progression and metastasis, liver disease (e.g., alcohol-associated liver disease (ALD), portal hypertension, nonalcoholic fatty liver disease (NAFLD) such as nonalcoholic steatohepatitis (NASH), and complications following liver transplantation), and various inflammatory lung diseases, including, e.g., cystic fibrosis, asthma, chronic obstructive pulmonary disease (COPD), and COVID-19-associated acute respiratory distress syndrome (ARDS) (see, e.g., id.; Brinkmann, J. Innate Immun. 10:422-431, 2018; Liu et al., Chinese Med. Journal 130:730-736, 2017; Twaddell et al., CHEST 156:774-782, 2019; Uddin et al., Front. Immunol. 10:47, 2019; Middleton et al., Blood 136:1169, 2020; Zuo et al., JCI Insight 5:e138999, 2020; Barnes et al., J. Exp. Med. 217:e20200652, 2020; Hilscher and Shah, Semin. Liver Dis. 40:171-179, 2020). The use of DNase for the treatment of diseases or disorders characterized by NETosis is further supported, for example, by the use of recombinant wild-type DNase (Pulmozyme@) for treatment of cystic fibrosis, a current clinical trial of Pulmozyme in COVID-19 patients (NCT04359654), and several animal model studies, including, e.g., models of acute asthma, silica-induced lung inflammation, acute lung injury, cancer, colitis, ischemia reperfusion, sepsis, and thrombosis (see, e.g., da Cunha et al., Exp. Lung Res. 42:66, 2016; Benmerzoug et al., Nat. Comm. 9:5226, 2018; Caudrillier et al., J. Clin. Invest. 122:2661, 2012; Cools-Lartigue et al., J. Clin. Invest. 123:3446, 2013; Park et al., Sci. Translational Med. 8:361ral38, 2016; Trejo-Becirril et al., Integrative Cancer Therapies 15:NP35-NP43, 2016; Thalin et al., PloS ONE 13:e0191231, 2018; Babicova et al., Folia Biolica (Praha) 64:10, 2018; Li et al., J. Crohns Colitis 2020, 14:240-253, 2020; Albadawi et al., J. Vasc. Surg. 64:484, 2016; Peer et al., Am. J. Nephrol. 43:195, 2016; Mai et al., Shock 44:166, 2015; Brill et al., J. Thrombosis Haemostasis 10:136, 2012; Manda-Handzlik and Demkow, Cells 8:1477, 2019; Delgado-Rizo et al., Front. Immunol. 8:81, 2017).

Bispecific fusions further comprising a DNase as described herein are also useful in treatment of antiphospholipid syndrome (APLS). Antiphospholipid syndrome is an autoimmune disease characterized by antibodies to phospholipids that cause a hypercoagulable state. Thrombosis occurs in both arteries and veins and causes pregnancy complications including miscarriage and stillbirths. Antiphospholipid syndrome can be primary (occurring in the absence of any other related disease) or secondary (occurring with another autoimmune disease such as, e.g., SLE). In rare cases, APLS leads to rapid organ failure due to generalized thrombosis; this is termed catastrophic antiphospholipid syndrome (CAPS or Asherson syndrome) and is associated with a high risk of death. Patients with acute respiratory distress associated with COVID-19 have antiphospholipid antibodies that promote thrombosis in vitro and in mouse thrombosis models (Zuo et al., JCI Insight 5:e138999, 2020; Zuo et al., Sci. Trans. Med. 12:eabd3876, 2020). The antiphospholipid antibodies stimulate neutrophil NET formation, providing a scaffold for thrombus formation. Digestion of NETs by DNase fusions as described herein can prevent thrombus formation and can help dissolve existing clots to restore blood flow. DNase fusion molecules of the present invention may also be used together with thrombolytics including, e.g., tPA, urokinase, and streptokinase.

Paraoxonase fusion molecules as described herein, including embodiments further comprising, e.g., a DNase, may also be used for treatment of an inflammatory skin disease. Many studies have reported the involvement of reactive oxygen species and lipid peroxidation in the pathogenesis of inflammatory skin diseases (see, e.g., Simonetti et al., Antioxidants 10:697, 2021). For example, significantly lower levels of paraoxonase 1 (PON1) and a significantly higher levels of myeloperoxidase (MPO) and lipid peroxides have been found in atopic dermatitis (see id.), and increased MPO and decreased PON1 activity have also been described in psoriatic children (see Bacchetti et al., Archives of Dermatological Research 312:33-39). In psoriasis and atopic dermatitis, neutrophil activation during inflammation leads to extruded NETs with attached MPO, causing the oxidation of lipids and damaging PON1 enzyme, which normally protects lipids from oxidation by digesting lipid peroxides. The digestion of NETs with DNase1 releases the attached MPO, allowing its clearance and removing a primary driver of oxidation, and therapy with PON1 restores protection from oxidative damage and rebalances the redox potential in patients. Thus, PON1 and DNase therapies are particularly effective as a combination.

Paraoxonase fusion molecules as described herein, including embodiments further comprising a DNase, may also be used to protect from aging. Oxidative stress is known to be a primary cause of aging. In particular, oxidized lipids are a key target for aging because of their promotion of inflammation through uptake by macrophage scavenger receptors and their damage to vascular endothelial cells. See, e.g., Moldogazieva et al. (Oxidative Medicine and Cellular Longevity, Volume 2019, Article ID 3085756) and Barrera et al. (Antioxidants 7:102, 2018) for discussions on the link between lipid oxidation and aging. See also Hajri (Frontiers In Bioscience, Landmark 23:1822-1847, 2018), which describes the toxic effects of oxidized lipids on inflammation and cardiac function. PON1 is known to protect lipids from oxidation by digestion of lipid peroxides. Thus, there is a direct link between increasing PON1 enzyme levels and slowing of aging. In addition, PON1 enzyme activity is known to be deficient in multiple age-related diseases including cardiovascular disease, autoimmune disease, and pulmonary disease, showing that there is a clinical need for increased PON1. In addition, embodiments further comprising a DNase are particularly useful for slowing aging because of the ability of DNase to digest NETs. NETs are a primary driver of inflammation and oxidative stress because they provide the scaffold for myeloperoxidase (MPO) and elastase produced by neutrophils and other cells. Digestion of the NETs by DNase allows the release and clearance of the inflammatory enzymes. DNase also eliminates inflammatory activation through other DNA sensing pathways (e.g., STING, TLR9).

Bispecific fusions further comprising an RNase as described herein have anti-inflammatory properties by digesting extracellular RNA derived from dead and dying cells, including, e.g., extracellular RNA associated with NETs. Extracellular RNA synergizes, for example, with TLR2 agonists to potentiate a macrophage inflammatory response and digestion of this RNA prevents inflammation through TLR2 (see Noll et al., PloS ONE 12:e0190002, 2017).

RNase-containing fusions provide additional therapeutic benefit for treatment of various diseases, including, e.g., autoimmune disease, inflammatory disease (e.g., inflammatory lung disease), type 2 diabetes, infectious disease, cardiovascular disease (e.g., coronary artery disease, stroke), neurodegenerative disease (e.g., Alzheimer's disease), and cancer. For example, several studies support use of an RNase for treatment of an inflammatory lung disease. One study has shown that TLR3, an RNA sensor, has a major role in the development of ARDS-like pathology in the absence of a viral pathogen (see Murray et al., Am. J. Respir. Crit. Care Med. 178:1227-1237, 2008). Oxygen therapy is a major therapeutic intervention in ARDS, but contributes to further lung damage and susceptibility to viral infection. Oxygen therapy was a major stimulus for increased TLR3 expression and activation in cultured human epithelial cells, and absence or blockade of TLR3 protected mice from lung injury and inflammation after exposure to hyperoxic conditions (see Murray et al., supra). Another study has shown that TLR3 activation by extracellular RNA occurs in response to acute hypoxia, and that therapy in mice with RnaseA diminished lung inflammation after acute hypoxia (see Biswas et al., Eur. J. Immunol. 45: 3158-3173, 2015).

Other studies support use of an RNase for treatment of an autoimmune disease such as, for example, systemic lupus erythematosus (SLE). Studies show, for example, a role of RNA immune complexes and RNA receptors, including TLR7, in SLE disease pathogenesis, as well as a protective effect of RNase overexpression in mouse models of SLE (see, e.g., Sun et al., J. Immunol. 190:2536-2543, 2013).

RNase-containing fusion molecules as described herein can digest RNA contained in exosomes. For a review of exosomes in lung disease, see Hough et al., Allergy 72:534-544, 2017. RNase1 can translocate through cellular membranes into the cytosol because of its basic charge (see Chao et al., Biochem. 50:8374-8382, 201.1; Haigas et al., J. Cell Sci. 116:313-324, 2003; Johnson et al., Biochem. 46:10308-10316, 2007). Cytoplasmic inhibitor binds to RNase1 with very high affinity and protects intact cells from RNase toxicity. However, extracellular RNA including RNA in extracellular vesicles, is not protected by cytoplasmic inhibitor (see Mironova et al., Oncotarget 8: 78796-78810, 2017).

Fusion molecules comprising an RNase as described herein can further target microRNA (miRNA), small, extracellular noncoding RNAs involved in posttranscriptional gene regulation. miRNA dysregulations are linked to a wide spectrum of diseases, including lung disease, proliferative vascular disease, cardiac disorders, kidney diseases, diabetes mellitus, fibrosis and cancer (see Rajasekaran et al., Front. Pharmacol. 6:254, 2015; Thum et al., Nature 456:980-984, 2008; Kato et al., Clin. J. Am. Soc. Nephrol. 4:1255-1266, 2009; Lee and Dutta, Annu. Rev. Pathol. 4:199-227, 2009; Kumar et al., Protein Cell. 3:726-738, 2012; Noetel et al., Front. Physiol. 3:49, 2012; Zampetaki and Mayr, Circ. Res. 110:508-522, 2012). For example, microRNA-33 is overexpressed in granulomatous lung tissue in murine models and in BAL fluid of patients with sarcoidosis (Barna et al., Am. J. Respir. Cell Mol. Biol. 54:865-871. 2016). microRNA-33 downregulates cholesterol transporters ABCA1 and ABCG1, promoting cholesterol accumulation in alveolar macrophages and foam cell formation. Another target is microRNA-466, which has been shown to be secreted in exosomes in a mouse model of acute respiratory distress syndrome (ARDS) where it activated the NLRP3 inflammasome (Shikano et al., BMC Pulmonary Med. 19:110, 2019).

Other targets of RNase-containing fusions as described herein include viral RNAs such as, e.g., proviral microRNAs released in the lung during influenza virus infection (see Scheller et al., J. Inf. Dis. 219:540-543, 2019) or HIV single-stranded RNA that was reported to stimulate macrophage foam cell formation through TLR8 binding (see Bernard et al., PloS ONE 9:e104039, 2014). Other viral RNA targets include coronavirus RNA (e.g., SARS-CoV-2 RNA).

Studies also support use of RNase for treatment of, e.g., cancer, ischemia/reperfusion injury, graft rejection, atherosclerotic plaque formation, myocardial infarction, acute stroke, and postoperative cognitive decline (see, e.g., Rutkoski et al., Translational Oncology 6:392-397, 2013; Mironova et al., Oncotarget. 8:78796-78810, 2017; Eller et al., ACS Central Sci. 1:181-190, 2015; Kleinert et al., J. Am. Heart Assn. doi: 10.1021, 2016; Simsekyilmaz et al., Circulation 129: 598-606, 2014; Steiger et al., JAMA 6:e004541, 2017; Walberer et al., Curr. Neovas. Res. 6:12-19, 2009; Chen et al., PloS One 10:e0134307, 2015).

A bispecific paraoxonase fusion molecule comprising a superoxide dismutase (SOD1) as described herein provides additional therapeutic benefit in protection from oxidative stress. The role of SOD1 in protection from oxidative damage is reviewed, e.g., by Ighodaro and Akinolye, Alexandria J. Med. 54:287-293, 2018. In its native form, SOD1 is an intracellular enzyme (see id.). In accordance with the present disclosure, a SOD1 polypeptide segment is used as an extracellular enzyme that works in conjunction with the paraoxonase segment of the fusion molecule to provide an improved antioxidant for therapy. For example, paraoxonase can digest hydrogen peroxide (H₂O₂), a product of the SOD1 enzymatic reaction. In addition, digestion of H₂O₂ will suppress the activity of myeloperoxidase that is associated with NETs. The coordinated activity of SOD1 and paraoxonase will improve the activity in protection from tissue damage and inflammation in patients with diseases associated with NET production. SOD1-PON molecules according to the present invention are particularly useful, for example, in treatment of inflammatory lung diseases such as, e.g., asthma, chronic obstructive pulmonary disease (COPD), sarcoidosis, and interstitial lung disease. In some preferred embodiments, the SOD1-PON fusion further includes a dimerizing domain such as, e.g., an immunoglobulin Fc region; such variations are particularly compatible with SOD1, which, in its native form, is a dimer stabilized by a disulfide bond.

A bispecific paraoxonase fusion molecule comprising a CTLA-4 extracellular domain as described herein is particularly useful for treatment of inflammatory and autoimmune disease, including, e.g., rheumatoid arthritis (RA) and inflammatory lung disease. Oxidized cholesterol activates inflammatory responses in macrophages and endothelial cells (see Miller and Shyy, Trends Endocrinol. Metabol. 28:143-152, 2017), and PON1 can suppress this inflammation by protecting lipoproteins from oxidation. The immunosuppressive properties of a soluble CTLA-4 (e.g., Abatacept or other CTLA4-Fc) are compatible with paraoxonase enzyme activity so that a CTLA4-PON molecule will retain the activity of both components and have improved activity in fibrotic lung disease and autoimmune/inflammatory disease. A CTLA4-PON1 molecule, for example, will bind to CD80 and CD86 that are expressed on activated antigen presenting cells, including monocytes and dendritic cells, where the PON1 enzyme will remain active. This molecule is effective in inhibiting activation of T cells, macrophages, and dendritic cells, and will also reduce oxidative stress in tissues such as, e.g., inflamed lungs.

A bispecific paraoxonase fusion molecule comprising a CD40 extracellular domain as described herein provides additional therapeutic benefit by suppressing the proinflammatory activation events associated with the CD40-CD154 signaling pathway. The CD40-CD154 pathway has been implicated, for example, in fibrotic disease, including fibrosis in inflammatory lung disease and injury (see. e.g., Kaufman et al., J. Immunol. 172:1862-1871, 2004), and studies support the use of molecules that suppress this pathway for treatment of fibrotic and inflammatory lung disease (see, e.g., Adawi et al., Clin. Immunol. Immunopathol. 89:222-230, 1998; Adawi et al., Am. J. Pathol. 152:651-657, 1998; Cheng et al., BioMed. Research Intl. Volume 2020, Article ID 7840652, 2020; Xiong et al., J. Cell. Mol. Med. 23:740-749, 2018). CD40-PON fusions of the present invention provide molecules that inhibit both inflammation and adaptive immunity, including e.g., molecules with improved activity in lung disease and autoimmune/inflammatory disease. For example, a CD40-PON1 fusion may provide improved activity in lung diseases where CD40 activation exacerbates inflammatory processes, and where paraoxonase levels are low or absent, including asthma, COPD, hypoxia, and interstitial lung disease (e.g., idiopathic pulmonary fibrosis (IPF) or sarcoidosis). CDO40-PON1 fusions are also beneficial, for example, for treatment of liver disease (e.g., chronic liver disease such as, for example, alcohol-associated liver disease (ALD), portal hypertension, nonalcoholic steatohepatitis (NASH), or complications following liver transplantation) and kidney disease (e.g., chronic kidney disease such as, for example, lupus nephritis, IgA nephropathy, or membranous glomerulonephritis).

In some embodiments, a paraoxonase fusion molecule of the present invention comprises a variant CD40 extracellular domain having increased binding to CD154. For example, one or more single amino acid substitutions can be made at specific residues to improve binding to CD40 ligand (see, e.g., US Patent Application Publication No. 2014/0120091, incorporated by reference herein). Such CD40 extracellular domain variants may be fused to a human IgG1 (γ1) Fc variant with impaired FcR binding to provide CD40-Fc variants that do not induce platelet activation or aggregation in vitro, avoiding toxicity from simultaneous binding to CD40 and FcγRIIa on human platelets. Activation of platelets and thrombosis has been shown to occur due to crosslinking of FcR on platelets with antigens on the platelet surface such as VEGF, CD40, and CD40L (see Taylor et al., Blood 96:4254, 2020; Meyer el al., J. Thromb. Haemost. 7:151, 2009).

Bispecific fusion molecules comprising a polypeptide that specifically binds and neutralizes TNFα or TGF-β as described herein provide additional therapeutic benefit through inhibition of inflammatory, immune, and/or other cellular responses mediated by these cytokines. A paraoxonase fusion comprising an anti-TNFα component may be used, for example, to treat an inflammatory disease or autoimmune disease such as, e.g., rheumatoid arthritis (RA), an inflammatory lung disease (e.g., asthma, chronic obstructive pulmonary disease (COPD), acute lung injury (ALI), acute respiratory distress syndrome (ARDS), or interstitial lung disease (e.g., idiopathic pulmonary fibrosis or sarcoidosis)), an inflammatory bowel disease (IBD) (e.g., Crohn's disease or ulcerative colitis), or an inflammatory skin disease (e.g., psoriasis or atopic dermatitis). A paraoxonase fusion comprising an anti-TGF-β component may be used, for example, to treat an inflammatory disease, a fibrotic disease, or type 1 diabetes. In some embodiments, an inflammatory or fibrotic disease for treatment using an antiTGFβ-PON fusion of the present invention is an inflammatory lung disease such as, for example, asthma, chronic obstructive pulmonary disease (COPD), acute lung injury (ALI), acute respiratory distress syndrome (ARDS), or interstitial lung disease (e.g., idiopathic pulmonary fibrosis (IPF) or sarcoidosis). In some variations for treatment of an inflammatory lung disease, a fusion molecule comprising an anti-TNFα or anti-TGF-β polypeptide as describe herein is delivered by nebulization directly to the lungs rather than systemically, which may achieve efficient therapeutic activity with fewer unwanted side effects that may arise due to the pleiotropic activity of these cytokines.

In some aspects, the present invention provides compositions and methods for producing fusion polyptides and dimeric proteins as described herein, including such compositions and methods for the constitutive expression of DNase-PON fusions in stable cells lines such as CHO. Previous efforts to obtain stable CHO cells that constitutively express an actin/salt-resistant and hyperactive DNase1 were unsuccessful despite screening thousands of clones (see Lam et al., Biotech. Progress 32: 523-533, 2017). In certain embodiments, DNase-PON fusions comprising an enhanced DNase1 as described herein are readily expressed in stable lines with minimal screening, thereby overcoming this problem in the art. The ability to readily generate stable cell lines expressing certain enhanced DNase1-PON molecules of the present disclosure indicate that these molecules have advantages in feasibility and cost of industrial scale biologic drug manufacturing.

### II. Fusion Polypeptides and Dimeric Proteins

In one aspect, the present invention provides a fusion polypeptide comprising, from an amino-terminal position to a carboxyl-terminal position, T-L1-X-L2-P, wherein T, L1, X, L2, and P are defined as follows: T is a first biologically active polypeptide selected from a DNase, an RNase, a superoxide dismutase 1 (SOD1), a cytotoxic T-lymphocyte associated molecule-4 (CLTA-4) extracellular domain, a CD40 extracellular domain, and a polypeptide that specifically binds and neutralizes an inflammatory cytokine; L1 is a first polypeptide linker that is optionally present; X is a second biologically active polypeptide that is optionally present; L2 is a second polypeptide linker that is optionally present; and P is a biologically active paraoxonase. In some embodiments of a fusion polypeptide as above wherein X is present, X is selected from a dimerizing domain and a domain that specifically binds to the neonatal Fc receptor (FcRn). Typically, the biologically active paraoxonase is a naturally occurring paraoxonase 1 (PON1) polypeptide or a functional variant or fragment thereof. In variations comprising a DNase, an RNase, a SOD1, a CLTA-4 extracellular domain, or a CD40 extracellular domain, any of the foregoing biologically active polypeptides may be selected from a naturally occurring polypeptide as specified or a functional variant or fragment thereof. In certain variations comprising a polypeptide that specifically binds and neutralizes an inflammatory cytokine (e.g., TNFα or TGF-β), the anti-cytokine polypeptide is a single-chain antibody or alternative scaffold binding protein.

In another aspect, the present invention provides a fusion polypeptide comprising, from an amino-terminal position to a carboxyl-terminal position, X-L2-P, wherein X is biologically active polypeptide selected from a dimerizing domain and a domain that specifically binds to the neonatal Fc receptor (FcRn), L2 is a polypeptide linker that is optionally present, and P is a biologically active paraoxonase, wherein the fusion polypeptide does not contain a biologically active polypeptide amino-terminal to X. Typically, the biologically active paraoxonase is a naturally occurring paraoxonase 1 (PON1) polypeptide or a functional variant or fragment thereof.

In yet another aspect, the present invention provides a fusion polypeptide comprising, from an amino-terminal position to a carboxyl-terminal position, P-L1-X, wherein P is a biologically active paraoxonase, L1 is a polypeptide linker, and X is biologically active polypeptide selected from a dimerizing domain and a domain that specifically binds to the neonatal Fc receptor (FcRn). Typically, the biologically active paraoxonase is a naturally occurring paraoxonase 1 (PON1) polypeptide or a functional variant or fragment thereof.

Functional variants of a naturally occurring paraoxonase or other biologically active polypeptides specified above can be readily identified using routine assays for assessing the variant for the corresponding biological activity. For example, paraoxonase 1 (PON1) variants may be assayed for phosphotriesterase activity using diethyl p-nitrophenol phosphate (paraoxon) as a substrate, or for arylesterase activity using phenyl acetate as a substrate (see, e.g., Graves and Scott, Curr Chem Genomics 2:51-61, 2008; see also Example 3, infra). In addition, DNase1 and DNase1L3 variants, including hyperactive DNase1 variants, may be assayed for nuclease activity using (i) a DNA-methyl green assay, which measures the decrease of A₂₆₀ as the methyl green dye is released from hydrolyzed DNA (see. e.g., Sinicropi et al., Anal. Biochem. 222:351-358; Pan and Lazarus, J. Biol. Chem. 273:11701-11708, 1998), (ii) a Kunitz hyperchromicity assay, in which the A₂₆₀, due to the DNA absorption, increases as a function of degradation (see, e.g., Kunitz, J. Gen. Physiol. 33:349-362, 1950: Pan and Lazarus, supra); (iii) a plasmid digestion assay, which uses either supercoiled or linear plasmid DNA as a substrate and measures the disappearance of substrate and/or appearance of digestion products (e.g., appearance of linear or relaxed products from supercoiled DNA) (see, e.g., Pan and Lazarus, supra), or (iv) a SYTOX^{T}M Green fluorescence assay, which measures the decrease in fluorescence as DNA labeled with SYTOX Green dye is hydrolyzed (see Example 2, infra). DNase1 variants may also be assayed for G-actin-induced inhibition of nuclease activity using DNase preincubated with G-actin prior to addition of substrate DNA (see, e.g., Pan el al., J. Biol. Chem. 273:18374-18381, 1998). In the case of RNase such as human RNase1, variants may be assayed for their ability to digest single or double-stranded RNA in known assays to assess ribonuclease activity (see, e.g., Libonati and Sorrentino, Methods Enzymol. 341:234-248, 2001).

SOD1 variants may be assayed using a commercially available superoxide dismutase colorimetric activity kit (ThermoFisher™ Catalog number: EIASODC). This kit is designed to measure all types of superoxide dismutase activity in a variety of sample types, including Cu/Zn, Mn, and Fc superoxide dismutase activities. Reactive oxygen species including superoxide, hydroxyl radical, and hydrogen peroxide, are produced during normal cellular aerobic metabolism, and free radicals are eliminated or converted to other products by several pathways, including the SOD enzymes. This assay should quantitatively measure SOD activity and uses a bovine erythrocyte SOD standard to generate a standard curve. A similar spectrophotometric kit is also available from Cayman Chemicals (Item number 706002), and SOD activity assays using this kit are described in Mason et al., Free Radic. Biol. Med. 77:130-138, 2014.

Soluble CLTA-4 and CD40 variants, as well as anti-inflammatory-cytokine polypeptides (e.g., single chain antibodies), may be assessed for desired binding activity against their respective targets (e.g., against CD80/CD86 in the case of CTLA-4, or against gp139 in the case of CD40) using any of various known assays. For example, one assay system employs a commercially available biosensor instrument (BIAcore™, Pharmacia Biosensor, Piscataway, NJ), wherein a candidate binding polypeptide (e.g., a candidate CTLA-4-Fc, CD40-Fc, or anti-cytokine antibody) is covalently attached, using amine or sulfhydryl chemistry, to dextran fibers that are attached to gold film within the flow cell. A test sample containing a soluble target molecule (e.g., CD80-Fc, CD86-Fc, CD40L-Fc (gp139-Fc), or soluble cytokine) is passed through the cell. If the immobilized protein has affinity for the target molecule, it will bind to the target causing a change in the refractive index of the medium, which is detected as a change in surface plasmon resonance of the gold film. This system allows the determination of on- and off-rates, from which binding affinity can be calculated, and assessment of stoichiometry of binding. Use of this instrument is disclosed, e.g., by Karlsson (J. Immunol. Methods 145:229-240, 1991) and Cunningham and Wells (J. Mol. Biol. 234:554-563, 1993). Binding activity of candidate polypeptides can also be assessed with other assay systems known in the art. Such systems include Scatchard analysis for determination of binding affinity (see Scatchard, Ann. NY Acad Sci. 51: 660-672, 1949) and calorimetric assays (see Cunningham et al., Science 253:545-548, 1991; Cunningham et al., Science 254:821-825, 1991).

Activity of soluble CTLA-4 and CD40 variants can also be assessed for function in appropriate in vitro cellular assays. For example, soluble CTLA-4 will inhibit T cell responses to stimulation by CD80- or CD86-expressing cells, such as B cells, dendritic cells, or CD80⁺ W or CD86⁺ CHO cells (see, e.g., U.S. Pat. No. 5,434,131; Linsley et al., J. Exp. Med. 174:561-569, 1991). Soluble CD40 will block stimulation of CD40 positive cells by CD154 (CD40L, gp39). CD40-responsive cells can be B cells, monocytes, or dendritic cells. The response can vary depending on the cell type, and may include proliferation, suppression of antibody production, or inflammatory cytokine production (see, e.g., Noelle et al., Proc. Natl. Acad. Sci. USA 89:6550-6554, 1992; Grammer et al., J. Immunol. 154:4996-5010, 1995). A CD40L reporter cell line is also available from Invivogen (San Diego, CA). This reporter system uses HEK293 cells transfected with human CD40 and an NF-kB response element fused to secreted embryonic alkaline phosphatase (SEAP). Binding of the CD40 receptor by its ligand or antibodies leads to NF-kB activation and inducible expression of the SEAP. (See Jerome et al., Anal. Biochem. 585:113402, 2019.)

Neutralizing activity of inflammatory-cytokine-binding polypeptides can also be assessed using cellular assays known in the art. For example, anti-TNFα-binding proteins can be assayed for neutralizing activity using commercially available TNFα reporter cell lines available from Invivogen (San Diego, CA). These cell lines are transfected HEK-293 cells with a secreted embryonic alkaline phosphatase (SEAP) reporter gene attached to TNFα-responsive promoters. (Gregory et al., Nature 488(7412):508-511, 2012; Idress et al., Molecules 25:922, 2020.) In addition, anti-TGFβ-binding proteins can be assessed for neutralizing activity using similar reporter cell lines available from Invivogen (San Diego, CA), that contain TGF-β-responsive elements to drive expression of SEAP. Use of this reporter system has been described by van Noort et al. (Cancer Res. 74:5690-5699, 2014). Similar reporter gene assays have been reported for TNFα antagonists by Lallemand et al. (J. Immunol. Methods 373:229-239, 2011). The study utilized human erythroleukemic K562 cells transfected with a reporter construct incorporating TNFα response elements attached to the firefly luciferase gene. TNFα was premixed with potential antagonists for 30 minutes prior to incubation with the reporter cell line. Residual TNFα activity was quantified relative to a standard curve of TNFα alone.

Naturally occurring polypeptide segments for use in accordance with the present disclosure (e.g., a naturally occurring paraoxonase, DNase, RNase, or SOD1) include naturally occurring variants such as, for example, allelic variants and interspecies homologs consistent with the disclosure.

Functional variants of a particular reference polypeptide (e.g., a wild-type human paraoxonase 1 (PON1) as shown in residues 16-355 or 26-355 of SEQ ID NO:4, a wild-type DNase1 as shown in residues 23-282 of SEQ ID NO:16, or an enhanced DNase1 as shown in residues 21-280 of SEQ ID NO:18) are generally characterized as having one or more amino acid substitutions, deletions, or additions relative to the reference polypeptide. These changes are preferably of a minor nature, that is conservative amino acid substitutions (see, e.g., Table 1, infra, which lists some exemplary conservative amino acid substitutions) and other substitutions that do not significantly affect the folding or activity of the protein or polypeptide; small deletions, typically of one to about 30 amino acids; and small amino- or carboxyl-terminal extensions. Conservative substitutions may also be selected from the following: 1) Alanine, Glycine; 2) Aspartate, Glutamate; 3) Asparagine, Glutamine; 4) Arginine, Lysine; 5) Isoleucine, Leucine, Methionine, Valine; 6) Phenylalanine, Tyrosine, Tryptophan; 7) Serine, Threonine; and 8) Cysteine, Methionine (see, e.g., Creighton, Proteins (1984)).

TABLE 1
Conservative amino acid substitutions

| Basic | Acidic | Polar | Hydrophobic | Aromatic | Small |
|---|---|---|---|---|---|
| Arginine | Glutamate | Glutamine | Leucine | Phenylalanine | Glycine |
| Lysine | Aspartate | Asparagine | Isoleucine | Tryptophan | Alanine |
| Histidine | | | Valine | Tyrosine | Serine |
| | | | Methionine | | Threonine |
| | | | | | Methionine |

Essential amino acids in a naturally occurring polypeptide can be identified according to procedures known in the art, such as site-directed mutagenesis or alanine-scanning mutagenesis (Cunningham and Wells, Science 244:1081-1085, 1989; Bass et al., Proc. Natl. Acad. Sci. USA 88:4498-4502, 1991). In the latter technique, single alanine mutations are introduced at every residue in the molecule, and the resultant mutant molecules are tested for biological activity (e.g., phosphotriesterase or arylesterase activity for PON1 variants, or nuclease activity for DNase1 variants) to identify amino acid residues that are critical to the activity of the molecule. In addition, sites of relevant protein interactions can be determined by analysis of crystal structure as determined by such techniques as nuclear magnetic resonance, crystallography or photoaffinity labeling. The identities of essential amino acids can also be inferred from analysis of homologies with related proteins (e.g., species orthologs retaining the same protein function).

Multiple amino acid substitutions can be made and tested using known methods of mutagenesis and screening, such as those disclosed by Reidhaar-Olson and Sauer Science 241:53-57, 1988 or Bowie and Sauer Proc. Natl. Acad. Sci. USA 86:2152-2156, 1989. Briefly, these authors disclose methods for simultaneously randomizing two or more positions in a polypeptide, selecting for functional polypeptide, and then sequencing the mutagenized polypeptides to determine the spectrum of allowable substitutions at each position. Another method that can be used is region-directed mutagenesis (Derbyshire et al., Gene 46:145, 1986; Ner et al., DNA 7:127, 1988).

Variant nucleotide and polypeptide sequences can also be generated through DNA shuffling. (See, e.g., Stemmer, Nature 370:389, 1994; Stemmer, Proc. Nat′l Acad. Sci. USA 91:10747, 1994; International PCT Publication No. WO 97/20078.) Briefly, variant DNA molecules are generated by in vitro homologous recombination by random fragmentation of a parent DNA followed by reassembly using PCR, resulting in randomly introduced point mutations. This technique can be modified by using a family of parent DNA molecules, such as allelic variants or DNA molecules from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

As previously discussed, a polypeptide fusion in accordance with the present invention can include a polypeptide segment corresponding to a “functional fragment” of a particular polypeptide. Routine deletion analyses of nucleic acid molecules can be performed to obtain functional fragments of a nucleic acid molecule encoding a given polypeptide. As an illustration, PON1-encoding DNA molecules having the nucleotide sequence of residues 46-1099 can be digested with Bal131 nuclease to obtain a series of nested deletions. The fragments are then inserted into expression vectors in proper reading frame, and the expressed polypeptides are isolated and tested for nuclease activity. One alternative to exonuclease digestion is to use oligonucleotide-directed mutagenesis to introduce deletions or stop codons to specify production of a desired fragment. Alternatively, particular fragments of a gene encoding a polypeptide can be synthesized using the polymerase chain reaction.

Accordingly, using methods such as discussed above, one of ordinary skill in the art can prepare a variety of polypeptides that (i) are substantially identical to a reference polypeptide (e.g., a PON1 as shown in residues 16-355 or 26-355 of SEQ ID NO:6, a human wild-type DNase1 as shown in residues 23-282 of SEQ ID NO:16, or an enhanced DNase1 as shown in residues 21-280 of SEQ ID NO:18) and (ii) retains the desired functional properties of the reference polypeptide.

Polypeptide segments used within the present invention (e.g., polypeptide segments corresponding to a paraoxonase, DNase, RNase, or dimerizing or FcRn-binding domain such as, e.g., an Fc fragment) may be obtained from a variety of species. If the protein is to be used therapeutically in humans, it is preferred that human polypeptide sequences be employed. However, non-human sequences can be used, as can variant sequences. For other uses, including in vitro diagnostic uses and veterinary uses, polypeptide sequences from humans or non-human animals can be employed, although sequences from the same species as the patient may be preferred for in vivo veterinary use or for in vitro uses where species specificity of intermolecular reactions is present. Thus, polypeptide segments for use within the present invention can be, without limitation, human, non-human primate, rodent, canine, feline, equine, bovine, ovine, porcine, lagomorph, and avian polypeptides, as well as variants thereof.

In some embodiments, the paraoxonase segment is a human paraoxonase 1 (PON1) or a functional variant or fragment thereof. For example, in some embodiments, the paraoxonase (a) has at least 80%, at least 85%, at least 90%, or at least 95% identity with amino acid residues 16-355 or 26-355 of SEQ ID NO:6, SEQ ID NO:8, or SEQ ID NO:4 and (b) does not contain an amino-terminal leader sequence corresponding to residues 1-15 of SEQ ID NO:6 (also shown as residues 1-15 of SEQ ID NO:8 or SEQ ID NO:4). In some such embodiments, the biologically active paraoxonase has at least 96%, at least 97%, at least 98%, or at least 99% identity with amino acid residues 16-355, 17-355, 18-355, 19-355, 20-355, 21-355, 22-355, 23-355, 24-355, 25-355, or 26-355 of SEQ ID NO:6, SEQ ID NO:8, or SEQ ID NO:4.

In some variations of a PON1 or functional variant or fragment thereof as above, the amino acid at a position corresponding to Q192 of the human paraoxonase 1 Q192 isoform (PON1-Q192; SEQ ID NO:4) is lysine or arginine. In other, non-mutually exclusive variations, the paraoxonase contains at least one of the following amino acid substitutions relative to hPON1-Q192: (i) aspartate at the position corresponding to 18R; (ii) arginine or glycine at the position corresponding to N19; (iii) glutamine at the position corresponding to H20; (iv) lysine at the position corresponding to Q21; (v) glutamate or phenylalanine at the position corresponding to Y24; (vi) phenylalanine at the amino acid corresponding to L28; (vii) valine at the position corresponding to A₃₀; (viii) histidine at the position corresponding to L31; (ix) threonine at the position corresponding to Q35; (x) valine at the position corresponding to 148; (xi) aspartate at the position corresponding to E49; (xii) asparagine at the position corresponding to T50; (xiii) leucine or isoleucine at the position corresponding to M55; (xiv) valine at the position corresponding to L69; (xv) methionine at the position corresponding to K75; (xvi) aspartate at the position corresponding to N78; (xvii) aspartate at the position corresponding to N80; (xviii) lysine at the position corresponding to S81; (xix) serine at the position corresponding to P82; (xx) valine at the position corresponding to T96; (xxi) serine at the position corresponding to L98; (xxii) glutamate at the position corresponding to G101; (xxiii) asparagine at the position corresponding to S105; (xxiv) threonine at the position corresponding to K106; (xxv) leucine at the position corresponding to F107; (xxvi) isoleucine at the position corresponding to V109; (xxvii) threonine at the position corresponding to S111; (xxviii) tryptophan or alanine at the position corresponding to H115; (xxix) threonine at the position corresponding to A₁₂₆; (xxx) valine at the position corresponding to M127; (xxxi) arginine at the position corresponding to H134; (xxxii) glutamine at the position corresponding to D136; (xxxiii) serine at the position corresponding to A₁₃₇; (xxxiv) serine at the position corresponding to K138; (xxxv) valine at the position corresponding to L143; (xxxvi) serine at the position corresponding to N166; (xxxvii) valine at the position corresponding to L167; (xxxviii) alanine at the position corresponding to G180; (xxxix) glutamate at the position corresponding to Y185; (xl) glutamine at the position corresponding to F186; (xli) lysine or alanine at the position corresponding to L187; (xlii) lysine at the position corresponding to Y190; (xxliii) glutamine at the position corresponding to L191; (xliv) lysine at the position corresponding to Q192; (xlv) lysine at the position corresponding to W194; (xlvi) histidine at the position corresponding to Y197; (xlvii) glutamate at the position corresponding to L198; (xlviii) glutamine at the position corresponding to L200; (xlix) lysine at the position corresponding to W202; (1) phenylalanine at the position corresponding to Y204; (li) threonine at the position corresponding to V206; (lii) asparagine at the position corresponding to S211; (liii) aspartate at the position corresponding to E212; (liv) serine or methionine at the position corresponding to F222; (lv) aspartate at the position corresponding to N265; (lvi) valine at the position corresponding to E276; (lvii) glutamine at the position corresponding to M289; (lviii) leucine at the position corresponding to 1291; (lix) glutamate or tyrosine at the position corresponding to F293; (lx) proline at the position corresponding to S296; (lxi) lysine at the position corresponding to E297; (lxii) glycine tha the position corresponding to A₃₀₁; (lxiii) aspartate at the position corresponding to N309; (lxiv) serine at the position corresponding to T312; (Ixv) valine at the position corresponding to Q319; (lxvi) serine at the position corresponding to T332; and alanine at the position corresponding to S335.

In some variations, the paraoxonase is a variant of human PON1 identified as G3C9 (Aharoni et al., Proc. Natl. Acad. Sci. USA 101:482-487, 2004; Harel et al., Nat. Struct. Mol. Biol. 11:412-419, 2004; Mukherjee and Gupta, J. Toxicol. 2020:1-16, 2020; Goldsmit et al., Chemistry and Biology 19:456-466, 2012), or a functional variant or fragment thereof. GC39 includes 55 amino acid substitutions from the human isoform, improving the catalytic activity against nerve agents and improving soluble expression in bacteria. The full length form of this paraoxonase sequence variant is listed in SEQ ID NO:123 (nucleotide) and SEQ ID NO:124 (amino acid). In some embodiments, a GC39 paraoxonase or functional variant thereof for use in accordance with the present invention has at least 90% or at least 95% identity with amino acid residues 16-355 or 26-355 of SEQ ID NO:124; in some such embodiments, the GC39 paraoxonase or functional variant thereof has at least 96%, at least 97%, at least 98%, or at least 99% identity with amino acid residues 16-355, 17-355, 18-355, 19-355, 20-355, 21-355, 22-355, 23-355, 24-355, 25-355, or 26-355 of SEQ ID NO:124. PON1 fusions comprising G3C9 or an active variant thereof (e.g., M-IIG1 discussed below) may be particularly useful in some short term therapy applications such as, e.g., treatment of exposure to sulfur mustard gas or exposure to an organophosphate.

In some embodiments of a fusion polypeptide as above, the paraoxonase is a modified or further variant form of GC39 identified as IIG1 (also referred to as M-IIG1 herein; see Goldsmith et al., Chemistry & Biology 19, 456-466, 2012), or a functional variant or fragment thereof. The full length sequence of M-IIG1 is shown in SEQ ID NO:125 (nucleotide) and SEQ ID NO:126 (amino acid). In some embodiments, a M-IIG1 paraoxonase or functional variant thereof for use in accordance with the present invention has at least 90% or at least 95% identity with amino acid residues 16-355 or 26-355 of SEQ ID NO:126; in some such embodiments, the M-IIG1 paraoxonase or functional variant thereof has at least 96%, at least 97%, at least 98%, or at least 99% identity with amino acid residues 16-355, 17-355, 18-355, 19-355, 20-355, 21-355, 22-355, 23-355, 24-355, 25-355, or 26-355 of SEQ ID NO:126.

In more specific variations, the paraoxonase has an amino acid sequence selected from (i) residues 16-355 of SEQ ID NO:6, (ii) residues 17-355 of SEQ ID NO:6, (iii) residues 18-355 of SEQ ID NO:6, (iv) residues 19-355 of SEQ ID NO:6, (v) residues 20-355 of SEQ ID NO:6, (vi) residues 21-355 of SEQ ID NO:6, (vii) residues 22-355 of SEQ ID NO:6, (viii) residues 23-355 of SEQ ID NO:6, (ix) residues 24-355 of SEQ ID NO:6, (x) residues 25-355 of SEQ ID NO:6, and (xi) residues 26-355 of SEQ ID NO:6 (i.e., an amino acid sequence selected from residues n-355 of SEQ ID NO:6, wherein n is an integer from 16 to 26, inclusive). In other variations, the paraoxonase has an amino acid sequence selected from (i) residues 16-355 of SEQ ID NO:8, (ii) residues 17-355 of SEQ ID NO:8, (iii) residues 18-355 of SEQ ID NO:8, (iv) residues 19-355 of SEQ ID NO:8, (v) residues 20-355 of SEQ ID NO:8, (vi) residues 21-355 of SEQ ID NO:8, (vii) residues 22-355 of SEQ ID NO:8, (viii) residues 23-355 of SEQ ID NO:8, (ix) residues 24-355 of SEQ ID NO:8, (x) residues 25-355 of SEQ ID NO:8, and (xi) residues 26-355 of SEQ ID NO:8 (i.e., an amino acid sequence selected from residues n-355 of SEQ ID NO:8, wherein n is an integer from 16 to 26, inclusive). In still other variations, the paraoxonase has an amino acid sequence selected from (i) residues 16-355 of SEQ ID NO:4, (ii) residues 17-355 of SEQ ID NO:4, (iii) residues 18-355 of SEQ ID NO:4, (iv) residues 19-355 of SEQ ID NO:4, (v) residues 20-355 of SEQ ID NO:4, (vi) residues 21-355 of SEQ ID NO:4, (vii) residues 22-355 of SEQ ID NO:4, (viii) residues 23-355 of SEQ ID NO:4, (ix) residues 24-355 of SEQ ID NO:4. (x) residues 25-355 of SEQ ID NO:4, and (xi) residues 26-355 of SEQ ID NO:4 (i.e., an amino acid sequence selected from residues n-355 of SEQ ID NO:4, wherein n is an integer from 16 to 26, inclusive). In still other variations, the paraoxonase has an amino acid sequence selected from (i) residues 16-355 of SEQ ID NO:124, (ii) residues 17-355 of SEQ ID NO:124, (iii) residues 18-355 of SEQ ID NO:124, (iv) residues 19-355 of SEQ ID NO:124, (v) residues 20-355 of SEQ ID NO: 124, (vi) residues 21-355 of SEQ ID NO:124, (vii) residues 22-355 of SEQ ID NO:124, (viii) residues 23-355 of SEQ ID NO:124, (ix) residues 24-355 of SEQ ID NO:124, (x) residues 25-355 of SEQ ID NO:124, and (xi) residues 26-355 of SEQ ID NO:124 (i.e., an amino acid sequence selected from residues n-355 of SEQ ID NO:124, wherein n is an integer from 16 to 26, inclusive). In yet other variations, the paraoxonase has an amino acid sequence selected from (i) residues 16-355 of SEQ ID NO:126, (ii) residues 17-355 of SEQ ID NO:126, (iii) residues 18-355 of SEQ ID NO:126, (iv) residues 19-355 of SEQ ID NO:126, (v) residues 20-355 of SEQ ID NO:126, (vi) residues 21-355 of SEQ ID NO:126, (vii) residues 22-355 of SEQ ID NO:126, (viii) residues 23-355 of SEQ ID NO:126 (ix) residues 24-355 of SEQ ID NO:126, (x) residues 25-355 of SEQ ID NO:126, and (xi) residues 26-355 of SEQ ID NO:126 (i.e., an amino acid sequence selected from residues n-355 of SEQ ID NO:126, wherein n is an integer from 16 to 26, inclusive).

In some embodiments of a fusion polypeptide comprising a DNase as the first biologically active polypeptide, the DNase is a human wild-type DNase1 or a functional variant or fragment thereof. For example, in some embodiments, the DNase comprises an amino acid sequence having at least 80%, at least 85%, at least 90%, or at least 95% identity with (i) amino acid residues 23-282 of SEQ ID NO:16, (ii) amino acid residues 21-280 of SEQ ID NO:18, (iii) amino acid residues 21-280 of SEQ ID NO:20, or (iv) amino acid residues 21-280 of SEQ ID NO:152. In more particular embodiments, the DNase comprises an amino acid sequence having at least 96%, at least 97%, at least 98%, at least 99%, or 100% sequence identity with (i) amino acid residues 23-282 of SEQ ID NO:16, (ii) amino acid residues 21-280 of SEQ ID NO:18, (iii) amino acid residues 21-280 of SEQ ID NO:20, or (iv) amino acid residues 21-280 of SEQ ID NO:152.

In certain preferred embodiments, the DNase segment is a hyperactive and/or actin-resistant variant of a naturally occurring DNase1 (an “enhanced DNase1”) such as, e.g., an enhanced human DNase1 variant. For example, in some variations, the DNase contains at least one amino acid substitution at a position corresponding to an amino acid of mature wild-type human DNase1 (SEQ ID NO:120) selected from N74, G105, and A114, wherein (1) the amino acid substitution at the position corresponding to N74 of human DNase1 (N74 substitution), if present, increases DNA binding of the DNase relative to human DNase1, (2) the amino acid substitution at the position corresponding to G105 of human DNase1 (G105 substitution), if present, increases DNA binding of the DNase relative to human DNase1, and (3) the amino acid substitution at the position corresponding to A₁₁₄ of human DNase1 (A₁₁₄ substitution), if present, decreases G-actin-induced inhibition of endonuclease activity of the DNase relative to human DNase1. Suitable amino acids for substitution at these positions include lysine at the position corresponding to N74 of human DNase1, arginine at the position corresponding to G105 of human DNase1, and/or phenylalanine at the positions corresponding to A₁₁₄ of human DNase1. In some variations, an enhanced human DNase1 variant as above contains at least two of the N74, G105, and A₁₁₄ substitutions (e.g., substitutions at at least the positions corresponding to both N74 and G105 or both G105 and A114 of human DNase1). In some embodiments, the enhanced human DNase1 contains each of the N74, G105, and A₁₁₄ substitutions. In other variations, the enhanced human DNase1 contains the N74 and G105 substitutions but does not contain the A₁₁₄ substitution. In certain embodiments, the enhanced DNase1 comprises an amino acid sequence having at least 80%, at least 85%, at least 90%, at least 95%, at least 96%, at least 97%, at least 98%, at least 99%, or 100% identity with (i) amino acid residues 21-280 of SEQ ID NO:18, (ii) amino acid residues 21-280 of SEQ ID NO:20, or (iii) residues 21-280 of SEQ ID NO:152. In some variations, the enhanced DNase1 comprises an amino acid sequence having at least 90%, at least 95%, at least 96%, at least 97%, at least 98%, or at least 99% identity with amino acid residues 21-280 of SEQ ID NO:18, wherein the enhanced DNase1 contains each of the N74, G105, and A114 substitutions but does not contain any other amino acid substitution that increases DNA binding of the DNase relative to human DNase1, and/or does not contain any other amino acid substitution that decreases G-actin-induced inhibition of endonuclease activity of the DNase relative to human DNase1. In other variations, the enhanced DNase1 comprises an amino acid sequence having at least 90%, at least 95%, at least 96%, at least 97%, at least 98%, or at least 99% identity with amino acid residues 21-280 of SEQ ID NO:152, wherein the enhanced DNase1 contains each of the N74 and G105 substitutions but does not contain any other amino acid substitution that increases DNA binding of the DNase relative to human DNase1, and/or does not contain the A114 substitution or any other amino acid substitution that decreases G-actin-induced inhibition of endonuclease activity of the DNase relative to human DNase1. Fusions comprising an enhanced DNase that lacks actin resistance are particularly beneficial for decreasing toxicity to host cells (e.g., CHO) to enable higher expression of the fusion molecule.

In other embodiments of a fusion polypeptide comprising a DNase as the first biologically active polypeptide, the DNase is a truncated form of human wild-type DNase1 L3 in which the carboxyl-terminal nuclear localization signal (referred to herein as NLS2, corresponding to amino acid residues 291-305 of SEQ ID NO:136) is deleted, or a functional variant or fragment of such truncated form of human DNase1 L3. For example, in some embodiments, the DNase comprises an amino acid sequence having at least 80%, at least 85%, at least 90%, or at least 95% identity with (i) amino acid residues 21-290 of SEQ ID NO:136, (ii) amino acid residues 21-290 of SEQ ID NO:138, or (iii) amino acid residues 21-290 of SEQ ID NO:140. In more particular embodiments, the DNase comprises an amino acid sequence having at least 96%, at least 97%, at least 98%, at least 99%, or 100% sequence identity with (i) amino acid residues 21-290 of SEQ ID NO:136, (ii) amino acid residues 21-290 of SEQ ID NO:138, or (iii) amino acid residues 21-290 of SEQ ID NO:140. In certain preferred embodiments comprising a truncated form of human DNase1 L3 or functional variant thereof, the DNase is a variant DNase1L3 containing one or more amino acid substitutions relative to wild-type human DNase1L3 (SEQ ID NO:136) that inactivate the nuclear localization signal in the N-terminal half of the molecule (referred to herein as NLS1, corresponding to amino acid residues 80-96 of SEQ ID NO:136); in some such embodiments, each of the amino acids at positions corresponding to R80, R95, and N96 of SEQ ID NO:136 is alanine or serine (e.g., each of R80, R95, and N96 is alanine, or each of R80, R95, and N96 is serine). In more specific embodiments of a variant, truncated form of DNase1 L3 as above in which the wild-type NLS1 consensus sequence is removed, the DNase has the amino acid sequence shown in (i) amino acid residues 21-290 of SEQ ID NO:138 or (ii) amino acid residues 21-290 of SEQ ID NO:140.

In some embodiments of a fusion polypeptide comprising an RNase as the first biologically active polypeptide, the RNase is a human wild-type RNase1 or a functional variant or fragment thereof. For example, in some embodiments, the RNase comprises an amino acid sequence having at least 80%, at least 85%, at least 90%, or at least 95% identity with amino acid residues 29-156 of SEQ ID NO:22. In more particular variations, the RNase comprises an amino acid sequence having at least 96%, at least 97%, at least 98%, at least 99%, or 100% identity with amino acid residues 29-156 of SEQ ID NO:22.

In some embodiments of a fusion polypeptide comprising a superoxide dismutase 1 (SOD1) as the first biologically active polypeptide, the SOD1 is a human wild-type SOD1 or a functional variant or fragment thereof. For example, in some embodiments, the SOD1 comprises an amino acid sequence having at least 80%, at least 85%, at least 90%, or at least 95% identity with amino acid residues 2-154 of SEQ ID NO:32. In more particular variations, the SOD1 comprises an amino acid sequence having at least 96%, at least 97%, at least 98%, at least 99%, or 100% identity with amino acid residues 2-154 of SEQ ID NO:32. In certain variations, the amino acid at a position corresponding to C7 of human SOD1 (SEQ ID NO:32) is not cysteine, and/or the amino acid a position corresponding to C112 of human SOD1 is not cysteine; in some such embodiments, the amino acid at a position corresponding to C7 of human SOD1 is alanine, and/or the amino acid a position corresponding to C112 of human SOD1 is serine. In more specific variations, the SOD1 comprises the amino acid sequence shown in residues 2-154 of SEQ ID NO:32 wherein the amino acid at position C7 is changed to alanine and the amino acid at position C112 is serine (also shown as residues 23-175 of SEQ ID NO:52 or residues 23-175 of SEQ ID NO:54).

In some embodiments of a fusion polypeptide comprising a CTLA-4 extracellular domain as the first biologically active polypeptide, the extracellular domain is a human wild-type CTLA-4 extracellular domain or a functional variant or fragment thereof. For example, in some embodiments, the CTLA-4 extracellular domain comprises an amino acid sequence having at least 80%, at least 85%, at least 90%, or at least 95% identity with amino acid residues 21-144 of SEQ ID NO:66. In more particular variations, the CTLA-4 extracellular domain comprises an amino acid sequence having at least 96%, at least 97%, at least 98%, at least 99%, or 100% identity with amino acid residues 21-144 of SEQ ID NO:66.

In some embodiments of a fusion polypeptide comprising a CD40 extracellular domain as the first biologically active polypeptide, the extracellular domain is a human wild-type CD40 extracellular domain or a functional variant or fragment thereof. For example, in some embodiments, the CD40 extracellular domain comprises an amino acid sequence having at least 80%, at least 85%, at least 90%, or at least 95% identity with (i) residues 21-188 of SEQ ID NO:74, (ii) residues 21-188 of SEQ ID NO:78, (iii) residues 21-188 of SEQ ID NO:82, (iv) residues 21-188 of SEQ ID NO:86, or (v) residues 21-188 of SEQ ID NO:90. In more particular embodiments, the CD40 extracellular domain comprises an amino acid sequence having at least 96%, at least 97%, at least 98%, or at least 99% identity with (i) residues 21-188 of SEQ ID NO:74, (ii) residues 21-188 of SEQ ID NO:78, (iii) residues 21-188 of SEQ ID NO:82, (iv) residues 21-188 of SEQ ID NO:86, or (v) residues 21-188 of SEQ ID NO:90.

In certain preferred embodiments, the CD40 extracellular domain contains at least one amino acid substitution at a position corresponding to an amino acid of wild-type human CD40 (SEQ ID NO:68) selected from E64, K81, P85, and L121, wherein the at least one amino acid substitution increases CD40 ligand binding relative to human CD40. Particularly suitable amino acid substitutions at these positions are tyrosine at the position corresponding to E64 of human CD40, threonine, histidine, or serine at the position corresponding to K81 of human CD40, tyrosine at the position corresponding to P85 of human CD40, and/or proline at the position corresponding to L121 of human CD40. In some variations, the CD40 extracellular domain contains at least two of the positions corresponding to E64, K81, P85, and L121 of human CD40 (e.g., substitutions at at least the positions corresponding to K81 and L121 of human CD40, or substitutions at at least the positions corresponding to E64, K81, and P85 of human CD40). In some embodiments, the amino acid at the position corresponding to K81 of human CD40 is selected from threonine, histidine, and serine. In other embodiments, the amino acid at the position corresponding to K81 of human CD40 is histidine and the amino acid the position corresponding to L121 of human CD40 is proline. In yet other embodiments, the amino acid at the position corresponding to E64 of human CD40 is tyrosine, the amino acid at the position corresponding to K81 of human CD40 is threonine, and the amino acid at the position corresponding to P85 of human CD40 is tyrosine. In specific variations, the CD40 extracellular domain comprises the amino acid sequence shown in (i) residues 21-188 of SEQ ID NO:74, (ii) residues 21-188 of SEQ ID NO:78, (iii) residues 21-188 of SEQ ID NO:82, (iv) residues 21-188 of SEQ ID NO:86, or (v) residues 21-188 of SEQ ID NO:90.

In some embodiments of a fusion polypeptide comprising a polypeptide that specifically binds and neutralizes an inflammatory cytokine as the first biologically active polypeptide, the inflammatory cytokine is a member of the tumor necrosis factor (TNF) superfamily, a member of the transforming growth factor (TGF) superfamily, an interleukin, a chemokine, a colony-stimulating factor (CSF), or an interferon. In more particular embodiments, the inflammatory cytokine is selected from the group consisting of tumor necrosis factor α (TNFα), transforming growth factor-β(TGFβ), interleukin-4 (IL-4), interleukin-5 (IL-5), interleukin-6 (IL-6), interleukin-8 (IL-8), interleukin-10 (IL-10), interleukin-11 (IL-11), interleukin-12 (IL-12), interleukin-13 (IL-13), interleukin-17A (IL-17A), interleukin-17F (IL-17F), interleukin-1α (IL-1α), interleukin-lβ (IL-1β), interleukin-18 (IL-18), interleukin-21 (IL-21), interleukin-31 (IL-31), interleukin-33 (IL-33), interleukin-36α (IL-36α), interleukin-36β (IL-36β), interleukin IL-36γ (IL-36γ), granulocyte-macrophage colony-stimulating factor (GM-CSF), and interferon γ (IFNγ). In some variations, a polypeptide that specifically binds and neutralizes an inflammatory cytokine is an antibody. In some alternative variations, a polypeptide that specifically binds and neutralizes an inflammatory cytokine is an alternative scaffold protein.

In certain embodiments of a fusion polypeptide comprising a polypeptide that specifically binds and neutralizes TNFα(“anti-TNFα polypeptide”), the anti-TNFα polypeptide competes for binding to TNFα with an antibody having the same heavy and light chain variable domains (VH and VL) as anti-TNFα monoclonal antibody adalimumab. The amino acid sequences of adalimumab VH and VL are shown herein as SEQ ID NO:108 and SEQ ID NO:110, respectively (see also GenBank Accession Nos. LQ961187 and LQ961186 for the heavy and light chain variable regions, respectively; International PCT Publication No. WO 2014/159579).

In certain embodiments of a fusion polypeptide comprising a polypeptide that specifically binds and neutralizes TGF-β(“anti-TGF-β polypeptide”), the anti-TGF-β polypeptide competes for binding to TGF-β with an antibody having the same heavy and light chain variable domains (VH and VL) as anti-TGF-β monoclonal antibody fresolimumab. The amino acid sequences of fresolimumab VH and VL are shown herein as SEQ ID NO:112 and SEQ ID NO: 114, respectively (see also GenBank Accession Nos. JC232803.1 and JC232805 for the heavy and light chain variable regions, respectively; International PCT Publication No. WO 2013/065869).

The ability of a binding protein to compete for binding to TNFα or TGF-β with an antibody having the VH and VL domains of adalimumab or fresolimumab, respectively, may be determined by an assay in which a test TNFα-binding protein (e.g., an antibody), or a functional binding fragment thereof, prevents or inhibits specific binding to TNFα of a reference antibody having the VH and VL domains of adalimumab (i.e., having VH and VL domains of SEQ ID NO:108 and SEQ ID NO:110, respectively), or in which a test TNF-β-binding protein (e.g., an antibody), or a functional binding fragment thereof, prevents or inhibits specific binding to TNF-β of a reference antibody having the VH and VL domains of fresolimumab (i.e., having VH and VL domains of SEQ ID NO:112 and SEQ ID NO: 114, respectively). Typically, such an assay involves the use of purified target protein bound to a solid surface or cells bearing the target protein, an unlabeled test protein (i.e., a TNFα-binding or TGFβ i-binding protein or candidate TNFα-binding or TGF-β-binding protein), and a labeled reference antibody. Competitive inhibition is measured by determining the amount of label bound to the solid surface or cells in the presence of the test protein. Usually the test protein is present in excess and/or allowed to bind first. Soluble TNFα-binding or TGFβ-binding proteins identified by competition assay (“competing TNFα-binding or TGFβ-binding proteins”) include proteins binding to the same epitope bound by the reference antibody, proteins binding to an epitope overlapping with the epitope bound by the reference antibody, and proteins binding to an epitope sufficiently proximal to the epitope bound by the reference antibody for steric hindrance to occur. Usually, when a competing TNFα-binding or TGFβ-binding protein (e.g., a competing anti-TNFα or anti-TGF-β antibody) is present in excess, it will inhibit specific binding of a reference antibody to TNFα or TGF-β target protein by at least 30%, at least 40%, at least 45%, at least 50%, at least 55%, at least 60%, at least 65%, at least 70%, or at least 75%; in some instances, binding is inhibited by at least 80%, at least 85%, at least 90%, at least 95%, at least 97%, or more. Conversely, when the reference antibody is bound, it will preferably inhibit binding of a subsequently added competing TNFα-binding or TGFβ-binding protein (e.g., a competing anti-TNFα or anti-TGF-β antibody) by at least 30%, at least 40%, at least 45%, at least 50%, at least 55%, at least 60%, at least 65%, at least 70%, or at least 75%; in some instances, binding is inhibited by at least 80%, at least 85%, at least 90%, at least 95%, at least 97%, or more.

In certain preferred embodiments, a polypeptide that specifically binds and neutralizes an inflammatory cytokine for use in accordance with the present invention is a single-chain antibody. Particularly suitable single-chain antibodies include single-chain Fvs (scFvs) and single-domain antibodies (sdAbs). A single-chain antibody may be, e.g., derived from an identified intact, native monoclonal antibody or antibody fragment having the desired specificity. Methods for preparing and isolating monoclonal antibodies and antigen-binding antibody fragments thereof are well-known in the art. See, e.g., Current Protocols in Immunology, (Cooligan et al. eds., John Wiley and Sons, Inc. 2006); Sambrook et al., Molecular Cloning: A Laboratory Manual (Cold Spring Harbor, NY, 2nd ed. 1989); and Monoclonal Hybridoma Antibodies: Techniques and Applications (Hurrell ed., CRC Press, Inc., Boca Raton, F L, 1982). Antigen-binding fragments, including scFv, can be prepared using, e.g., phage display libraries according to methods known in the art. As will be evident to persons of ordinary skill in the art, these methods are equally applicable to production of antibodies against an inflammatory cytokine for use in accordance with the present invention.

The amino acid sequence of native antibody variable regions can be varied through the application of recombinant DNA techniques. Thus, antibodies can be redesigned to obtain desired characteristics. Typically, changes in the variable region will be made in order to improve the antigen-binding characteristics, improve variable region stability, or reduce the risk of immunogenicity. Phage display techniques can also be employed. See, e.g., Huse et al., Science 246:1275-1281, 1989; Ladner et al., U.S. Pat. No. 5,571,698.

For therapeutic antibodies for use in humans, it is usually desirable to humanize non-human regions of an antibody according to known procedures. Methods of making humanized antibodies are disclosed, for example, in U.S. Pat. Nos. 5,530.101; 5,821,337; 5,585,089; 5,693,762; and 6,180,370. Methods of making humanized antibodies are also disclosed in, e.g., U.S. Pat. No. 7,732,578. Typically, a humanized anti-inflammatory-cytokine antibody comprises the complementarity determining regions (CDRs) of a murine donor immunoglobulin and heavy chain and light chain frameworks of a human acceptor immunoglobulin. Often, framework residues in the human framework regions will be substituted with the corresponding residue from the donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. See, e.g., Queen et al., U.S. Pat. No. 5,585,089; Riechmann et al., Nature 332:323, 1988.

The present invention also encompasses use of fully human antibodies such as, for example, those selected from human antibody expression libraries (e.g., phage display libraries); those made in non-human animals (e.g., mice) transgenic for a human heavy chain locus and a human light chain locus with the corresponding endogenous immunoglobulin loci inactivated; or those prepared by immortalizing human B lymphocytes producing an antibody against an inflammatory cytokine target antigen.

Antibodies for use in accordance with the present invention have binding affinities that include a dissociation constant (Kₐ) that is, e.g., less than 10₋₄ M, less than 10₋₄ M, less than 5×10₋₅ M, less than 10₋₅ M, less than 5×10₋₆ M, less than 10₋₆ M, less than 5×10₋₇ M, less than 10₋₇ M, less than 5×10₋₈ M, less than 10₋₈ M, less than 5×10₋₉ M, less than 10₋₉ M, less than 5×10₋₁₀ M, less than 10₋₁₀ M, less than 5×10₋₁₁ M, less than 10₋₁₁ M, less than 5×10₋₁₂ M, less than 10₋₁₂ M, less than 5×10₋₁₃ M, less than 10₋₁₃ M, less than 5×10₋₁₄ M, less than 10₋₁₄ M, less than 5×10₋₁₅ M, or less than 10₋₁₅ M.

Antibodies for use in accordance with the present invention include variants having one or more amino acid substitutions, deletions, or additions relative to a reference antibody such that the variant retains one or more biological properties of the reference antibody. In certain embodiments, an antibody is a variant having one or more amino acid substitutions, deletions, or additions relative to a reference anti-TNFα neutralizing antibody having VH and/or VL sequences as shown in SEQ ID NO:108 and SEQ ID NO:110, respectively, such that the antibody retains the ability of the reference antibody to specifically bind TNFα and neutralize TNFα activity. In other embodiments, an antibody is a variant having one or more amino acid substitutions, deletions, or additions relative to a reference anti-TNF-β neutralizing antibody having VH and/or VL sequences as shown in SEQ ID NO:112 and SEQ ID NO: 114, respectively, such that the antibody retains the ability of the reference antibody to specifically bind TNF-β and neutralize TNF-β activity. A skilled person can readily produce variants having one or more amino acid substitutions, deletions, or additions relative to a reference antibody. The techniques for obtaining these variants, including genetic (suppressions, deletions, mutations, etc.), chemical, and enzymatic techniques, are known to the person having ordinary skill in the art.

In some embodiments of a fusion polypeptide as above comprising a single-chain antibody that specifically binds and neutralizes TNFα(“anti-TNFα single-chain antibody”), the anti-TNFα single-chain antibody comprises one or more CDRs of anti-TNFα monoclonal antibody adalimumab. Thus, in certain variations, an anti-TNFα single-chain antibody comprises a heavy chain CDR (at least one of the CDR-H1, CDR-H2, and CDR-H3 regions) of the adalimumab VH domain (SEQ ID NO:108) and/or a light chain CDR (at least one of CDR-L1, CDR-L2, and CDR-L3 regions) of the adalimumab VL domain (SEQ ID NO:110). In typical embodiments, the anti-TNFα single-chain antibody has two or three CDRs of the adalimumab VH domain (SEQ ID NO:108) and/or two or three CDRs of the adalimumab VL domain (SEQ ID NO:110). In some variations, where an anti-TNFα single-chain antibody has at least one CDR of the adalimumab VH domain, the antibody further comprises at least one CDR of the adalimumab VL domain; in some such embodiments, an antibody has all three heavy chain CDRs and all three light chain CDRs of adalimumab (i.e., CDR-H1, CDR-H2, and CDR-H3 of SEQ ID NO:108 and CDR-L1, CDR-L2, and CDR-L3 of SEQ ID NO:110). The one or more CDRs may be defined, for example, according to the Chothia definition, the Kabat definition, the AbM definition, the IMGT database definition, or the contact definition of CDR. Under the IMGT database definition of CDR, CDR-H1, CDR-H2, and CDR-H3 of adalimumab correspond, respectively, to residues 26-33, 50-59, and 97-110 of SEQ ID NO:108, and CDR-L1, CDR-L2, and CDR-L3 of adalimumab correspond, respectively, to residues 27-32, 50-52, and 89-97 of SEQ ID NO:110. Under the Kabat definition of CDR, CDR-H1, CDR-H2, and CDR-H3 of adalimumab correspond, respectively, to residues 31-35, 50-66, and 99-110 of SEQ ID NO:108, and CDR-L1, CDR-L2, and CDR-L3 of adalimumab correspond, respectively, to residues 24-34, 50-56, and 89-97 of SEQ ID NO:110. In particular variations of an anti-TNFα single-chain antibody as above, the single-chain antibody comprises an VH domain and/or a VL domain having a human immunoglobulin framework region, or a variant thereof having at least 85%, at least 90%, or at least 95% amino acid sequence identity to the human immunoglobulin framework region.

In some embodiments, an anti-TNFα single-chain antibody includes (a) a heavy chain variable domain that is at least 60%, at least 65%, at least 70%, at least 75%, at least 80%, at least 85%, at least 90%, at least 95%, at least 96%, at least 97%, at least 98%, at least 99%, or 100% identical to the amino acid sequence of SEQ ID NO:108, and/or (b) a light chain variable domain that is at least 60%, at least 65%, at least 70%, at least 75%, at least 80%, at least 85%, at least 90%, at least 95%, at least 96%, at least 97%, at least 98%, at least 99%, or 100% identical to the amino acid sequence of SEQ ID NO:110.

In some embodiments, an anti-TNFα single-chain antibody for use in accordance with the present invention comprises heavy chain CDRs CDR-H1_{TNFα}, CDR-H2_{TNFα}, and CDR-H3_{TNFα}, wherein the set of VH CDRs has 10 or fewer amino acid substitutions relative to a set of reference CDRs CDR-H1, CDR-H2, and CDR-H3 of the adalimumab VH domain (SEQ ID NO:108). In other, non-mutually exclusive embodiments, the anti-TNFα single-chain comprises light chain CDRs CDR-L1_{TNFα}, CDR-L2_{TNFα}, and CDR-L3_{TNFα}, wherein the set of VL CDRs has 10 or fewer amino acid substitutions relative to a set of reference CDRs CDR-L1, CDR-L2, and CDR-L3 of the adalimumab VL domain (SEQ ID NO:110). In certain embodiments, an anti-TNFα single-chain antibody comprises both sets of heavy chain and light chain CDRs as above. Particularly suitable anti-TNFα single-chain antibodies comprise a heavy chain variable domain comprising CDRs CDR-H1_{TNFα}, CDR-H2_{TNFα}, and CDR-H3_{TNFα} and a light chain variable domain comprising CDRs CDR-L1_{TNFα}, CDR-L2_{TNFα}, and CDR-L3_{TNFα}, wherein the set of heavy chain CDRs has six or fewer, typically five or fewer, more typically four or fewer, an most typically three or fewer amino acid substitutions relative to CDR-H1, CDR-H2, and CDR-H3 of adalimumab, and wherein the set of light chain CDRs has six or fewer, typically five or fewer, more typically four or fewer, an most typically three or fewer amino acid substitutions relative to CDR-L1, CDR-L2, and CDR-L3 of adalimumab. The CDRs as above may be defined, for example, according to the Chothia definition, the Kabat definition, the AbM definition, the IMGT database definition, or the contact definition of CDR. In particular variations of an anti-TNFα antibody as above, each of the VH and VL domains has a human immunoglobulin framework region, or a variant thereof having at least 85%, at least 90%, or at least 95% amino acid sequence identity to the human immunoglobulin framework region.

In some embodiments of a fusion polypeptide as above comprising a single-chain antibody that specifically binds and neutralizes TGF-β(“anti-TGF-β single-chain antibody”), the anti-TGF-β single-chain antibody comprises one or more CDRs of anti-TGF-β monoclonal antibody fresolimumab. Thus, in certain variations, an anti-TGF-0 single-chain antibody comprises a heavy chain CDR (at least one of the CDR-H1, CDR-H2, and CDR-H3 regions) of the fresolimumab VH domain (SEQ ID NO:112) and/or a light chain CDR (at least one of CDR-L1, CDR-L2, and CDR-L3 regions) of the fresolimumab VL domain (SEQ ID NO:114). In typical embodiments, the anti-TGF-β single-chain antibody has two or three CDRs of the fresolimumab VH domain (SEQ ID NO:112) and/or two or three CDRs of the fresolimumab VL domain (SEQ ID NO:114). In some variations, where an anti-TGF-β single-chain antibody has at least one CDR of the fresolimumab VH domain, the antibody further comprises at least one CDR of the fresolimumab VL domain; in some such embodiments, an antibody has all three heavy chain CDRs and all three light chain CDRs of fresolimumab (i.e., CDR-H1, CDR-H2, and CDR-H3 of SEQ ID NO:112 and CDR-L1, CDR-L2, and CDR-L3 of SEQ ID NO: 114). The one or more CDRs may be defined, for example, according to the Chothia definition, the Kabat definition, the AbM definition, the IMGT database definition, or the contact definition of CDR. Under the Kabat definition of CDR, CDR-H1, CDR-H2, and CDR-H3 of fresolimumab correspond, respectively, to residues 31-35, 50-66, and 99-106 of SEQ ID NO: 112, and CDR-L1, CDR-L2, and CDR-L3 of fresolimumab correspond, respectively, to residues 24-40, 56-62, and 95-102 of SEQ ID NO:114. In particular variations of an anti-TGF-β single-chain antibody as above, the single-chain antibody comprises an VH domain and/or a VL domain having a human immunoglobulin framework region, or a variant thereof having at least 85%, at least 90%, or at least 95% amino acid sequence identity to the human immunoglobulin framework region.

In some embodiments, an anti-TGF-β single-chain antibody includes (a) a heavy chain variable domain that is at least 60%, at least 65%, at least 70%, at least 75%, at least 80%, at least 85%, at least 90%, at least 95%, at least 96%, at least 97%, at least 98%, at least 99%, or 100% identical to the amino acid sequence of SEQ ID NO:112, and/or (b) a light chain variable domain that is at least 60%, at least 65%, at least 70%, at least 75%, at least 80%, at least 85%, at least 90%, at least 95%, at least 96%, at least 97%, at least 98%, at least 99%, or 100% identical to the amino acid sequence of SEQ ID NO: 114.

In some embodiments, an anti-TGF-β single-chain antibody for use in accordance with the present invention comprises heavy chain CDRs CDR-H1_{TNFα}, CDR-H2_{TNFα}, and CDR-H3_{TNFα}, wherein the set of VH CDRs has 10 or fewer amino acid substitutions relative to a set of reference CDRs CDR-H1, CDR-H2, and CDR-H3 of the fresolimumab VH domain (SEQ ID NO:112). In other, non-mutually exclusive embodiments, the anti-TGF-β single-chain comprises light chain CDRs CDR-H1_{TGFβ}, CDR-H2_{TGFβ}, and CDR-H3_{TGFβ}, wherein the set of VL CDRs has 10 or fewer amino acid substitutions relative to a set of reference CDRs CDR-L1, CDR-L2, and CDR-L3 of the fresolimumab VL domain (SEQ ID NO:114). In certain embodiments, an anti-TGF-β single-chain antibody comprises both sets of heavy chain and light chain CDRs as above. Particularly suitable anti-TGF-β single-chain antibodies comprise a heavy chain variable domain comprising CDRs CDR-H1_{TGFβ}, CDR-H2_{TGFβ}, and CDR-H3_{TGFβ} and a light chain variable domain comprising CDRs CDR-L1_{TGFβ}, CDR-L2_{TGFβ}, and CDR-L3_{TGFβ}, wherein the set of heavy chain CDRs has six or fewer, typically five or fewer, more typically four or fewer, and most typically three or fewer amino acid substitutions relative to CDR-H1. CDR-H2, and CDR-H3 of fresolimumab, and wherein the set of light chain CDRs has six or fewer, typically five or fewer, more typically four or fewer, and most typically three or fewer amino acid substitutions relative to CDR-L1, CDR-L2, and CDR-L3 of fresolimumab. The CDRs as above may be defined, for example, according to the Chothia definition, the Kabat definition, the AbM definition, the IMGT database definition, or the contact definition of CDR. In particular variations of an anti-TGF-β antibody as above, the each of the VH and VL domains has a human immunoglobulin framework region, or a variant thereof having at least 85%, at least 90%, or at least 95% amino acid sequence identity to the human immunoglobulin framework region.

Anti-inflammatory-cytokine antibodies for use in accordance with the present invention include affinity matured embodiments. Affinity matured antibodies can be produced by procedures known in the art (see, e.g., Marks et al., Bio/Technology 10:779-783, 1992; Barbas et al., Proc Nat. Acad. Sci. USA 91:3809-3813, 1994; Schier et al., Gene 169:147-155, 1995; Yelton et al., J. Immunol. 155:1994-2004, 1995; Jackson et al., J. Immunol. 154:3310-9, 1995; Hawkins et al., J. Mol. Biol. 226:889-896, 1992; and PCT Publication No. WO 2004/058184). One method for adjusting the affinity of an antibody is termed “library scanning mutagenesis.” Generally, library scanning mutagenesis is carried out as follows. One or more amino acid positions in at least one CDR (e.g., in two, three, four, five, or six CDRs) are replaced with two or more (such as 3, 4, 5, 6, 7, 8, 9, 10, 11, 12, 13, 14, 15, 16, 17, 18, 19, or 20) amino acids using art recognized methods. This generates small libraries of clones (in some embodiments, one for every amino acid position that is analyzed), each with a complexity of two or more members (if two or more amino acids are substituted at every position). Generally, the library also includes a clone comprising the native (unsubstituted) amino acid. A small number of clones, e.g., about 20-80 clones (depending on the complexity of the library), from each library are screened for binding affinity to the target polypeptide (or other binding target), and candidates with increased, the same, decreased, or no binding are identified. Binding affinity may be determined, e.g., using Biacore™ surface plasmon resonance analysis, which detects differences in binding affinity of about 2-fold or greater. Biacore™ is particularly useful when the starting antibody already binds with a relatively high affinity, for example a K_{D} of about 10 nM or lower.

In some embodiments of a fusion polypeptide comprising an anti-TNFα single-chain antibody as the first biologically active polypeptide, the anti-TNFα single-chain antibody comprises an amino acid sequence having at least 80%, at least 85%, at least 90%, or at least 95% identity with (i) amino acid residues 21-268 of SEQ ID NO:92 or (ii) amino acid residues 21-268 of SEQ ID NO:96. In more particular variations, the anti-TNFα single-chain antibody comprises an amino acid sequence having at least 96%, at least 97%, at least 98%, at least 99%, or 100% identity with (i) amino acid residues 21-268 of SEQ ID NO:92 or (ii) amino acid residues 21-268 of SEQ ID NO:96.

In some embodiments of a fusion polypeptide comprising an anti-TGF-β single-chain antibody as the first biologically active polypeptide, the anti-TGF-β single-chain antibody comprises an amino acid sequence having at least 80%, at least 85%, at least 90%, or at least 95% identity with (i) amino acid residues 21-269 of SEQ ID NO:100 or (ii) amino acid residues 21-269 of SEQ ID NO:104. In more particular variations, the anti-TGF-β single-chain antibody comprises an amino acid sequence having at least 96%, at least 97%, at least 98%, at least 99%, or 100% identity with (i) amino acid residues 21-269 of SEQ ID NO:100 or (ii) amino acid residues 21-269 of SEQ ID NO:104.

Polypeptide linkers for use in accordance with the present invention can be naturally occurring, synthetic, or a combination of both. The linker joins two separate polypeptide regions (e.g., an Fc region and a paraoxonase, or an Fc region and a DNase) and maintains the linked polypeptide regions as separate and discrete domains of a longer polypeptide. The linker can allow the separate, discrete domains to cooperate yet maintain separate properties (e.g., in the case of an Fc region linked to a paraoxonase or DNase, Fc receptor (e.g., FcRn) binding may be maintained for the Fc region, while functional properties of the paraoxonase (e.g., organophosphatase or arylesterase activity) or DNase (e.g., DNA binding and nuclease activity) will be maintained. For examples of the use of naturally occurring as well as artificial peptide linkers to connect heterologous polypeptides, see. e.g., Hallewell et al., J. Biol. Chem. 264, 5260-5268, 1989; Alfthan et al., Protein Eng. 8, 725-731, 1995; Robinson and Sauer, Biochemistry 35, 109-116, 1996; Khandekar et al., J. Biol. Chem. 272, 32190-32197, 1997; Fares et al., Endocrinology 139, 2459-2464, 1998; Smallshaw et al., Protein Eng. 12, 623-630, 1999; U.S. Pat. No. 5,856,456.

Typically, residues within the linker polypeptide are selected to provide an overall hydrophilic character and to be non-immunogenic and flexible. As used herein, a “flexible” linker is one that lacks a substantially stable higher-order conformation in solution, although regions of local stability are permissible. In general, small, polar, and hydrophilic residues are preferred, and bulky and hydrophobic residues are undesirable. Areas of local charge are to be avoided; if the linker polypeptide includes charged residues, they will ordinarily be positioned so as to provide a net neutral charge within a small region of the polypeptide. It is therefore preferred to place a charged residue adjacent to a residue of opposite charge. In general, preferred residues for inclusion within the linker polypeptide include Gly, Ser, Ala, Thr, Asn, and Gln; more preferred residues include Gly, Ser, Ala, and Thr, and the most preferred residues are Gly and Ser. In general, Phe, Tyr, Trp, Pro, Leu, Ile, Lys, and Arg residues will be avoided (unless present within an immunoglobulin hinge region of the linker), Pro residues due to their hydrophobicity and lack of flexibility, and Lys and Arg residues due to potential immunogenicity. The sequence of the linker will also be designed to avoid unwanted proteolysis.

In certain embodiments, linker L1 comprises at least two or at least three amino acid residues (e.g., at least five, at least 10, at least 16, at least 26, or at least 36 amino acid residues). In particular variations, L1 consists of from two to 60 amino acid residues, from three to 60 amino acid residues, from five to 40 amino acid residues, or from 15 to 40 amino acid residues. In other variations, L1 consists of from two to 50, from two to 40, from two to 36, from two to 35, from two to 30, from two to 26, from three to 50, from three to 40, from three to 36, from three to 35, from three to 30, from three to 26, from five to 60, from five to 50, from five to 40, from five to 36, from five to 35, from five to 30, from five to 26, from 10 to 60, from 10 to 50, from 10 to 40, from 10 to 36, from 10 to 35, from 10 to 30, from 10 to 26, from 15 to 60, from 15 to 50, from 15 to 36, from 15 to 35, from 15 to 30, or from 15 to 26 amino acid residues. In other variations, L1 consists of from 16 to 60, from 16 to 50, from 16 to 40, or from 16 to 36 amino acid residues. In yet other variations, L1 consists of from 20 to 60, from 20 to 50, from 20 to 40, from 20 to 36, from 25 to 60, from 25 to 50, from 25 to 40, or from 25 to 36 amino acid residues. In still other variations, L1 consists of from 26 to 60, from 26 to 50, from 26 to 40, or from 26 to 36 amino acid residues. In more specific variations, L1 consists of 16 amino acid residues, 21 amino acid residues, 26 amino acid residues, 31 amino acid residues, or 36 amino acid residues. In some embodiments, L1 comprises or consists of the amino acid sequence shown in residues SEQ ID NO:10, residues 268-288 of SEQ ID NO:12, or SEQ ID NO:14.

In typical variations of a fusion polypeptide comprising a DNase as the first biologically active polypeptide, L1 is present and comprises at least 15 amino acid residues. For example, an L1 linker joining the carboxyl-terminus of a DNase to the amino-terminus of another fusion component (e.g., an immunoglobulin Fc region) may comprise at least 20, at least 21, at least 22, at least 23, at least 24, at least 25, or at least 26 amino acid residues. In more particular variations, L1 consists of from 15 to 60 amino acid residues, from 15 to 51 amino acid residues, from 15 to 50 amino acid residues, from 15 to 46 amino acid residues, from 15 to 45 amino acid residues, from 15 to 41 amino acid residues, from 15 to 40 amino acid residues, from 15 to 36 amino acid residues, from 26 to 60 amino acid residues, from 26 to 51 amino acid residues, from 26 to 50 amino acid residues, from 26 to 46 amino acid residues, from 26 to 45 amino acid residues, from 26 to 41 amino acid residues, from 26 to 40 amino acid residues, or from 26 to 36 amino acid residues. In still more specific variations, L1 linking a DNase consists of 26 amino acid residues, 27 amino acid residues, 28 amino acid residues, 29 amino acid residues, 30 amino acid residues, 31 amino acid residues, 32 amino acid residues, 33 amino acid residues, 34 amino acid residues, 35 amino acid residues, or 36 amino acid residues. In some embodiments, L1 linking a DNase comprises or consists of the amino acid sequence shown in SEQ ID NO:12 or SEQ ID NO:14.

In some variations of a fusion polypeptide comprising an RNase or a SOD1 as the first biologically active polypeptide, L1 is present and comprises at least two amino acid residues. For example, an L1 linker joining the carboxyl-terminus of an RNase or a SOD1 to the amino-terminus of another fusion component (e.g., an immunoglobulin Fc region) may comprise at least three, at least five, or at least 10 amino acid residues. In more particular variations, L1 linking an RNase or a SOD1 consists of from two to 60 amino acid residues, from two to 51 amino acid residues, from two to 50 amino acid residues, from two to 46 amino acid residues, from two to 45 amino acid residues, from two to 41 amino acid residues, from two to 40 amino acid residues, from two to 36 amino acid residues, from 10 to 60 amino acid residues, from 10 to 51 amino acid residues, from 10 to 50 amino acid residues, from 10 to 46 amino acid residues, from 10 to 45 amino acid residues, from 10 to 41 amino acid residues, from 10 to 40 amino acid residues, or from 10 to 36 amino acid residues. In more specific variations, L1 linking an RNase or SOD1 consists of 16 or 26 amino acid residues. In some embodiments, L1 linking an RNase or a SOD1 comprises or consists of the amino acid sequence shown in SEQ ID NO:10 or SEQ ID NO:12.

In some variations of a fusion polypeptide comprising the formula P-L1-X as described herein. L1 comprises at least 20 amino acid residues. For example, an L1 linker joining the carboxyl-terminus of a paraoxonase to the amino-terminus of another fusion component (e.g., an immunoglobulin Fc region) may comprise at least 25, at least 26, at least 27, at least 28, at least 29, at least 30, at least 31, at least 32, at least 33, at least 34, at least 35, or at least 36 amino acid residues. In certain variations, L1 consists of from 20 to 60 amino acid residues, from 20 to 50 amino acid residues, from 20 to 45 amino acid residues, from 20 to 40 amino acid residues, from 20 to 36 amino acid residues, from 26 to 60 amino acid residues, from 26 to 50 amino acid residues, from 26 to 45 amino acid residues, from 26 to 40 amino acid residues, from 26 to 36 amino acid residues, from 36 to 60 amino acid residues, from 36 to 50 amino acid residues, from 36 to 45 amino acid residues, or from 36 to amino acid residues. In some embodiments, L1 linking the carboxyl-terminus of a paraoxonase comprises or consists of the amino acid sequence shown in SEQ ID NO:12 or SEQ ID NO:14.

Exemplary L2 linkers comprise at least three amino acid residues and are typically up to 60 amino acid residues. In certain variations, L2 linkers comprise at least four, at least five, at least six, at least seven, at least eight, at least 9, or at least 10 amino acid residues. In more specific variations, L2 consists of from six to 30, from six to 25, from six to 20, from seven to 30, from seven to 25, from seven to 20, from eight to 30, from eight to 25, from eight to 20, from nine to 30, from nine to 25, from nine to 20, from 10 to 30, from to 25, from 10 to 20, from 11 to 30, from 11 to 25, from 11 to 20, from 12 to 30, from 12 to 25, or from 12 to 20 amino acid residues. In some embodiments, L2 comprises or consists of the amino acid sequence shown in SEQ ID NO:56.

In certain embodiments, a polypeptide linker comprises a plurality of glycine residues. For example, in some embodiments, a polypeptide linker (e.g., L1) comprises a plurality of glycine residues and optionally at least one serine residue. In particular variations, a polypeptide linker (e.g., L1) comprises the sequence Gly-Gly-Gly-Gly-Ser (SEQ ID NO:29), such as, e.g., two or more tandem repeats of the amino acid sequence of SEQ ID NO:29. In some embodiments, a linker comprises the sequence [Gly-Gly-Gly-Gly-Ser]ₙ ([SEQ ID NO:29]n), where n is a positive integer such as, for example, an integer from 2 to 8, from 2 to 7, from 2 to 6, from 3 to 8, from 3 to 7, from 3 to 6, from 4 to 8, from 4 to 7, or from 4 to 6. In a specific variation of a polypeptide linker comprising the formula [Gly-Gly-Gly-Gly-Ser]ₙ, n is 4. In another specific variation of a polypeptide linker comprising the formula [Gly-Gly-Gly-Gly-Ser]ₙ, n is 6. In yet another specific variation of a polypeptide linker comprising the formula [Gly-Gly-Gly-Gly-Ser]ₙ, n is 5. In certain embodiments, a polypeptide linker comprises a series of glycine and serine residues (e.g., [Gly-Gly-Gly-Gly-Ser]n, where n is defined as above) inserted between two other sequences of the polypeptide linker (e.g., inserted between Asp-Leu-Ser at the N-terminal end of the linker and Thr-Gly-Leu at the C-terminal end of the linker). In other embodiments, a polypeptide linker includes glycine and serine residues (e.g., [Gly-Gly-Gly-Gly-Ser]ₙ, where n is defined as above) attached at one or both ends of another sequence of the polypeptide linker.

In some embodiments of a fusion polypeptide as above wherein X is present, X is a dimerizing domain. Various dimerization domains are suitable for use in accordance with certain fusion polypeptide embodiments and dimeric fusion proteins as described herein. In certain embodiments of a fusion polypeptide comprising a dimerizing domain, the dimerizing domain is an immunoglobulin heavy chain constant region. The immunoglobulin heavy chain constant region may be a native sequence constant region or a variant constant region. In typical variations, an immunoglobulin heavy chain constant region is capable of binding to the neonatal Fc receptor (FcRn) with sufficient affinity to confer improved half-life to the fusion polypeptide in vivo. A particularly suitable immunoglobulin heavy chain constant region for use in accordance with the present invention is an immunoglobulin Fc region. In some embodiments, the heavy chain constant region lacks one or more effector functions (e.g., one or both of ADCC and CDC effector functions).

In some embodiments of a fusion polypeptide comprising an immunoglobulin Fc region, the immunoglobulin Fc region is a human IgG Fc region having, relative to the wild-type human IgG sequence, an amino acid substitution in the CH2 region so that the molecule is not glycosylated, including but not limited to an amino acid substitution at N297 (Eu numbering for human IgG heavy chain constant region) (corresponding to amino acid position 82 of SEQ ID NO:26, SEQ ID NO:28, SEQ ID NO: 116, or SEQ ID NO: 118). In another embodiment, the Fc region is human IgG1 (γ1) with the three cysteines of the hinge region (C220, C226, C229) each changed to a non-cysteine residue (e.g., serine) and, optionally, the proline at position 238 of the CH2 domain changed to a non-proline residue (e.g., serine or aspartate). In another embodiment, the Fc region is human γ1 with cysteine C220 changed to a non-cysteine residue (e.g., serine) and, optionally, the proline at position of the CH2 domain changed to a non-proline residue (e.g., serine or aspartate). In another embodiment, the Fc region is human γ1 with N297 changed to a non-asparagine residue (e.g., alanine, glutamine, or glycine). In another embodiment, the Fc region is human γ1 with one or more amino acid substitutions between Eu positions 292 and 300. In another embodiment, the Fc region is human γ1 with one or more amino acid additions or deletions at any position between residues 292 and 300. In another embodiment, the Fc region is human γ1 with an SCC hinge (i.e., with cysteine C220 changed to serine and with a cysteine at each of Eu positions 226 and 229) or an SSS hinge (i.e., each of the three cysteines at Eu positions 220, 226, and 229 changed to serine). In further embodiments, the Fc region is human γ1 with an SCC hinge and an amino acid substitution at P238. In another embodiment, the Fc domain is human γ1 with amino acid substitutions that alter binding by Fc gamma receptors (I, II, III) without affecting FcRn binding important for half-life. In further embodiments, an Fc region is as disclosed in Ehrhardt and Cooper, Curr. Top. Microbiol. Immunol. 2010 Aug. 3 (Immunoregulatory Roles for Fc Receptor-Like Molecules); Davis et al., Ann. Rev. Immunol. 25:525-60, 2007 (Fc receptor-like molecules); or Swainson et al., J. Immunol. 184:3639-47, 2010.

In some embodiments, the Fc region comprises an amino acid substitution that alters the antigen-independent effector functions of the fusion protein. In some such embodiments, the Fc region includes an amino acid substitution that alters the circulating half-life of the resulting molecule. Such Fc variants exhibit either increased or decreased binding to FcRn when compared to an Fc region lacking these substitutions and, therefore, confer increased or decreased half-life, respectively, of the resulting molecule in serum. Fc variants with improved affinity for FcRn are anticipated to have longer serum half-lives, and such Fc variants have useful applications in methods of treating mammals where long half-life of the administered Fc fusion is desired and where increased transport through the lungs to the circulation is desired. In contrast, Fc variants with decreased FcRn binding affinity are expected to have shorter half-lives, and such variants are also useful, for example, for administration to a mammal where a shortened circulation time may be advantageous (e.g., where the fusion protein has toxic side effects when present in the circulation for prolonged periods). For treatment of conditions characterized by NETosis, fine tuning of the half-life of a DNase-Fc fusion protein through altering FcRn binding may be useful since NETs, while contributing to pathogenesis, are also beneficial in certain settings; digestion of pathogenic NETs may not require as long a half life, so more rapid clearance of the DNase may allow neutrophils to form NETs in their protective anti-microbial function. Fc variants with decreased FcRn binding affinity are also less likely to cross the placenta and, thus, are also useful in the treatment of diseases or disorders in pregnant women. In addition, other applications in which reduced FcRn binding affinity may be desired include those applications in which localization to the brain, kidney, and/or liver is desired. In one exemplary embodiment, the fusion molecules of the invention exhibit reduced transport across the epithelium of kidney glomeruli from the vasculature. In another embodiment, the fusion molecules of the invention exhibit reduced transport across the blood brain barrier (BBB) from the brain, into the vascular space. In one embodiment, a fusion molecule with altered FcRn binding comprises an Fc region having one or more amino acid substitutions within the “FcRn binding loop” of the Fc domain. Exemplary amino acid substitutions that alter FcRn binding activity are disclosed in International PCT Publication No. WO 05/047327, which is incorporated by reference herein. Exemplary amino acid substitutions that increase FcRn binding activity are also described, e.g., by Wang et al., Protein Cell 9:63-73, 2018 (see, e.g., Table 1). In some embodiments, an Fc variant with increased FcRn binding activity has an amino acid substitution at each of Eu positions 252, 254, and 256 (e.g., M252Y, S254T, and T256E). In other variations, an Fc variant with increased FcRn binding activity has an amino acid substitution at each of Eu positions 428 and 434 (e.g., M428L and N434S).

In other embodiments, a fusion polypeptide of the present invention comprises an Fc variant comprising an amino acid substitution that alters one or more antigen-dependent effector functions of the polypeptide, in particular antibody-dependent cellular cytotoxicity (ADCC) or complement activation, e.g., as compared to a wild type Fc region. In an exemplary embodiment, such fusion polypeptides exhibit altered binding to an Fc gamma receptor (FcγR, e.g., CD16). Such fusion polypeptides exhibit either increased or decreased binding to Fcγ when compared to wild-type polypeptides and, therefore, mediate enhanced or reduced effector function, respectively. Fc variants with improved affinity for FcγR are anticipated to enhance effector function, and such variants have useful applications in methods of treating mammals where target molecule destruction is desired. In contrast, Fc variants with decreased FcγR binding affinity are expected to reduce effector function, and such fusion proteins are also useful, for example, for treatment of conditions in which target cell destruction is undesirable, e.g., where normal cells may express target molecules, or where chronic administration of the fusion molecule might result in unwanted immune system activation. In one embodiment, the fusion polypeptide comprising an Fc region exhibits at least one altered antigen-dependent effector function selected from the group consisting of opsonization, phagocytosis, complement dependent cytotoxicity (CDC), antibody-dependent cellular cytotoxicity (ADCC), or effector cell modulation as compared to a polypeptide comprising a wild-type Fc region. In typical embodiments of a DNase fusion molecule comprising an Fc variant with altered antigen-dependent effector function, the Fe variant has one or more reduced effector functions relative to the corresponding wild-type Fc region.

In one embodiment, a fusion polypeptide comprising an Fc region exhibits altered binding to an activating FcγR (e.g., FcγI, FcγIIa, or FcγRIIIa). In another embodiment, the fusion protein exhibits altered binding affinity to an inhibitory FcγR (e.g., FcγRIIb). Exemplary amino acid substitutions which alter FcR or complement binding activity are disclosed in International PCT Publication No. WO 05/063815, which is incorporated by reference herein. Exemplary Fc variants with reduced effector function are also described, e.g., by Tam et al., Antibodies 6:12, 2017 (describing variants of human IgG1 (γ1) and IgG4(γ4)); Wang et al., Protein Cell 9:63-73, 2018 (see, e.g., Table 1); Lo et al., J. Biol. Chem. 292:3900-3908, 2017; Idusogie et al., J. Immunol. 164:4178-4184, 2000 (each of which is incorporated by reference herein). Suitable Fc variants that reduce antigen-dependent effector function include, for example, variants having an amino acid substitution at Eu position 238 and/or position 331 (e.g., P238S and/or P331S or P331A). In addition, amino acid substitutions at Eu positions 234 and 235 of human Fc (e.g., L234A/L235A in IgG1, or F234A/L235A in IgG4) reduce FcγR binding and have been shown to reduce cytokine storm when introduced into anti-CD3 mAb (see, e.g., Wang et al., supra), and an amino acid substitution at Eu position 329 (e.g., P329A) is highly effective at reducing Clq binding (see, e.g., Lo et al., supra). Other exemplary approaches to removing ADCC and CDC effector functions is to make hybrid Fc domains derived from human IgG2 (Eu positions 118-260) and IgG4 (Eu positions 261-447), or to modify human IgG2 to contain selected amino acid substitutions from IgG4. See Wang et al., supra.

A fusion polypeptide comprising an Fc region may also comprise an amino acid substitution that alters the glycosylation of the Fc region. For example, the Fc domain of the fusion protein may have a mutation leading to reduced glycosylation (e.g., N- or O-linked glycosylation) or may comprise an altered glycoform of the wild-type Fc domain (e.g., a low fucose or fucose-free glycan). In another embodiment, the molecule has an amino acid substitution near or within a glycosylation motif, for example, an N-linked glycosylation motif that contains the amino acid sequence NXT or NXS. Exemplary amino acid substitutions which reduce or alter glycosylation are disclosed in International PCT Publication No. WO 05/018572 and US Patent Application Publication No. 2007/0111281, which are incorporated by reference herein.

Particularly suitable amino acid substitutions to reduce glycosylation and which also reduce ADCC and CDC effector functions of Fc include amino acid substitutions at Eu position 297 (e.g., N297A, N297Q, or N297G). See, e.g., Wang et al., supra. N297 substitutions may also be paired with substitutions at position 265 (e.g., D265A) to further reduce CDC. See, e.g., Lo et al., supra.

It will be understood by those of skill in the art that various embodiments of Fc variants as described herein can be combined in the fusion polypeptides of the present invention, unless the context clearly indicates otherwise.

In some embodiments, an immunoglobulin Fc region comprises an amino acid sequence having at least 80%, at least 85%, at least 90%, or at least 95% identity with an amino acid sequence selected from sequence shown in (i) residues 1-232 or 1-231 of SEQ ID NO:26, (ii) residues 1-232 or 1-231 of SEQ ID NO:28, (iii) residues 159-390 or 159-389 of SEQ ID NO:42, (iv) residues 1-232 or 1-231 of SEQ ID NO: 116, or (v) residues 1-232 or 1-231 of SEQ ID NO:118. In yet other embodiments, the Fc region comprises an amino acid sequence having at least 96%, at least 97%, at least 98%, at least 99%, or 100% identity with the amino acid sequence shown in (i) residues 1-232 or 1-231 of SEQ ID NO:26, (ii) residues 1-232 or 1-231 of SEQ ID NO:28, (iii) residues 159-390 or 159-389 of SEQ ID NO:42, (iv) residues 1-232 or 1-231 of SEQ ID NO:116, or (v) residues 1-232 or 1-231 of SEQ ID NO: 118.

In some embodiments, an immunoglobulin heavy chain constant region comprises an amino acid sequence having at least 80%, at least 85%, at least 90%, or at least 95% identity with an amino acid sequence selected from sequence shown in (i) residues 16-232 or 16-231 of SEQ ID NO:26, (ii) residues 16-232 or 16-231 of SEQ ID NO: 116, or (iii) residues 16-232 or 16-231 of SEQ ID NO: 118. In yet other embodiments, the immunoglobulin heavy chain constant region comprises an amino acid sequence having at least 96%, at least 97%, at least 98%, at least 99%, or 100% identity with the amino acid sequence shown in (i) residues 16-232 or 16-231 of SEQ ID NO:26, (ii) residues 16-232 or 16-231 of SEQ ID NO: 116, or (iii) residues 16-232 or 16-231 of SEQ ID NO:118.

In some variations of a fusion polypeptide as above wherein X is present, X is an FcRn-binding domain. Particularly suitable FcRn-binding domains include immunoglobulin heavy chain constant regions that retain FcRn-binding activity such as, e.g., an immunoglobulin Fc region; in some such variations, the Fc region is an Fc region as described herein (e.g., as described above in the context of a dimerizing domain). In other embodiments, an FcRn-binding domain is an albumin (e.g., human albumin). Yet other suitable FcRn-binding domains include single-chain antibodies (e.g., scFvs), peptide aptamers, or alternative scaffold proteins having binding affinity for FcRn; such alternative FcRn-binding molecules are readily created using, for example, display technologies that allow for selection of binding agents through screening of large expression libraries (e.g., libraries of immunoglobulin domains, randomized peptides, or other protein structures). Such display technologies are generally well-known in the art and include, for example, phage display. See, e.g., Antibody Engineering: A Practical Approach, McCafferty, Hoogenboom, and Chiswell Eds., IRL Press 1996. Alternative scaffold proteins for generating FcRn-binding domains include, e.g., avimers, ankyrin repeats, and adnectins, as well as other proteins with domains that can be evolved to generate specific affinity for a desired molecular target (see, e.g., Silverman et al., Nature Biotechnology 23:1556-1561, 2005; Zahnd et al., J. Mol. Biol. 369:1015-1028, 2007; U.S. Pat. No. 7,115,396 to Lipovsek et al.).

In some embodiments of a paraoxonase fusion polypeptide comprising a DNase as described above, the fusion polypeptide comprises an amino acid sequence having at least 80%, at least 85%, at least 90%, or at least 95% identity with the amino acid sequence shown in (i) residues 21-896 or 1-896 of SEQ ID NO:2,
(ii) residues 21-896 or 1-896 of SEQ ID NO:128, (iii) residues 21-896 or 1-896 of SEQ ID NO:130, (iv) residues 21-906 or 1-906 of SEQ ID NO:148, residues 21-896 or 1-896 of SEQ ID NO:158, (vi) residues 21-906 or 1-906 of SEQ ID NO:160, (vii) residues 21-906 or 1-906 of SEQ ID NO:162, or (viii) residues 21-906 or 1-906 of SEQ ID NO:164. In yet other embodiments, the fusion polypeptide comprises an amino acid sequence having at least 96%, at least 97%, at least 98%, at least 99%, or 100% sequence identity with the amino acid sequence shown in (i) residues 21-896 or 1-896 of SEQ ID NO:2, (ii) residues 21-896 or 1-896 of SEQ ID NO:128, (iii) residues 21-896 or 1-896 of SEQ ID NO:130, (iv) residues 21-906 or 1-906 of SEQ ID NO:148, (v) residues 21-896 or 1-896 of SEQ ID NO:158, (vi) residues 21-906 or 1-906 of SEQ ID NO:160, (vii) residues 21-906 or 1-906 of SEQ ID NO:162, or (viii) residues 21-906 or 1-906 of SEQ ID NO:164.

In some embodiments of a paraoxonase fusion polypeptide comprising an RNase as described above, the fusion polypeptide comprises an amino acid sequence having at least 80%, at least 85%, at least 90%, or at least 95% identity with an amino acid sequence selected from the sequence shown in (i) residues 21-764 or 1-764 of SEQ ID NO:40 or (ii) residues 21-740 or 1-740 of SEQ ID NO:48. In yet other embodiments, the fusion polypeptide comprises an amino acid sequence having at least 96%, at least 97%, at least 98%, at least 99%, or 100% sequence identity with the amino acid sequence shown in (i) residues 21-764 or 1-764 of SEQ ID NO:40 or (ii) residues 21-740 or 1-740 of SEQ ID NO:48.

In some embodiments of a paraoxonase fusion polypeptide comprising a superoxide dismutase 1 (SOD1) as described above, the fusion polypeptide comprises an amino acid sequence having at least 80%, at least 85%, at least 90%, or at least 95% identity with an amino acid sequence selected from the sequence shown in (i) residues 23-781 or 1-781 of SEQ ID NO:50, (ii) residues 23-781 or 1-781 of SEQ ID NO:52, or (iii) residues 23-791 or 1-791 of SEQ ID NO:54. In yet other embodiments, the fusion polypeptide comprises an amino acid sequence having at least 96%, at least 97%, at least 98%, at least 99%, or 100% sequence identity with the amino acid sequence shown in (i) residues 23-781 or 1-781 of SEQ ID NO:50, (ii) residues 23-781 or 1-781 of SEQ ID NO:52, or (iii) residues 23-791 or 1-791 of SEQ ID NO:54.

In some embodiments of a paraoxonase fusion polypeptide comprising a CTLA-4 extracellular domain as described above, the fusion polypeptide comprises an amino acid sequence having at least 80%, at least 85%, at least 90%, or at least 95% identity with an amino acid sequence selected from the sequence shown in residues 21-736 or 1-736 of SEQ ID NO:66. In yet other embodiments, the fusion polypeptide comprises an amino acid sequence having at least 96%, at least 97%, at least 98%, at least 99%, or 100% sequence identity with the amino acid sequence shown in residues 21-736 or 1-736 of SEQ ID NO:66.

In some embodiments of a paraoxonase fusion polypeptide comprising a CD40 extracellular domain as described above, the fusion polypeptide comprises an amino acid sequence having at least 80%, at least 85%, at least 90%, or at least 95% identity with an amino acid sequence selected from the sequence shown in (i) residues 21-804 or 1-804 of SEQ ID NO:74, (ii) residues 21-804 or 1-804 of SEQ ID NO:78, (iii) residues 21-804 or 1-804 of SEQ ID NO:82, (iv) residues 21-804 or 1-804 of SEQ ID NO:86, or (v) residues 21-804 or 1-804 of SEQ ID NO:90. In yet other embodiments, the fusion polypeptide comprises an amino acid sequence having at least 96%, at least 97%, at least 98%, at least 99%, or 100% sequence identity with the amino acid sequence shown in (i) residues 21-804 of SEQ ID NO:74 or 1-804, (ii) residues 21-804 or 1-804 of SEQ ID NO:78, (iii) residues 21-804 or 1-804 of SEQ ID NO:82, (iv) residues 21-804 or 1-804 of SEQ ID NO:86, or (v) residues 21-804 or 1-804 of SEQ ID NO:90.

In some embodiments of a paraoxonase fusion polypeptide comprising a polypeptide that specifically binds and neutralizes TNFα as described above, the fusion polypeptide comprises an amino acid sequence having at least 80%, at least 85%, at least 90%, or at least 95% identity with an amino acid sequence selected from the sequence shown in (i) residues 21-860 or 1-860 of SEQ ID NO:94 or (ii) residues 21-860 or 1-860 of SEQ ID NO:98. In yet other embodiments, the fusion polypeptide comprises an amino acid sequence having at least 96%, at least 97%, at least 98%, at least 99%, or 100% sequence identity with the amino acid sequence shown in (i) residues 21-860 or 1-860 of SEQ ID NO:94 or (ii) residues 21-860 or 1-860 of SEQ ID NO:98.

In some embodiments of a paraoxonase fusion polypeptide comprising a polypeptide that specifically binds and neutralizes TGF-β as described above, the fusion polypeptide comprises an amino acid sequence having at least 80%, at least 85%, at least 90%, or at least 95% identity with an amino acid sequence selected from the sequence shown in (i) residues 21-861 or 1-861 of SEQ ID NO:102 or (ii) residues 21-861 or 1-861 of SEQ ID NO:106. In yet other embodiments, the fusion polypeptide comprises an amino acid sequence having at least 96%, at least 97%, at least 98%, at least 99%, or 100% sequence identity with the amino acid sequence shown in (i) residues 21-861 or 1-861 of SEQ ID NO:102 or (ii) residues 21-861 or 1-861 of SEQ ID NO:106.

In some embodiments of a paraoxonase fusion polypeptide comprising the formula X-L2-P as described herein, the fusion polypeptide comprises an amino acid sequence having at least 80%, at least 85%, at least 90%, or at least 95% identity with the amino acid sequence shown in (i) residues 21-613 or 1-613 of SEQ ID NO:122, (ii) residues 21-613 or 1-613 of SEQ ID NO:132, (iii) 21-613 or 1-613 of SEQ ID NO:134, (iv) residues 21-610 or 1-610 of SEQ ID NO:142, (v) residues 21-610 or 1-610 of SEQ ID NO:144, or (vi) residues 21-610 or 1-610 of SEQ ID NO:146. In yet other embodiments, the fusion polypeptide comprises an amino acid sequence having at least 96%, at least 97%, at least 98%, at least 99%, or 100% sequence identity with the amino acid sequence shown in (i) residues 21-613 or 1-613 of SEQ ID NO:122, (ii) residues 21-613 or 1-613 of SEQ ID NO:132, (iii) 21-613 or 1-613 of SEQ ID NO:134, (iv) residues 21-610 or 1-610 of SEQ ID NO:142, (v) residues 21-610 or 1-610 of SEQ ID NO:144, or (vi) residues 21-610 or 1-610 of SEQ ID NO:146.

The present invention also provides dimeric proteins comprising first and second polypeptide fusions, each of the polypeptide fusions comprising a dimerizing domain, as described above. Accordingly, in another aspect, the present invention provides a dimeric protein comprising a first fusion polypeptide and a second fusion polypeptide, wherein each of the first and second fusion polypeptides comprises, from an amino-terminal position to a carboxyl-terminal position, T-L1-X-L2-P, wherein T is a first biologically active polypeptide is selected from a DNase, and RNase, a superoxide dismutase 1 (SOD1), a cytotoxic T-lymphocyte associated molecule-4 (CTLA-4) extracellular domain, a CD40 extracellular domain, and a polypeptide that specifically binds and neutralizes an inflammatory cytokine (e.g., a polypeptide that specifically binds and neutralizes tumor necrosis factor α (TNFα) or transforming growth factor-β (TGFβ)); L1 is a first polypeptide linker that is optionally present; X is a dimerizing domain; L2 is a second polypeptide linker that is optionally present; and P is a biologically active paraoxonase. In another aspect, the present invention provides a dimeric protein comprising a first fusion polypeptide and a second fusion polypeptide, wherein each of the first and second fusion polypeptides comprises, from an amino-terminal position to a carboxyl-terminal position, X-L2-P, wherein X is a dimerizing domain, L2 is a polypeptide linker that is optionally present, and P is a biologically active paraoxonase. In another aspect, the present invention provides a dimeric protein comprising a first fusion polypeptide and a second fusion polypeptide, wherein each of the first and second fusion polypeptides comprises, from an amino-terminal position to a carboxyl-terminal position, P-L1-X, wherein P is a biologically active paraoxonase, L1 is a polypeptide linker, and X is a dimerizing domain.

### III. Materials and Methods for Making Polypeptide Fusions and Dimeric Proteins

The present invention also provides polynucleotide molecules, including DNA and RNA molecules, that encode the fusion polypeptides disclosed above. The polynucleotides of the present invention include both single-stranded and double-stranded molecules. Polynucleotides encoding various segments of a fusion polypeptide (e.g., an Fc fragment; DNase and P polypeptide segments) can be generated and linked together to form a polynucleotide encoding a fusion polypeptide as described herein using known methods for recombinant manipulation of nucleic acids.

DNA sequences encoding fusion components in accordance with the present disclosure (for example, DNases (e.g., DNase1), paraoxonases (e.g., PON1), RNases (e.g., RNase1), superoxide dismutase 1 (SOD1), CTLA-4 and CD40 extracellular domains, and immunoglobulin Fc regions) are generally known in the art. Exemplary DNA sequences are disclosed herein (see Sequence Listing). Additional DNA sequences encoding any of these polypeptides can be readily generated by those of ordinary skill in the art based on the genetic code. Counterpart RNA sequences can be generated by substitution of U for T. Those skilled in the art will readily recognize that, in view of the degeneracy of the genetic code, considerable sequence variation is possible among polynucleotide molecules encoding a given polypeptide. DNA and RNA encoding functional variants and fragments of such polypeptides can also be obtained using known recombinant methods to introduce variation into a polynucleotide sequence, followed by expression of the encoded polypeptide and determination of functional activity (e.g., nuclease activity) using an appropriate screening assay.

Methods for preparing DNA and RNA are well known in the art. For example, complementary DNA (cDNA) clones can be prepared from RNA that is isolated from a tissue or cell that produces large amounts of RNA encoding a polypeptide of interest. Total RNA can be prepared using guanidine HCl extraction followed by isolation by centrifugation in a CsCl gradient (Chirgwin et al., Biochemistry 18:52-94, 1979). Poly (A)₊ RNA is prepared from total RNA using the method of Aviv and Leder (Proc. Natl. Acad. Sci. USA 69:1408-1412, 1972). Complementary DNA is prepared from poly(A)₊ RNA using known methods. In the alternative, genomic DNA can be isolated. Methods for identifying and isolating cDNA and genomic clones are well known and within the level of ordinary skill in the art, and include the use of the sequences disclosed herein, or parts thereof, for probing or priming a library. Polynucleotides encoding polypeptides of interest are identified and isolated by, for example, hybridization or polymerase chain reaction (“PCR,” Mullis, U.S. Pat. No. 4,683.202). Expression libraries can be probed with antibodies to the polypeptide of interest, receptor fragments, or other specific binding partners.

The polynucleotides of the present invention can also be prepared by automated synthesis. The production of short, double-stranded segments (60 to 80 bp) is technically straightforward and can be accomplished by synthesizing the complementary strands and then annealing them. Longer segments (typically >300 bp) are assembled in modular form from single-stranded fragments that are from 20 to 100 nucleotides in length. Automated synthesis of polynucleotides is within the level of ordinary skill in the art, and suitable equipment and reagents are available from commercial suppliers. See generally Glick and Pasternak, Molecular Biotechnology, Principles & Applications of Recombinant DNA, ASM Press, Washington, D.C., 1994; Itakura et al., Ann. Rev. Biochem. 53:323-356, 1984; and Climie et al., Proc. Natl. Acad. Sci. USA 87:633-637, 1990.

In another aspect, materials and methods are provided for producing the polypeptide fusions of the present invention, including dimeric proteins comprising the fusion polypeptides. The fusion polypeptides can be produced in genetically engineered host cells according to conventional techniques. Suitable host cells are those cell types that can be transformed or transfected with exogenous DNA and grown in culture, and include bacteria, fungal cells, and cultured higher eukaryotic cells (including cultured cells of multicellular organisms), particularly cultured mammalian cells. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N Y, 1989, and Ausubel et al., eds., Current Protocols in Molecular Biology, Green and Wiley and Sons, N Y, 1993.

In general, for production of a fusion polypeptide in a host cell, a DNA sequence encoding the fusion polypeptide is operably linked to other genetic elements required for its expression, typically including a transcription promoter and terminator, within an expression cassette. Typically, the expression cassette is contained within an expression vector for delivery into a host cell. The vector will also commonly contain one or more selectable markers and one or more origins of replication, although those skilled in the art will recognize that within certain systems selectable markers may be provided on separate vectors, and replication of the exogenous DNA may be provided by integration of an expression cassette into the host cell genome such as, e.g., in the generation of stable cell lines. Selection of promoters, terminators, selectable markers, vectors and other elements is a matter of routine design within the level of ordinary skill in the art. Many such elements are described in the literature and are available through commercial suppliers.

To direct a DNase fusion polypeptide into the secretory pathway of a host cell, a secretory signal sequence is provided in the expression cassette. The encoded secretory peptide may be that of a corresponding native protein (e.g., a native DNase secretory peptide as shown in amino acid residues 1-22 of SEQ ID NO:16, a native RNase secretory peptide as shown in amino acid residues 1-28 of SEQ ID NO:38, or a native CD40 secretory peptide as shown in amino acid residues 1-20 of SEQ ID NO:68), or may be derived from another secreted protein (e.g., t-PA; see U.S. Pat. No. 5,641,655) or synthesized de novo. An engineered cleavage site may be included at the junction between the secretory peptide and the remainder of the polypeptide fusion to optimize proteolytic processing in the host cell. The secretory signal sequence is operably linked to the DNA sequence encoding the polypeptide fusion, i.e., the two sequences are joined in the correct reading frame and positioned to direct the newly synthesized polypeptide fusion into the secretory pathway of the host cell. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830). Secretory signal sequences suitable for use in accordance with the present invention include, for example, polynucleotides encoding the human VK3 leader peptide (SEQ ID NO:58).

Expression of fusion polypeptides comprising a dimerizing domain as described herein, via a host cell secretory pathway, is expected to result in the production of dimeric proteins. Accordingly, in another aspect, the present invention provides dimeric proteins comprising first and second fusion polypeptides as described above (e.g., a dimeric protein comprising a first fusion polypeptide and a second fusion polypeptide, wherein each of the first and second fusion polypeptides comprises, from an amino-terminal position to a carboxyl-terminal position, T-L1-X-L2-P, X-L2-P, or P-L1-X as described herein). Dimers may also be assembled in vitro upon incubation of component polypeptides under suitable conditions. In general. In vitro assembly will include incubating the protein mixture under denaturing and reducing conditions followed by refolding and reoxidation of the polypeptides to form dimers. Recovery and assembly of proteins expressed in bacterial cells is disclosed below.

Cultured mammalian cells are suitable hosts for use within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann el al., EMBO J. 1:841-845, 1982), DEAE-dextran mediated transfection (Ausubel et al., supra), and liposome-mediated transfection (Hawley-Nelson et al., Focus 15:73, 1993; Ciccarone et al., Focus 15:80, 1993). The production of recombinant polypeptides in cultured mammalian cells is disclosed by, for example, Levinson et al., U.S. Pat. No. 4,713,339; Hagen et al., U.S. Pat. No. 4,784,950; Palmiter el al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g., CHO-K1, ATCC No. CCL 61; CHO-DG44, Urlaub et al., Proc. Natl. Acad. Sci. USA 77:4216-4220, 1980) cell lines. Additional suitable cell lines are known in the art and available from public depositories such as the American Type Culture Collection, Manassas, Virginia. Strong transcription promoters can be used, such as promoters from SV-40, cytomegalovirus, or myeloproliferative sarcoma virus. See, e.g., U.S. Pat. No. 4,956,288 and U.S. Patent Application Publication No. 20030103986. Other suitable promoters include those from metallothionein genes (U.S. Pat. Nos. 4,579,821 and 4,601,978) and the adenovirus major late promoter. Expression vectors for use in mammalian cells include pZP-1, pZP-9, and pZMP21, which have been deposited with the American Type Culture Collection, 10801 University Blvd., Manassas, VA USA under accession numbers 98669, 98668, and PTA-5266, respectively, and derivatives of these vectors.

Drug selection is generally used to select for cultured mammalian cells into which foreign DNA has been inserted. Such cells are commonly referred to as “transfectants.” Cells that have been cultured in the presence of the selective agent and are able to pass the gene of interest to their progeny—by virtue of integration of the expression cassette into its genomic DNA—are referred to as “stable transfectants.” An exemplary selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. An exemplary amplifiable selectable marker is dihydrofolate reductase, which confers resistance to methotrexate. Other drug resistance genes (e.g., hygromycin resistance, multi-drug resistance, puromycin acetyltransferase) can also be used. Cell-surface markers and other phenotypic selection markers can be used to facilitate identification of transfected cells (e.g., by fluorescence-activated cell sorting), and include, for example, CD8, CD4, nerve growth factor receptor, green fluorescent protein, and the like.

In some aspects, the present invention provides a stable cell line containing, within its genomic DNA, an expression cassette encoding a paraoxonase fusion polypeptide as described herein, wherein the stable cell line constitutively expresses the encoded paraoxonase fusion. Stable cell lines can be generated by methods generally known in the art, which generally include the identification of single stable cell clones from a polyclonal colony of stable transfectants by limited dilution and expansion. Protein expression of selected clones can then be assessed to identify high-expressing clones for expansion. In some embodiments, the stable cell line is a mammalian cell line such as, e.g., a Chinese hamster ovary (CHO) cell line. In some preferred embodiments of a stable cell line constitutively expressing a DNase-PON fusion polypeptide in accordance with the present disclosure, the DNase is an enhanced DNase1 as described herein; in some such embodiments, the DNase-PON fusion polypeptide is an enhanced DNase1-Fc-PON1 fusion polypeptide comprising an amino acid sequence having at least 90%, at least 95%, at least 96%, at least 97%, at least 98%, at least 99%, or 100% identity with the amino acid sequence shown in (i) residues 21-896 or 1-896 of SEQ ID NO:2, (ii) residues 21-906 or 1-906 of SEQ ID NO:148, (iii) residues 21-896 or 1-896 of SEQ ID NO:158, or (iv) residues 21-906 or 1-906 of SEQ ID NO:160. In some variations, the stable cell line is a mammalian cell line (e.g., CHO) capable of producing the fusion polypeptide (e.g., a DNase1-Fc-PON1 fusion polypeptide) at a concentration of at least 25 mg/L, at least 50 mg/L, at least 100 mg/L, at least 200 mg/L, or at least 500 mg/L of the cell cuture. Typically, a DNase1-Fc fusion polypeptide as described herein can be expressed at levels of approximately 25-100 mg/L from initial isolated clones. Recloning of the initial cultures can often stabilize and increase expression by 2-3 fold. Amplification of the expression level can also be achieved by further plating of cells at low density in increasing levels of methotrexate from an initial concentration of 50 nM up to as much as 1 μM. Once cells have adapted, further rounds of limiting dilution cloning are required to maintain high expression levels.

Other higher eukaryotic cells can also be used as hosts, including insect cells, plant cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and International PCT Publication No. WO 94/06463.

Insect cells can be infected with recombinant baculovirus, commonly derived from Autographa californica nuclear polyhedrosis virus (AcNPV) (see King and Possee, The Baculovirus Expression System: A Laboratory Guide, Chapman & Hall, London; O'Reilly et al., Baculovirus Expression Vectors: A Laboratory Manual, Oxford University Press., New York, 1994; and Richardson, Ed., Baculovirus Expression Protocols. Methods in Molecular Biology, Humana Press, Totowa, N J, 1995). Recombinant baculovirus can also be produced through the use of a transposon-based system described by Luckow et al. (J. Virol. 67:4566-4579, 1993). This system, which utilizes transfer vectors, is commercially available in kit form (BAC-TO-BAC kit; Life Technologies, Gaithersburg, MD). The transfer vector (e.g., PFASTBAC1; Life Technologies) contains a Tn7 transposon to move the DNA encoding the protein of interest into a baculovirus genome maintained in E. coli as a large plasmid called a “bacmid” (see Hill-Perkins and Possee, J. Gen. Virol. 71:971-976, 1990; Bonning et al., J. Gen. Virol. 75:1551-1556, 1994; Chazenbalk and Rapoport, J. Biol. Chem. 270:1543-1549, 1995). Using techniques known in the art, a transfer vector encoding a polypeptide fusion is transformed into E. coli host cells, and the cells are screened for bacmids which contain an interrupted lacZ gene indicative of recombinant baculovirus. The bacmid DNA containing the recombinant baculovirus genome is isolated, using common techniques, and used to transfect Spodoptera frugiperda cells, such as Sf9 cells. Recombinant virus that expresses the polypeptide fusion is subsequently produced. Recombinant viral stocks are made by methods commonly used in the art.

For protein production, the recombinant virus is used to infect host cells, typically a cell line derived from the fall armyworm, Spodoptera frugiperda (e.g., Sf9 or Sf21 cells) or Trichoplusia ni (e.g., HIGH FIVE cells; Invitrogen, Carlsbad, CA) (see generally Glick and Pasternak, supra; see also U.S. Pat. No. 5,300,435). Serum-free media are used to grow and maintain the cells. Suitable media formulations are known in the art and can be obtained from commercial suppliers. The cells are grown up from an inoculation density of approximately 2-5×10⁵ cells to a density of 1-2×10⁶ cells, at which time a recombinant viral stock is added at a multiplicity of infection (MOI) of 0.1 to 10, more typically near 3. Procedures used are generally described in available laboratory manuals (e.g., King and Possee, supra; O'Reilly et al., supra.; Richardson, supra).

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). An exemplary vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936; and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guilliermondii, and Candida maltosa are known in the art. See, e.g., Gleeson et al., J. Gen. Microbiol. 132:3459-3465, 1986; Cregg, U.S. Pat. No. 4,882,279; and Raymond et al., Yeast 14:11-23, 1998. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533. Production of recombinant proteins in Pichia methanolica is disclosed in U.S. Pat. Nos. 5,716,808; 5,736,383; 5,854,039; and 5,888,768.

Prokaryotic host cells, including strains of the bacteria Escherichia coli, Bacillus and other genera are also useful host cells within the present invention. Techniques for transforming these hosts and expressing foreign DNA sequences cloned therein are well-known in the art (see, e.g., Sambrook et al., supra). When expressing a fusion polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine HCl or urea. The denatured polypeptide can then be refolded and dimerized by diluting the denaturant, such as by dialysis against a solution of urea and a combination of reduced and oxidized glutathione, followed by dialysis against a buffered saline solution. In the alternative, the protein may be recovered from the cytoplasm in soluble form and isolated without the use of denaturants. The protein is recovered from the cell as an aqueous extract in, for example, phosphate buffered saline. To capture the protein of interest, the extract is applied directly to a chromatographic medium, such as an immobilized antibody or heparin-Sepharose column. Secreted polypeptides can be recovered from the periplasmic space in a soluble and functional form by disrupting the cells (by, for example, sonication or osmotic shock) and recovering the protein, thereby obviating the need for denaturation and refolding. See, e.g., Lu et al., J. Immunol. Meth. 267:213-226, 2002.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

In some variations, for production of an actin-resistant DNase1-PON fusion polypeptide as described herein (e.g., an actin-resistant DNase1-L1-Fc-L2-PON1 fusion polypeptide comprising an amino acid sequence having at least 90%, at least 95%, at least 96%, at least 97%, at least 98%, at least 99%, or 100% identity with the amino acid sequence shown in (i) residues 21-896 or 1-896 of SEQ ID NO:2 or (ii) residues 21-906 or 1-906 of SEQ ID NO:148), host cells are cultured in the presence of a DNase1 inhibitor to reduce toxicity of the enhanced DNase during its expression. DNase1 inhibitors are generally known in the art and include, for example, the inhibitor rutin (see, e.g., Kolarevic el al., Chem. Biodiversity 16:e1900060, 2019). In certain embodiments, the host cells cultured in the presence of a DNase1 inhibitor are mammalian cells such as, e.g., CHO.

Proteins of the present invention are purified by conventional protein purification methods, typically by a combination of chromatographic techniques. See generally Affinity Chromatography: Principles & Methods, Pharmacia LKB Biotechnology, Uppsala, Sweden. 1988; and Scopes, Protein Purification: Principles and Practice, Springer-Verlag, New York, 1994. Proteins comprising an immunoglobulin Fc region can be purified by affinity chromatography on immobilized protein A. Additional purification steps, such as gel filtration, can be used to obtain the desired level of purity or to provide for desalting, buffer exchange, and the like.

For example, fractionation and/or conventional purification methods can be used to obtain fusion polypeptides and dimeric proteins of the present invention purified from recombinant host cells. In general, ammonium sulfate precipitation and acid or chaotrope extraction may be used for fractionation of samples. Exemplary purification steps may include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are suitable. Exemplary chromatographic media include those media derivatized with phenyl, butyl, or octyl groups, such as Phenyl-Sepharose FF (Pharmacia), Toyopearl butyl 650 (Toso Haas, Montgomeryville, PA), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties.

Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well-known and widely used in the art, and are available from commercial suppliers. Selection of a particular method for polypeptide isolation and purification is a matter of routine design and is determined in part by the properties of the chosen support. See, e.g., Affinity Chromatography: Principles & Methods (Pharmacia LKB Biotechnology 1988); Doonan, Protein Purification Protocols (The Humana Press 1996).

Additional variations in protein isolation and purification can be devised by those of skill in the art. For example, antibodies that specifically bind a fusion polypeptide or dimeric protein as described herein (e.g., an antibody that specifically binds a polypeptide segment corresponding to a DNase) can be used to isolate large quantities of protein by immunoaffinity purification.

The proteins of the present invention can also be isolated by exploitation of particular properties. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (see, e.g., M. Deutscher, (ed.), Meth. Enzymol. 182:529, 1990). Within additional embodiments of the invention, a fusion of the polypeptide of interest and an affinity tag (e.g., maltose-binding protein) may be constructed to facilitate purification. Moreover, receptor- or ligand-binding properties of a fusion polypeptide or dimer thereof can be exploited for purification.

In some variations, a DNase1-PON fusion molecule comprising an enhanced but non-actin-resistant DNase1 as described herein (i.e., a DNase that does not contain any amino acid substitution, relative to human DNase1, that decreases G-actin-induced inhibition of endonuclease activity of the DNase relative to human DNase1) is treated with ethylenediamine (EDA) following purification in order to reduce the molecule's actin binging, thereby providing a DNase-Fc fusion molecule that is resistant to actin. The EDA-treated DNase1-PON fusion molecule is then further treated to remove EDA using methods generally known in the art (see, e.g., U.S. Pat. No. 11,225,648). Such embodiments are advantageous for achieving higher expression of the DNase1-PON fusion for pharmaceutical use, since expression of the non-actin-resistant form of enhanced DNase1 would be less toxic to host cells (e.g., CHO). In particular embodiments, the purified DNase1-PON to be treated with EDA comprises an amino acid sequence having at least 90%, at least 95%, at least 96%, at least 97%, at least 98%, at least 99%, or 100% identity with the amino acid sequence shown in (i) residues 21-896 of SEQ ID NO:158 or (ii) residues 21-906 of SEQ ID NO:160.

The polypeptides of the present invention are typically purified to at least about 80% purity, more typically to at least about 90% purity and preferably to at least about 95%, at least about 96%, at least about 97%, at least about 98%, or at least about 99% purity with respect to contaminating macromolecules, particularly other proteins and nucleic acids, and free of infectious and pyrogenic agents. The polypeptides of the present invention may also be purified to a pharmaceutically pure state, which is greater than 99.9% pure. In certain preparations, purified polypeptide is substantially free of other polypeptides, particularly other polypeptides of animal origin.

### IV. Methods of Use and Pharmaceutical Compositions

The fusion polypeptides and dimeric proteins of the present invention can be used to provide therapy for the treatment of various diseases or disorders. The paraoxonase fusion polypeptides are particularly useful, e.g., for treatment of inflammatory, autoimmune, neurological, cardiovascular, and/or fibrotic diseases and disorders. In aspects relating to bispecific fusions further comprising a first biologically active polypeptide as described herein (e.g., a DNase, RNase, or SOD1), the fusion polypeptides and dimeric proteins may further provide one or more additional biological activities for treatment. For example, paraoxonase fusion polypeptides comprising a DNase or an RNase are particularly useful, e.g., for treatment of diseases and disorders characterized by NETosis. In addition, paraoxonase fusion polypeptides comprising a CTLA-4 or CD40 extracellular domain are particularly useful, e.g., for treatment of diseases or disorders characterized by an aberrant adaptive immune response. As yet another example, paraoxonase fusion polypeptides comprising a CD40 extracellular domain or a polypeptide that specifically binds and neutralizes TGF-β are particularly useful, e.g., for treatment of diseases and disorders characterized by a fibrotic inflammatory response.

In some aspects, the present invention provides methods for treating a disease or disorder selected from an inflammatory disease, an autoimmune disease, a neurological disease, an infectious disease, a metabolic disease, a cardiovascular disease, a liver disease, a fibrotic disease, thrombosis, sepsis, ischemia repurfusion, biofilm formation by a gram-negative bacteria, exposure to sulfur mustard gas, exposure to an organophosphate, and cancer. The methods generally include administering to a subject having the disease or disorder an effective amount of a fusion polypeptide or dimeric protein as described herein.

Inflammatory diseases amenable to treatment in accordance with the present invention include, for example, inflammatory lung diseases such as, for example, asthma, cystic fibrosis (CF), chronic obstructive pulmonary disease (COPD), bronchiectasis, hypoxia, interstitial lung disease (e.g., idiopathic pulmonary fibrosis (IPF) or sarcoidosis), and acute respiratory distress syndrome (ARDS). In particular embodiments comprising treatment of ARDS, the ARDS is associated with COVID-19. In some embodiments, the inflammatory lung disease is characterized by Pseudomonas aeruginosa infection. In some variations, a patient having the inflammatory lung disease is a patient that has been exposed to sulfur mustard gas (SM). In other variations, a patient having the inflammatory lung disease is a patient that has been exposed to an organophosphate, such as an insecticide (e.g., parathion, malathion, chlorpyrifos, diazinon, dichlorvos, phosmet, fenitrothion, terbufos, tetrachlorvinphos, azamethiphos, or azinphos-methyl) or other neurotoxin (e.g., tabun, sarin, soman, or cyclosarin).

Other inflammatory diseases amenable to treatment in accordance with the present invention include autoinflammatory diseases (i.e., innate immune system activation disorders characterized by seemingly unprovoked episodes of inflammation and a relative lack of obvious autoimmune pathology). Exemplary autoinflammatory diseases include inflammatory bowel disease (IBD) (e.g., Crohn's disease, ulcerative colitis), Behcet's disease, systemic onset juvenile idiopathic arthritis (JIA), gout, pseudogout, storage (Gaucher's) disorders, hereditary angioedema (HAE), atypical hemolytic uremic syndrome, familial Mediterranean fever (FMF), TNF-receptor associated periodic fever syndrome (TRAPS), cryopyrin-associated periodic syndromes (CAPS)), NOD2-associated autoinflammatory disease (NAID), and Blau syndrome.

In yet other embodiments, an inflammatory disease or disorder for treatment in accordance with the present invention is selected from rheumatoid arthritis, systemic lupus erythematosus, multiple sclerosis, type 1 diabetes, type 2 diabetes, hepatitis (e.g., nonalcoholic steatohepatitis (NASH)), ankylosing spondylitis, psoriasis, psoriatic arthritis, dermatitis (e.g., atopic dermatitis), diverticulitis, irritable bowel syndrome, and nephritis. In still other variations, the inflammatory disease or disorder is an inflammatory skin disease such as, e.g., psoriasis or atopic dermatitis.

In more particular embodiments of a method for treating an inflammatory disease, a fusion molecule for the inflammatory disease treatment is a polypeptide having the structure DNase1-L1-Fc-L2-PON1, DNase1L3-L1-Fc-L2-PON1, RNase1-L1-Fc-L2-PON1, RNase1-Fc-L2-PON1, SOD1-L1-Fc-L2-PON1, SOD1-Fc-L2-PON1, CTLA4-L1-Fc-L2-PON1, CTLA4-Fc-L2-PON1, CD40-L1-Fc-L2-PON1, CD40-Fc-L2-PON1, antiTNFα-L1-Fc-L2-PON1, antiTNFα-Fc-L2-PON1, antiTGFβ-L1-Fe-L2-PON1, antiTGFβ-Fc-L2-PON1, Fc-L2-PON1, or PON1-L1-Fc, or a dimeric protein formed by dimerization of any of the foregoing fusion polypeptides; in some such embodiments, the fusion polypeptide comprises or consists of an amino acid sequence having at least 90%, at least 95%, or 100% identity with the amino acid sequence shown in (i) residues 21-896 of SEQ ID NO:2, (ii) residues 21-764 of SEQ ID NO:40, (iii) residues 21-740 of SEQ ID NO:48, (iv) residues 23-781 of SEQ ID NO:50, (v) residues 23-781 of SEQ ID NO:52, (vi) residues 23-791 of SEQ ID NO:54, (vii) residues 21-736 of SEQ ID NO:66, (viii) residues 21-804 of SEQ ID NO:74, (ix) residues 21-804 of SEQ ID NO:78, (x) residues 21-804 of SEQ ID NO:82, (xi) residues 21-804 of SEQ ID NO:86, (xii) residues 21-804 of SEQ ID NO:90, (xiii) residues 21-860 of SEQ ID NO:94, (xiv) residues 21-860 of SEQ ID NO:98, (xv) residues 21-861 of SEQ ID NO:102, (xvi) residues 21-861 of SEQ ID NO:106, (xvii) residues 21-613 of SEQ ID NO:122, (xviii) residues 21-610 of SEQ ID NO:142, (xix) residues 21-906 of SEQ ID NO:148, (xx) residues 21-896 of SEQ ID NO:158, (xxi) residues 21-906 of SEQ ID NO:160, (xxii) residues 21-906 of SEQ ID NO:162, or (xxiii) residues 21-906 of SEQ ID NO:164. In some embodiments of a method for treating an autoinflammatory disease, the fusion molecule is a polypeptide having the structure DNase1-L1-Fc-L2-PON1, DNase1L3-L1-Fc-L2-PON1, RNase1-L1-Fc-L2-PON1, or RNase1-Fc-L2-PON1, or a dimeric protein formed by dimerization of any of the foregoing fusion polypeptides. In some embodiments of a method for treating rheumatoid arthritis, psoriatic arthritis, or juvenile idiopathic arthritis, the fusion molecule is a polypeptide having the structure CTLA4-L1-Fc-L2-PON1 or CTLA4-Fc-L2-PON1, or a dimeric protein formed by dimerization of either of the foregoing fusion polypeptides. In some embodiments of a method for treating an inflammatory bowel disease (IBD) (e.g., Crohn's disease or ulcerative colitis), the fusion molecule is a polypeptide having the structure DNase1-L1-Fc-L2-PON1, DNase1L3-L1-Fc-L2-PON1, antiTNFα-L1-Fc-L2-PON1, or antiTNFα-Fc-L2-PON1, or a dimeric protein formed by dimerization of any of the foregoing fusion polypeptides. In some embodiments of a method for treating an inflammatory skin disease (e.g., psoriasis or atopic dermatitis), the fusion molecule molecule is a polypeptide having the structure DNase1-L1-Fc-L2-PON1, DNase1L3-L1-Fc-L2-PON1, antiTNFα-L1-Fc-L2-PON1, antiTNFα-Fc-L2-PON1, Fc-L2-PON1, or PON1-L1-Fc, or a dimeric protein formed by dimerization of any of the foregoing fusion polypeptides.

Autoimmune diseases amenable to treatment in accordance with the present invention include, for example, rheumatoid arthritis, systemic lupus erythematosus, psoriasis, multiple sclerosis, type 1 diabetes, vasculitis, and systemic sclerosis (also known as scleroderma). In other embodiments, the autoimmune disease is selected from coeliac disease, neuritis, polymyositis, juvenile rheumatoid arthritis, psoriatic arthritis, vitiligo, Sjogren's syndrome, autoimmune pancreatitis, autoimmune hepatitis, glomerulonephritis, lupus nephritis, scleroderma, antiphospholipid syndrome, autoimmune vasculitis, sarcoidosis, autoimmune thyroid diseases, Hashimoto's thyroiditis, Graves disease, Wegener's granulomatosis, myasthenia gravis, Addison's disease, autoimmune uveoretinitis, pemphigus vulgaris, primary biliary cirrhosis, pernicious anemia, sympathetic opthalmia, uveitis, autoimmune hemolytic anemia, pulmonary fibrosis, chronic beryllium disease, and idiopathic pulmonary fibrosis. In some variations comprising treatment of vasculitis, the vasculitis is selected from small vessel vasculitis and medium vessel vasculitis; in other variations, the vasculitis is large vessel vasculitis.

In more particular embodiments of a method for treating an autoimmune disease, a fusion molecule for the autoimmune disease treatment is a polypeptide having the structure DNase1-L1-Fc-L2-PON1, DNase1L3-L1-Fc-L2-PON1, RNase1-L1-Fc-L2-PON1, RNase1-Fc-L2-PON1, SOD1-L1-Fc-L2-PON1, SOD1-Fc-L2-PON1, CTLA4-L1-Fc-L2-PON1, CTLA4-Fc-L2-PON1, CD40-L1-Fc-L2-PON1, CD40-Fc-L2-PON1, antiTNFα-L1-Fc-L2-PON1, antiTNFα-Fc-L2-PON1, antiTGFβ-L1-Fc-L2-PON1, antiTGFβ-Fc-L2-PON1, Fc-L2-PON1, or PON1-L1-Fc, or a dimeric protein formed by dimerization of any of the foregoing fusion polypeptides; in some such embodiments, the fusion polypeptide comprises or consists of an amino acid sequence having at least 90%, at least 95%, or 100% identity with the amino acid sequence shown in (i) residues 21-896 of SEQ ID NO:2, (ii) residues 21-764 of SEQ ID NO:40, (iii) residues 21-740 of SEQ ID NO:48, (iv) residues 23-781 of SEQ ID NO:50, (v) residues 23-781 of SEQ ID NO:52, (vi) residues 23-791 of SEQ ID NO:54, (vii) residues 21-736 of SEQ ID NO:66, (viii) residues 21-804 of SEQ ID NO:74, (ix) residues 21-804 of SEQ ID NO:78, (x) residues 21-804 of SEQ ID NO:82, (xi) residues 21-804 of SEQ ID NO:86, (xii) residues 21-804 of SEQ ID NO:90, (xiii) residues 21-860 of SEQ ID NO:94, (xiv) residues 21-860 of SEQ ID NO:98, (xv) residues 21-861 of SEQ ID NO:102, (xvi) residues 21-861 of SEQ ID NO:106, (xvii) residues 21-613 of SEQ ID NO:122, (xviii) residues 21-610 of SEQ ID NO:142, (xix) residues 21-906 of SEQ ID NO:148, (xx) residues 1-896 of SEQ ID NO:158, (xxi) residues 21-906 of SEQ ID NO:160, (xxii) residues 21-906 of SEQ ID NO:162, or (xxiii) residues 21-906 of SEQ ID NO:164. In some embodiments of a method for treating systemic lupus erythematosus, rheumatoid arthritis, psoriasis, type 1 diabetes, or small vessel vasculitis, the fusion molecule is a fusion polypeptide having the structure DNase1-L1-Fc-L2-PON1 or DNase1 L3-L1-Fc-L2-PON1, or a dimeric protein formed by dimerization thereof. In some embodiments of a method for treating systemic lupus erythematosus, Sjogren's syndrome, or type 1 diabetes, the fusion molecule is a polypeptide having the structure RNase1-L1-Fc-L2-PON1 or RNase1-Fc-L2-PON1, or a dimeric protein formed by dimerization of either of the foregoing fusion polypeptides. In some embodiments of a method for treating rheumatoid arthritis or psoriatic arthritis, the fusion molecule is a polypeptide having the structure CLTA4-L1-Fc-L2-PON1 or CTLA4-Fc-L2-PON1, or a dimeric protein formed by dimerization of either of the foregoing fusion polypeptides.

Neurological diseases amenable to treatment in accordance with the present invention include, for example, neurodegenerative diseases characterized by inflammation in the CNS such as, e.g., multiple sclerosis, Alzheimer's disease, Parkinson's disease, or amylotrophic lateral sclerosis (ALS). In some embodiments, the neurological disease is a neurodegenerative disease characterized by dementia such as, e.g., Alzheimer's disease. In more specific variations of a method for treating multiple sclerosis (MS), the MS is spino-optical MS, primary progressive MS (PPMS), or relapsing remitting MS (RRMS). In other embodiments, a neurological disease for treatment in accordance with the present invention is a CNS infection such as, e.g., meningitis, encephalitis, or cerebral malaria. In more particular variations of a method for treating meningitis, the meningitis is a bacterial meningitis; in some such embodiments, the CNS infection is an S. pneumoniae, N. meningitis, S. aureus, E. coli, A. baumanii, S. oralis, S. capitis, or S. epidermidis infection. Other neurological diseases or disorders amenable to treatment with fusion molecules as described herein include, for example, acute brain injury such as, e.g., ischemic stroke. In still other embodiments, the neurological disease is a brain cancer such as, e.g., an intracranial tumor selected from astrocytoma, anaplastic astrocytoma, glioblastoma, oligodendroglioma, anaplastic oligodendroglioma, ependymoma, primary CNS lymphoma, medulloblastoma, germ cell tumor, pineal gland neoplasm, meningioma, pituitary tumor, tumor of the nerve sheath (e.g., schwannoma), chordoma, craniopharyngioma, and a choroid plexus tumor (e.g., choroid plexus carcinoma).

In more particular embodiments of a method for treating a neurological disease, a fusion molecule for the neurological disease treatment is a polypeptide having the structure DNase1-L1-Fc-L2-PON1, DNase1L3-L1-Fc-L2-PON1, RNase1-L1-Fc-L2-PON1, RNase1-Fc-L2-PON1, SOD1-L1-Fc-L2-PON1, SOD1-Fc-L2-PON1, CTLA4-L1-Fc-L2-PON1, CTLA4-Fc-L2-PON1, CD40-L1-Fc-L2-PON1, CD40-Fc-L2-PON1, antiTNFα-L1-Fc-L2-PON1, antiTNFα-Fc-L2-PON1, antiTGFβ-L1-Fc-L2-PON1, antiTGFβ-Fc-L2-PON1, Fc-L2-PON1, or PON1-L1-Fc, or a dimeric protein formed by dimerization of any of the foregoing fusion polypeptides; in some such embodiments, the fusion polypeptide comprises or consists of an amino acid sequence having at least 90%, at least 95%, or 100% identity with the amino acid sequence shown in (i) residues 21-896 of SEQ ID NO:2, (ii) residues 21-764 of SEQ ID NO:40, (iii) residues 21-740 of SEQ ID NO:48, (iv) residues 23-781 of SEQ ID NO:50, (v) residues 23-781 of SEQ ID NO:52, (vi) residues 23-791 of SEQ ID NO:54, (vii) residues 21-736 of SEQ ID NO:66, (viii) residues 21-804 of SEQ ID NO:74, (ix) residues 21-804 of SEQ ID NO:78, (x) residues 21-804 of SEQ ID NO:82, (xi) residues 21-804 of SEQ ID NO:86, (xii) residues 21-804 of SEQ ID NO:90, (xiii) residues 21-860 of SEQ ID NO:94, (xiv) residues 21-860 of SEQ ID NO:98, (xv) residues 21-861 of SEQ ID NO:102, (xvi) residues 21-861 of SEQ ID NO:106, (xvii) residues 21-613 of SEQ ID NO:122, (xviii) residues 21-610 of SEQ ID NO:142, (xix) residues 21-906 of SEQ ID NO:148, (xx) residues 21-896 of SEQ ID NO:158, (xxi) residues 21-906 of SEQ ID NO:160, (xxii) residues 21-906 of SEQ ID NO:162, or (xxiii) residues 21-906 of SEQ ID NO:164. In some embodiments of a method for treating a neurodegenerative disease characterized by dementia (e.g., Alzheimer's disease), acute brain injury (e.g., ischemic stroke), or a brain cancer, the fusion molecule is a polypeptide having the structure DNase-L1-Fc-L2-PON1, DNase1L3-L1-Fc-L2-PON1, RNase1-L1-Fc-L2-PON1, or RNase1-Fc-L2-PON1, or a dimeric protein formed by dimerization of any of the foregoing fusion polypeptides. In some embodiments of a method for treating multiple sclerosis, Alzheimer's disease, Parkinson's disease, amylotrophic lateral sclerosis, or a CNS infection, the fusion molecule is a polypeptide having the structure DNase1-L1-Fc-L2-PON1 or DNase L3-L1-Fc-L2-PON1, or a dimeric protein formed by dimerization thereof.

Infectious diseases amenable to treatment in accordance with the present invention include, for example, bacterial infections, viral infections, fungal infections, and parasitic infections. In some embodiments comprising treatment of a parasitic infection, the infection is a Trypanosoma brucei, Leishmania, Plasmodium falciparum, or Toxoplasma gondii infection. In some embodiments comprising treatment of a bacterial infection, the infection is a Staphylococcus aureus, Streptococcus pneumoniae, or Mycobacterium tuberculosis infection. In other embodiments, the bacterial infection is a Pseudomonas aeruginosa infection. In yet other embodiments, the bacterial infection is a Borrelia burgdorferi infection (Lyme disease). In some embodiments comprising treatment of a viral infection, the infection is an influenza virus (e.g., influenza A virus) or respiratory syncytial virus (RSV) infection. In still other embodiments, the infection is a CNS infection such as, for example, meningitis (e.g., a bacterial meningitis), encephalitis, or cerebral malaria.

Paraoxonase fusion molecules for treating an infectious disease typically comprise a DNase, RNase, SOD1, or anti-TGF-β neutralizing polypeptide as the first biologically active polypeptide, or may be a monospecific paraoxonase fusion molecule. In more particular variations, a fusion molecule for infectious disease treatment is a polypeptide having the structure DNase1-L1-Fc-L2-PON1, DNase1L3-L1-Fc-L2-PON1, RNase1-L1-Fc-L2-PON1, RNase1-Fc-L2-PON1, SOD1-L1-Fc-L2-PON1, SOD1-Fc-L2-PON1, antiTNFα-L1-Fc-L2-PON1, antiTNFα-Fc-L2-PON1, antiTGFβ-L1-Fc-L2-PON1, antiTGFβ-Fc-L2-PON1, Fc-L2-PON1, or PON1-L1-Fc, or a dimeric protein formed by dimerization of any of the foregoing fusion polypeptides. In some such embodiments, the fusion polypeptide comprises or consists of an amino acid sequence having at least 90%, at least 95%, or 100% identity with the amino acid sequence shown in (i) residues 21-896 of SEQ ID NO:2, (ii) residues 21-764 of SEQ ID NO:40, (iii) residues 21-740 of SEQ ID NO:48, (iv) residues 23-781 of SEQ ID NO:50, (v) residues 23-781 of SEQ ID NO:52, (vi) residues 23-791 of SEQ ID NO:54, (vii) residues 21-860 of SEQ ID NO:94, (viii) residues 21-860 of SEQ ID NO:98, (ix) residues 21-861 of SEQ ID NO:102, (x) residues 21-861 of SEQ ID NO:106, (xi) residues 21-613 of SEQ ID NO:122, (xii) residues 21-610 of SEQ ID NO:142, (xiii) residues 21-906 of SEQ ID NO:148, (xiv) residues 21-896 of SEQ ID NO:158, (xv) residues 21-906 of SEQ ID NO:160, (xvi) residues 21-906 of SEQ ID NO:162, or (xvii) residues 21-906 of SEQ ID NO:164.

In some embodiments of a method for treating biofilm formation by a gram negative bacteria, the gram negative bacteria is Pseudomonas aeruginosa. In particular variations for treating biofilm formation by a gram negative bacteria, a fusion molecule for the biofilm formation treatment is a polypeptide having the structure DNase1-L1-Fc-L2-PON1, DNase1 L3-L1-Fc-L2-PON1, Fc-L2-PON1, or PON1-L1-Fc, or a dimeric protein formed by dimerization of either of the foregoing fusion polypeptides; in some such embodiments, the fusion polypeptide comprises or consists of an amino acid sequence having at least 90%, at least 95%, or 100% identity with the amino acid sequence shown in (i) residues 21-896 of SEQ ID NO:2, (ii) residues 21-613 of SEQ ID NO:122, (iii) residues 21-610 of SEQ ID NO:142, (xix) residues 21-906 of SEQ ID NO:148, (xx) residues 21-896 of SEQ ID NO:158, (xxi) residues 21-906 of SEQ ID NO:160, (xxii) residues 21-906 of SEQ ID NO:162, or (xxiii) residues 21-906 of SEQ ID NO:164.

Metabolic diseases that may be treated in accordance with the present invention include, for example, type 2 diabetes and obesity. In particular variations of a method for treating a metabolic disease, a fusion molecule for the metabolic disease treatment is a polypeptide having the structure DNase1-L1-Fc-L2-PON1, DNase1L3-L1-Fc-L2-PON1, RNase1-L1-Fc-L2-PON1, RNase1-Fc-L2-PON1, Fc-L2-PON1, or PON1-L1-Fc, or a dimeric protein formed by dimerization of any of the foregoing fusion polypeptides; in some such embodiments, the fusion polypeptide comprises or consists of an amino acid sequence having at least 90%, at least 95%, or 100% identity with the amino acid sequence shown in (i) residues 21-896 of SEQ ID NO:2, (ii) residues 21-764 of SEQ ID NO:40, (iii) residues 21-740 of SEQ ID NO:48, (iv) residues 21-613 of SEQ ID NO:122, (v) residues 21-610 of SEQ ID NO:142, (vi) residues 21-906 of SEQ ID NO:148, (vii) residues 21-896 of SEQ ID NO:158, (viii) residues 21-906 of SEQ ID NO:160, (ix) residues 21-906 of SEQ ID NO:162, or (x) residues 21-906 of SEQ ID NO:164.

Cardiovascular diseases that may be treated in accordance with the present invention include, for example, cardiovascular diseases characterized by atherosclerosis. In some embodiments, the cardiovascular disease characterized by atherosclerosis is coronary heart disease or ischemic stroke. In more particular variations comprising treatment of coronary heart disease, the coronary heart disease is characterized by acute coronary syndrome. In certain variations of a method for treating a cardiovascular disease, a fusion molecule for the cardiovascular disease treatment is a polypeptide having the structure DNase1-L1-Fc-L2-PON1, DNase1L3-L1-Fc-L2-PON1, RNase1-L1-Fc-L2-PON1, RNase1-Fc-L2-PON1, Fc-L2-PON1, or PON1-L1-Fc, or a dimeric protein formed by dimerization of any of the foregoing fusion polypeptides; in some such embodiments, the fusion polypeptide comprises or consists of an amino acid sequence having at least 90%, at least 95%, or 100% identity with the amino acid sequence shown in (i) residues 21-896 of SEQ ID NO:2, (ii) residues 21-764 of SEQ ID NO:40, (iii) residues 21-740 of SEQ ID NO:48, (iv) residues 21-613 of SEQ ID NO:122, (v) residues 21-610 of SEQ ID NO:142, (vi) residues 21-906 of SEQ ID NO:148, (vii) residues 21-896 of SEQ ID NO:158, (viii) residues 21-906 of SEQ ID NO:160, (ix) residues 21-906 of SEQ ID NO:162, or (x) residues 21-906 of SEQ ID NO:164.

In some embodiments of a method for treating thrombosis, sepsis, or ischemia reperfusion, a fusion molecule for such treatment is a polypeptide having the structure DNase1-L1-Fc-L2-PON1, DNase1L3-L1-Fc-L2-PON1, RNase1-L1-Fc-L2-PON1, or RNase1-Fc-L2-PON1, or a dimeric protein formed by dimerization of any of the foregoing fusion polypeptides; in some such embodiments, the fusion polypeptide comprises or consists of an amino acid sequence having at least 90%, at least 95%, or 100% identity with the amino acid sequence shown in (i) residues 21-896 of SEQ ID NO:2, (ii) residues 21-764 of SEQ ID NO:40, (iii) residues 21-740 of SEQ ID NO:48, (iv) residues 21-906 of SEQ ID NO:148, (v) residues 21-896 of SEQ ID NO:158, (vi) residues 21-906 of SEQ ID NO:160, (vii) residues 21-906 of SEQ ID NO:162, or (viii) residues 21-906 of SEQ ID NO:164.

In some embodiments of a method for treating antiphospholipid syndrome, thrombosis, or ischemic stroke, a DNase-containing fusion molecule as described herein is administered to a patient as one of the distinct therapies of a combination therapy comprising a thrombolytic agent. In some such embodiments, the thrombolytic agent is selected from anistreplase, reteplase, streptokinase, t-PA, tenecteplase, and rokinase.

In some embodiments of a method for treating exposure to sulfur mustard gas (SM) or exposure to an organophosphate (e.g., tabun, sarin, soman, or cyclosarin), a fusion molecule for such exposure treatment is a polypeptide having the structure DNase1-L1-Fc-L2-PON1, DNase1L3-L1-Fc-L2-PON1, RNase1-L1-Fc-L2-PON1, RNase1-Fc-L2-PON1, SOD1-L1-Fc-L2-PON1. SOD1-Fc-L2-PON1, CTLA4-L1-Fc-L2-PON1, CTLA4-Fc-L2-PON1, CD40-L1-Fc-L2-PON1, CD40-Fc-L2-PON1, antiTNFα-L1-Fc-L2-PON1, antiTNFα-Fc-L2-PON1, antiTGFβ-L1-Fc-L2-PON1, antiTGFβ-Fc-L2-PON1, Fc-L2-PON1, or PON1-L1-Fc, or a dimeric protein formed by dimerization of any of the foregoing fusion polypeptides; in some such embodiments, the fusion polypeptide comprises or consists of an amino acid sequence having at least 90%, at least 95%, or 100% identity with the amino acid sequence shown in (i) residues 21-896 of SEQ ID NO:2, (ii) residues 21-896 of SEQ ID NO:128, (iii) residues 21-896 of SEQ ID NO:130, (iv) residues 21-764 of SEQ ID NO:40, (v) residues 21-740 of SEQ ID NO:48, (vi) residues 23-781 of SEQ ID NO:50, (vii) residues 23-781 of SEQ ID NO:52, (viii) residues 23-791 of SEQ ID NO:54, (ix) residues 21-736 of SEQ ID NO:66, (x) residues 21-804 of SEQ ID NO:74, (xi) residues 21-804 of SEQ ID NO:78, (xii) residues 21-804 of SEQ ID NO:82, (xiii) residues 21-804 of SEQ ID NO:86, (xiv) residues 21-804 of SEQ ID NO:90, (xv) residues 21-860 of SEQ ID NO:94, (xvi) residues 21-860 of SEQ ID NO:98, (xvii) residues 21-861 of SEQ ID NO:102, (xviii) residues 21-861 of SEQ ID NO:106, (xix) residues 21-613 of SEQ ID NO:122, (xx) residues 21-613 of SEQ ID NO:132, (xxi) residues 21-613 of SEQ ID NO:134, (xxii) residues 21-610 of SEQ ID NO:142, (xxiii) residues 21-610 of SEQ ID NO:144, (xxiv) residues 21-610 of SEQ ID NO:146, (xxv) residues 21-906 of SEQ ID NO:148, (xxvi) residues 21-896 of SEQ ID NO:158, (xxvii) residues 21-906 of SEQ ID NO:160, (xxviii) residues 21-906 of SEQ ID NO:162, or (xxix) residues 21-906 of SEQ ID NO:164.

Liver diseases or disorders that may be treated in accordance with the present invention include chronic liver diseases such as, e.g., nonalcoholic fatty liver disease (NAFLD), alcohol-associated liver disease (ALD), portal hypertension, and complications following liver transplantation. In some variations comprising treatment of nonalcoholic fatty liver disease (NAFLD), the NAFLD is nonalcoholic steatohepatitis (NASH). In particular variations of a method for treating a liver disease, a fusion molecule for the liver disease treatment is a polypeptide having the structure DNase1-L1-Fc-L2-PON1, DNase1L3-L1-Fc-L2-PON1, CD40-L1-Fc-L2-PON1, CD40-Fc-L2-PON1, antiTGFβ-L1-Fc-L2-PON1, antiTGFβ-Fc-L2-PON1, Fc-L2-PON1, or PON1-L1-Fc, or a dimeric protein formed by dimerization of any of the foregoing fusion polypeptides; in some such embodiments, the fusion polypeptide comprises or consists of an amino acid sequence having at least 90%, at least 95%, or 100% identity with the amino acid sequence shown in (i) residues 21-896 of SEQ ID NO:2, (ii) residues 21-804 of SEQ ID NO:74, (iii) residues 21-804 of SEQ ID NO:78, (iv) residues 21-804 of SEQ ID NO:82, (v) residues 21-804 of SEQ ID NO:86, (vi) residues 21-804 of SEQ ID NO:90, (vii) residues 21-861 of SEQ ID NO:102, (viii) residues 21-861 of SEQ ID NO:106, (ix) residues 21-613 of SEQ ID NO: 122, (x) residues 21-610 of SEQ ID NO:142, (xi) residues 21-906 of SEQ ID NO:148, (xii) residues 21-896 of SEQ ID NO:158, (xiii) residues 21-906 of SEQ ID NO:160, (xiv) residues 21-906 of SEQ ID NO:162, or (xv) residues 21-906 of SEQ ID NO:164.

Fibrotic diseases or disorders amenable to treatment in accordance with the present invention include systemic sclerosis, systemic lupus erythematosus, inflammatory lung diseases, chronic liver diseases, and chronic kidney diseases. In some variations comprising treatment of an inflammatory lung disease, the fibrotic disease is cystic fibrosis, chronic obstructive pulmonary disease, interstitial lung disease (e.g., idiopathic pulmonary fibrosis or sarcoidosis), acute respiratory distress syndrome, or asthma. In some variations comprising treatment of a chronic liver disease, the fibrotic disease is nonalcoholic steatohepatitis, alcohol-associated liver disease, portal hypertension, or a complication following liver transplantation. In some variations comprising treatment of a chronic kidney disease, the fibrotic disease is lupus nephritis, IgA nephropathy, or membranous glomerulonephritis. In particular variations of a method for treating a liver disease, a fusion molecule for the liver disease treatment is a polypeptide having the structure DNase1-L1-Fc-L2-PON1, DNase1L3-L1-Fc-L2-PON1, CD40-L1-Fc-L2-PON1, CD40-Fc-L2-PON1, antiTGFβ-L1-Fc-L2-PON1, antiTGFβ-Fc-L2-PON1, Fc-L2-PON1, or PON1-L1-Fc, or a dimeric protein formed by dimerization of any of the foregoing fusion polypeptides; in some such embodiments, the fusion polypeptide comprises or consists of an amino acid sequence having at least 90%, at least 95%, or 100% identity with the amino acid sequence shown in (i) residues 21-896 of SEQ ID NO:2, (ii) residues 21-804 of SEQ ID NO:74, (iii) residues 21-804 of SEQ ID NO:78, (iv) residues 21-804 of SEQ ID NO:82, (v) residues 21-804 of SEQ ID NO:86, (vi) residues 21-804 of SEQ ID NO:90, (vii) residues 21-861 of SEQ ID NO:102, (viii) residues 21-861 of SEQ ID NO:106, (ix) residues 21-613 of SEQ ID NO:122, (x) residues 21-610 of SEQ ID NO:142, (xi) residues 21-906 of SEQ ID NO:148, (xii) residues 21-896 of SEQ ID NO:158, (xiii) residues 21-906 of SEQ ID NO:160, (xiv) residues 21-906 of SEQ ID NO:162. or (xv) residues 21-906 of SEQ ID NO:164.

Paraoxonase fusion molecules for treating cancer typically comprise a DNase, RNase, or anti-TGF-β neutralizing polypeptide as the first biologically active polypeptide. In more particular variations, a fusion molecule for cancer treatment is a polypeptide having the structure DNase1-L1-Fc-L2-PON1, DNase1L3-L1-Fc-L2-PON1, RNase1-L1-Fc-L2-PON1, RNase1-Fc-L2-PON1, antiTGFβ-L1-Fc-L2-PON1, or antiTGFβ-Fc-L2-PON1, or a dimeric protein formed by dimerization of any of the foregoing fusion polypeptides; in some such embodiments, the fusion polypeptide comprises or consists of an amino acid sequence having at least 90%, at least 95%, or 100% identity with the amino acid sequence shown in (i) residues 21-896 of SEQ ID NO:2, (ii) residues 21-764 of SEQ ID NO:40, (iii) residues 21-740 of SEQ ID NO:48, (ix) residues 21-861 of SEQ ID NO:102, (v) residues 21-861 of SEQ ID NO:106, (vi) residues 21-906 of SEQ ID NO:148, (vii) residues 21-896 of SEQ ID NO:158, (viii) residues 21-906 of SEQ ID NO:160, (ix) residues 21-906 of SEQ ID NO:162, or (x) residues 21-906 of SEQ ID NO:164.

Cancers that may be treated in accordance with the present invention include, for example, the following: a cancer of the head and neck (e.g., a cancer of the oral cavity, oropharynx, nasopharynx, hypopharynx, nasal cavity or paranasal sinuses, larynx, lip, or salivary gland); a lung cancer (e.g., non-small cell lung cancer, small cell carcinoma, or mesothelioma); a gastrointestinal tract cancer (e.g., colorectal cancer, gastric cancer, esophageal cancer, or anal cancer); gastrointestinal stromal tumor (GIST); pancreatic adenocarcinoma; pancreatic acinar cell carcinoma; a cancer of the small intestine; a cancer of the liver or biliary tree (e.g., liver cell adenoma, hepatocellular carcinoma, hemangiosarcoma, extrahepatic or intrahepatic cholangiosarcoma, cancer of the ampulla of vater, or gallbladder cancer); a breast cancer (e.g., metastatic breast cancer or inflammatory breast cancer); a gynecologic cancer (e.g., cervical cancer, ovarian cancer, fallopian tube cancer, peritoneal carcinoma, vaginal cancer, vulvar cancer, gestational trophoblastic neoplasia, or uterine cancer, including endometrial cancer or uterine sarcoma); a cancer of the urinary tract (e.g., prostate cancer; bladder cancer; penile cancer; urethral cancer, or kidney cancer such as, for example, renal cell carcinoma or transitional cell carcinoma, including renal pelvis and ureter); testicular cancer; a cancer of the central nervous system (CNS) such as an intracranial tumor (e.g., astrocytoma, anaplastic astrocytoma, glioblastoma, oligodendroglioma, anaplastic oligodendroglioma, ependymoma, primary CNS lymphoma, medulloblastoma, germ cell tumor, pineal gland neoplasm, meningioma, pituitary tumor, tumor of the nerve sheath (e.g., schwannoma), chordoma, craniopharyngioma, a chloroid plexus tumor (e.g., chloroid plexus carcinoma), or other intracranial tumor of neuronal or glial origin) or a tumor of the spinal cord (e.g., schwannoma, meningioma); an endocrine neoplasm (e.g., thyroid cancer such as, for example, thyroid carcinoma, medullary cancer, or thyroid lymphoma; a pancreatic endocrine tumor such as, for example, an insulinoma or glucagonoma; an adrenal carcinoma such as, for example, pheochromocytoma; a carcinoid tumor; or a parathyroid carcinoma); a skin cancer (e.g., squamous cell carcinoma; basal cell carcinoma; Kaposi's sarcoma; or a malignant melanoma such as, for example, an intraocular melanoma); a bone cancer (e.g., a bone sarcoma such as, for example, osteosarcoma, osteochondroma, or Ewing's sarcoma); multiple myeloma; a chloroma; a soft tissue sarcoma (e.g., a fibrous tumor or fibrohistiocytic tumor); a tumor of the smooth muscle or skeletal muscle; a blood or lymph vessel perivascular tumor (e.g., Kaposi's sarcoma); a synovial tumor; a mesothelial tumor; a neural tumor; a paraganglionic tumor; an extraskeletal cartilaginous or osseous tumor; and a pluripotential mesenchymal tumor. In some such embodiments, a paraoxonase fusion molecule as described herein is administered to a cancer patient as one of the distinct therapies of a combination therapy such as, for example, a combination therapy comprising an immunomodulatory therapy (e.g., a CAR T cell therapy (see, e.g., June et al., Science 359:1361-1365, 2018) or a therapy comprising an immune checkpoint inhibitor), a radiation therapy, or a chemotherapy.

In certain embodiments, a combination cancer therapy comprises a DNase-PON, RNase-PON, or antiTGFβ-PON fusion molecule as described herein and a targeted therapy such as, e.g., a therapeutic monoclonal antibody targeting a specific cell-surface or extracellular antigen, or a small molecule targeting an intracellular protein (e.g., an intracellular enzyme). Exemplary antibody targeted therapies include anti-VEGF (e.g., bevacizumab), anti-EGFR (e.g., cetuximab), anti-CTLA-4 (e.g., ipilimumab), anti-PD-1 (e.g., nivolumab), and anti-PD-L1 (e.g., pembrolizumab). Exemplary small molecule targeted therapies include proteasome inhibitors (e.g., bortezomib), tyrosine kinase inhibitors (e.g., imatinib), cyclin-dependent kinase inhibitors (e.g., seliciclib); BRAF inhibitors (e.g., vemurafenib or dabrafenib); and MEK kinase inhibitors (e.g., trametnib).

In some cancer combination therapy variations comprising an immune checkpoint inhibitor, the combination therapy includes an anti-PD-1/PD-L1 therapy, an anti-CTLA-4 therapy, or both. In certain aspects, a DNase-PON, RNase-PON, or antiTGFβ-PON fusion molecule as described herein can increase the response rate to either anti-CTLA-4 or anti-PD-1/PD-L1 therapy, as well as the response rate to the combination of anti-CTLA-4 plus anti-PD-1/PD-L1 therapy. Fusion molecules of the invention may also be useful for reducing the toxicity associated with anti-CTLA-4, anti-PD-1/PD-L1, or the combination thereof.

In certain variations, a cancer treated in accordance with the present invention is selected from malignant melanoma, renal cell carcinoma, non-small cell lung cancer, bladder cancer, and head and neck cancer. These cancers have shown responses to immune checkpoint inhibitors anti-PD-1/PD-L1 and anti-CTLA-4. See Grimaldi et al., Expert Opin. Biol. Ther. 16:433-41, 2016; Gunturi et al., Curr. Treat. Options Oncol. 15:137-46, 2014; Topalian et al., Nat. Rev. Cancer 16:275-87, 2016. Thus, in some more specific variations, any of these cancers is treated with a DNase-PON, RNase-PON, or antiTGFβ-PON fusion molecule as described herein in combination with an anti-PD-1/PD-L1 therapy, an anti-CTLA-4 therapy, or both.

In other aspects, the present invention provides methods for reducing lipid oxidation in a subject. The method generally includes administering to the subject an effective amount of a fusion polypeptide or dimeric protein as described herein, wherein one or more oxidized lipids in the subject are reduced. In some embodiments, the method reduces one or more oxidized lipids associated with the presence of a disease or disorder in the subject (e.g., a disease or disorder discussed above). In other embodiments, the one or more oxidized lipids are associated with a risk of developing such a disease or disorder; in particular variations, treatment with the fusion molecule reduces the risk of developing the disease or disorder in the subject.

In another aspect, the present invention provides a method for protecting a subject from aging. The method generally includes administering to the subject an effective amount of a fusion polypeptide of a fusion polypeptide or dimeric protein as described herein. In some embodiments, the subject has an age-related disease or disorder (e.g., an inflammatory disease, an autoimmune disease, a neurodegenerative disease, a cardiovascular disease, or a fibrotic disease). In other embodiments, the subject is at risk of developing such an age-related disease or disorder, and treatment with the fusion molecule reduces the risk of the disease or disorder in the subject.

In some embodiments of a method for treating a disease or disorder, reducing lipid oxidation, or protecting from aging as above, the method is a combination therapy comprising administering to the patient (a) an effective amount of a paraoxonase fusion polypeptide having the formula X-L2-P or P-L1-X as described above, or a dimeric protein formed by dimerization of the fusion polypeptide, and (b) an effective amount of a biologically active DNase. Such embodiments are particularly useful, e.g., for treatment of diseases and disorder characterized by NETosis. In some variations, the method comprises administration of a paraoxonase fusion polypeptide having the structure Fc-L2-PON1 or PON1-L1-Fc, or a dimeric protein formed by dimerization thereof; in some such embodiments, the paraoxonase fusion polypeptide has the structure Fc-L2-PON1 and comprises or consists of an amino acid sequence having at least 90%, at least 95%, or 100% identity with the amino acid sequence shown in (i) residues 21-613 of SEQ ID NO:122, (ii) residues 21-613 of SEQ ID NO:132, (iii) residues 21-613 of SEQ ID NO:134, (iv) residues 21-610 of SEQ ID NO:142, (v) residues 21-610 of SEQ ID NO:144, or (vi) residues 21-610 of SEQ ID NO:146. The biologically active DNase may be, for example, a DNase1 or DNase1 L3 polypeptide as described herein (e.g., a polypeptide corresponding to the DNase component of a DNase-containing paraoxonase fusion as described herein such as, for example, an enhanced DNase1 polypeptide comprising or consisting of an amino acid sequence having at least 90%, at least 95%, or 100% identity with the amino acid sequence shown in residues 21-280 of SEQ ID NO:18, residues 21-280 of SEQ ID NO:20, or residues 21-280 of SEQ ID NO:152. In some embodiments, the DNase is contained within a fusion polypeptide comprising, from an amino terminal position to a carboxyl terminal position, D-L1-X_{d}, wherein D is the DNase, L1 is a polypeptide linker (e.g., an L1 linker as described herein for a DNase-containing paraoxonase fusion molecule), and Xa is an immunoglobulin Fc region as described herein (or contained with a dimeric protein formed by dimerization of the fusion polypeptide); in some such embodiments, the DNase fusion polypeptide comprises or consists of an amino acid sequence having at least 90%, at least 95%, or 100% identity with the amino acid sequence shown in (i) residues 21-538 or 21-537 of SEQ ID NO:60, (ii) residues 21-548 or 21-547 of SEQ ID NO:62, (iii) residues 21-538 or 21-537 of SEQ ID NO:155, (iv) residues 21-548 or 21-547 of SEQ ID NO:156, (v) residues 21-548 or 21-547 of SEQ ID NO:138, or (vi) residues 21-548 or 21-547 of SEQ ID NO:140.

In some embodiments of a combination therapy comprising administration of a paraoxonase fusion molecule and a DNase as described above, the DNase is contained within a DNase1-L1-Fc fusion polypeptide comprising the amino acid sequence shown in SEQ ID NO:149, SEQ ID NO:150, SEQ ID NO:153, or SEQ ID NO:154. In certain variations of the combination therapy method wherein DNase fusion polypeptide comprises the amino acid sequence shown in SEQ ID NO:149, SEQ ID NO:150, SEQ ID NO:153, or SEQ ID NO:154, (i) the amino acid at each of positions 315 and 316 is alanine; (ii) the amino acid at position 319 is serine; (iii) the amino acid at position 378 is alanine, glutamine, or glycine, and the amino acid at position 346 is optionally alanine; (iv) the amino acid at position 410 is alanine, glycine, or serine; (v) the amino acid at position 412 is serine or alanine; (vi) the amino acid at position 333 is tyrosine, the amino acid at position 335 is threonine, and the amino acid at position 337 is glutamate; and/or (vii) the amino acid at position 509 is leucine, and the amino acid at position 515 is serine. In other, non-mutually exclusive variations of the combination therapy method wherein the DNase fusion polypeptide comprises the amino acid sequence shown in SEQ ID NO:149, SEQ ID NO:150, SEQ ID NO:153, or SEQ ID NO154, the amino acid sequence corresponding to positions 261-296 includes a Gly-Ser tandem repeat sequence having a formula selected from the group consisting of (i) [Gly-Gly-Gly-Gly-Ser]ₙ, wherein n is an integer from 4 to 7, (ii) [Gly-Gly-Gly-Ser]ₙ, wherein n is an integer from 5 to 9, and (iii) [Gly-Gly-Ser]ₙ, wherein n is an integer from 6 to 12; in some such embodiments, the amino acid sequence corresponding to positions 261-296 includes a Gly-Ser tandem repeat sequence having a formula selected from the group consisting of (i) [Gly-Gly-Gly-Gly-Ser]ₙ, wherein n is an integer from 4 to 6, (ii) [Gly-Gly-Gly-Ser]ₙ, wherein n is an integer from 5 to 7, and (iii) [Gly-Gly-Ser]ₙ, wherein n is an integer from 6 to 10. In yet other, non-mutually exclusive variations of the combination therapy method wherein the DNase fusion polypeptide comprises the amino acid sequence shown in SEQ ID NO:149, SEQ ID NO:150, SEQ ID NO:153, or SEQ ID NO:154, the amino acid at position 261 is aspartate, the amino acid at position 262 is leucine, the amino acid at position 263 is serine, the amino acid at position 294 is threonine, the amino acid at position is 295 is glycine, and/or the amino acid at position 296 is leucine. In other, non-mutually exclusive variations of the combination therapy method where the DNase fusion polypeptide comprises the amino acid sequence shown in SEQ ID NO:149 or SEQ ID NO:153, (i) the amino acid at each of positions 74 and 105 is independently lysine or arginine, and/or (ii) the amino acid at position 114 is phenylalanine.

In some embodiments of a method for treating a disease or disorder, reducing lipid oxidation, or protecting from aging as above, the method is a combination therapy comprising administering to the patient (a) an effective amount of a paraoxonase fusion polypeptide having the formula X-L2-P or P-L1-X as described above, or a dimeric protein formed by dimerization of the fusion polypeptide, and (b) an effective amount of a biologically active apolipoprotein A-1 (ApoA1) (e.g., an ApoA1-Fc fusion polypeptide as described in International PCT Publication No. WO 2017/044424, or a dimeric protein formed by dimerization of the fusion polypeptide).

For therapeutic use, a fusion polypeptide or dimeric protein as described herein is delivered in a manner consistent with conventional methodologies associated with management of the disease or disorder for which treatment is sought. In accordance with the disclosure herein, an effective amount of the fusion polypeptide or dimeric protein is administered to a subject in need of such treatment for a time and under conditions sufficient to prevent or treat the disease or disorder.

Subjects for administration of fusion polypeptides or dimeric proteins as described herein include patients at high risk for developing a particular disease or disorder as well as patients presenting with an existing disease or disorder. In certain embodiments, the subject has been diagnosed as having the disease or disorder for which treatment is sought. Further, subjects can be monitored during the course of treatment for any change in the disease or disorder (e.g., for an increase or decrease in clinical symptoms of the disease or disorder). Also, in some variations, the subject does not suffer from another disease or disorder requiring treatment that involves administration of a protein selected from a paraoxonase, a DNase, an RNase, a SOD1, a CTLA-4 extracellular domain, a CD40 extracellular domain, or a polypeptide (e.g, antibody) that specifically binds and neutralizes TNFα or TGFβ.

In prophylactic applications, pharmaceutical compositions or medicants are administered to a patient susceptible to, or otherwise at risk of, a particular disease in an amount sufficient to eliminate or reduce the risk or delay the onset of the disease. In therapeutic applications, compositions or medicants are administered to a patient suspected of, or already suffering from such a disease in an amount sufficient to cure, or at least partially arrest, the symptoms of the disease and its complications. An amount adequate to accomplish this is referred to as a therapeutically or pharmaceutically effective dose or amount. In both prophylactic and therapeutic regimes, agents are usually administered in several dosages until a sufficient response (e.g., enhanced alveolar fluid clearance in inflammatory lung disease) has been achieved. Typically, the response is monitored and repeated dosages are given if the desired response starts to fade.

To identify subject patients for treatment according to the methods of the invention, accepted screening methods may be employed to determine risk factors associated with a specific disease or to determine the status of an existing disease identified in a subject. Such methods can include, for example, determining whether an individual has relatives who have been diagnosed with a particular disease. Screening methods can also include, for example, conventional work-ups to determine familial status for a particular disease known to have a heritable component. Toward this end, nucleotide probes can be routinely employed to identify individuals carrying genetic markers associated with a particular disease of interest. In addition, a wide variety of immunological methods are known in the art that are useful to identify markers for specific diseases. Screening may be implemented as indicated by known patient symptomology, age factors, related risk factors, etc. These methods allow the clinician to routinely select patients in need of the methods described herein for treatment. In accordance with these methods, treatment using a fusion polypeptide or dimeric protein of the present invention may be implemented as an independent treatment program or as a follow-up, adjunct, or coordinate treatment regimen to other treatments.

For treatment of a disease or disorder characterized by NETosis, subject patients likely to benefit from NET-targeted therapy may be identified, for example, by measuring NETs in candidate patients. There are multiple ways to measure NETs in vivo (for a review, see, e.g., Masuda et al., Clin. Chim. Acta 459:89-93, 2016). One method uses flow cytometry to measure cell-associated DNA by staining with a non-cell permeable DNA dye (Zharkova et al., Cytometry A 95:268-278, 2019). Other methods include measuring myeloperoxidase activity associated with DNA or measuring citrullinated histone H3 (see, e.g., Matsuda et al., supra). Yet another method measures NETosis by incubating blood neutrophils with autologous plasma (Abrams et al., Am. J. Resp. Crit. Care Med. 200:869, 2019). This method was able to predict disseminated intravascular coagulation in critically ill patients (id.).

For administration, a fusion polypeptide or dimeric protein in accordance with the present invention is formulated as a pharmaceutical composition. A pharmaceutical composition comprising a fusion polypeptide or dimeric protein as described herein can be formulated according to known methods to prepare pharmaceutically useful compositions, whereby the therapeutic molecule is combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. See, e.g., Gennaro (ed.), Remington's Pharmaceutical Sciences (Mack Publishing Company, 19th ed. 1995). Formulations may further include one or more excipients, preservatives, solubilizers, buffering agents, albumin to prevent protein loss on vial surfaces, etc.

A pharmaceutical composition comprising a fusion polypeptide or dimeric protein of the present invention is administered to a subject in an effective amount. The fusion polypeptide or dimeric protein may be administered to subjects by a variety of administration modes, including, for example, by intramuscular, subcutaneous, intravenous, intra-atrial, intra-articular, parenteral, intranasal, intrapulmonary, transdermal, intrapleural, intrathecal, and oral routes of administration. For prevention and treatment purposes, the fusion polypeptide or dimeric protein may be administered to a subject in a single bolus delivery, via continuous delivery (e.g., continuous transdermal delivery) over an extended time period, or in a repeated administration protocol (e.g., on an hourly, daily, or weekly basis).

In some embodiments, a pharmaceutical composition comprising a fusion molecule or dimeric protein of the present invention is formulated for delivery to the lung by nebulization. Previous studies of pulmonary delivery of Fc fusion proteins, including erythropoietin-Fc, interferon Beta1a-Fc, and FSH-Fc, have shown that the immunoglobulin transport FcRn pathway is active in the lungs and provides 20% to 50% bioavailability (see Bitonti et al., Proc. Natl. Acad. Sci. USA 101:9763-9768, 2004: Bitonti and Dumont, Adv. Drug Deliv. Rev. 58:1106, 2006; Valle et al., J. Interferon Cytokine Res. 32:178-184, 2012). Pulmonary delivery of FcRn-binding paraoxonase fusion molecules as described herein (e.g., paraoxonase fusion molecules containing an Fc region) could act both locally and in circulation and peripheral organs. In certain embodiments wherein the treatment is a combination therapy with a paraoxonase fusion molecule and a DNase as described herein, both the paraoxonase fusion molecule (e.g., an Fc-L2-PON1 fusion as described herein) and the DNase (e.g., a DNase-Fc fusion as described herein) are delivered by a nebulizer. In some variations comprising delivery to the lung by a nebulizer, the disease or disorder to the treated is selected from an inflammatory lung disease (e.g., cystic fibrosis, interstitial lung disease (e.g., idiopathic pulmonary fibrosis or sarcoidosis), acute respiratory distress syndrome (ARDS), chronic obstructive pulmonary disease (COPD), asthma, exposure to sulfur mustard gas, or exposure to an organophosphate), biofilm formation by a gram-negative bacteria (e.g., Pseudomonas aeruginosa), and sepsis. In other, non-mutually exclusive variations, an FcRn-binding fusion molecule for delivery to the lung by nebulization (e.g., an Fc-L2-PON1 fusion as described herein) comprises an Fc variant with increased FcRn-binding affinity relative to the corresponding wild-type Fc, thereby increasing fusion molecule half-life and increasing concentration in the blood after inhalation with a nebulizer.

Multiple biologics have been successfully formulated to retain biologic activity after nebulization (see, e.g., Hertel et al., Adv. Drug Deliv. Rev. 93:79-94, 2015). Formulations for delivery of a paraoxonase fusion molecule of the present invention may include an excipient suitable for pulmonary delivery such as, e.g., Polysorbate 80 (PS80) or Polysorbate 20 (PS20), surfactants that are included in many biopharmaceutical formulations. Such stabilizing excipients protect proteins from degradation at the air-liquid interface when applied above their critical micelle concentration (for example, PS80 above 0.01% was effective in stabilizing G-CSF, LDH, rhConIFN, and t-Pa; and PS20 applied at 0.04% was effective for protection of Fc-gamma RIIb). Another suitable stabilizing excipient is HP-beta-cyclodextrin (HP-beta-CD) applied at, e.g., 0.35% or above (see Hertel et al., supra). In some variations, a stabilizing excipient is not required for nebulized delivery of a paraoxonase fusion molecule (e.g., DNase1-Fc-PON1) as described herein (for example, wild-type human DNase1 (Pulmozyme@) has been shown to not aggregate and remain stable after nebulization with either jet or vibrating mesh (VM) nebulizers without requiring excipients, see Cipolla et al., Pharm. Res. 11:491-498, 1994; Scherer et al., J. Pharm. Sci. 100:98-109, 2011).

Determination of effective dosages is typically based on animal model studies followed up by human clinical trials and is guided by determining effective dosages and administration protocols that significantly reduce the occurrence or severity of the subject disease or disorder in model subjects. Effective doses of the compositions of the present invention vary depending upon many different factors, including means of administration, target site, physiological state of the patient, whether the patient is human or an animal, other medications administered, whether treatment is prophylactic or therapeutic, as well as the specific activity of the composition itself and its ability to elicit the desired response in the individual. Usually, the patient is a human, but in some diseases, the patient can be a nonhuman mammal. Typically, dosage regimens are adjusted to provide an optimum therapeutic response, i.e., to optimize safety and efficacy. Accordingly, a therapeutically or prophylactically effective amount is also one in which any undesired collateral effects are outweighed by beneficial effects (e.g., in the case of treatment of inflammatory lung disease, where any undesired collateral effects are outweighed by any beneficial effects such as, for example, improved alveolar fluid clearance, improved lung physiology and function, etc.). For administration of a fusion polypeptide or dimeric protein of the present invention, a dosage typically ranges from about 0.1 μg to 100 mg/kg or 1 μg/kg to about 50 mg/kg, and more usually 10 μg to 5 mg/kg of the subject's body weight. In more specific embodiments, an effective amount of the agent is between about 1 μg/kg and about 20 mg/kg, between about 10 μg/kg and about 10 mg/kg, or between about 0.1 mg/kg and about 5 mg/kg. Dosages within this range can be achieved by single or multiple administrations, including, e.g., multiple administrations per day or daily, weekly, bi-weekly, or monthly administrations. For example, in certain variations, a regimen consists of an initial administration followed by multiple, subsequent administrations at weekly or bi-weekly intervals. Another regimen consists of an initial administration followed by multiple, subsequent administrations at monthly or bi-monthly intervals. Alternatively, administrations can be on an irregular basis as indicated by monitoring of clinical symptoms of the disease or disorder and/or monitoring of disease biomarkers or other disease correlates.

In some specific embodiments comprising delivery by inhalation with a nebulizer (for example, for treatment of an inflammatory lung disease such as, e.g., cystic fibrosis or interstitial lung disease), a fusion polypeptide or dimeric protein of the present invention is administered to the lung by a nebulizer at a dose of from about 1 mg to about 5 mg or from about 2 mg to about 4 mg per day. In other specific embodiments comprising systemic administration (for example, for treatment of systemic lupus erythematosus, lupus nephritis, rheumatoid arthritis, or inflammatory bowel disease), a fusion polypeptide or dimeric protein of the present invention is administered (e.g., by intravenous or subcutaneous injection) at a dose of from about 50 mg to about 1,000 mg, from about 50 mg to about 800 mg, from about 50 mg to about 500 mg, from about 100 mg to about 500 mg, from about 100 mg to about 400 mg, from about 100 mg to about 300 mg, from about 150 mg to about 250 mg, or about 200 mg every month to six weeks. For combination therapy with a paraoxonase fusion molecule and a DNase as described herein, each of the paraoxonase fusion molecule (e.g., an Fc-L2-PON1 fusion as described herein) and the DNase (e.g., a DNase-Fc fusion as described herein) may be administered at these doses.

Particularly suitable animal models for evaluating efficacy of a paraoxonase fusion composition of the present invention for treatment of inflammatory lung disease include, for example, a murine ovalbumin-induced acute asthma model as described by da Cunha et al. (Exp. Lung Res. 42:66, 2016) (showing significantly reduced airway resistance with wild-type (wt) DNase1 treatment), a murine silica-induced lung inflammation model as described by Benmerzoug et al. (Nat. Comm. 9:5226, 2018) (showing prevention of DNA-mediated STING activation and blockade of the downstream type I IFN response with wt DNase1 treatment), and a murine model of transfusion-related acute lung injury as described by Caudrillier et al. (J. Clin. Invest. 122:2661, 2012) (showing protection from lung edema and lung vascular permeability as well as reduced NET formation and platelet sequestration in the lung with wt DNase1 treatment).

Suitable animal models for evaluating efficacy of a paraoxonase fusion composition as described herein for treatment of exposure to exposure to sulfur mustard gas or an organophosphate include, for example, a guinea pig model as describe by Valiyaveettil et al. (Biochem. Pharmocol. 81:800-809, 2011; Toxicol. Letters 202:203-208, 2011) (showing protection from sarin and soman inhalation toxicity with recombinant human PON1 injection) and mouse models as described by Bajaj et al. (Appl. Biochem. Biotechnol. 180:165-176, 2016) and Stevens et al. (Proc. Natl. Acad. Sci. USA 105:12780-12784, 2008) (showing protection from organophosphate poisoning using a recombinant Q192K variant of PON1).

Particularly suitable animal models for evaluating efficacy of a paraoxonase composition as described herein for treatment of an inflammatory bowel disease include a TNBS-induced colitis model and a chronic colitis model with CD4⁺CD45RB^{high} cell transfer in mice. See, e.g., Yamashita et al., J. Immunol. 191:949-960, 2013 (showing efficacy of PON1 therapy in the TNBS-induced colitis model and a PON1 variant (G3C9) in the chronic colitis model). Another suitable model is a dextran sulfate sodium (DSS)-induced colitis model in mice. See, e.g., Babicova et al., Folia Biolica (Praha) 64:10, 2018 (showing reduction in TNFα and myeloperoxidase in the colon with wt DNase1 treatment of DSS-induced colitis); Li et al., J. Crohns Colitis 2020, 14:240-253, 2020 (showing decreased cytokine production and attenuated accelerated thrombus formation and platelet activation with DNase1 treatment of DSS-induced colitis).

Also known is the collagen-induced arthritis (CIA) model for rheumatoid arthritis (RA) (see, e.g., Brand et al., Nat. Protoc. 2:1269-1275, 2007). CIA shares similar immunological and pathological features with RA, making it an ideal model for evaluating efficacy of DNase compositions. Another suitable model for RA is PG-PS (proteoglycan-polysaccharide)-induced arthritis in Lewis rats (see, e.g., Esser et al., Arthritis and Rheumatism 28:1402-1411, 1985; Brooks et al., Proc. Intl. Soc. Mag. Reason. Med. 11:1526, 2003).

Suitable animal models for multiple sclerosis (MS) include, for example, experimental allergic encephalomyelitis (EAE) models that rely on the induction of an autoimmune response in the CNS by immunization with a CNS antigen (also referred to as an “encephalitogen” in the context of EAE), which leads to inflammation, demyelination, and weakness (see, e.g., Constantinescu et al., British Journal of Pharmacology 164:1079-1106, 2011).

Other known animal models for evaluating efficacy of paraoxonase fusion molecules of the present invention include, e.g., a hind-limb ischemia reperfusion model as described by Albadawi et al. (J. Vasc. Surg. 64:484, 2016) (showing increased perfusion, decreased infiltrating inflammatory cells, and reduction of a local thrombosis marker with wt DNase1 therapy), a rat model of ischemia-reperfusion-induced acute kidney injury as described by Peer et al. (Am. J. Nephrol. 43:195, 2016) (showing renoprotective effects with wt DNase1 therapy), a cecal ligation and puncture sepsis model in mice as described by Mai et al. (Shock 44:166, 2015) (showing prevention of organ damage and protection from death with wt DNase1 therapy), and a model of inferior vena cava stenosis as described by Brill et al. (J. Thrombosis Haemostasis 10:136, 2012) (showing prevention of thrombosis with wt DNase1 therapy). Still other exemplary animal models include models of heart transplantation rejection, plaque formation, and myocardial infarction used to show efficacy of RNase therapy (see. e.g., Kleinert et al., J. Am. Heart Assn. doi:10.1021, 2016; Simsekyilmaz et al., Circulation 129:598-606, 2014; Steiger et al., JAMA 6:e004541, 2017) as well as an acute stroke model as described by Walberer et al. (Curr. Neovas. Res. 6:12-19, 2009) (showing reduced cerebral edema and infarction size with RNase1 therapy).

Fusion molecules of the present invention can also be evaluated for anti-tumor activity in animal tumor models. For example, efficacy of DNase-PON treatment in reducing tumor metastasis associated with NET formation can be evaluated in mouse models as described by, e.g., Cools-Lartigue et al. (J. Clin. Invest. 123:3446, 2013) (showing reduction in metastasis of injected tumor (lung carcinoma) cells with systemic administration of wt DNase1 in a model of severe postoperative infection) and Park et al. (Sci. Translational Med. 8:361ra138, 2016) (showing reduction in metastasis of breast cancer cells to the lung with systemic administration of wt DNase1-coated nanoparticles). Also known is a model utilizing nude mice subcutaneously grafted with the human colon cancer cell line SW480 as described, e.g., by Trejo-Becirril et al. (Integrative Cancer Therapies 15:NP35-NP43, 2016) (showing inhibition of tumor growth with a combination of DNase1 and proteases). Other suitable models include a human lung tumor xenograft model described by Rutkoski et al. (Translational Oncology 6:392-397, 2013) (showing inhibition of tumor growth in mice using PEG-RNase1) and a syngeneic mouse tumor model with Lewis lung carcinoma described by Mironova et al., Oncotarget 8:78796-78810, 2017 (showing anti-tumor activity with RNase therapy).

Another known animal tumor model is B16 melanoma, a poorly immunogenic tumor. Multiple models of tumor immunotherapy have been studied. See Ngiow et al., Adv. Immunol. 130:1-24, 2016. The B16 melanoma model has been studied extensively with checkpoint inhibitors anti-CTLA-4, anti-PD-1, and the combination thereof. Anti-CTLA-4 alone has a potent therapeutic effect in this model only when combined with GM-CSF transduced tumor vaccine, or combined with anti-PD-1. See Weber, Semin. Oncol. 37:430-439, 2010. Ai et al., Cancer Immunol. Immunother. 64:885-92, 2015; Haanen et al., Prog. Tumor Res. 42:55-66, 2015. Efficacy of a DNase-PON, RNase-PON, or antiTGFP-PON fusion molecule for treatment of malignant melanoma is shown, for example, by slowed tumor growth following administration to B16 melanoma mice that have formed palpable subcutaneous tumor nodules. Efficacy of a paraoxonase fusion molecule can be evaluated in B16 melanoma mice either alone or, alternatively, in combination with another anti-cancer therapy (e.g., anti-CTLA-4, with or without tumor vaccine or with or without anti-PD-1/PD-L1). For example, tumor rejection in B16 melanoma mice using a combination of a DNase-PON, RNase-PON, or antiTGFβ-PON fusion molecule as described herein and anti-CTLA-4, in the absence of tumor vaccine, demonstrates an enhanced response to anti-CTLA-4 using the paraoxonase fusion therapy. In exemplary studies to evaluate paraoxonase fusion molecules comprising human protein sequences, which are functionally active in mice but are expected to be immunogenic in these models (and thereby likely to result in formation of neutralizing antibodies after 7-10 days), mice may be administered a fusion molecule of the present invention for a short period (for example, one week, administered in, e.g., two doses of about 40 mg/kg three days apart), and tumor growth then monitored, typically for two to three weeks after injection with the fusion molecule.

Dosage of the pharmaceutical composition may be varied by the attending clinician to maintain a desired concentration at a target site. For example, if an intravenous mode of delivery is selected, local concentration of the agent in the bloodstream at the target tissue may be between about 1-50 nanomoles of the composition per liter, sometimes between about 1.0 nanomole per liter and 10, 15, or 25 nanomoles per liter depending on the subject's status and projected measured response. Higher or lower concentrations may be selected based on the mode of delivery, e.g., trans-epidermal delivery versus delivery to a mucosal surface. Dosage should also be adjusted based on the release rate of the administered formulation, e.g., nasal spray versus powder, sustained release oral or injected particles, transdermal formulations, etc. To achieve the same serum concentration level, for example, slow-release particles with a release rate of 5 nanomolar (under standard conditions) would be administered at about twice the dosage of particles with a release rate of nanomolar. Dosing may also vary, e.g., depending on the activity of the fusion molecule being administered. For example, in the context of a DNase1-PON fusion molecule in which the DNase1 is not actin-resistant (i.e., in which the DNase1 does not contain any amino acid substitution, relative to human DNase1, that decreases G-actin-induced inhibition of endonuclease activity of the DNase relative to human DNase1), inhibition by actin may be overcome by increasing the dose relative to a dose that is effective for a similar but actin-resistant DNase1-PON fusion molecule.

A pharmaceutical composition comprising a fusion polypeptide or dimeric protein as described herein can be furnished in liquid form, in an aerosol, or in solid form. Liquid forms, are illustrated by injectable solutions, aerosols, droplets, topological solutions and oral suspensions. Exemplary solid forms include capsules, tablets, and controlled-release forms. The latter form is illustrated by miniosmotic pumps and implants. See, e.g., Bremer et al., Pharm. Biotechnol. 10:239, 1997; Ranade, “Implants in Drug Delivery,” in Drug Delivery Systems 95-123 (Ranade and Hollinger, eds., CRC Press 1995); Bremer et al., “Protein Delivery with Infusion Pumps,” in Protein Delivery: Physical Systems 239-254 (Sanders and Hendren, eds., Plenum Press 1997); Yewey et al., “Delivery of Proteins from a Controlled Release Injectable Implant,” in Protein Delivery: Physical Systems 93-117 (Sanders and Hendren, eds., Plenum Press 1997). Other solid forms include creams, pastes, other topological applications, and the like.

Degradable polymer microspheres have been designed to maintain high systemic levels of therapeutic proteins. Microspheres are prepared from degradable polymers such as poly(lactide-co-glycolide) (PLG), polyanhydrides, poly (ortho esters), nonbiodegradable ethylvinyl acetate polymers, in which proteins are entrapped in the polymer. See, e.g., Gombotz and Pettit, Bioconjugate Chem. 6:332, 1995; Ranade, “Role of Polymers in Drug Delivery,” in Drug Delivery Systems 51-93 (Ranade and Hollinger, eds., CRC Press 1995); Roskos and Maskiewicz, “Degradable Controlled Release Systems Useful for Protein Delivery,” in Protein Delivery: Physical Systems 45-92 (Sanders and Hendren, eds., Plenum Press 1997); Bartus et al., Science 281:1161, 1998; Putney and Burke, Nature Biotechnology 16:153, 1998; Putney, Curr. Opin. Chem. Biol. 2:548, 1998. Polyethylene glycol (PEG)-coated nanospheres can also provide carriers for intravenous administration of therapeutic proteins. See, e.g., Gref et al., Pharm. Biotechnol. 10:167, 1997.

Other dosage forms can be devised by those skilled in the art, as shown by, e.g., Ansel and Popovich, Pharmaceutical Dosage Forms and Drug Delivery Systems (Lea & Febiger, 5th ed. 1990); Gennaro (ed.), Remington's Pharmaceutical Sciences (Mack Publishing Company, 19th ed. 1995), and Ranade and Hollinger, Drug Deliverv Systems (CRC Press 1996).

In some embodiments comprising systemic administration of a fusion polypeptide or dimeric protein as described herein, the fusion molecule is formulated in a buffered saline (for example, saline buffered with 2 mM carbonate, pH 7.5, plus 1 mM calcium chloride).

Pharmaceutical compositions as described herein may also be used in the context of combination therapy. For example, for a combination therapy comprising administration of a paraoxonase fusion molecule and a DNase as described herein, each of the paraoxonase fusion molecule and the DNase (e.g., a DNase-Fc fusion as described herein) may be formulated in a buffered saline (for example, saline buffered with 2 mM carbonate, pH 7.5, plus 1 mM calcium chloride), either separately or as a mixture.

Pharmaceutical compositions may be supplied as a kit comprising a container that comprises a fusion polypeptide or dimeric protein as described herein. A therapeutic molecule can be provided, for example, in the form of an injectable solution for single or multiple doses, or as a sterile powder that will be reconstituted before injection. Alternatively, such a kit can include a dry-powder disperser, liquid aerosol generator, or nebulizer for administration of a therapeutic protein. Such a kit may further comprise written information on indications and usage of the pharmaceutical composition.

The invention is further illustrated by the following non-limiting examples.

### Example 1: Construction and Expression of DNase1-Fc and DNase1-Fc-PON1 Fusion Proteins

Constructs were designed and synthesized and cassettes with the correct sequences were combined to generate synthetic fusion genes encoding unique DNase1 fusion molecules. Each construct included a variant of wild-type human DNase1 (nucleotide and encoded amino acid sequences as shown in SEQ ID NOs:15 and 16; see also GenBank accession number NM_005223). The DNase1 variant contained amino acid substitutions, relative to the mature wild-type human sequence (SEQ ID NO:120), at positions N74 (N74K), G105 (G105R), and A114 (A114F); the variant DNase1 nucleotide and encoded amino acid sequences corresponding to the mature protein are shown in residues 61-840 of SEQ ID NO:17 and residues 21-280 of SEQ ID NO:18, respectively). The signal peptide sequence utilized was the human VK3 leader peptide (nucleotide and amino acid sequences as shown in SEQ ID NO:57 and SEQ ID NO:58), an efficient heterologous leader peptide that drives high level expression and secretion of fusion proteins. The DNase1 cassette was fused to the N-terminus of a human IgG1 Fc variant (SSS hinge, P238S, P331S) using a peptide linker, either a (Gly₄Ser)₄ linker ((g4s)4; nucleotide and amino acid sequences as shown in SEQ ID NO: 11 and SEQ ID NO:12) or a (Gly₄Ser)₆ linker ((g4s)6; nucleotide and amino acid sequences as shown in SEQ ID NO:13 and SEQ ID NO:14). The Fc variant nucleotide and amino acid sequences are shown in SEQ ID NO:27 and SEQ ID NO:28. The completed DNase1-Fc constructs generated are as follows:
[hVK3LP]-[hDNase1 N74K-G105R-A114F]-(g4s)4-[SSShinge-P238S-P331S Fc] (also referred to hereinbelow as hDNase™-(g4s)4-Fc; nucleotide and amino acid sequences as shown in SEQ ID NO:59 and SEQ ID NO:60); and
[hVK3LP]-[hDNase1 N74K-Gl05R-A114F]-(g4s)6-[SSShinge-P238S-P331S Fc](also referred to hereinbelow as hDNase™-(g4s)6-Fc; nucleotide and amino acid sequences as shown in SEQ ID NO:61 and SEQ ID NO:62).

Bispecific constructs further comprising a functional PON1 enzyme were also generated by fusing the carboxyl end of hDNase™-(g4s)4-Fc or hDNase™-(gs)6-Fc to a peptide linker containing an N-linked glycosylation site (NGS; nucleotide and amino acid sequences as shown in SEQ ID NO:55 and SEQ ID NO:56) followed by a variant of human PON1 (PON1 Q192K; nucleotide and amino acid sequences as shown in SEQ ID NO:5 and SEQ ID NO:6) in which the first 15 amino acids of the uncleaved leader peptide were deleted. The completed DNase-Fc-PON1 constructs are as follows:
[Human VK3LP]-[hDNase1 N974K-G105R-A114F]-(g4s)4-[SSShinge-P238S-P331S Fc]-NGS-[PON1 Q192K], (nucleotide and amino acid sequences as shown in SEQ ID NO:1 and SEQ ID NO:2) (also referred to hereinbelow as hDNase™-(g4s)4-Fc-PON1-Q192K, hDNase™-(g4s)4-Fe-PON1-K, or DNase™-(g4s)4-Fc-P1-K); and
[Human VK3LP]-[hDNase1 N974K-G105R-A114F]-(g4s)6-[SSShinge-P238S-P331S Fc]-NGS-[PON1 Q192K], (nucleotide and amino acid sequences as shown in SEQ ID NO:147 and SEQ ID NO:148) (also referred to hereinbelow as DNase™-(g4s)6-Fc-P1-K).

### Transient Expression of DNase1-Fc and DNase1-Fc-PON1 Fusion Proteins

All fusion genes were assembled in a pUC based vector, then inserted into a multiple cloning site of the mammalian expression vector pDG, a pcDNA3 plasmid derivative containing a CMV promoter to drive expression of the fusion gene. Plasmid DNA was prepared using QIAGEN (Germantown, MD) mini or maxiprep plasmid DNA kits. Purified plasmid DNA was transfected into HEK293 cells plated at approximately 50-75% confluence, using Polyfect (QIAGEN, Germantown, MD) transfection reagent according to the manufacturer's instructions. Culture media was changed to DMEM Fluorobrite™ (Life Technologies, Carlsbad, CA) serum free media on the day after transfections, and transfected cells incubated for an additional 48 hours prior to harvest of culture supernatants.

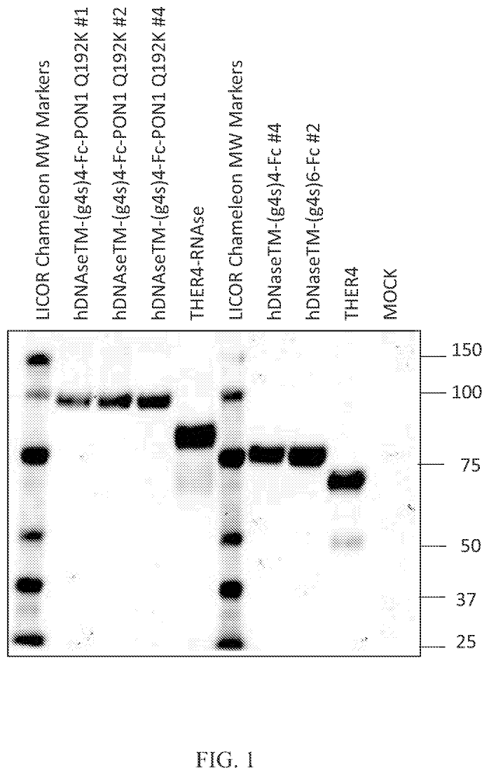

FIG. 1 shows the results of Western blot analysis of fusion protein expression from a representative set of HEK293 transient transfections. Culture supernatants were loaded directly for gel electrophoresis as follows: supernatants were collected, % sample volume 4X reducing LDS sample buffer added (Life Technologies, Carlsbad, CA), samples heated for 10 minutes at 72° C., the samples centrifuged for 1 minute at 3000 rpm in a microfuge, and 30 μl sample in loading buffer (one half) from each sample was loaded onto 4-12% Bis-Tris NuPAGE gels (Life Technologies, Carlsbad, CA). Protein molecular weight markers were included on each gel (Chameleon® DUO molecular weight markers; L1-COR Biosciences, Lincoln, NE). SDS-PAGE gels were run in NuPAGE MOPS running buffer (Life Technologies, Carlsbad, CA) under reducing conditions at 185 volts for approximately 1-1.5 hours. Gels were blotted to nitrocellulose membranes using the XCell blot module (Life Technologies, Carlsbad, CA) and NUPAGE-MOPS (Life Technologies, Carlsbad, CA) transfer buffer containing 10% methanol. Blots were blocked in L1-COR Odyssey Intercept™ blocking buffer, followed by incubation with diluted goat anti-human IgG (Fc specific) secondary antibodies (Jackson Immunoresearch, West Grove, PA). Secondary antibody was conjugated with Alexafluor 790 near IR dye (L1-COR Odyssey detection) and diluted in blocking buffer 1:25,000 at 4° C. and rocked overnight. Blots were washed four times with Tris buffered saline (TBS) containing 0.1% Tween 20. Blots were then rinsed in TBS buffer and scanned with a LICOR Odyssey™ scanner. Transfection samples were loaded as follows: Lane 1—LICOR Chameleon™ molecular weight markers; Lane 2, Lane 3, Lane 4—transfection supernatants from three different individual clones (#1, #2, #4) of hDNase™-(g4s)4-Fc-PON1-Q192K; Lane 5—THER4-RNase (control; apoA1-(g4s)4-SSShinge-P238S-P331S-Fc-NGS-RNase1; see US Patent Application Publication No. 2018/0201664); Lane 6—LICOR Chameleon MW Markers; Lane 7—hDNase™-(g4s)4-Fc #4; Lane 8—hDNase™-(g4s)6-Fc #2; Lane 9—THER4 (control; apoA1-(g4s)4-SSShinge-P238S-P331S-Fc; see International PCT Publication No. WO 2017/044424); Lane 10—mock transfection. The approximate molecular weight (kDa) of each marker band is indicated on the right side of the Western blot.

### Stable Expression of DNase1-Fc and DNase1-Fc-PON1 Fusion Proteins

Stable production of the DNase1 fusion proteins was achieved by electroporation of a selectable, amplifiable plasmid, pDG, containing the DNase1 fusion molecule cDNAs under the control of the CMV promoter, into Chinese Hamster Ovary (CHO) DG44 cells. The pDG vector is a modified version of pcDNA3 encoding the DHFR selectable marker with an attenuated promoter to increase selection pressure for the plasmid. Plasmid DNA (200 μg) was prepared using QIAGEN HISPEED maxiprep kits. Purified plasmid was linearized at a unique Asci site (New England Biolabs, Ipswich, MA Catalog #R0558), purified by phenol extraction (Sigma-Aldrich, St Louis, MO), ethanol precipitated, washed, and resuspended in tissue culture media, Excell 302 (Catalog #14324, SAFC/Sigma Aldrich, St Louis, MO). Salmon sperm DNA (Sigma-Aldrich, St. Louis, MO) was added as carrier DNA just prior to phenol extraction and ethanol precipitation. Plasmid and carrier DNA were coprecipitated, and the 400 μg was used to transfect 2×10⁷ CHO DG44 cells by electroporation.

For electroporation, cells were grown to logarithmic phase in Excell 302 media (Catalog #13424C, SAFC Biosciences/Sigma-Aldrich, St. Louis, MO) containing glutamine (4 mM), pyruvate, recombinant insulin (1 μg/ml), penicillin-streptomycin, and 2xDMEM nonessential amino acids (all from Life Technologies, Grand Island, NY), hereinafter referred to as “Excell 302 complete” media. Media for untransfected cells and cells to be transfected also contained HT (diluted from a 100× solution of hypoxanthine and thymidine) (Invitrogen/Life Technologies, Grand Island, NY). Electroporations were performed at 280 volts, 950 microFarads, using a BioRad (Hercules, CA) GenePulser electroporation unit with capacitance extender. Electroporation was performed in 0.4 cm gap sterile, disposable cuvettes. Electroporated cells were incubated for 5 minutes after electroporation prior to transfer of the treated cells to non-selective Excell 302 complete media in T75 flasks. Transfected cells were incubated overnight at 37° C., 5% C02 in non-selective media to permit recovery, prior to selective plating in 96-well flat-bottom plates (Costar) at varying serial dilutions ranging from 250 cells/well (2500 cells/ml) to 2000 cells/well (20,000 cells/ml). Culture media for cell cloning was Excell 302 complete containing 50 nM methotrexate. Transfection plates were fed at five-day intervals with 80 μl fresh media. After the first couple of feedings, 100 pd media was removed and replaced with fresh media. Plates were monitored and individual wells with clones were fed until clonal outgrowth became close to confluent, after which clones were expanded into 24-well dishes containing 1 ml media/well. Aliquots of the culture supernatants from the original 96-well plate were harvested to a second 96-well plate prior to transfer and expansion of the cells in the 24-well plates. This second plate was frozen until performing dilutions for ELISA analysis to estimate IgG concentrations.

Screening Culture Supernatants for Production Levels of Recombinant Fusion Proteins:

Once clonal outgrowth of initial transfectants was sufficient, serial dilutions of culture supernatants from master wells were thawed and the dilutions screened for expression of DNase1 fusion protein by use of an —IgG sandwich ELISA. Briefly, NUNC Maxisorp plates were coated overnight at 4° C. with 2 microgram/ml F(ab′2) goat anti-human IgG (Jackson Immunoresearch, West Grove, PA; Catalog #109-006-098) in PBS. Plates were blocked in PBS/3% BSA, and serial dilutions of culture supernatants incubated at room temperature for 2-3 hours or overnight at 4° C. Plates were washed three times in PBS/0.05% Tween 20, and incubated with horseradish-peroxidase-conjugated F(ab′2) goat anti-human IgG (Jackson Immunoresearch, West Grove, PA, Catalog #109-036-098) at 1:7500-1:10,000 in PBS/0.5% BSA, for 1-2 hours at room temperature. Plates were washed five times in PBS/0.05% Tween 20, and binding detected with SureBlue Reserve™ TMB substrate (KPL Labs/SeraCare, Gaithersburg, MD; catalog #53-00-02). Reactions were stopped by addition of equal volume of 1N HCl, and absorbance per well on each plate was read at 450 nM on a VarioSkan LUX plate reader (ThermoFisher Scientific, Waltham, MA). Concentrations were estimated by comparing the OD450 of the dilutions of culture supernatants to a standard curve generated using serial dilutions of a known standard, a protein-A-purified human —IgG fusion protein with the identical —Ig tail to clones described in this disclosure, typically THER4 (apoA1-(g4s)4-SSShinge-P238S-P331S Fc; see International PCT Publication No. WO 2017/044424) or an scFv-Fc targeted to human CD180 (G28-8). Data was collected and analyzed using platereader software and Microsoft Office Excel or GraphPad Prism 8.4.3 (San Diego, CA). The best expressing DNase-Ink-Fc clones expressed in the range of 25-55 μg/ml, while the best expressing DNase-Ink-Fc-PON1-K clones expressed at approximately 10-30 μg/ml.

Protein A purification supernatants were collected from spent CHO cell cultures expressing the fusion proteins, filtered through 0.2 μm PES express filters (Nalgene, Rochester, NY) and subjected to affinity chromatography using slow rotation of culture supernatants with Protein A-agarose (IPA 300 crosslinked agarose) slurry in 50 ml sterile, conical centrifuge tubes at 4° C. (Repligen, Waltham, MA). Fusion protein bound to protein A agarose was recovered by centrifugation, and culture supernatants removed, replaced, and the incubation process repeated until the desired volume of supernatant was processed. The final protein A agarose slurry was then loaded into sterile, acid-washed econocolumns (BioRad, Hercules, CA) to wash the resin. Columns were then washed with several column volumes of column wash buffer (Gentle Ag-Ab binding buffer, Pierce/ThermoFisher, Waltham, MA) to remove any residual culture supernatant, prior to elution. Bound protein was then eluted from the resin using gentle Ag/Ab elution buffer (Pierce/ThermoFisher, Waltham, MA). Fractions (0.8-1.0 ml) were collected and protein concentration of aliquots (2 μl) from each fraction were determined at 280 nM using a Nanodrop (Wilmington DE) microsample spectrophotometer, with blank determination using elution buffer alone. Fractions containing fusion protein were pooled, and buffer exchange was performed by dialysis using Spectrum Laboratories G2 (Ranch Dominguez, CA, Catalog #G235057, Fisher Scientific catalog #08-607-007) float-a-lyzer units (MWCO 20 kDa) against [0.9% sodium chloride, 5 mM sodium bicarbonate, 1 mM HEPES buffer, 1 mM calcium chloride, pH 7.5]. Dialysis was performed in sterile, 2.2-liter Corning roller bottles at 4° C. overnight. After dialysis, protein was filtered using 0.2 μM filter units, and aliquots tested for endotoxin contamination using Pyrotell LAL gel clot system single test vials (STV) (Catalog #G2006, Associates of Cape Cod, East Falmouth, MA).

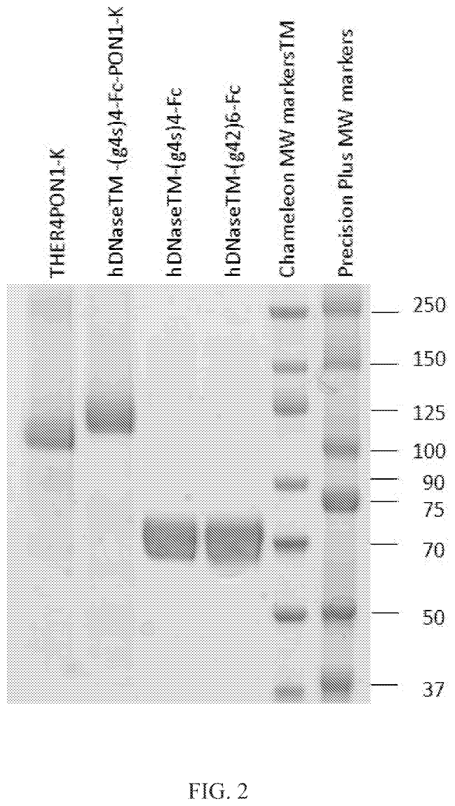

FIG. 2 shows purified proteins from stable CHO transfections after SDS-PAGE electrophoresis and staining with Imperial Protein Stain (Pierce/ThermoFisher, Waltham, MA/Rockville, MD. Catalog #24615). Five micrograms each protein-A-purified fusion protein was incubated in 1X LDS reducing sample buffer at 72° C. for 10 minutes followed by loading onto 4-12% Bis-Tris NuPAGE gels (Life Technologies, Carlsbad, CA). Protein molecular weight markers were included on each gel (Chameleon® DUO molecular weight markers (L1-COR Biosciences, Lincoln, NE) and Precision Plus molecular weight markers (BioRad, Hercules, CA)). SDS-PAGE gels were run in NuPAGE MOPS running buffer (Life Technologies, Carlsbad, CA) under reducing conditions at 175 volts for approximately 2 hours. Gels were washed three times in 100 ml distilled water, then stained in 30 ml Imperial Stain (Pierce/ThermoFisher, Waltham, MA) at room temperature with rocking for 2.5 hours. Gels were destained by washing 4 times in 100-200 ml distilled water and placed in a plastic sleeve prior to photographing/scanning of the stained gel. Lanes were loaded as follows: Lane 1—THER4PON1-K (apoA1-(g4s)4-SSShinge-P238S-P331S-Fc-NGS-PON1-K; see International PCT Publication No. WO 2017/044424); Lane 2—hDNase™-(g4s)4-Fc-PON1-K; Lane 3—hDNase™-(g4s)4-Fc; Lane 4—hDNase™-(g4s)6-Fc; Lane 5—L1-COR Chameleon® MW markers; Lane 6—BioRad Precision Plus Kaleidoscope MW markers. The approximate molecular weight (kDa) of each marker band is indicated on the right side of the stained gel.

### Example 2: DNase Activity of DNase1—Fc and DNase1-Fc-PON1 Fusion Proteins

Nuclease activity of DNase1 fusion proteins was assessed using a modified SYTOX™ Green fluorescence assay. SYTOX Green dye (5 mM solution in DMSO, Catalog #S7020) was obtained from Molecular Probes/Invitrogen (ThermoFisher Scientific, Waltham, MA). Salmon sperm DNA was also obtained from ThermoFisher Scientific (UltraPure, sheared, phenol purified, Catalog #15632011). Enzyme activity assays were performed either by titrating the substrate concentration with a fixed enzyme concentration, or by titrating the enzyme concentration with a fixed substrate concentration. For substrate titration experiments, salmon sperm DNA was mixed with SYTOX Green stain at a ratio of 14 μM sytox green to 400 μM (140 μg/ml) salmon sperm DNA in 1X DNase reaction buffer (10 mM Tris, pH 7.5, 5 mM MgCl₂, 1 mM CaCl₂), and the mixture was incubated at 37° C. for approximately 2 hours to label and equilibrate. Appropriate dilutions of the stock labeled DNA were made for enzyme activity assays. One-third fold serial dilutions of SYTOX Green labeled DNA were prepared in a separate plate and 50 pd of each dilution was transferred to the assay plate to generate the following substrate concentrations in the final reactions: 1) 220 μM salmon sperm DNA (SS DNA) and 10 μM SYTOX Green; 2) 147 μM SS DNA and 6.7 μM SYTOX Green; 3) 98.78 μM SS DNA and 4.4 μM SYTOX Green; 4) μM SS DNA and 3.01 μM SYTOX Green; and 5) 44 μM SS DNA and 2.05 μM SYTOX Green.

Each fusion protein or recombinant DNase1 enzyme was prepared at two times the desired final concentration (9 nM initial, or 4.5 nM final concentration) to be assessed in 1X DNase reaction buffer. Fifty microliters of each fusion protein or recombinant enzyme were added to each well of the assay plate containing the labeled substrate. Plates were immediately transferred to the plate reader, and fluorescence was measured using 485 nm excitation and 528 nm emission wavelengths. Sample fluorescence was monitored using a kinetic assay on a VarioSkan LUX fluorescent plate reader (Thermo Scientific, Waltham, MA) with readings every 45 seconds for a total of 40 minutes. RFU (Relative Fluorescence Units) were plotted as a function of time for each well. The fluorescence signal in each well decreased more rapidly with increasing nuclease activity.

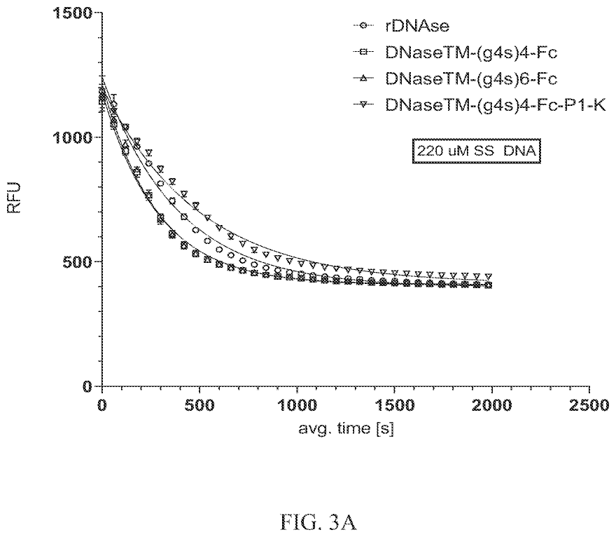

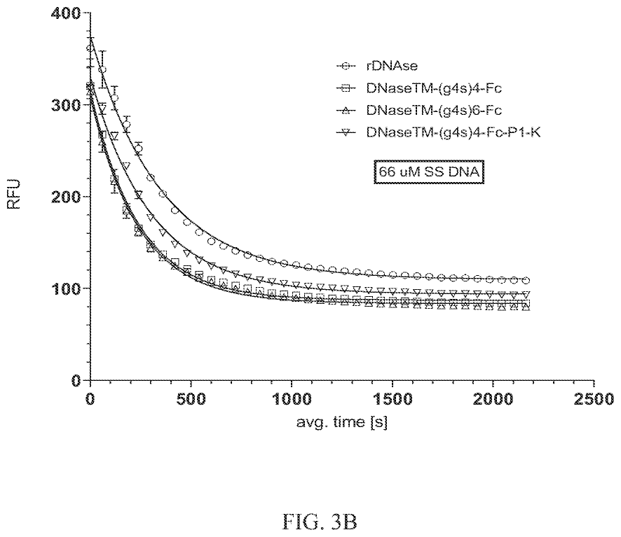

FIG. 3 shows a graphical representation of the raw data analyzing the relative fluorescence units (RFU) as a function of time at two different substrate concentrations, 220 μM or 66 μM salmon sperm DNA. For these experiments, the enzyme concentration used was fixed at 4.5 nM. The specific activity of the DNase1™-(g4s)4-Fc and DNase1TM-(g4s)6-Fc fusion proteins was higher than the recombinant wild-type human DNase1 at all substrate concentrations. At lower concentrations of substrate (the more physiologically relevant levels), the bispecific fusion protein (DNase1 ™-(g4s)4-Fc-P1-K) showed higher activity than the recombinant wild-type DNase1, while at higher concentrations of DNA substrate, the wild-type DNase1 had higher activity than the bispecific fusion protein.

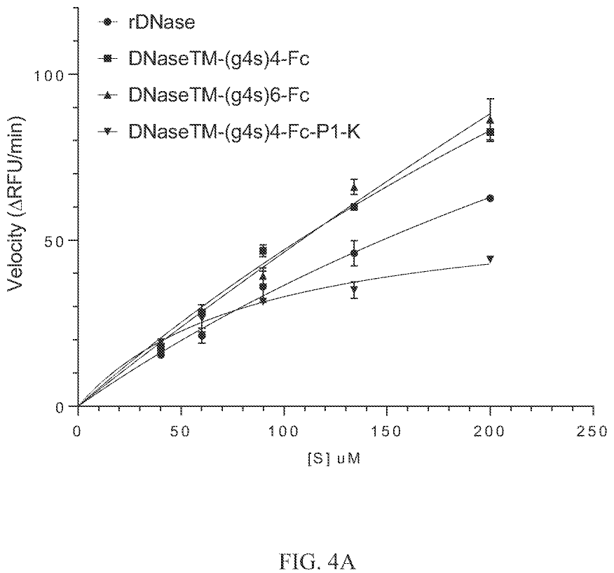

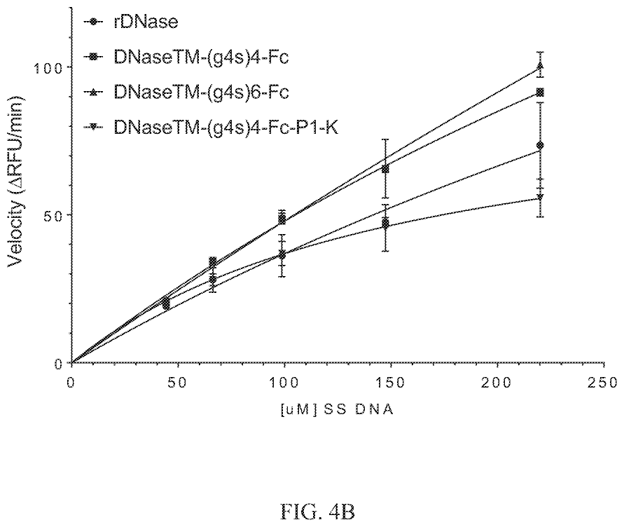

FIGS. 4A and 4B show Michaelis Menten plots of the data from two different DNase activity experiments (“Experiment #1” and “Experiment #2”) using these conditions. The estimated Km and Vmax for each fusion protein in Experiment #1 and Experiment #2 are shown below in Table 2, with the tabulated data for each experiment listed. The units for the Vmax or maximal velocity are in pmoles SYTOX-labeled salmon DNA digested/min, and the units for the Km are in pmoles labeled salmon sperm DNA. Raw data was analyzed using the Skanlt™ software for the VarioSkan LUX plate reader (Thermo Scientific, Waltham, MA), then the data further analyzed using Microsoft Excel (Redmond, WA) and GraphPad Prism 8.4.3 (San Diego CA).

TABLE 2
Tabulated Michaelis Menten Data for
DNase Fusion Proteins at 4.5 nM

| | | DNaseTM- | DNaseTM- | DNaseTM- |
|---|---|---|---|---|
| | rDNase | (g4s)4-Fc | (g4s)6-Fc | (g4s)4Fc-P1-K |
| Experiment #1 | | | | |
| Vmax | 332.5 | 374.7 | 968.9 | 95.91 |
| Km | 533.6 | 681.9 | 1919 | 159.7 |
| Experiment #2 | | | | |
| Vmax | 230.8 | 349,4 | 874.3 | 61.13 |
| Km | 533.6 | 641.6 | 1785 | 85.29 |

Vmax: μmol salmon sperm DNA digested/min
Km: μmol salmon sperm DNA

Enzyme activity assays were also performed in the presence or absence of actin to assess whether the DNase fusion proteins were sensitive to actin inhibition. These assays were performed either by titrating the substrate concentration with a fixed enzyme concentration, or by titrating the enzyme concentration with a fixed substrate concentration. For substrate titration experiments, salmon sperm DNA was mixed with SYTOX Green stain at a ratio of 14 μM sytox green to 400 μM (140 μg/ml) salmon sperm DNA in 1X DNase reaction buffer (10 mM Tris, pH 7.5, 5 mM MgCl₂, 1 mM CaCl₂)), and the mixture was incubated at 37° C. for approximately 2 hours to label and equilibrate.

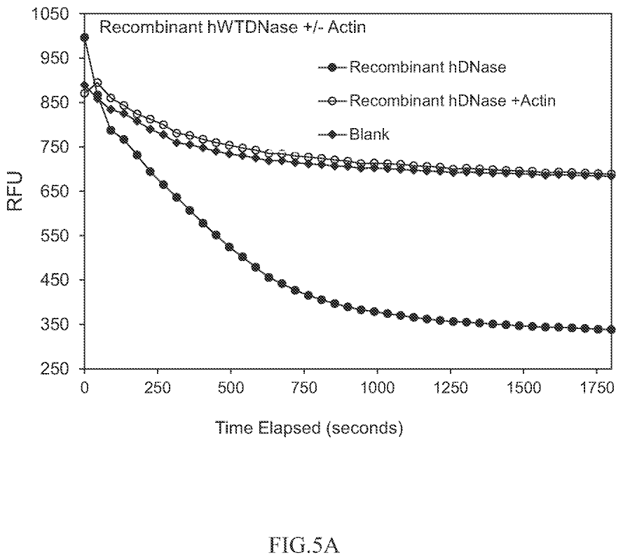

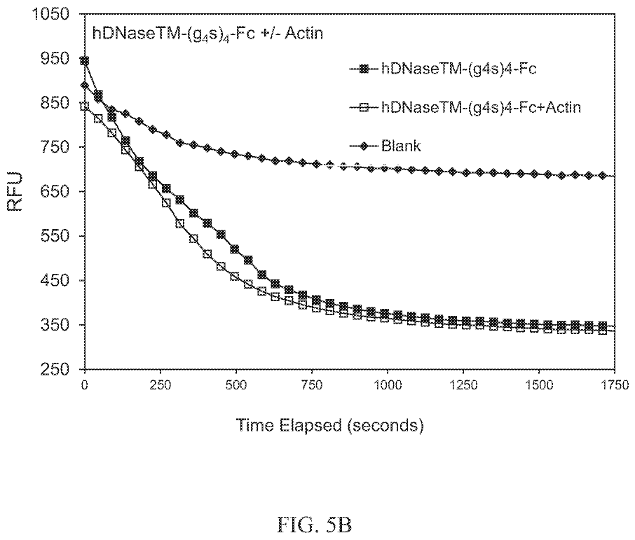

Each fusion protein or recombinant DNase1 enzyme was prepared at four times the desired final concentration (18 nM initial, or 4.5 nM final concentration) to be assessed in 1X DNase reaction buffer. Each 4X sample was then mixed 1:1 with a solution containing rabbit skeletal muscle actin protein (Cytoskeleton, Denver, CO., Catalog #AKL99-A) at 4X=160 μg/ml, or a final concentration in the nuclease reaction of 40 μg/ml; or reaction buffer containing no actin. Samples containing 9 nM fusion protein with 80 μg/ml actin were incubated at 37° C. for 45-60 minutes prior to addition of the labeled SYTOX green substrate. Sytox green substrate was prepared by adding 160 μg/ml salmon sperm DNA to 14 μM Sytox green dye in 1X DNase reaction buffer and equilibrating at 37° C. for 60-120 minutes. Fifty microliters of each actin-treated fusion protein or recombinant enzyme were added to each well of the assay plate containing 50 μl of the labeled substrate. Plates were immediately transferred to the plate reader, and fluorescence was measured using nm excitation and 528 nm emission wavelengths. Sample fluorescence was monitored using a kinetic assay on a VarioSkan LUX fluorescent plate reader (Thermo Scientific, Waltham, MA) with readings every 45 seconds for a total of 40 minutes. RFU (Relative Fluorescence Units) were plotted as a function of time for each well. The fluorescence signal in each well decreased more rapidly with increasing nuclease activity. FIGS. 5A and 5B show graphs plotting the RFU as a function of time for the DNase™-(g4s)4-Fc molecule and for the recombinant human DNase. The nuclease activity of the recombinant human DNase1 was inhibited by actin, while the fusion protein variants were not inhibited under these conditions.

### Example 3: Paraoxonase Activity of DNase1-Fc-PON1 Fusion Protein Arylesterase Activity

Phenyl acetate was used as a substrate to assess arylesterase activity of the DNase1TM-(g4s)4-Fc-PON1-Q192K fusion protein. Enzyme activity assays were performed either by titrating the substrate concentration with a fixed enzyme concentration, or by titrating the enzyme concentration with a fixed substrate concentration. For the phenyl acetate activity assays, reaction buffer contained 20 mM Tris-HCl, pH 8.0, 1 mM CaCl₂). Substrate solution contained reaction butter and 10 mM phenyl acetate, while enzyme solution contained reaction buffer and enzyme(s) at the appropriate concentrations. Substrate solution was diluted 1:1 with enzyme solutions for a final concentration of 5 mM phenyl acetate substrate in the reactions. Fusion protein (enzyme) concentration was present in the assays titrated in 1/3 serial dilutions from 40 μg/ml (40, 26.8, 17.96, 12.05, 8.06, 5.4, 3.62, and 2.4 μg/ml). Sample absorption was monitored using a kinetic assay on a VarioSkan LUX fluorescent plate reader, with readings every 45 seconds for a total of 10 minutes. Molar absorptivity at 270 nm serves as a measure of phenol formed as a function of time. The change in absorption was converted into a reaction rate using the molar extinction coefficient of 1310 M⁻¹ cm¹ for phenol.

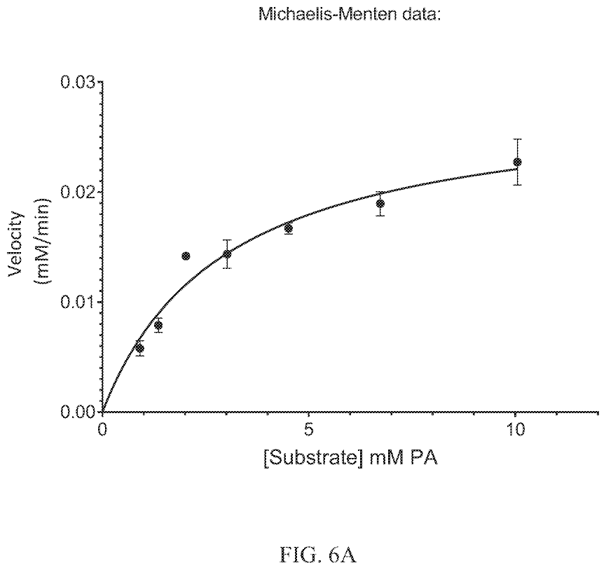

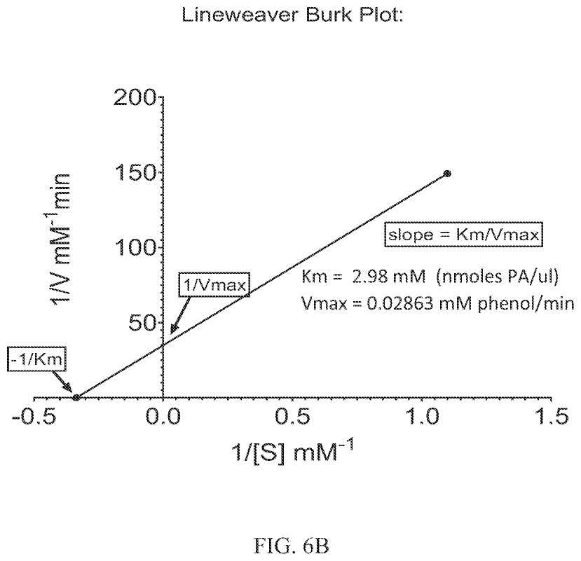

In addition, arylesterase activity assays were performed titrating the amount of phenyl acetate substrate present in 1/3-fold serial increments as follows: 15 mM, 10.05 mM, 6.73 mM, 4.51 mM, 3.02 mM, 2.03 mM, 1.36 mM, and 0.91 mM substrate. In each case, the amount of fusion protein (enzyme) present was 7.5 μg/ml. For the DNase1TM-(g4s)4-Fc-PON1-Q192K molecules, this concentration corresponds to 75 nM. FIGS. 6A and 6B show Michaelis Menten analysis and Lineweaver Burk plots for a representative experiment measuring the arylesterase activity of the DNase™-(g4s)4-Fc-PON1-Q192K fusion protein. When multiple experiments were compared, the Vmax average was approximately 0.027 mM phenol/min (mmoles/liter=nmoles/μl) and the Km was approximately 2.7 mM phenyl acetate (mmoles/liter=nmoles phenyl acetate/μl).

Organophosphatase Activity

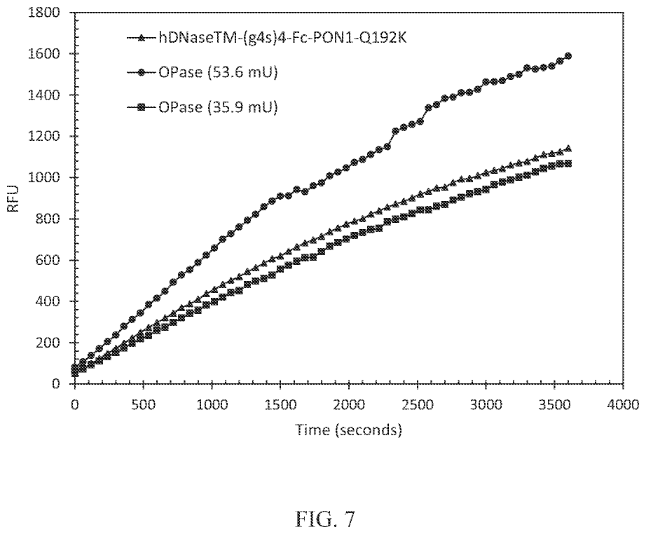

The Enzcheck paraoxonase activity assay (Catalog #E33702, ThermoFisher Scientific, Waltham, MA) was used to assess organophosphatase activity of the DNase1TM-(g4s)4-Fc-PON1-Q192K fusion protein. This assay is a very sensitive fluorometric assay for the organophosphatase activity of paraoxonase that uses excitation/emission maxima of 360/450 nm to measure the conversion of a fluorogenic organophosphate analog provided with the kit. The assay can either be set up as a kinetic assay or terminated after a particular period of time for an endpoint assay. The change in relative fluorescence units (RFU) per unit time is converted to the units of paraoxonase in the sample using the standard curve generated from the fluorescent standard and the conversion factor that 1 U unit of paraoxonase generates 1 nmol of fluorescent product per minute at 37° C. The amount of paraoxonase present in the fusion protein samples can be compared to the paraoxonase positive control provided with the kit. FIG. 7 shows a graphical representation of the organophosphatase activity of the DNase1TM-(g4s)4-Fc-PON1-Q192K fusion protein compared to the RFU curves generated by two different dilutions of the organophosphatase positive control in the average to above average activity range for human serum sample comparisons. The fusion protein tested had been kept at 4° C. for 11 months, but still had activity above average in the PON1 assay.

Similar activity assays to the arylesterase and organophosphatase can be performed for the PON1 enzyme activities present in fusion proteins as described herein, such as, e.g., peroxidase activity assays (Amplex Red peroxide/peroxidase assay, ThermoFisher Scientific, Waltham MA, Catalog number: A22188) or a general phosphatase assay (ENzChek phosphatase assay, ThermoFisher Scientific, Waltham MA, Catalog number: 12020).

### Example 4: Assays to Assess Inhibition of Biofilm Formation by DNase-Fc and DNase-Fc-PON1 Fusion Proteins

DNase-Fc and DNase-Fc-PON1 fusion proteins are characterized in biofilm formation assays to evaluate inhibition of Pseudomonas aeruginosa biofilm formation. Pseudomonas aeruginosa cultures are grown from an isolated bacterial colony by inoculation into rich media such as 2xYT or LB media. Overnight cultures are diluted 1:100 in tryptic soy broth/0.5% glucose (Teknova, Hollister, CA), and the diluted cultures used to inoculate 100 μl/well into a 96 well tissue culture plate. Wells are precoated with each fusion protein of interest at different concentrations, prior to inoculation with the diluted bacterial culture. Alternatively, fusion proteins are added directly to diluted bacterial cultures to assess inhibition activity in solution. Plates are incubated at 37° C. for 4-8 hours prior to assay. All conditions are performed in replicates of 6-8 in order to control for well-to-well variation. After the incubation, cultures are removed by aspiration or turning the plate over and shaking out the liquid. The plate is then washed with PBS to remove unattached cells and media. After washing, 125 μl of 0.1% solution crystal violet is added to each well, and the plate is incubated for 15 minutes at room temperature. Plates are rinsed 3-4 times with water or PBS using a plate washer. After the final wash, microtiter plates are inverted and left to dry for a few hours or overnight. To quantify biofilms, 125 μl 30% acetic acid is added to each dried well in order to solubilize the crystal violet. Plates are incubated at room temperature for 15 minutes, and the 125 μl of crystal violet is transferred to a new flat bottom microtiter dish. Well absorbance is then measured at 550 nm in a Varioskan plate reader using 30% acetic acid as a blank solution. Percent biofilm inhibition is estimated by subtracting the ratio of [fusion protein treated absorbance average/untreated absorbance average] from 1 and multiplying by 100.

A commercially available biofilm formation kit is also available from Dojindo (Donjindo Laboratories, Kumamoto, Japan) that uses pegged microtiter plates for biofilm formation, simplifying washing steps.

### Example 5: Construction of SOD1-Fc-PON1 Fusion Proteins

The sequence for wild-type human SOD1 (see GenBank Accession Nos. NM_000454.5, CR450355.1, and CR4541742.1) is obtained from the NCBI sequence database and is designed and synthesized as an expression cassette for insertion into a molecule already containing a partial fusion gene encoding a (Gly₄Ser)₂ linker ((g4s)2; nucleotide and amino acid sequences as shown in SEQ ID NO:9 and SEQ ID NO:10) attached to the N-terminus of a human IgG1 Fc variant (SSS hinge, P238S, P331S Fc; nucleotide and amino acid sequences as shown in SEQ ID NO:27 and SEQ ID NO:28). The carboxyl end of the human Fc variant is fused to a peptide linker containing an N-linked glycosylation site (NGS; nucleotide and amino acid sequences as shown in SEQ ID NO:55 and SEQ ID NO:56) followed by a variant of human PON1 (PON1 Q192K; nucleotide and amino acid sequences as shown in SEQ ID NO:5 and SEQ ID NO:6) in which the first 15 amino acids of the uncleaved leader peptide are deleted. The predicted amino acid sequence of SOD1 does not contain a signal peptide for secretion, so a modified version of the human VK3 signal sequence is designed with additional amino acids at the junction site with SOD1 in order to achieve cleavage of the heterologous signal peptide at the correct location. The amino terminal methionine for SOD1 is absent in the mature secreted version of this modified SOD1, so that the predicted N-terminal amino acid after cleavage of the modified signal peptide is alanine. The encoded, modified VK3 leader and N-terminal SOD1 sequence is METPAQLLFLLLLWLPDTTGMA/ATKAVCVL (residues 1-30 of SEQ ID NO:50), where the slash mark indicates the junction between the VK3 leader peptide (VK3LP) and SOD1. The completed SOD1 fusion construct is [Human VK3LP]-[hSOD1]-(g4s)2-[SSShinge-P238S-P331S Fc]-NGS-[PON1 Q192K](nucleotide and amino acid sequences as shown in SEQ ID NO:49 and SEQ ID NO:50).

Additional SOD1 fusion genes containing a sequence variant of SOD1 are also constructed. The variant substitutes alternative amino acids for cysteines at positions 7 and of human SOD1 (amino acid numbering according to the full-length immature SOD1 sequence of SEQ ID NO:32), creating a C7A and C112S double mutant form of the predicted amino acid sequence. This sequence was originally described by Parge et al. (Proc. Natl. Acad. Sci. USA 89:6109-6113, 1992) as a thermostable mutant of SOD1 with improved solubility and stability. The SOD1 double mutant variant is fused to the N-terminus of the human Fc variant (SSS hinge, P238S, P331S Fc; see above) using either the (Gly₄Ser)₂ linker (see above) or a (Gly₄Ser)₄ linker ((g4s)4; nucleotide and amino acid sequences as shown in SEQ ID NO:11 and SEQ ID NO:12). The carboxyl end of the Fc is fused to the NGS linker followed by the PON1 Q192K variant as described above for the wild-type SOD1 fusion. The completed double mutant SOD1 fusion constructs are [Human VK3LP]-[hSOD1 C7A C112S]-(g4s)2-[SSShinge-P238S-P331S Fc]-NGS-[PON1 Q192K](nucleotide and amino acid sequences as shown in SEQ ID NO:51 and SEQ ID NO:52) and [Human VK3LP]-[hSOD1 C7A C112S]-(g4s)4-[SSShinge-P238S-P331S Fc]-NGS-[PON1 Q192K](also referred to hereinbelow as SOD1-P1-K: nucleotide and amino acid sequences as shown in SEQ ID NO:53 and SEQ ID NO:54).

Superoxide dismutase activity of the SOD1-Fc-PON1 fusion proteins is measured using a commercially available kit such as the SOD colorimetric activity kit (ThermoFisher Scientific, Waltham MA, Catalog number: EIASODC) or a similar kit available from Cayman Chemical, (Cayman Chemical, Ann Arbor, MI; Catalog number: 706002).

### Example 6: Construction of RNase1-Fc-PON1 Fusion Proteins

The sequence for wild-type human RNase 1 (see GenBank Accession Nos. NM_198235.3, NM_198234.3, NM_198232.3, and NM_002933.5) is obtained from the NCBI sequence database and is designed and synthesized as an expression cassette encoding wild-type RNase 1 with its native leader sequence (nucleotide and amino acid sequences as shown in SEQ ID NO:21 and SEQ ID NO:22) or RNase 1 attached to the human VK3 leader peptide (VK3LP) sequence (nucleotide and amino acid sequences as shown in SEQ ID NO:23 and SEQ ID NO:24). These RNase1-encoding cassettes are then fused to a human IgG1 Fc variant with C220S, C226S, C229S, and P238S amino acid substitutions, but wild-type CH2 and CH3 domains (SSS hinge, P238S Fc; nucleotide and amino acid sequences as shown in residues 475-1170 of SEQ ID NO:41 and residues 159-390 of SEQ ID NO:42). The nucleotide and amino acid sequences for the wild-type RNase1-Fc construct (with the native leader) are shown in SEQ ID NO:41 and SEQ ID NO:42, and the nucleotide and amino acid sequences for the VK3LP-RNase1-Fc construct are shown in SEQ ID NO:43 and SEQ ID NO:44. These constructs contain two heterologous, restriction-site-encoded amino acids (Asp-Leu; residues 157-158 of SEQ ID NO:42 or residues 149-150 of SEQ ID NO:44) linking the carboxyl end of RNase1 to the N-terminus of the Fc.

The RNase1-encoding cassettes are also fused to the SSS hinge, P238S Fc variant via a (Gly₄Ser)₄ linker peptide ((g4s)4; nucleotide and amino acid sequences as shown in SEQ ID NO:11 and SEQ ID NO:12). The resulting RNase1-(g4s)4-Fc nucleotide and predicted amino acid sequences are shown in SEQ ID NO:33 and SEQ ID NO:34 (for the wild-type RNase1 with the native leader) and SEQ ID NO:35 and SEQ ID NO:36 (for the mature wild-type RNase1 fused to the human VK3 leader).

The carboxyl end of the human Fc variant in each of the RNase1-Fc constructs described above is fused to a peptide linker containing an N-linked glycosylation site (NGS; nucleotide and amino acid sequences as shown in SEQ ID NO:55 and SEQ ID NO:56) followed by a variant of human PON1 (PON1 Q192K; nucleotide and amino acid sequences as shown in SEQ ID NO:5 and SEQ ID NO:6) in which the first 15 amino acids of the uncleaved leader peptide are deleted. The completed RNase1-Fc-PON1 constructs generated are as follows:
hRNase1-[SSShinge-P238S Fc]-NGS-[PON1 Q192K](nucleotide and amino acid sequences as shown in SEQ ID NO:45 and SEQ ID NO:46);
hVK3LP-hRNase1-[SSShinge-P238S Fc]-NGS-[PON1 Q192K](nucleotide and amino acid sequences as shown in SEQ ID NO:47 and SEQ ID NO:48);
hRNase1-(g4s)4-[SSShinge-P238S Fc]-NGS-[PON1 Q192K](nucleotide and amino acid sequences as shown in SEQ ID NO:37 and SEQ ID NO:38); and
hVK3LP-hRNase1-(g4s)4-[SSShinge-P238S Fc]-NGS-[PON1 Q192K](nucleotide and amino acid sequences as shown in SEQ ID NO:39 and SEQ ID NO:40).

### Example 7: Construction of CTLA4-Fc-PON1 Fusion Proteins

The sequence for wild-type, full length, human CTLA-4 (see GenBank Accession Nos. AF414120.1 and L15006.1) is obtained from the NCBI sequence database and is used in the design and synthesis of an expression cassette encoding the human CTLA-extracellular domain attached to the human VK3 leader peptide (VK3LP) sequence (nucleotide and amino acid sequences as shown in SEQ ID NO: 57 and SEQ ID NO: 58). These CTLA-4-encoding cassettes are then fused to a human IgG1 Fc variant with C220S, C226S, C229S, P238S and P331S amino acid substitutions in the Fc domain (SSS hinge, P238S, P331S Fe; nucleotide and amino acid sequences as shown in SEQ ID NO:27 and SEQ ID NO:28). The nucleotide and amino acid sequences for the VK3LP-CTLA4-Fc construct are shown in SEQ ID NO:63 and SEQ ID NO:64. These constructs contain two heterologous, restriction-site-encoded amino acids (Asp-Leu or DL; residues 433-438 of SEQ ID NO:63 or residues 145-146 of SEQ ID NO:64) linking the carboxyl end of CTLA-4 to the N-terminus of the Fc region.

The carboxyl end of the human Fc variant in each of the CTLA4-Fc constructs described above is fused to a peptide linker containing an N-linked glycosylation site (NGS; nucleotide and amino acid sequences as shown in SEQ ID NO:55 and SEQ ID NO:56) followed by a variant of human PON1 (PON1 Q92K; nucleotide and amino acid sequences as shown in SEQ ID NO:5 and SEQ ID NO:6) in which the first 15 amino acids of the uncleaved leader peptide are deleted. The nucleotide and amino acid sequences of the completed CTLA4-Fc-PON1 construct generated (hVK3LP-[hCTLA-4EC]-[SSShinge-P238S-P331S Fc]-NGS-[PON1 Q192K]; also referred to hereinbelow as CTLA4-Fc-P1-K) are listed in SEQ ID NO:65 and SEQ ID NO:66.

### Example 8: Construction of CD40-Fc-PON1 Fusion Proteins

The sequence for wild-type, full length, human CD40 (nucleotide and amino acid sequences shown in SEQ ID NO:67 and SEQ ID NO:68; see GenBank Accession No. NM_001250) is obtained from the NCBI sequence database and is used in the design and synthesis of an expression cassette encoding the human CD40 extracellular domain with its native leader sequence or the human CD40 extracellular domain attached to the human VK3 leader peptide (VK3LP) sequence (nucleotide and amino acid sequences as shown in SEQ ID NO:57 and SEQ ID NO:58). These CD40-encoding cassettes are then fused to a (Gly₄Ser)₄ peptide linker (nucleotide and amino acid sequences as shown in SEQ ID NO:11 and SEQ ID NO:12, including flanking restriction sites and encoded amino acids) and a human IgG1 Fc variant with C220S, C226S, C229S, P238S and P331S amino acid substitutions in the Fc domain (SSS hinge, P238S, P331S Fc; nucleotide and amino acid sequences as shown in SEQ ID NO:27 and SEQ ID NO:28). The nucleotide and amino acid sequences for the wild-type CD40-Fc construct with the native leader ([hCD40 EC]-(g4s)4-[SSShinge-P238S Fc]) are shown in SEQ ID NO:69 and SEQ ID NO:70, and the nucleotide and amino acid sequences for the CD40-Fc construct with the VK3LP leader (hVK3LP-[hCD40 EC]-(g4s)4-[SSShinge-P238S Fc]) are shown in SEQ ID NO:71 and SEQ ID NO:72.

In addition to the wild-type form of CD40, several variants were generated which show improved binding avidity for the CD40 ligand (CD154 or gp39). Using the amino acid numbering according to the wild-type, full-length human CD40 (SEQ ID NO:68), the amino acid substitution(s) for each of these CD40 variants relative to the wild-type sequence are (i) K81T, (ii) K81H, L121P, (iii) K81S, and (iv) E64Y, K81T, P85Y. The variant CD40-Fc constructs are as follows:
hVK3LP-[hCD40 EC K81T]-(g4s)4-[SSShinge-P238S Fc](nucleotide and amino acid sequences as shown in SEQ ID NO:75 and SEQ ID NO:76);
hVK3LP-[hCD40 EC K81H-L121P]-(g4s)4-[SSShinge-P238S Fc](nucleotide and amino acid sequences as shown in SEQ ID NO:79 and SEQ ID NO:80);
hVK3LP-[hCD40 EC K81S]-(g4s)4-[SSShinge-P238S Fe](nucleotide and amino acid sequences as shown in SEQ ID NO:83 and SEQ ID NO:84); and
hVK3LP-[hCD40 EC E64Y-K81T-P85Y]-(g4s)4-[SSShinge-P238S Fc](nucleotide and amino acid sequences as shown in SEQ ID NO:87 and SEQ ID NO:88).

The carboxyl end of the human Fc variant in each of the CD40-Fc constructs described above is fused to a peptide linker containing an N-linked glycosylation site (NGS; nucleotide and amino acid sequences as shown in SEQ ID NO:55 and SEQ ID NO:56) followed by a variant of human PON1 (PON1 Q192K; nucleotide and amino acid sequences as shown in SEQ ID NO:5 and SEQ ID NO:6) in which the first 15 amino acids of the uncleaved leader peptide are deleted. The completed CD40-Fc-PON1 constructs generated are as follows:
hVK3LP-[hCD40 EC]-(g4s)4-[SSShinge-P238S Fc]-NGS-[PON1 Q192K](nucleotide and amino acid sequences as shown in SEQ ID NO:73 and SEQ ID NO:74);
hVK3LP-[hCD40 EC K81T]-(g4s)4-[SSShinge-P238S Fc]-NGS-[PON1 Q192K](nucleotide and amino acid sequences as shown in SEQ ID NO:77 and SEQ ID NO:78);
hVK3LP-[hCD40 EC K81H-L121P]-(g4s)4-[SSShinge-P238S Fc]-NGS-[PON1 Q192K](nucleotide and amino acid sequences as shown in SEQ ID NO:81 and SEQ ID NO:82);
hVK3LP-[hCD40 EC K81S]-(g4s)4-[SSShinge-P238S Fc]-NGS-[PON1 Q192K](nucleotide and amino acid sequences as shown in SEQ ID NO:85 and SEQ ID NO:86); and hVK3LP-[hCD40EC E64Y-K81T-P85Y]-(g4s)4-[SSShinge-P238S Fc]-NGS-[PON1 Q192K](nucleotide and amino acid sequences as shown in SEQ ID NO:89 and SEQ ID NO:90) (also referred to hereinbelow as CD40TM-Fc-P1-K).

### Example 9: Construction of Anti-TNF-α scFv-Fc-PON1 Fusion Proteins

The sequence for Humira™ or the adalimumab antibody targeted to human TNF-α is obtained from the IMGT database INN number 7860, chain numbers INN 7860_H and INN 7860_L. The GenBank Accession numbers for the adalimumab anti-TNF-α antibody sequences are LQ961187 for the heavy chain variable region and LQ961186 for the light chain variable region. See also International PCT Publication No. WO 2014/159579. The sequences are used to design and synthesize an expression cassette encoding an scFv including the heavy and light chain V regions for the adalimumab antibody attached to the human VK3 leader peptide (VK3LP) sequence (nucleotide and amino acid sequences as shown in SEQ ID NO:57 and SEQ ID NO:58). The V region sequences are joined by a (gly4ser)4 linker segment to construct the scFv. The anti-TNF-α scFv is constructed in both the VL-VH and VH-VL orientations. The nucleotide and amino acid sequences for the human VK3LP anti-TNF-α VL-VH scFv are shown in SEQ ID NO:91 and SEQ ID NO:92, and the nucleotide and amino acid sequences for the human VK3LP anti-TNF-α VH-VL scFv are shown in SEQ ID NO:95 and SEQ ID NO:96.

These TNF-α scFv-encoding cassettes are then linked at the carboxyl end of the scFv to the N-terminus of the Fc domain using two heterologous, restriction-site-encoded amino acids (Asp-Leu; residues 816-822 of SEQ ID NO:93 and SEQ ID NO:97 or residues 269-270 of SEQ ID NO:94 and SEQ ID NO:98). The Fc domain encodes a human IgG1 Fc variant with C220S, C226S, C229S, and P238S amino acid substitutions, but wild-type for the remaining residues of the CH2 and CH3 domains (SSS hinge, P238S Fc; nucleotide and amino acid sequences as shown in residues 475-1170 of SEQ ID NO:41 and residues 159-390 of SEQ ID NO:42). The carboxyl end of the human Fc variant in each of the scFv-Fc constructs is fused to a peptide linker containing an N-linked glycosylation site (NGS; nucleotide and amino acid sequences as shown in SEQ ID NO:55 and SEQ ID NO:56) followed by a variant of human PON1 (PON1 Q192K; nucleotide and amino acid sequences as shown in SEQ ID NO:5 and SEQ ID NO:6) in which the first 15 amino acids of the uncleaved leader peptide are deleted. The completed anti-TNF-α scFv-Fc-PON1 constructs generated are as follows:
hVK3LP-[VL-VH anti-TNFalpha scFv]-[SSShinge-P238S-P331S Fc]-NGS-[PON1 Q192K](nucleotide and amino acid sequences as shown in SEQ ID NO:93 and SEQ ID NO:94) (also referred to hereinbelow as Adalimumab scFv-Fc-P1-K); and
hVK3LP-[VH-VL anti-TNFalpha scFv]-[SSShinge-P238S-P331S Fc]-NGS-[PON1 Q192K](nucleotide and amino acid sequences as shown in SEQ ID NO:97 and SEQ ID NO:98).

### Example 10: Construction of Anti-TGF-β scFv-Fc-PON1 Fusion Proteins

The sequence for humanized fresolimumab antibody (GC1008) targeted to human TGF-β cytokine isoforms is obtained from the IMGT sequence database. The chain ID designations are INN 9158_H and INN 9158_L. The GenBank Accession numbers for the fresolimumab anti-TGF-β antibody sequences are JC 232803.1 for the heavy chain and JC for the light chain. The sequences for the antibody are also listed in U.S. Pat. No. 7,723,486 and International PCT Publication No. WO 2013/065869. The sequences are used to design and synthesize an expression cassette encoding an scFv including the heavy and light chain V regions for the GC1008/fresolimumab antibody attached to the human VK3 leader peptide (VK3LP) sequence (nucleotide and amino acid sequences as shown in SEQ ID NO:57 and SEQ ID NO:58). The V region sequences are joined by a (gly4ser)4 linker segment to construct the scFv. The anti-TGF-β scFv is constructed in both the VL-VH and VH-VL orientations. The nucleotide and amino acid sequences for the human VK3LP anti-TGF-β VH-VL scFv are shown in SEQ ID NO:99 and SEQ ID NO:100, and the nucleotide and amino acid sequences for the human VK3LP anti-TGF-β VL-VH scFv are shown in SEQ ID NO:103 and SEQ ID NO:104.

These TGF-β scFv-encoding cassettes are then fused at the carboxyl end of the scFv to the N-terminus of the Fc domain using two heterologous, restriction-site-encoded amino acids (Asp-Leu; residues 819-825 of SEQ ID NO: 101 and SEQ ID NO:105 or residues 270-271 of SEQ ID NO:102 and SEQ ID NO:106). The Fc domain encodes a human IgG1 Fc variant with C220S, C226S, C229S, and P238S amino acid substitutions, but wild-type for the remaining residues of the CH2 and CH3 domains (SSS hinge, P238S Fc; nucleotide and amino acid sequences as shown in residues 475-1170 of SEQ ID NO:41 and residues 159-390 of SEQ ID NO:42). The carboxyl end of the human Fc variant in each of the scFc-Fc constructs is fused to a peptide linker containing an N-linked glycosylation site (NGS; nucleotide and amino acid sequences as shown in SEQ ID NO:55 and SEQ ID NO:56) followed by a variant of human PON1 (PON1 Q192K; nucleotide and amino acid sequences as shown in SEQ ID NO:5 and SEQ ID NO:6) in which the first 15 amino acids of the uncleaved leader peptide are deleted. The completed anti-TGF-β scFv-Fc-PON1 constructs generated are as follows:
hVK3LP-[anti-hTGFbeta VH-VL scFv]-SSShinge-P238S-P331S Fc]-NGS-PON1 Q192K](nucleotide and amino acid sequences as shown in SEQ ID NO:101 and SEQ ID NO:102); and
hVK3LP-[anti-hTGFbeta VL-VH scFv]-SSShinge-P238S-P331S Fc]-NGS-PON1 Q192K](nucleotide and amino acid sequences as shown in SEQ ID NO:105 and SEQ ID NO:106).

### Example 11: Construction of DNase1 L3-Fc and DNase1L3-Fc-PON1 Fusion Proteins

Constructs were designed and synthesized and cassettes with the correct sequences are combined to generate two synthetic fusion genes encoding unique DNase1 L3 fusion molecules. Each construct includes a variant of wild-type human DNase1 L3 (nucleotide and encoded amino acid sequences as shown in SEQ ID NOs:135 and 136). Each DNase1 L3 sequence is modified to remove amino acid residues 291-305 of the wild-type sequence, corresponding to the C-terminal nuclear localization signal (NLS2). Each DNase1 L3 variant also contains amino acid substitutions, relative to the wild-type human sequence (SEQ ID NO:136), at positions R80, R95, and N96 (either all three positions changed to alanine or all three changed to serine) to inactivate a nuclear localization signal (NLS1) located in the N-terminal half of the molecule. The nucleotide and encoded amino acid sequences corresponding to the mature DNase1 L3 variant containing the R80A/R95A/N96A mutations are shown in residues 61-870 of SEQ ID NO:137 and residues 21-290 of SEQ ID NO:138, respectively; the nucleotide and encoded amino acid sequences corresponding to the mature DNase1L3 variant containing the R80S/R95S/N96S mutations are shown in residues 61-870 of SEQ ID NO:139 and residues 21-290 of SEQ ID NO:140, respectively. The signal peptide sequence utilized was the human VK3 leader peptide (nucleotide and amino acid sequences as shown in SEQ ID NO:57 and SEQ ID NO:58). The DNase1 cassette was fused to the N-terminus of a human IgG1 Fc variant (SSS hinge, P238S, P331S) using a (Gly₄Ser)₄ peptide linker ((g4s)4; nucleotide and amino acid sequences as shown in SEQ ID NO:11 and SEQ ID NO:12). The Fc variant nucleotide and amino acid sequences are shown in SEQ ID NO:27 and SEQ ID NO:28. The completed DNase1 L3-Fc constructs generated are as follows:
hVK3LP-[hDNase1 L3 R80A-R95A-N96A NLS2delta]-(g4s)4-[SSShinge-P238S-P331S Fc](nucleotide and amino acid sequences as shown in SEQ ID NO:137 and SEQ ID NO:138); and
hVK3LP-[hDNase1L3 R80S-R95S-N96S NLS2delta]-(g4s)4-[SSShinge-P238S-P331S Fc](nucleotide and amino acid sequences as shown in SEQ ID NO:139 and SEQ ID NO:140).

Bispecific constructs further comprising a functional PON1 enzyme are also generated by fusing the carboxyl end of each variant DNase1 L3-Fc sequence above to a peptide linker containing an N-linked glycosylation site (NGS; nucleotide and amino acid sequences as shown in SEQ ID NO:55 and SEQ ID NO:56) followed by a variant of human PON](PON1 Q192K; nucleotide and amino acid sequences as shown in SEQ ID NO:5 and SEQ ID NO:6) in which the first 15 amino acids of the uncleaved leader peptide are deleted. The completed DNase1 L3-Fc-PON1 constructs are as follows:
hVK3LP-[hDNase1L3 R80A-R95A-N96A NLS2delta]-(g4s)4-[SSShinge-P238S-P331S Fc]-NGS-[PON1 Q192K](nucleotide and amino acid sequences as shown in SEQ ID NO:161 and SEQ ID NO:162); and
hVK3LP-[hDNase1L3 R80S-R95S-N96S NLS2delta]-(g4s)4-[SSShinge-P238S-P331S Fc]-NGS-[PON1 Q192K](nucleotide and amino acid sequences as shown in SEQ ID NO:163 and SEQ ID NO:164) (also referred to hereinbelow as DNase1 L3-Fc-P1-K).

### Example 12: Construction and Expression of Bispecific and Monospecific PON1 Fusion Proteins

Various Fc fusion genes as described in Examples 1, 5, 7-9, and 11 were assembled in a pUC based vector, and assembled genes screened by DNA sequencing prior to further manipulations. Assembled genes included both a monospecific (T-L1-Fc) and a bispecific PON1 (T-L1-Fc-L2-PON1) form for each of the DNase1, SOD1, CTLA-4, CD40, anti-TNFα scFv, and DNase1 L3 “T” region fusion partners. The assembled PON1 fusion constructs included the following: DNase™-(g4s)6-Fc-P1-K, SOD1-Fc-P1-K, CTLA4-Fc-P1-K, CD40TM-Fc-P1-K, Adalimumab scFv-Fc-P1-K, and DNase1 L3-Fc-P1-K. In addition, a monospecific Fc-PON1 fusion construct was assembled (nucleotide and amino acid sequences as shown in SEQ ID NO:121 and SEQ ID NO:122; also referred to hereinbelow as Fc-PON1-K or Fc-P1-K). Also assembled were two ApoA1-Fc-PON1 fusions: THER4-P1-K (nucleotide and amino acid sequences shown in SEQ ID NO:45 and SEQ ID NO:46 of PCT Publication No. WO 2017,044424) and THER6-P1-K (identical to THER4-P1-K but with six repeats of (Gly₄Ser) in the linker sequence instead of four (Gly₄Ser) repeats). Fusion gene cassettes containing the desired sequences were then inserted into a multiple cloning site of the mammalian expression vector pDG, a pcDNA3 plasmid derivative containing a CMV promoter to drive expression of the fusion gene. Plasmid DNA was prepared using QIAGEN (Germantown, MD) mini or maxiprep plasmid DNA kits. Purified plasmid DNA was transfected into HEK293 cells plated at approximately 50-75% confluence, using Polyfect (QIAGEN, Germantown, MD) transfection reagent according to the manufacturer's instructions. Culture media was changed to DMEM Fluorobrite™ (Life Technologies, Carlsbad, CA) serum free media on the day after transfections, and transfected cells incubated for an additional 48 hours prior to harvest of culture supernatants. Culture supernatants were filtered through 0.2 μm PES syringe filters prior to analysis.

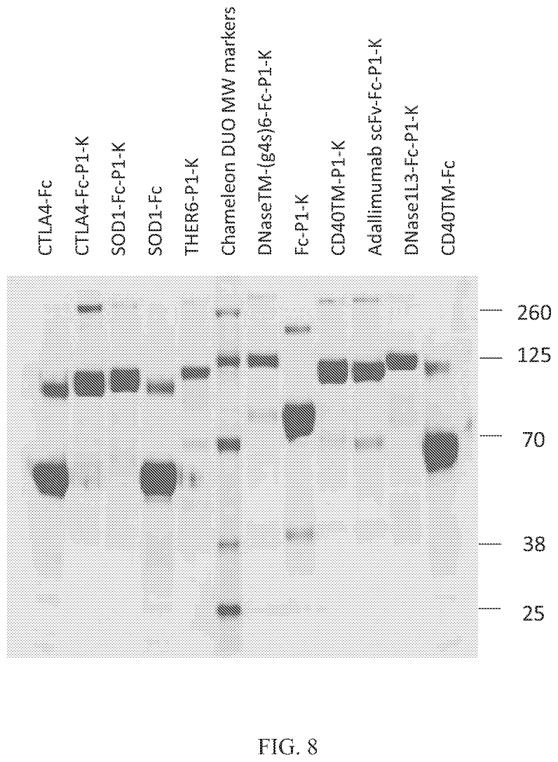

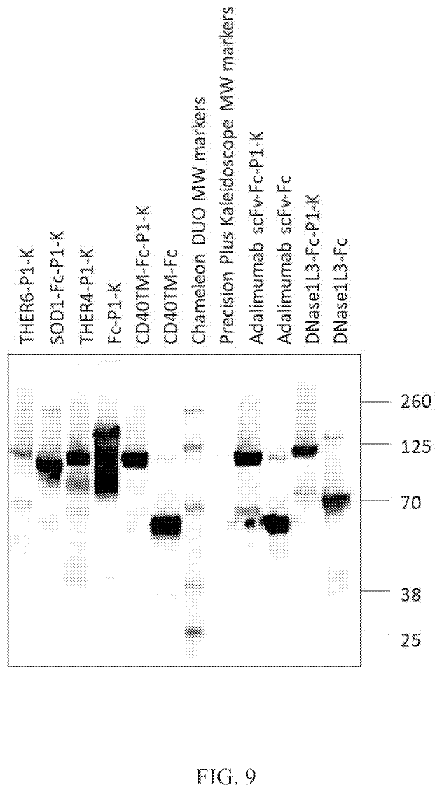

FIG. 8 and FIG. 9 show the results of Western blot analysis of fusion protein expression from a representative set of HEK293 transient transfections. Culture supernatants were immunoprecipitated with 60 μl protein A agarose (Repligen, Waltham MA) washed in gentle antigen-antibody binding buffer pH 8.0, (Pierce/Life Technologies, Carlsbad, CA) and 0.5 ml 293 transfection supernatants in Fluorobrite DMEM added to each microfuge tube. Immunoprecipitates were rotated overnight at 4° C., centrifuged at 3000 rpm, and washed in Binding Buffer pH 8.0, prior to addition of 60 μl 2X LDS sample buffer (Life Technologies, Carlsbad, CA), samples heated for 10 minutes at 72° C., the samples centrifuged for 1 minute at 3000 rpm in a microfuge, and 15 μl sample in loading buffer (one fourth) from each sample was loaded onto 4-12% Bis-Tris NuPAGE gels (Life Technologies, Carlsbad, CA). Protein molecular weight markers were included on each gel (Chameleon® DUO molecular weight markers; L1-COR Biosciences, Lincoln, NE and/or Precision Plus Kaleidoscope MW markers; BIORAD, Hercules, CA). SDS-PAGE gels were run in NuPAGE MOPS running buffer (Life Technologies, Carlsbad, CA) under reducing conditions at 185 volts for approximately 1-1.5 hours. Gels were blotted to nitrocellulose membranes using the XCell blot module (Life Technologies, Carlsbad, CA) and NUPAGE-MOPS (Life Technologies, Carlsbad, CA) transfer buffer containing 10% methanol. Blots were blocked in L1-COR Odyssey Intercept™ blocking buffer, followed by incubation with diluted goat anti-human IgG (Fc specific) secondary antibodies (Jackson Immunoresearch, West Grove, PA). Secondary antibody was conjugated with Alexafluor 790 near IR dye (L1-COR Odyssey detection) and diluted in blocking buffer 1:35,000 at 4° C. and rocked overnight. Blots were washed four times with Tris buffered saline (TBS) containing 0.1% Tween 20. Blots were then rinsed in TBS buffer and scanned with a LICOR Odyssey™ scanner. For the Western Blot shown in FIG. 8, transfection samples were loaded as follows: Lane 1-CTLA4-Fc, Lane 2-CTLA4-Fc-P1-K, Lane 3-SOD1-Fc-P1-K, Lane 4-SOD1-Fc, Lane 5—THER6-P1-K, Lane 6—LICOR Chameleon DUO MW Markers, Lane 7—DNase™-(g4s)6-Fc-P1-K, Lane 8-Fc-P1-K, Lane 9—CD40TM-Fc-P1-K; Lane 10—Adalimumab scFv-Fc-P1-K, Lane 11-DNase1 L3-Fc-P1-K, Lane 12—CD40TM-Fc. The approximate molecular weight (kDa) of each marker band is indicated on the right side of the Western blot. For the Western Blot shown in FIG. 9, transfection samples were loaded as follows: Lane 1—THER6-P1-K, Lane 2—SOD1-Fc-P1-K, Lane 3—THER4-P1-K, Lane 4—Fc-P1-K, Lane 5—CD40TM-Fc-P1-K; Lane 6—CD40TM-Fc, Lane 7-LICOR Chameleon DUO MW Markers, Lane 8—BIORAD Precision Plus Kaleidoscope MW markers, Lane 9—Adalimumab scFv-Fc-P1-K, Lane 10—Adalimumab scFv-Fe, Lane 11—DNase1L3-Fc-P1-K, Lane 12—DNase1 L3-Fc. The approximate molecular weight (kDa) of each marker band is indicated on the right side of the Western blot.

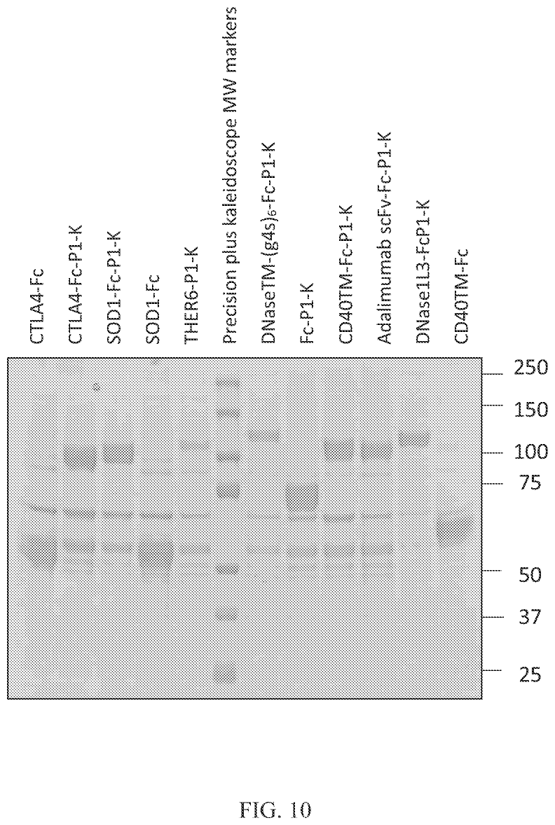

FIG. 10 shows the results of SDS-PAGE analysis of immunoprecipitated protein from HEK293 transfection supernatants. Culture supernatants were immunoprecipitated with 60 μl protein A agarose (Repligen, Waltham, MA), washed in gentle antigen-antibody binding buffer pH 8.0, (Pierce/Life Technologies, Carlsbad, CA) and 0.5 ml 293 transfection supernatants in Fluorobrite DMEM (ThermoFisher Scientific, Waltham, MA) added to each microfuge tube. Immunoprecipitates were rotated overnight at 4° C., centrifuged at 3000 rpm, and washed in Binding Buffer pH 8.0, prior to addition of 60 μl 2X LDS sample buffer (Life Technologies, Carlsbad, CA), samples heated for 10 minutes at 72° C., the samples centrifuged for 1 minute at 3000 rpm in a microfuge, and 15 μl sample in loading buffer (one fourth) from each sample was loaded onto 4-12% Bis-Tris NuPAGE gels (Life Technologies, Carlsbad, CA). SDS-PAGE gels were run in NuPAGE MOPS running buffer (Life Technologies, Carlsbad, CA) under reducing conditions at 185 volts for approximately 1-1.5 hours. After electrophoresis, gels were washed in distilled water several times, then incubated for 2 hours in Imperial Protein Stain (Pierce/ThermoFisher Scientific, Waltham MA). Gels were destained by washing several times in distilled water to remove nonspecific stain. Gels were then scanned using a Canon PIXMA MG8220 scanner to visualize the bands. Lanes were loaded as follows: Lane 1—CTLA4-Fc, Lane 2—CTLA4-Fc-P1-K, Lane 3—SOD1-Fc-P1-K, Lane 4—SOD1-Fc, Lane 5—THER6-P1-K, Lane 6—BIORAD Precision Plus Kaleidoscope molecular weight markers, Lane 7—DNase™-(g4s)6-Fc-P1-K, Lane 8—Fc-P1-K, Lane 9—CD40TM-Fc-P1-K; Lane 10—Adalimumab scFv-Fc-P1-K, Lane 11—DNase1 L3-Fc-P1-K, Lane 12—CD40TM-Fc. The approximate molecular weight (kDa) of each marker band is indicated on the right side of the gel image.

### Example 13: In Vitro Assessment of PON1 Fusion Proteins for Functional Activity

Functional activity of the PON1 fusion proteins was assessed with a series of in vitro assays. For several of the assays, protein activity was often analyzed directly from culture supernatants of transiently transfected HEK 293 cells. For other in vitro assays, fusion proteins were first purified from culture supernatants (either HEK 293 or CHO DG44 transfected cells) prior to assessment of functional activity. Fusion proteins were purified from culture supernatants by protein A affinity chromatography. Culture supernatants were filtered through 0.2 μm PES express filters (Nalgene, Rochester, NY) and subjected to affinity chromatography using slow rotation of culture supernatants with Protein A-agarose (IPA 300 crosslinked agarose) slurry in 50 ml sterile, conical centrifuge tubes at 4° C. (Repligen, Waltham, MA). Fusion protein bound to protein A agarose was recovered by centrifugation, and culture supernatants removed, replaced, and the incubation process repeated until the desired volume of supernatant was processed. The final protein A agarose slurry was then loaded into sterile, acid-washed econocolumns (BioRad, Hercules, CA) to wash the resin. Columns were then washed with several column volumes of column wash buffer (Gentle Ag-Ab binding buffer, Pierce/ThermoFisher, Waltham, MA) to remove any residual culture supernatant, prior to elution. Bound protein was then eluted from the resin using gentle Ag/Ab elution buffer (Pierce/ThermoFisher, Waltham, MA). Fractions (0.8-1.0 ml) were collected and protein concentration of aliquots (2 μl) from each fraction were determined at 280 nM using a Nanodrop (Wilmington DE) microsample spectrophotometer, with blank determination using elution buffer alone. Fractions containing fusion protein were pooled, and buffer exchange was performed by dialysis using Spectrum Laboratories G2 (Ranch Dominguez, CA, Catalog #G235057, Fisher Scientific catalog #08-607-007) float-a-lyzer units (MWCO 20 kDa) against [0.9% sodium chloride, 5 mM sodium bicarbonate, 1 mM HEPES buffer, 1 mM calcium chloride, pH 7.5]. Dialysis was performed in sterile, 2.2-liter Corning roller bottles at 4° C. overnight. After dialysis, protein was filtered using 0.2 μM filter units, and aliquots tested for endotoxin contamination using Pyrotell LAL gel clot system single test vials (STV) (Catalog #G2006, Associates of Cape Cod, East Falmouth, MA).

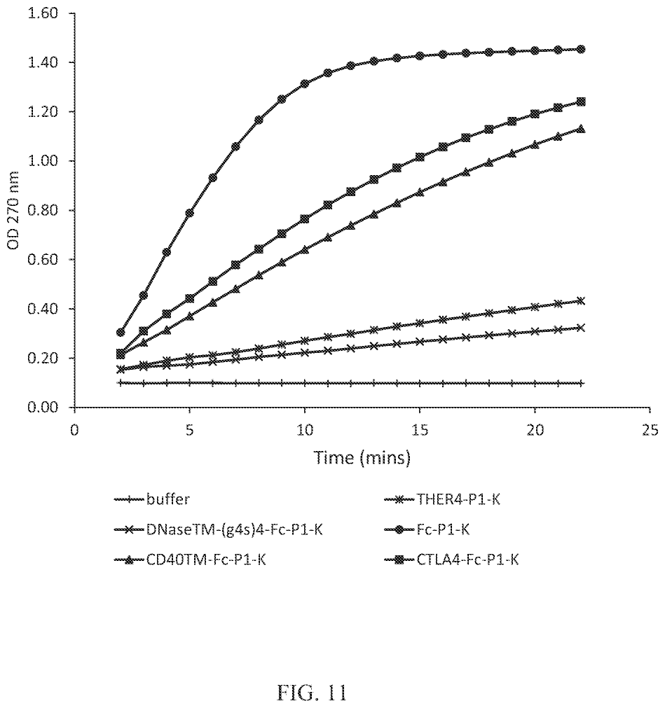

FIG. 11 shows the results from a representative assay of the PON1 arylesterase activity directly from HEK 293 culture supernatants. For this assay, culture supernatants were harvested in Fluorobrite DMEM (serum free) 72 hours after transfection, and sterile filtered with 0.2 μm syringe filters prior to use. Supernatants were diluted with reaction buffer for the assay to two times the final desired dilution. Diluted supernatants were aliquoted to individual wells of a UV transparent, 96 well flat bottom microtiter plate (ThermoFisher Scientific, Waltham, MA). Reactions were performed at 25° C. Just prior to measurement, appropriate dilutions of the arylesterase substrate phenyl acetate were added to each well with a multichannel pipettor. For the data shown in the graph, culture supernatants were diluted 1:8 and 50 μl added to each well. The substrate was then added in 50 μl/well to begin the assay. For most assays, 6-10 mM phenyl acetate was used, so that the final concentration of substrate was 3-5 mM. To monitor conversion of phenyl acetate to phenol, the absorbance change at 270 nm was monitored in a kinetic assay, with readings taken every seconds for 22.5 minutes (30 measurements/well). The absorbance at each time point was then plotted as a function of time.

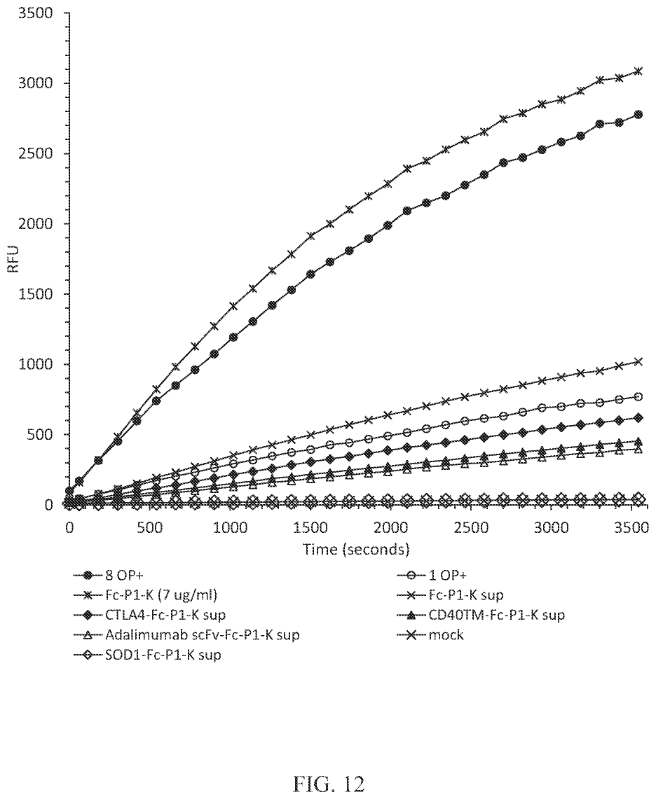

FIG. 12 shows a similar assessment measuring organophosphatase activity of the PON1 moeity in bispecific and monospecific PON1 fusion proteins. In these assays, the substrate used was a proprietary organophosphatase substrate contained in the EnzCHEK paraoxonase assay kit (Molecular Probes/ThermoFisher Scientific, Waltham, MA). This assay is a very sensitive fluorometric assay for the organophosphatase activity of paraoxonase that uses excitation/emission maxima of 360/450 nm to measure the conversion of a fluorogenic organophosphate analog provided with the kit. The assay can either be set up as a kinetic assay or terminated after a particular period of time for an endpoint assay. The change in relative fluorescence units (RFU) per unit time is converted to the units of paraoxonase in the sample using the standard curve generated from the fluorescent standard and the conversion factor that 1 U unit of paraoxonase generates 1 nmol of fluorescent product per minute at 37° C. The amount of paraoxonase present in the fusion protein samples can be compared to the paraoxonase positive control provided with the kit. FIG. 12 shows a graphical representation of the organophosphatase activity present in serial dilutions of culture supernatant from several of the SOD1-Fc-P1-K, CTLA4-Fc-P1-K, and CD40TM-Fc-P1-K fusion proteins expressed in HEK 293 transient transfections and from a purified batch of the Fc-PON1-K (Fc-P1-K) fusion protein. The organophosphatase activity is compared to the RFU curves generated by two different dilutions of the organophosphatase positive control in the average to above average activity range for human serum sample comparisons. Fc-PON1-K purified fusion protein showed high activity in this assay, and calculations relative to the fluorescent standard curve indicate that the PON1 organophosphatase activity is 700 mU/μg, or 40U/umole, Fc-P1-K. Culture supernatants from HEK293 transfections were also included in the assay. Although they are not shown on the graph, the supernatant from the Adalimumab scFv-Fc-P1-K transfections mirrored the activity profile observed for the CD40TM-Fc-P1-K transfection. Similarly, the supernatant from the DNase1L3-Fc-PJ-K transfection mirrored the negligible activity level observed for the SOD1-Fc-P1-K transfection shown on the graph.

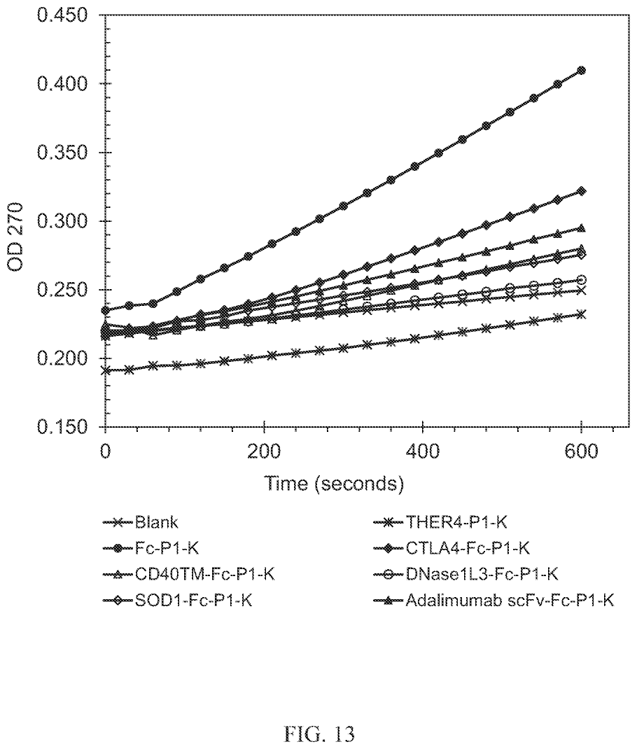

FIG. 13 shows results of an assay measuring lactonase activity of the PON1 moeity in bispecific and monospecific PON1 fusion proteins. In these assays, the substrate used was dihydrocoumarin (DHC) (Millipore-Sigma, St. Louis, MO) at a final concentration of 1 mM in 1X reaction buffer. Reaction buffer for the DHC assays was 50 mM Tris HCl (pH 7.4), 1 mM CaCl₂). HEK293 culture supernatants were diluted 1:4 in reaction buffer and 25 μl added to individual wells of a 96 well UV STAR (Greiner BioOne, Thomas Scientific) microtiter plate. Substrate dihydrocoumarin (DHC) solution in reaction buffer was added to each well (75 μl) to generate a final volume of 100 μl. Hydrolysis of the substrate was monitored at OD 270 for 15 minutes. Hydrolysis of DHC by the fusion proteins was monitored as the change in OD270 as a function of time.

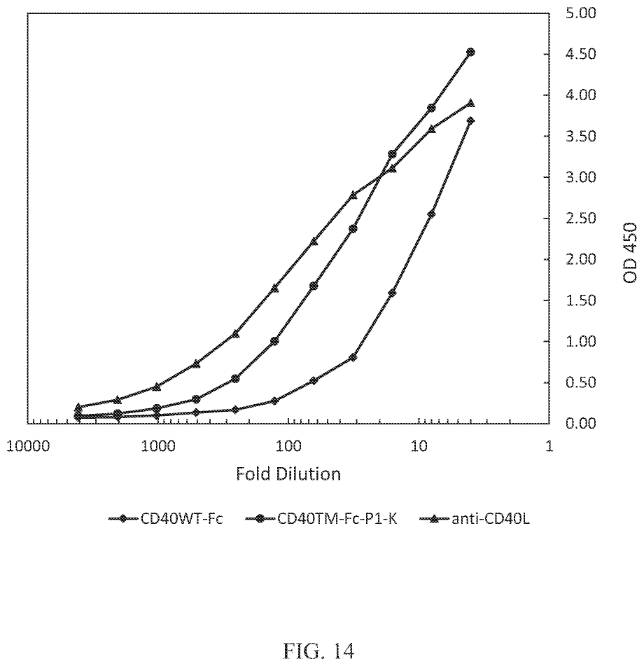

In addition to the paraoxonase enzyme activity, the functional properties of the different “T” domains positioned at the amino terminus of the T-L1-X-L2-P fusion proteins were also assessed. Depending on the identity of the “T” domain, binding to a target antigen or enzyme activity was monitored. FIG. 14 shows the results of an antigen binding assay to CD40 ligand for the human CD40 receptor extracellular domain triple mutant in the CD40TM-Fc-P1-K fusion proteins. The antigen binding ELISA assay was performed as follows: Human CD40 ligand was obtained from BioLegend (San Diego, CA). The human CD40 ligand was diluted in 0.1 M carbonate buffer (pH 9.0) to a concentration of 2 μg/ml and 100 μl/well aliquoted to each well to be used in the assay in a 96 well NUNC immulon II MaxiSorp plate (ThermoFisher Scientific, Waltham, MA). Plates were incubated overnight at 4° C., prior to blocking in 200 μl/well PBS/2.0% BSA, overnight at 4° C. Plates were washed in wash buffer (PBS, 0.5% Tween-20, 0.005% Kathon), and serial dilutions of the HEK 293 transfection supernatants added to wells of the plate. As a positive control for antigen binding, a mouse antibody targeted to the CD40 ligand was also serially diluted on the plate Plates were incubated with supernatant or antibody dilutions at 4° C., overnight. Plates were washed four times in wash buffer, then incubated with horseradish peroxidase conjugated goat anti-human IgG Fc specific, or goat anti-mouse IgG1 at a dilution of 1:10,000 (Jackson Immunoresearch, West Grove, PA). FIG. 14 shows the CD40 ligand binding curves for the CD40WT-Fc, the CD40TM-Fc-PJ-K transfection supernatants, and the CD40L specific antibody dilutions by plotting the absorbance at 450 nm as a function of dilution. The CD40 mutant form shows improved binding activity to CD40 ligand when compared to the wild type CD40Fc containing supernatants.

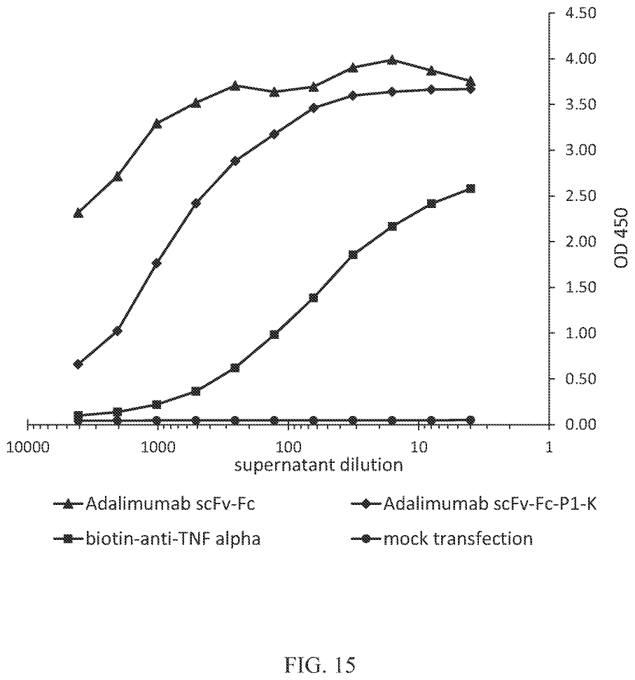

FIG. 15 shows binding data for the Humira™ (adalimumab) scFv containing fusion protein supernatants to TNF-alpha (TNF-α) in an antigen binding ELISA with immobilized TNF-α. The antigen binding ELISA assay was performed as follows: Human TNF-α was obtained from BioLegend (San Diego, CA). The TNF-α protein was diluted in 0.1 M carbonate buffer (pH 9.0) to a concentration of 1 μg/ml and 100 μl/well aliquoted to each well to be used in the assay in a 96 well NUNC immulon II MaxiSorp plate (ThermoFisher Scientific, Waltham, MA). Plates were incubated overnight at 4° C., prior to blocking in 200 μl/well blocking buffer (PBS/2.0% BSA), overnight at 4° C. Plates were washed in wash buffer (PBS, 0.5% Tween-20, 0.005% Kathon), and serial dilutions of either the HEK293 transfection supernatants or mouse anti-human TNF antibody (BioLegend, San Diego, CA) added to wells of the plate. Plates were incubated with supernatant or antibody dilutions at 4° C., overnight. Plates were washed four times in wash buffer, then incubated with horseradish peroxidase conjugated goat anti-human IgG Fc specific, or goat anti-mouse IgG1 at a dilution of 1:10,000 (Jackson Immunoresearch, West Grove, PA). FIG. 15 shows the TNF-α binding curves for the adalimumab scFv-Fc transfection supernatant, the adalimumab scFv-Fc-P1-K transfection supernatant, or the TNF-α specific antibody, in which the absorbance at 450 nm was plotted as a function of dilution. The adalimumab scFv-containing fusion proteins expressed in HEK293 transient transfections bind to human TNF-α immobilized on the plate(s).

### Example 15: Treatment of Lung Fibrosis by Administration of PON1 Fusion Proteins

Effects of different PON1 fusion proteins on lung fibrosis are assessed using in vivo mouse models of disease. One such model involves in vivo assessment of the efficacy of the purified fusion proteins for treatment of pulmonary fibrosis in a bleomycin induced lung fibrosis mouse model. Bleomycin (BLM)-induced pulmonary fibrosis is the most well-established disease model for IPF and is widely used to investigate the efficacy and mechanism of therapeutic candidates. In the model, alveolar injury and interstitial inflammation/fibrosis are induced by intratracheal BLM administration. The aim of this study is to examine the effect of PON1 fusion test compounds on lung inflammation and fibrosis in BLM-induced pulmonary fibrosis model and compare responses to a control preparation of nintedanib. At day 0, mice are induced to develop pulmonary fibrosis by a single intratracheal administration of bleomycin hydrochloride in saline at a dose of 3.0 mg/kg, in a volume of 50 Id per animal using Microsprayer. Mice are divided into groups of 12 mice based on the body weight changes on the day before the start of treatments with the test compounds on day 7. Mice are intranasally/intravenously/intraperitoneally administered test compounds (depending on the test group). Compounds are administered daily from Day 7 to 20, animals sacrificed on Day 21 and samples analyzed for fibrotic markers.

At study termination, whole blood samples without anticoagulant are obtained via abdominal vena cava under a mixture of medetomidine, midazolam and butorphanol anesthesia. The supernatants from separated blood are collected and stored at −80° C. until further processing. Lungs are harvested from the mice and two lobes left unfixed, one lobe is frozen and another harvested for biochemical assays such as measurements of hydroxyproline or inflammatory markers. Remaining lung lobes are fixed in 10% neutral buffered formalin, paraffin emedded for Masson's trichrome staining and histology. For quantitative analysis of lung fibrosis area, bright field images of Masson's Trichrome-stained sections are randomly captured using a digital camera at 100-fold magnification, and the subpleural regions in 20 fields/mouse are evaluated according to the criteria for grading lung fibrosis (Ashcroft, T., et al., J Clin Pathol, 1988; 41:467-70), to generate an Ashcroft score. All sections are blindly analyzed by an experimenter. Statistics are performed on each treatment group, calculating an average Aschroft score, and comparing the scores in the different treatment groups to the Vehicle group.

Alternative methods of fusion protein delivery may be utilized. As an example, rather than intranasal spray delivery which is confined to the upper airways, nebulization of the fusion proteins may improve delivery of the fusion molecules deeper into the lungs, thereby more effectively targeting all areas of the lung. Fusion proteins can be nebulized with or without excipients and tested for protein recovery, aggregation, and enzyme activity. A vibrating mesh (VM) nebulizer (PAR1 eFlow. Omron NE-U100, Lake Forest, IL) can be used since other proteins have been successfully nebulized using this apparatus and because VM nebulizers are small and portable. Nebulized proteins (500 μl) at 1 mg/ml in saline plus 1 mM CaCl₂ (Pulmozyme@ buffer) are collected on a sterile 60 mm low protein binding tissue culture dish. Recovered fusion proteins are analyzed by SDS gels, size exclusion HPLC (SEC), and for protein concentration and enzyme activity before and after nebulization to evaluate the effects of the excipients and the effects of nebulization on the protein.

From the foregoing, it will be appreciated that, although specific embodiments of the invention have been described herein for purposes of illustration, various modifications may be made without deviating from the spirit and scope of the invention. Accordingly, the invention is not limited except as by the appended claims. All publications, patents, and patent applications cited herein are hereby incorporated by reference in their entireties for all purposes.

1. A fusion polypeptide comprising, from an amino terminal position to a carboxyl terminal position, T-L1-X-L2-P, wherein:
T is a first biologically active polypeptide selected from the group consisting of,
a cytotoxic T-lymphocyte associated molecule-4 (CTLA-4) extracellular domain, and
a CD40 extracellular domain;
L1 is a first polypeptide linker, wherein L1 is optionally present;
X is an immunoglobulin heavy chain constant region, wherein the immunoglobulin heavy chain constant region is capable of forming dimers and specifically binding the neonatal Fc receptor (FcRn);
L2 is a second polypeptide linker comprising at least eight amino acid residues; and
P is a biologically active paraoxonase, wherein the paraoxonase has at least 95% identity with the amino acid sequence shown in residues (i) 116-355 or 26-355 of SEQ ID NO:6, (ii) residues 16-355 or 26-355 of SEO ID NO:124, or (iii) residues 16-355 or 26-355 of SEO ID NO:126, and wherein the paraoxonase does not contain an amino terminal leader sequence corresponding to residues 1-15 of SEQ ID NO:6.

2-72. (canceled)

73. A fusion polypeptide comprising, from an amino terminal position to a carboxyl terminal position, X-L2-P, wherein:
X is an immunoglobulin heavy chain constant region, wherein the immunoglobulin heavy chain constant region is capable of forming dimers and specifically binding the neonatal Fc receptor (FcRn);
L2 is a polypeptide linker comprising at least eight amino acid residues; and
P is a biologically active paraoxonase, wherein the paraoxonase has at least 95% identity with the amino acid sequence shown in (i) residues 16-355 or 26-355 of SEQ ID NO:6, (ii) residues 16-355 or 26-355 of SEO ID NO:124, or (iii) residues 16-355 or 26-355 of SEO ID NO:126, and wherein the paraoxonase does not contain an amino terminal leader sequence corresponding to residues 1-15 of SEQ ID NO:6,
wherein the fusion polypeptide does not comprise a biologically active polypeptide N-terminal to the immunoglobulin heavy chain constant region.

74-81. (canceled)

82. The fusion polypeptide of claim 73, wherein the immunoglobulin heavy chain constant region is an immunoglobulin Fc region.

83. The fusion polypeptide of claim 82, wherein the Fc region is a human Fc region optionally wherein the human Fc region is an Fc variant comprising one or more amino acid substitutions relative to the wild-type human sequence.

84. (canceled)

85. The fusion polypeptide of claim 83, wherein the Fc region is a human γ1 Fc region or a human γ4 Fc region.

86. The fusion polypeptide of claim 83, wherein the Fc region is a human γ1 Fc variant in which
Eu residue C220 is replaced by serine,
Eu residue P238 is replaced by serine, and/or
Eu residue P331 is replaced by serine.

87-90. (canceled)

91. The fusion polypeptide of claim 82, wherein the Fc region has the amino acid sequence shown in
(i) residues 1-232 or 1-231 of SEQ ID NO:26,
(ii) residues 1-232 or 1-231 of SEQ ID NO:116, or
(iii) residues 1-232 or 1-231 of SEQ ID NO:118.

92-99. (canceled)

100. The fusion polypeptide of claim 1, wherein the fusion polypeptide comprises an amino acid sequence having at least 95% identity with the amino acid sequence shown in
(i) residues 21-736 of SEQ ID NO:66,
(ii) residues 21-804 of SEO ID NO:74,
(iii) residues 21-804 of SEO ID NO:78,
(iv) residues 21-804 of SEO ID NO:82,
(v) residues 21-804 of SEO ID NO:86, or
(vi) residues 21-804 of SEO ID NO:90.

101. The fusion polypeptide of claim 100, wherein the fusion polypeptide comprises the amino acid sequence shown in
(i) residues 21-736 of SEQ ID NO:66,
(ii) residues 21-804 of SEO ID NO:74,
(iii) residues 21-804 of SEO ID NO:78,
(iv) residues 21-804 of SEO ID NO:82,
(v) residues 21-804 of SEO ID NO:86, or
(vi) residues 21-804 of SEO ID NO:90.

102-107. (canceled)

108. The fusion polypeptide of claim 73, wherein the fusion polypeptide comprises an amino acid sequence having at least 95% identity with the amino acid sequence shown in
(i) residues 21-613 of SEQ ID NO:122,
(ii) residues 21-613 of SEQ ID NO:132,
(iii) residues 21-613 of SEQ ID NO:134,
(iv) residues 21-610 of SEQ ID NO:142,
(v) residues 21-610 of SEQ ID NO:144, or
(vi) residues 21-610 of SEQ ID NO:146.

109. The fusion polypeptide of claim 108, wherein the fusion polypeptide comprises the amino acid sequence shown in
(i) residues 21-613 of SEQ ID NO:122,
(ii) residues 21-613 of SEQ ID NO:132,
(iii) residues 21-613 of SEQ ID NO:134,
(iv) residues 21-610 of SEQ ID NO:142,
(v) residues 21-610 of SEQ ID NO:144, or
(vi) residues 21-610 of SEQ ID NO:146.

110-141. (canceled)

142. A dimeric protein comprising a first fusion polypeptide and a second fusion polypeptide, wherein each of the first and second fusion polypeptides is a fusion polypeptide as defined in claim 1.

143. A polynucleotide encoding the fusion polypeptide of claim 1.

144. An expression cassette comprising a DNA segment encoding the fusion polypeptide of claim 1, wherein the DNA segment is operably linked to a promoter,
optionally wherein the expression cassette is contained within a vector.

145. A cultured cell into which has been introduced the expression cassette of claim 144, wherein the cell expresses the DNA segment.

146. A method of making a fusion polypeptide, the method comprising:
culturing a cell into which has been introduced the expression cassette of claim 144, wherein the cell expresses the DNA segment and the encoded fusion polypeptide is produced; and
recovering the fusion polypeptide.

147. (canceled)

148. The method of claim 146, wherein the encoded fusion polypeptide is produced and recovered as a dimeric protein.

149-156. (canceled)

157. A composition comprising:
a dimeric protein of claim 142; and
a pharmaceutically acceptable carrier.

158-228. (canceled)

229. A dimeric protein comprising a first fusion polypeptide and a second fusion polypeptide, wherein each of the first and second fusion polypeptides is a fusion polypeptide as defined in claim 73.

230. A polynucleotide encoding the fusion polypeptide of claim 73.

231. An expression cassette comprising a DNA segment encoding the fusion polypeptide of claim 73, wherein the DNA segment is operably linked to a promoter,
optionally wherein the expression cassette is contained within a vector.

232. A cultured cell into which has been introduced the expression cassette of claim 231, wherein the cell expresses the DNA segment.

233. A method of making a fusion polypeptide, the method comprising:
culturing a cell into which has been introduced the expression cassette of claim 231, wherein the cell expresses the DNA segment and the encoded fusion polypeptide is produced; and
recovering the fusion polypeptide.

234. The method of claim 233, wherein the encoded fusion polypeptide is produced and recovered as a dimeric protein.

235. A composition comprising:
a dimeric protein of claim 229; and
a pharmaceutically acceptable carrier.

236. A method for treating disease selected from the group consisting of an inflammatory disease, an autoimmune disease, a neurological disease, a cardiovascular disease, a chronic liver disease, and a fibrotic disease, or for treating exposure to sulfur mustard gas or an organophosphate, the method comprising:
administering to a subject having the disease, or to a subject exposed to the sulfur mustard gas or to the organophosphate, an effective amount of a dimeric protein of claim 142.

237. A method for treating disease selected from the group consisting of an inflammatory disease, an autoimmune disease, a neurological disease, a cardiovascular disease, a chronic liver disease, and a fibrotic disease, or for treating exposure to sulfur mustard gas or an organophosphate, the method comprising:
administering to a subject having the disease, or to a subject exposed to the sulfur mustard gas or to the organophosphate, an effective amount of a dimeric protein of claim 229.
